(19) United States
(12) Patent Application Publication
(10) Pub. No.: US 2012/0202856 A1
(43) Pub. Date: Aug. 9, 2012

(54) SUBSTITUTED BENZIMDAZOLE DERIVATIVES USEFUL AS TRPM8 RECEPTOR MODULATORS

(76) Inventors: Mark R. PLAYER, Phoenixville, PA (US); Raul CALVO, Royersford, PA (US); Jinsheng CHEN, Exton, PA (US); Carl ILLIG, Phoenixville, PA (US); Sanath MEEGALLA, Gamet Valley, PA (US); Daniel PARKS, Downingtown, PA (US); William PARSONS, Belle Mead, NJ (US)

(21) Appl. No.: 13/364,352

(22) Filed: Feb. 2, 2012

Related U.S. Application Data
(60) Provisional application No. 61/439,969, filed on Feb. 7, 2011.

(51) Int. Cl. A61K 31/4184; C07D 413/04; A61K 31/422; C07D 405/04; A61P 9/12; A61K 31/427; A61K 31/4196; A61K 31/4245; A61P 25/00; A61P 9/00; C07D 403/04; C07D 417/04

(52) U.S. Cl. 514/364; 548/306.1; 514/394; 548/247; 514/378; 548/305.1; 548/304.7; 548/301.1; 548/181; 514/365; 548/266.4; 514/383; 548/143; 548/235; 514/374

(57) ABSTRACT

The present invention is directed to benzimidazole derivatives, pharmaceutical compositions containing them and their use in the treatment of disorders and conditions modulated by TRP M8, including for example, inflammatory pain, inflammatory hyperalgesia, inflammatory hypersensitivity condition, neuropathic pain, neuropathic cold allodynia, inflammatory somatic hyperalgesia, inflammatory visceral hyperalgesia, cardiovascular disease aggravated by cold and pulmonary disease aggravated by cold.

### CROSS-REFERENCE TO RELATED APPLICATIONS

The present application claims the benefits of the filing of U.S. Provisional Application No. 61/439,969 filed Feb. 7, 2011. The complete disclosures of the aforementioned related patent applications are hereby incorporated herein by reference for all purposes.

### FIELD OF THE INVENTION

The present invention is directed to substituted benzimidazole derivatives, pharmaceutical compositions containing them and their use in the treatment of disorders and conditions modulated by the TRPM8 (transient receptor potential, melastatin subfamily, type 8) channel. More particularly, the compounds of the present invention are useful in the treatment of inflammatory pain, inflammatory hyperalgesia, inflammatory hypersensitivity condition, neuropathic pain, neuropathic cold allodynia, inflammatory somatic hyperalgesia, inflammatory visceral hyperalgesia, cardiovascular disease aggravated by cold and pulmonary disease aggravated by cold.

### BACKGROUND OF THE INVENTION

Transient receptor potential (TRP) channels are non-selective cation channels that are activated by a variety of stimuli. Numerous members of the ion channel family have been identified to date, including the cold-menthol receptor, also called TRPM8 (MCKEMY, D. D., et al “Identification of a cold receptor reveals a general role for TRP channels in thermosensation”, Nature, 2002, pp 52-58, vol. 416 (6876)). Collectively, the thermosensitive TRP channels and related TRP-like receptors, such as TRPV1/2/3 and TRPM8, connote sensory responsivity to the entire continuum of thermal exposure, selectively responding to threshold temperatures ranging from noxious hot through noxious cold as well as to certain chemicals that mimic these sensations. Specifically, TRPM8 is known to be stimulated by cool to cold temperatures as well as by chemical agents, such as menthol and icilin, which may be responsible for the therapeutic cooling sensation that these agents provoke.

TRPM8 is located on primary nociceptive neurons (Aδ- and C-fibers) and is also modulated by inflammation-mediated second messenger signals (ABE, J., et al. “Ca2+-dependent PKC activation mediates menthol-induced desensitization of transient receptor potential M8”, Neurosci. Lett., 2006, pp 140-144, Vol. 397(1-2); PREMKUMAR, L. S., et al. “Downregulation of Transient Receptor Potential Melastatin 8 by Protein Kinase C-Mediated Dephosphorylation”, J. Neurosci., 2005, pp 11322-11329, Vol. 25(49)). The localization of TRPM8 on both A Aδ- and C-fibers may provide a basis for abnormal cold sensitivity in pathologic conditions wherein these neurons are altered, resulting in pain, often of a burning nature (KOBAYASHI, K., et al. “Distinct expression of TRPM8, TRPA1 and TRPV1 mRNAs in rat primary afferent neurons with a c-fibers and colocalization with trk receptors” J. Comp. Neurol., 2005, pp 596-606, Vol. 493(4), 596-606; ROZA, C. et al., “Cold sensitivity in axotomized fibers of experimental neuromas in mice”, Pain, 2006, pp 24-36, Vol 120(1-2); and XING, H., et al., “Chemical and Cold Sensitivity of Two Distinct populations of TRPM8-Expressing Somatosensory Neurons”, J. Neurophysiol., 2006, pp 1221-1230, Vol. 95(2)). Cold intolerance and paradoxical burning sensations induced by chemical or thermal cooling closely parallel symptoms seen in a wide range of clinical disorders and thus provide a strong rationale for the development of TRPM8 modulators as novel antihyperalgesic or antiallodynic agents. TRPM8 is also known to be expressed in the brain, lung, bladder, gastrointestinal tract, blood vessels, prostate and immune cells, thereby providing the possibility for therapeutic modulation in a wide range of maladies.

There remains a need in the art for TRPM8 antagonists that can be used to treat a disease or condition in a mammal in which the disease or condition is affected by the modulation of TRPM8 receptors, such as chronic or acute pain, or the diseases that lead to such pain, as well as pulmonary or vascular dysfunction.

### SUMMARY OF THE INVENTION

The present invention is directed to compounds of formula (I)

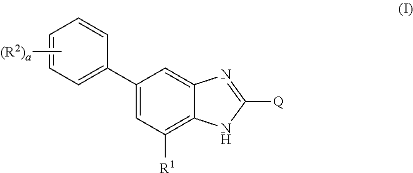

wherein

R¹ is selected from the group consisting of hydrogen, chloro, methyl and trifluoromethyl;

a is an integer from 0 to 2;

each R² is independently selected from the group consisting of fluoro, chloro, C₁₋₄alkyl, C₁₋₄alkoxy, fluorinated C₁₋₄alkyl, fluorinated C₁₋₄alkoxy and cyano;

Q is a substituted ring structure selected from the group consisting of (a) through (l):

(a)

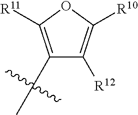

(substituted fur-3-yl);

(b)

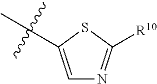

(substituted thiazol-5-yl);

(c)

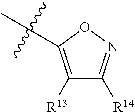

(substituted isoxazol-5-yl);

(d)

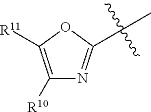

(substituted oxazol-2-yl);

(e)

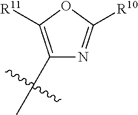

(substituted oxazol-4-yl);

(f)

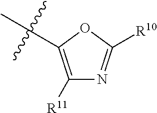

(substituted oxazol-5-yl);

(g)

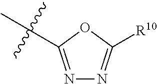

(substituted 1,3,4-oxadiazol-2-yl);

(h)

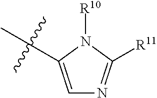

(substituted imidazol-5-yl);

(i)

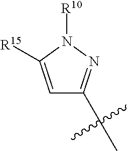

(substituted pyrazol-3-yl);

(j)

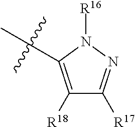

(substituted pyrazol-5-yl);

(k)

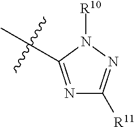

(substituted 1,2,4-triazol-5-yl); and

(l) a fused multi-ring structure selected from the group consisting of

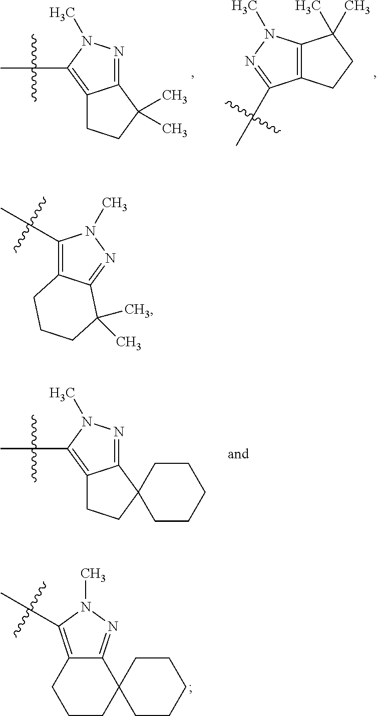

wherein

R¹⁰ and R¹¹ are each independently selected from the group consisting C₁₋₄alkyl;

R¹² is selected from the group consisting of hydrogen and cyano;

R¹³ is selected from the group consisting of hydrogen and C₁₋₄alkyl;

R¹⁴ is selected from the group consisting of chloro, bromo, C₁₋₆alkyl and C₃₋₆cycloalkyl;

R¹⁵ is selected from the group consisting of C₃₋₆cycloalkyl;

R¹⁶ is selected from the group consisting of C₁₋₄alkyl, hydroxy substituted C₁₋₄alkyl and benzyl;

R¹⁷ is selected from the group consisting of C₁₋₄alkyl, trifluoromethyl, C₃₋₆cycloalkyl and 1-methyl-cyclopropyl;

R¹⁸ is selected from the group consisting of hydrogen, chloro, C₁₋₄alkyl, trifluoromethyl and cyano;

and solvates, hydrates, tautomers and pharmaceutically acceptable salts thereof.

The present invention is further directed to a process for the preparation of the compounds of formula (I). The present invention is further directed to a product prepared according to the process described herein.

Illustrative of the invention is a pharmaceutical composition comprising a pharmaceutically acceptable carrier and the product prepared according to the process described herein. An illustration of the invention is a pharmaceutical composition made by mixing the product prepared according to the process described herein and a pharmaceutically acceptable carrier. Illustrating the invention is a process for making a pharmaceutical composition comprising mixing the product prepared according to the process described herein and a pharmaceutically acceptable carrier.

Exemplifying the invention are methods of treating a disorder modulated by TRPM8 (selected from the group consisting of inflammatory pain, including visceral pain, neuropathic pain, including neuropathic cold allodynia, cardiovascular disease aggravated by cold and pulmonary disease aggravated by cold, in a subject in need thereof, comprising administering to the subject a therapeutically effective amount of any of the compounds or pharmaceutical compositions described above.

The present invention is further directed to compounds of formula (I), as herein described) for use as a medicament, preferably for the treatment of a disorder selected form the group consisting of inflammatory pain, neuropathic pain, cardiovascular disease aggravated by cold, and pulmonary disease aggravated by cold.

Another example of the invention is the use of any of the compounds described herein in the preparation of a medicament wherein the medicament is prepared for treating: (a) inflammatory pain, (b) neuropathic pain, (c) cardiovascular disease aggravated by cold, or (d) pulmonary disease aggravated by cold, in a subject in need thereof.

### DETAILED DESCRIPTION OF THE INVENTION

The present invention is directed to compounds of formula (I)

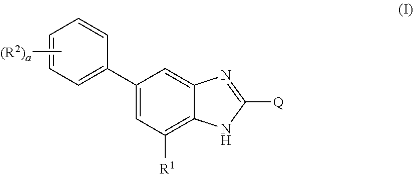

wherein R¹, a, R² and Q are as herein defined, and solvates, hydrates, tautomers and pharmaceutically acceptable salts thereof. The compounds of the present invention are useful in the treatment of disorders mediated by TRPM8, including inflammatory pain (including visceral pain), inflammatory hyperalgesia, neuropathic pain (including neuropathic cold allodynia), inflammatory somatic hyperalgesia, inflammatory visceral hyperalgesia, cardiovascular disease aggravated by cold and pulmonary disease aggravated by cold.

In an embodiment, the present invention is directed to compounds of formula (I) wherein R¹ is selected from the group consisting of hydrogen, chloro, methyl and trifluoromethyl. In another embodiment, the present invention is directed to compounds of formula (I) wherein R¹ is selected from the group consisting of hydrogen, chloro and trifluoromethyl. In another embodiment, the present invention is directed to compounds of formula (I) wherein R¹ is selected from the group consisting of hydrogen and chloro.

In an embodiment, the present invention is directed to compounds of formula (I) wherein a is an integer from 0 to 2. In another embodiment, the present invention is directed to compounds of formula (I) wherein a is an integer from 1 to 2.

In an embodiment, the present invention is directed to compounds of formula (I) wherein a is 1 and the R² group is bound at the 2-position on the phenyl ring. In another embodiment, the present invention is directed to compounds of formula (I) wherein a is 1 and the R² group is bound at the 3-position on the phenyl ring. In another embodiment, the present invention is directed to compounds of formula (I) wherein a is 2 and the R² groups bound at the 2- and 5-positions on the phenyl ring. In another embodiment, the present invention is directed to compounds of formula (I) wherein a is 2 and the R² groups bound at the 2- and 6-positions on the phenyl ring.

In an embodiment, the present invention is directed to compounds of formula (I) wherein each R² is independently selected from the group consisting of fluoro, chloro, C₁₋₄alkyl, C₁₋₄alkoxy, fluorinated C₁₋₄alkyl, fluorinated C₁₋₄alkoxy and cyano. In another embodiment, the present invention is directed to compounds of formula (I) wherein each R² is independently selected from the group consisting of fluoro, chloro, C₁₋₂alkyl, C₁₋₂alkoxy, fluorinated C₁₋₂alkyl, fluorinated C₁₋₂alkoxy and cyano.

In another embodiment, the present invention is directed to compounds of formula (I) wherein each R² is independently selected from the group consisting of fluoro, chloro, methyl, ethyl, methoxy, ethoxy, trifluoromethyl, trifluoromethoxy, difluoromethoxy and cyano. In another embodiment, the present invention is directed to compounds of formula (I) wherein each R² is independently selected from the group consisting of fluoro, chloro, methoxy, trifluoromethyl and trifluoromethoxy. In another embodiment, the present invention is directed to compounds of formula (I) wherein each R² is independently selected from the group consisting of fluoro, chloro, trifluoromethyl and trifluoromethoxy. In another embodiment, the present invention is directed to compounds of formula (I) wherein each R² is independently selected from the group consisting of fluoro, chloro and trifluoromethyl.

In an embodiment, the present invention is directed to compounds of formula (I) wherein Q is a substituted ring structure selected from the group consisting of (a) through (l):

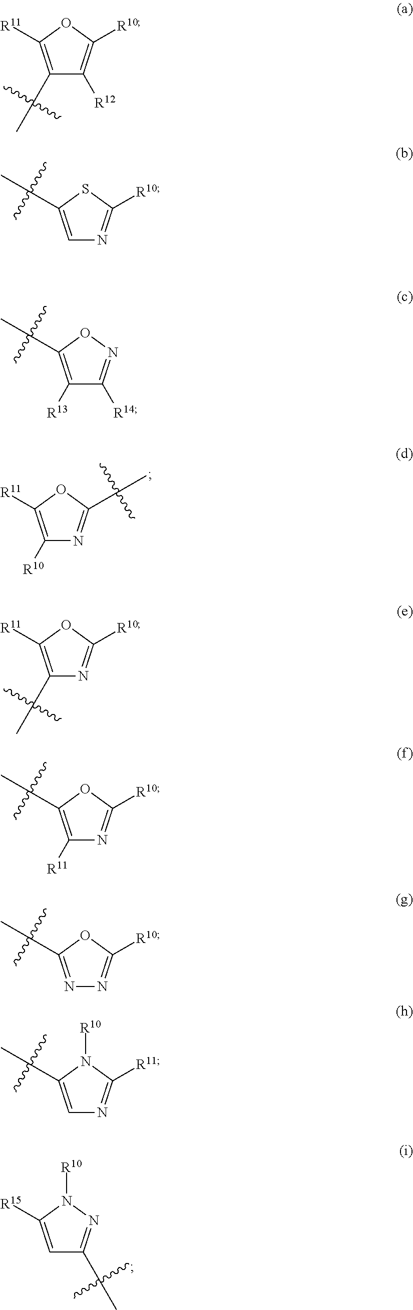

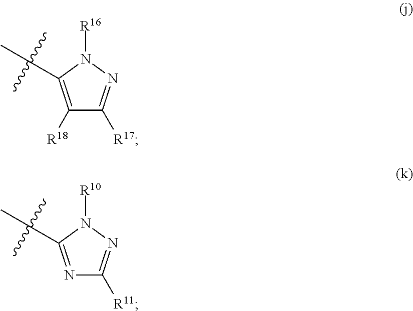

and (l) a fused multi-ring structure selected from the group consisting of

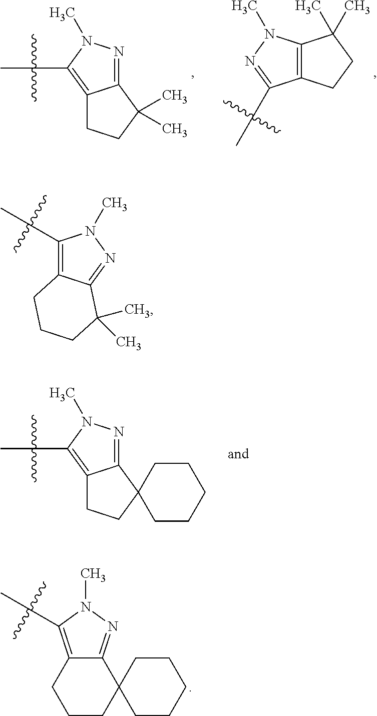

In an embodiment, the present invention is directed to compounds of formula (I) wherein Q is (a)

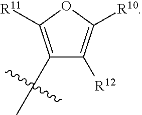

In another embodiment, the present invention is directed to compounds of formula (I) wherein Q is (b)

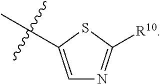

In another embodiment, the present invention is directed to compounds of formula (I) wherein Q is (c)

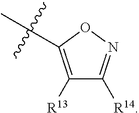

In another embodiment, the present invention is directed to compounds of formula (I) wherein Q is (d)

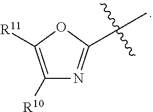

In another embodiment, the present invention is directed to compounds of formula (I) wherein Q is (e)

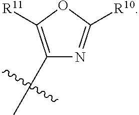

In another embodiment, the present invention is directed to compounds of formula (I) wherein Q is (f)

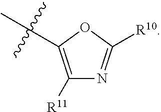

In another embodiment, the present invention is directed to compounds of formula (I) wherein Q is (g)

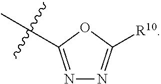

In another embodiment, the present invention is directed to compounds of formula (I) wherein Q is (h)

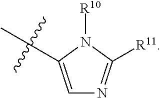

In another embodiment, the present invention is directed to compounds of formula (I) wherein Q is (i)

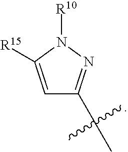

In another embodiment, the present invention is directed to compounds of formula (I) wherein Q is (j)

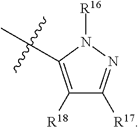

In another embodiment, the present invention is directed to compounds of formula (I) wherein Q is (k)

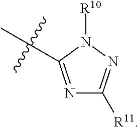

In another embodiment, the present invention is directed to compounds of formula (I) wherein Q is selected from the group consisting of

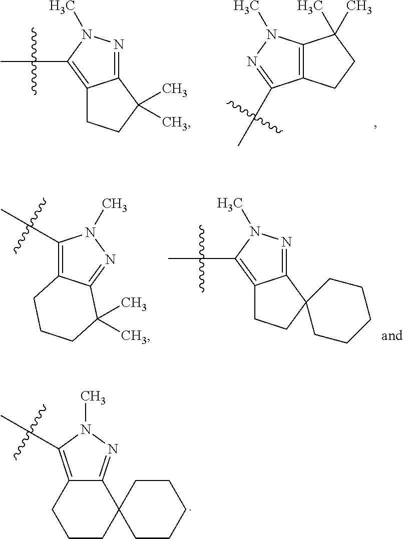

In an embodiment, the present invention is directed to compounds of formula (I) wherein Q is a substituted ring structure selected from the group consisting of 2-methyl-5-tert-butyl-fur-3-yl, 2-methyl-4-cyano-5-tert-butyl-fur-3-yl, 2-tert-butyl-thiazol-5-yl, 3-chloro-isoxazol-5-yl, 3-bromo-isoxazol-5-yl, 3-n-propyl-isoxazol-5-yl, 3-isopropyl-isoxazol-5-yl; 3-isobutyl-isoxazol-5-yl, 3-tert-butyl-isoxazol-5-yl, 3-(2,2-dimethyl-propyl)-isoxazol-5-yl, 3-(pentan-3-yl)-isoxazol-5-yl, 3-cyclopropyl-isoxazol-5-yl, 3-cyclopentyl-isoxazol-5-yl, 3-cyclohexyl-isoxazol-5-yl, 3-tert-butyl-4-methyl-isoxazol-5-yl, 4-tert-butyl-5-methyl-oxazol-2-yl, 2-tert-butyl-5-methyl-oxazol-4-yl, 2-tert-butyl-4-methyl-oxazol-5-yl, 5-tert-butyl-(1,3,4-oxadiazol-2-yl), 1-methyl-2-tert-butyl-imidazol-5-yl, 1-methyl-5-cyclobutyl-pyrazol-3-yl, 1-methyl-3-tert-butyl-pyrazol-5-yl, 1-methyl-3-trifluoromethyl-pyrazol-5-yl, 1-methyl-3-tert-butyl-4-trifluoromethyl-pyrazol-5-yl, 1-ethyl-3-tert-butyl-pyrazol-5-yl, 1-isopropyl-3-tert-butyl-pyrazol-5-yl, 1-methyl-3-cyclobutyl-pyrazol-5-yl, 1-methyl-3-(1-methyl-cyclopropyl)-pyrazol-5-yl, 1-benzyl-3-tert-butyl-pyrazol-5-yl, 1-methyl-3-tert-butyl-4-chloro-pyrazol-5-yl, 1,4-dimethyl-3-tert-butyl-pyrazol-5-yl, 1-methyl-3-tert-butyl-4-cyano-pyrazol-5-yl, 1-(3-hydroxy-n-propyl)-3-tert-butyl-pyrazol-5-yl, 1-(2-hydroxy-ethyl)-3-tert-butyl-pyrazol-5-yl, 1-methyl-3-tert-butyl-(1,2,4-triazol-5-yl),

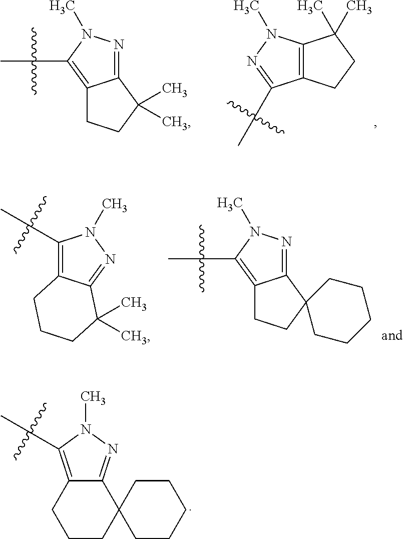

In another embodiment, the present invention is directed to compounds of formula (I) wherein Q is a substituted ring structure selected from the group consisting of 2-methyl-5-tert-butyl-fur-3-yl, 2-methyl-4-cyano-5-tert-butyl-fur-3-yl, 2-tert-butyl-thiazol-5-yl, 3-n-propyl-isoxazol-5-yl, 3-isopropyl-isoxazol-5-yl; 3-isobutyl-isoxazol-5-yl, 3-tert-butyl-isoxazol-5-yl, 3-(2,2-dimethyl-propyl)-isoxazol-5-yl, 3-(pentan-3-yl)-isoxazol-5-yl, 3-cyclopentyl-isoxazol-5-yl, 3-tert-butyl-4-methyl-isoxazol-5-yl, 5-tert-butyl-(1,3,4-oxadiazol-2-yl), 1-methyl-3-tert-butyl-pyrazol-5-yl, 1-methyl-3-tert-butyl-4-trifluoromethyl-pyrazol-5-yl, 1-isopropyl-3-tert-butyl-pyrazol-5-yl, 1-methyl-3-cyclobutyl-pyrazol-5-yl, 1-methyl-3-(1-methyl-cyclopropyl)-pyrazol-5-yl, 1-ethyl-3-tert-butyl-pyrazol-5-yl, 1-benzyl-3-tert-butyl-pyrazol-5-yl, 1-methyl-3-tert-butyl-4-chloro-pyrazol-5-yl, 1,4-dimethyl-3-tert-butyl-pyrazol-5-yl, 1-methyl-3-tert-butyl-4-cyano-pyrazol-5-yl, 1-methyl-3-tert-butyl-(1,2,4-triazol-5-yl),

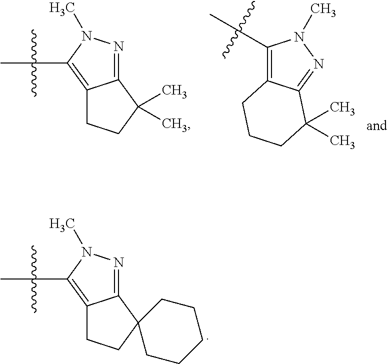

In another embodiment, the present invention is directed to compounds of formula (I) wherein Q is a substituted ring structure selected from the group consisting of 2-methyl-4-cyano-5-tert-butyl-fur-3-yl, 3-tert-butyl-isoxazol-5-yl, 3-tert-butyl-4-methyl-isoxazol-5-yl, 5-tert-butyl-(1,3,4-oxadiazol-2-yl), 1-methyl-3-tert-butyl-pyrazol-5-yl, 1-methyl-3-tert-butyl-4-trifluoromethyl-pyrazol-5-yl, 1-methyl-3-tert-butyl-4-chloro-pyrazol-5-yl, 1,4-dimethyl-3-tert-butyl-pyrazol-5-yl, 1-methyl-3-tert-butyl-4-cyano-pyrazol-5-yl, 1-methyl-3-tert-butyl-(1,2,4-triazol-5-yl), and

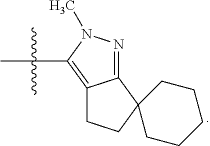

In another embodiment, the present invention is directed to compounds of formula (I) wherein Q is a substituted ring structure selected from the group consisting of 3-tert-butyl-isoxazol-5-yl, 5-tert-butyl-(1,3,4-oxadiazol-2-yl), 1-methyl-3-tert-butyl-pyrazol-5-yl, 1-methyl-3-tert-butyl-4-chloro-pyrazol-5-yl, 1,4-dimethyl-3-tert-butyl-pyrazol-5-yl and 1-methyl-3-tert-butyl-4-cyano-pyrazol-5-yl. In another embodiment, the present invention is directed to compounds of formula (I) wherein Q is a substituted ring structure selected from the group consisting of 3-tert-butyl-isoxazol-5-yl, 1-methyl-3-tert-butyl-pyrazol-5-yl, 1-methyl-3-tert-butyl-4-chloro-pyrazol-5-yl and 1-methyl-3-tert-butyl-4-cyano-pyrazol-5-yl. In another embodiment, the present invention is directed to compounds of formula (I) wherein Q is a substituted ring structure selected from the group consisting of 1-methyl-3-tert-butyl-4-chloro-pyrazol-5-yl, 1,4-dimethyl-3-tert-butyl-pyrazol-5-yl and 1-methyl-3-tert-butyl-4-cyano-pyrazol-5-yl.

In an embodiment, the present invention is directed to compounds of formula (I) wherein R¹⁰ and R¹¹ are each independently selected from the group consisting C₁₋₄alkyl. In another embodiment, the present invention is directed to compounds of formula (I) wherein R¹⁰ and R¹¹ are each independently selected from the group consisting of methyl and tert-butyl.

In an embodiment, the present invention is directed to compounds of formula (I) wherein R¹² is selected from the group consisting of hydrogen and cyano.

In an embodiment, the present invention is directed to compounds of formula (I) wherein R¹³ is selected from the group consisting of hydrogen and C₁₋₄alkyl. In another embodiment, the present invention is directed to compounds of formula (I) wherein R¹³ is selected from the group consisting of hydrogen and tert-butyl.

In an embodiment, the present invention is directed to compounds of formula (I) wherein R¹⁴ is selected from the group consisting of chloro, bromo, C₁₋₆alkyl and C₃₋₆cycloalkyl.

In an embodiment, the present invention is directed to compounds of formula (I) wherein R¹⁵ is selected from the group consisting of C₃₋₆cycloalkyl. In another embodiment, the present invention is directed to compounds of formula (I) wherein R¹⁵ is cyclobutyl.

In an embodiment, the present invention is directed to compounds of formula (I) wherein R¹⁶ is selected from the group consisting of C₁₋₄alkyl, —(C₁₋₄alkyl)-OH and benzyl. In another embodiment, the present invention is directed to compounds of formula (I) wherein R¹⁶ is selected from the group consisting of C₁₋₄alkyl, —(C₂₋₃alkyl)-OH and benzyl.

In an embodiment, the present invention is directed to compounds of formula (I) wherein R¹⁷ is selected from the group consisting of C₁₋₄alkyl, trifluoromethyl, C₃₋₄cycloalkyl and 1-methyl-cyclopropyl. In another embodiment, the present invention is directed to compounds of formula (I) wherein R¹⁷ is selected from the group consisting of tert-butyl, trifluoromethyl, cyclobutyl and 1-methyl-cyclopropyl.

In an embodiment, the present invention is directed to compounds of formula (I) wherein R¹⁸ is selected from the group consisting of hydrogen, chloro, C₁₋₂alkyl, trifluoromethyl and cyano. In another embodiment, the present invention is directed to compounds of formula (I) wherein R¹⁸ is selected from the group consisting of hydrogen, chloro, methyl, trifluoromethyl and cyano.

Additional embodiments of the present invention, include those wherein the substituents for one or more of the variables defined herein (i.e. R¹, a, R², Q, etc.) are independently selected to be any individual substituent or any subset of substituents selected from the complete list as defined herein.

Representative compounds of formula (I) of the present invention are listed in Table 1, below. In another embodiment, the present invention is directed to any single compound or subset of compounds selected from the representative compounds listed in Table 1, below.

TABLE 1
Representative Compounds of Formula (I)

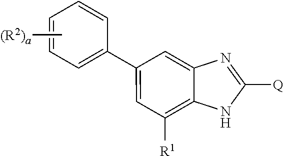

| ID No | R¹ | (R²)ₐ | Q |
|---|---|---|---|
| 1 | H | a = 0 | 3-isopropyl-isoxazol-5-yl |
| 2 | H | 2-methoxy | 3-isopropyl-isoxazol-5-yl |
| 3 | H | 2-trifluoromethyl | 3-isopropyl-isoxazol-5-yl |
| 5 | H | 2-trifluoromethoxy | 3-isopropyl-isoxazol-5-yl |
| 6 | H | 2-ethoxy | 3-isopropyl-isoxazol-5-yl |
| 7 | H | 3-trifluoromethyl | 3-isopropyl-isoxazol-5-yl |
| 8 | H | 2-methoxy | 3-tert-butyl-isoxazol-5-yl |
| 9 | H | 2-trifluoromethyl | 3-tert-butyl-isoxazol-5-yl |
| 10 | H | 2-trifluoromethoxy | 3-tert-butyl-isoxazol-5-yl |
| 12 | H | 2-methoxy | 1-methyl-3-tert-butyl- |
| | | | pyrazol-5-yl |
| 13 | H | 2-trifluoromethyl | 3-tert-butyl-isoxazol-5-yl |
| 14 | H | 2-trifluoromethoxyl | 3-tert-butyl-isoxazol-5-yl |
| 15 | H | 2-methoxy | 3-cyclopropyl-isoxazol-5-yl |
| 16 | H | 2-trifluoromethyl | 3-cyclopropyl-isoxazol-5-yl |
| 17 | H | 2-trifluoromethoxy | 3-cyclopropyl-isoxazol-5-yl |
| 18 | H | 2-trifluoromethyl | 3-n-propyl-isoxazol-5-yl |
| 19 | H | 2-trifluoromethoxy | 3-n-propyl-isoxazol-5-yl |
| 20 | H | 2-trifluoromethyl | 3-(2,2-dimethyl-propyl)- |
| | | | isoxazol-5-yl |
| 21 | H | 2-trifluoromethoxy | 3-(2,2-dimethyl-propyl)- |
| | | | isoxazol-5-yl |
| 22 | H | 2-trifluoromethyl | 3-isobutyl-isoxazol-5-yl |
| 23 | H | 2-trifluoromethoxy | 3-isobutyl-isoxazol-5-yl |
| 24 | H | 2-trifluoromethyl | 3-cyclohexyl-isoxazol-5-yl |
| 25 | H | 2-trifluoromethoxy | 3-cyclohexyl-isoxazol-5-yl |
| 26 | H | 2-trifluoromethyl | 3-(pentan-3-yl)-isoxazol-5-yl |
| 27 | H | 2-trifluoromethoxy | 3-(pentan-3-yl)-isoxazol-5-yl |
| 30 | H | 2-trifluoromethyl | 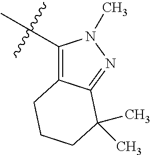 |
| 31 | H | 2-trifluoromethoxy | 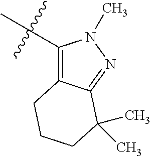 |
| 32 | H | 2-chloro | 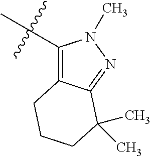 |
| 33 | H | 2-trifluoromethyl | 3-chloro-isoxazol-5-yl |
| 34 | H | 2-trifluoromethoxy | 3-chloro-isoxazol-5-yl |
| 37 | H | 2-trifluoromethyl | 1-methyl-3-cyclobutyl- |
| | | | pyrazol-5-yl |
| 38 | H | 2-trifluoromethoxy | 1-methyl-3-cyclobutyl- |
| | | | pyrazol-5-yl |
| 39 | H | 2-chloro | 1-methyl-3-cyclobutyl- |
| | | | pyrazol-5-yl |
| 40 | H | 2-trifluoromethyl | 1-methyl-3-trifluoro-methyl- |
| | | | pyrazol-5-yl |
| 41 | H | 2-trifluoromethoxy | 1-methyl-3-trifluoro-methyl- |
| | | | pyrazol-5-yl |
| 42 | H | 2-trifluoromethyl | 3-bromo-isoxazol-5-yl |
| 43 | H | 2-trifluoromethoxy | 3-bromo-isoxazol-5-yl |
| 44 | H | 2-trifluoromethyl | 1-ethyl-3-tert-butyl-pyrazol- |
| | | | 5-yl |
| 45 | H | 2-trifluoromethoxy | 1-ethyl-3-tert-butyl-pyrazol- |
| | | | 5-yl |
| 46 | H | 2-trifluoromethyl | 1-isopropyl-3-tert-butyl- |
| | | | pyrazol-5-yl |
| 47 | H | 2-trifluoromethoxy | 1-isopropyl-3-tert-butyl- |
| | | | pyrazol-5-yl |
| 48 | H | 2-trifluoromethyl | 1-benzyl-3-tert-butyl- |
| | | | pyrazol-5-yl |
| 49 | H | 2-trifluoromethoxy | 1-benzyl-3-tert-butyl- |
| | | | pyrazol-5-yl |
| 50 | H | 2-trifluoromethyl | 1-methyl-3-tert-butyl-4- |
| | | | chloro-pyrazol-5-yl |
| 51 | H | 2-trifluoromethoxy | 1-methyl-3-tert-butyl-4- |
| | | | chloro-pyrazol-5-yl |
| 52 | H | 2-trifluoromethyl | 1-methyl-3-(1-methyl- |
| | | | cyclopropyl)-pyrazol-5-yl |
| 53 | H | 2-trifluoromethoxy | 1-methyl-3-(1-methyl- |
| | | | cyclopropyl)-pyrazol-5-yl |
| 54 | H | 2,6-dimethoxy | 1-methyl-3-tert-butyl- |
| | | | pyrazol-5-yl |
| 55 | H | 2-methyl | 1-methyl-3-tert-butyl- |
| | | | pyrazol-5-yl |
| 56 | H | 2-ethyl | 1-methyl-3-tert-butyl- |
| | | | pyrazol-5-yl |
| 57 | H | 2-fluoro | 1-methyl-3-tert-butyl- |
| | | | pyrazol-5-yl |
| 58 | H | 2,6-difluoro | 1-methyl-3-tert-butyl- |
| | | | pyrazol-5-yl |
| 59 | H | 2-chloro | 1-methyl-3-tert-butyl- |
| | | | pyrazol-5-yl |
| 60 | H | 2-cyano | 1-methyl-3-tert-butyl- |
| | | | pyrazol-5-yl |
| 61 | H | 2-fluoro-6- | 1-methyl-3-tert-butyl- |
| | | trifluoromethyl | pyrazol-5-yl |
| 63 | H | 2,6-dimethoxy | 3-tert-butyl-isoxazol-5-yl |
| 64 | H | 2-fluoro-6-methoxy | 3-tert-butyl-isoxazol-5-yl |
| 65 | H | 2-methyl | 3-tert-butyl-isoxazol-5-yl |
| 66 | H | 2-fluoro | 3-tert-butyl-isoxazol-5-yl |
| 67 | H | 2,6-difluoro | 3-tert-butyl-isoxazol-5-yl |
| 68 | H | 2-chloro | 3-tert-butyl-isoxazol-5-yl |
| 69 | H | 2-cyano | 3-tert-butyl-isoxazol-5-yl |
| 70 | H | 2-fluoro-6-methoxy | 1-methyl-2-tert-butyl- |
| | | | imidazol-5-yl |
| 76 | H | 2-trifluoromethyl | 1,4-dimethyl-3-tert-butyl- |
| | | | pyrazol-5-yl |
| 78 | H | 2-fluoro-6- | 1,4-dimethyl-3-tert-butyl- |
| | | trifluoromethyl | pyrazol-5-yl |
| 79 | CH₃ | 2-trifluoromethyl | 1-methyl-3-tert-butyl- |
| | | | pyrazol-5-yl |
| 81 | H | 2-difluoromethoxy | 1-methyl-3-tert-butyl- |
| | | | pyrazol-5-yl |
| 82 | H | 2-fluoro-6- | 3-tert-butyl-isoxazol-5-yl |
| | | trifluoromethyl | |
| 85 | H | 2,6-difluoro | 1-methyl-3-tert-butyl-4- |
| | | | chloro-pyrazol-5-yl |
| 86 | H | 2-fluoro-6- | 1-methyl-3-tert-butyl-4- |
| | | trifluoromethyl | chloro-pyrazol-5-yl |
| 87 | H | 2,6-bis- | 1-methyl-3-tert-butyl-4- |
| | | (trifluoromethyl) | chloro-pyrazol-5-yl |
| 88 | H | 2-fluoro-6- | 1-methyl-3-tert-butyl-4- |
| | | trifluoromethoxy | chloro-pyrazol-5-yl |
| 89 | H | 2-chloro | 1-methyl-3-tert-butyl-4- |
| | | | chloro-pyrazol-5-yl |
| 90 | H | 2-fluoro | 1-methyl-3-tert-butyl-4- |
| | | | chloro-pyrazol-5-yl |
| 91 | H | 2-trifluoromethyl | 1-methyl-5-cyclobutyl- |
| | | | pyrazol-3-yl |
| 92 | H | 2-trifluoromethyl | 3-cyclopentyl-isoxazol-5-yl |
| 93 | H | 2-fluoro | 1,4-dimethyl-3-tert-butyl- |
| | | | pyrazol-5-yl |
| 94 | H | 2-chloro | 1,4-dimethyl-3-tert-butyl- |
| | | | pyrazol-5-yl |
| 95 | H | 2,6-difluoro | 1,4-dimethyl-3-tert-butyl- |
| | | | pyrazol-5-yl |
| 96 | H | 2-fluoro-6- | 1,4-dimethyl-3-tert-butyl- |
| | | trifluoromethoxy | pyrazol-5-yl |
| 97 | H | 2,6-bis- | 1,4-dimethyl-3-tert-butyl- |
| | | (trifluoromethyl) | pyrazol-5-yl |
| 98 | CH₃ | 2-trifluoromethyl | 1-ethyl-3-tert-butyl-pyrazol- |
| | | | 5-yl |
| 100 | H | 2-trifluoromethyl | 2-tert-butyl-thiazol-5-yl |
| 101 | H | 2-trifluoromethyl | 1-(3-hydroxypropyl)-3-tert- |
| | | | butyl-pyrazol-5-yl |
| 102 | H | 2-trifluoromethyl | 1-(2-hydroxyethyl)-3-tert- |
| | | | butyl-pyrazol-5-yl |
| 103 | H | 2-fluoro-6- | 3-tert-butyl-isoxazol-5-yl |
| | | trifluoromethoxy | |
| 104 | H | 2,6-bis- | 3-tert-butyl-isoxazol-5-yl |
| | | (trifluoromethyl) | |
| 105 | H | 2-methoxy-5-fluoro | 3-tert-butyl-isoxazol-5-yl |
| 106 | H | 2-fluoro | 3-tert-butyl-4-methyl- |
| | | | isoxazol-5-yl |
| 107 | H | 2-chloro | 3-tert-butyl-4-methyl- |
| | | | isoxazol-5-yl |
| 108 | H | 2-trifluoromethyl | 3-tert-butyl-4-methyl- |
| | | | isoxazol-5-yl |
| 109 | H | 2,6-difluoro | 3-tert-butyl-4-methyl- |
| | | | isoxazol-5-yl |
| 110 | H | 2-fluoro-6- | 3-tert-butyl-4-methyl- |
| | | trifluoromethoxy | isoxazol-5-yl |
| 111 | H | 2-fluoro-6- | 3-tert-butyl-4-methyl- |
| | | trifluoromethyl | isoxazol-5-yl |
| 112 | Cl | 2,6-difluoro | 1-methyl-3-tert-butyl-4- |
| | | | chloro-pyrazol-5-yl |
| 113 | Cl | 2-fluoro-6- | 1-methyl-3-tert-butyl-4- |
| | | trifluoromethyl | chloro-pyrazol-5-yl |
| 114 | Cl | 2-fluoro | 1-methyl-3-tert-butyl-4- |
| | | | chloro-pyrazol-5-yl |
| 115 | Cl | 2-chloro | 1-methyl-3-tert-butyl-4- |
| | | | chloro-pyrazol-5-yl |
| 116 | Cl | 2-trifluoromethyl | 1-methyl-3-tert-butyl-4- |
| | | | chloro-pyrazol-5-yl |
| 117 | Cl | 2-trifluoromethoxy | 1-methyl-3-tert-butyl-4- |
| | | | chloro-pyrazol-5-yl |
| 118 | CF₃ | 2-fluoro | 1,4-dimethyl-3-tert-butyl- |
| | | | pyrazol-5-yl |
| 119 | CF₃ | 2-fluoro-6- | 1,4-dimethyl-3-tert-butyl- |
| | | trifluoromethyl | pyrazol-5-yl |
| 120 | CF₃ | 2-trifluoromethyl | 1,4-dimethyl-3-tert-butyl- |
| | | | pyrazol-5-yl |
| 121 | CF₃ | 2-chloro | 1,4-dimethyl-3-tert-butyl- |
| | | | pyrazol-5-yl |
| 122 | CF₃ | 2,6-difluoro | 1,4-dimethyl-3-tert-butyl- |
| | | | pyrazol-5-yl |
| 123 | CF₃ | 2-fluoro-6- | 1,4-dimethyl-3-tert-butyl- |
| | | trifluoromethoxy | pyrazol-5-yl |
| 124 | CF₃ | 2,6-difluoro | 1-methyl-3-tert-butyl-4- |
| | | | chloro-pyrazol-5-yl |
| 125 | CF₃ | 2-fluoro-6- | 1-methyl-3-tert-butyl-4- |
| | | trifluoromethyl | chloro-pyrazol-5-yl |
| 126 | CF₃ | 2-fluoro-6- | 1-methyl-3-tert-butyl-4- |
| | | trifluoromethoxy | chloro-pyrazol-5-yl |
| 127 | CF₃ | 2-fluoro | 1-methyl-3-tert-butyl-4- |
| | | | chloro-pyrazol-5-yl |
| 128 | CF₃ | 2-chloro | 1-methyl-3-tert-butyl-4- |
| | | | chloro-pyrazol-5-yl |
| 129 | CF₃ | 2-trifluoromethyl | 1-methyl-3-tert-butyl-4- |
| | | | chloro-pyrazol-5-yl |
| 130 | Cl | 2-fluoro-6- | 1-methyl-3-tert-butyl-4- |
| | | trifluoromethoxy | chloro-pyrazol-5-yl |
| 131 | H | 2-fluoro | 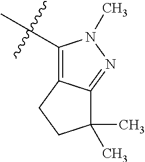 |
| 132 | H | 2-fluoro | 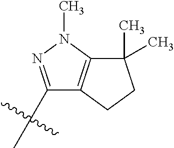 |
| 133 | Cl | 2-fluoro | 1,4-dimethyl-3-tert-butyl- |
| | | | pyrazol-5-yl |
| 134 | Cl | 2-chloro | 1,4-dimethyl-3-tert-butyl- |
| | | | pyrazol-5-yl |
| 135 | Cl | 2-trifluoromethyl | 1,4-dimethyl-3-tert-butyl- |
| | | | pyrazol-5-yl |
| 136 | Cl | 2,6-difluoro | 1,4-dimethyl-3-tert-butyl- |
| | | | pyrazol-5-yl |
| 137 | Cl | 2-fluoro-6- | 1,4-dimethyl-3-tert-butyl- |
| | | trifluoromethyl | pyrazol-5-yl |
| 138 | Cl | 2-fluoro-6- | 1,4-dimethyl-3-tert-butyl- |
| | | trifluoromethoxy | pyrazol-5-yl |
| 139 | CF₃ | 2-fluoro | 3-tert-butyl-isoxazol-5-yl |
| 140 | CF₃ | 2,6-difluoro | 3-tert-butyl-isoxazol-5-yl |
| 141 | CF₃ | 2-chloro | 3-tert-butyl-isoxazol-5-yl |
| 142 | CF₃ | 2-trifluoromethyl | 3-tert-butyl-isoxazol-5-yl |
| 143 | CF₃ | 2-trifluoromethoxy | 3-tert-butyl-isoxazol-5-yl |
| 144 | CF₃ | 2-fluoro-6- | 3-tert-butyl-isoxazol-5-yl |
| | | trifluoromethyl | |
| 145 | CF₃ | 2-fluoro-6- | 3-tert-butyl-isoxazol-5-yl |
| | | trifluoromethoxy | |
| 146 | CF₃ | 2-fluoro-6-cyano | 1-methyl-3-tert-butyl-4- |
| | | | chloro-pyrazol-5-yl |
| 147 | CF₃ | 2-fluoro | 1-methyl-3-tert-butyl-4- |
| | | | cyano-pyrazol-5-yl |
| 148 | CF₃ | 2-chloro | 1-methyl-3-tert-butyl-4- |
| | | | cyano-pyrazol-5-yl |
| 149 | CF₃ | 2-trifluoromethyl | 1-methyl-3-tert-butyl-4- |
| | | | cyano-pyrazol-5-yl |
| 150 | CF₃ | 2-trifluoromethoxy | 1-methyl-3-tert-butyl-4- |
| | | | cyano-pyrazol-5-yl |
| 151 | CF₃ | 2,6-difluoro | 1-methyl-3-tert-butyl-4- |
| | | | cyano-pyrazol-5-yl |
| 152 | CF₃ | 2-fluoro-6- | 1-methyl-3-tert-butyl-4- |
| | | trifluoromethyl | cyano-pyrazol-5-yl |
| 153 | CF₃ | 2-trifluoromethyl | 2-tert-butyl-5-methyl-oxazol- |
| | | | 4-yl |
| 154 | CF₃ | 2-fluoro | 2-tert-butyl-5-methyl-oxazol- |
| | | | 4-yl |
| 155 | CF₃ | 2-chloro | 2-tert-butyl-5-methyl-oxazol- |
| | | | 4-yl |
| 156 | CF₃ | 2-trifluoromethoxy | 2-tert-butyl-5-methyl-oxazol- |
| | | | 4-yl |
| 157 | CF₃ | a = 0 | 2-tert-butyl-5-methyl-oxazol- |
| | | | 4-yl |
| 158 | CF₃ | 2-fluoro-6- | 2-tert-butyl-5-methyl-oxazol- |
| | | trifluoromethyl | 4-yl |
| 159 | Cl | 2-trifluoromethoxy | 2-tert-butyl-5-methyl-oxazol- |
| | | | 4-yl |
| 161 | Cl | 2-fluoro | 2-tert-butyl-5-methyl-oxazol- |
| | | | 4-yl |
| 163 | Cl | 2-chloro | 2-tert-butyl-5-methyl-oxazol- |
| | | | 4-yl |
| 165 | Cl | 2-trifluoromethyl | 2-tert-butyl-5-methyl-oxazol- |
| | | | 4-yl |
| 167 | Cl | 2-fluoro | 1-methyl-3-tert-butyl-4- |
| | | | cyano-pyrazol-5-yl |
| 168 | Cl | 2-chloro | 1-methyl-3-tert-butyl-4- |
| | | | cyano-pyrazol-5-yl |
| 169 | Cl | 2-trifluoromethyl | 1-methyl-3-tert-butyl-4- |
| | | | cyano-pyrazol-5-yl |
| 170 | Cl | 2-trifluoromethoxy | 1-methyl-3-tert-butyl-4- |
| | | | cyano-pyrazol-5-yl |
| 171 | Cl | 2,6-difluoro | 1-methyl-3-tert-butyl-4- |
| | | | cyano-pyrazol-5-yl |
| 172 | Cl | 2-fluoro-6- | 1-methyl-3-tert-butyl-4- |
| | | trifluoromethyl | cyano-pyrazol-5-yl |
| 173 | Cl | 2-fluoro-6- | 1-methyl-3-tert-butyl-4- |
| | | trifluoromethoxy | cyano-pyrazol-5-yl |
| 174 | H | 2-fluoro | 2-methyl-5-tert-butyl-fur-3-yl |
| 175 | CF₃ | 2-fluoro | 2-tert-butyl-4-methyl-oxazol- |
| | | | 5-yl |
| 176 | CF₃ | 2-trifluoromethoxy | 2-tert-butyl-4-methyl-oxazol- |
| | | | 5-yl |
| 177 | CF₃ | 2-trifluoromethyl | 2-tert-butyl-4-methyl-oxazol- |
| | | | 5-yl |
| 178 | CF₃ | a = 0 | 2-tert-butyl-4-methyl-oxazol- |
| | | | 5-yl |
| 179 | CF₃ | 2-chloro | 2-tert-butyl-4-methyl-oxazol- |
| | | | 5-yl |
| 180 | Cl | a = 0 | 2-tert-butyl-4-methyl-oxazol- |
| | | | 5-yl |
| 181 | Cl | 2-chloro | 2-tert-butyl-4-methyl-oxazol- |
| | | | 5-yl |
| 182 | Cl | 2-fluoro | 2-tert-butyl-4-methyl-oxazol- |
| | | | 5-yl |
| 183 | Cl | 2-trifluoromethoxy | 2-tert-butyl-4-methyl-oxazol- |
| | | | 5-yl |
| 184 | CF₃ | 2-trifluoromethyl | 2-methyl-4-cyano-5-tert- |
| | | | butyl-fur-3-yl |
| 185 | CF₃ | 2-fluoro | 1-methyl-3-tert-butyl-4- |
| | | | trifluoromethyl-pyrazol-5-yl |
| 186 | CF₃ | 2-chloro | 1-methyl-3-tert-butyl-4- |
| | | | trifluoromethyl-pyrazol-5-yl |
| 187 | CF₃ | 2-trifluoromethyl | 1-methyl-3-tert-butyl-4- |
| | | | trifluoromethyl-pyrazol-5-yl |
| 188 | CF₃ | 2-trifluoromethoxy | 1-methyl-3-tert-butyl-4- |
| | | | trifluoromethyl-pyrazol-5-yl |
| 189 | CF₃ | 2-trifluoromethyl | 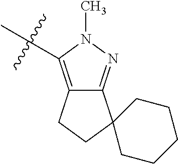 |
| 190 | CF₃ | 2-trifluoromethyl | 5-tert-butyl-(1,3,4-oxadiazol- |
| | | | 2-yl) |
| 191 | CF₃ | 2-trifluoromethyl | 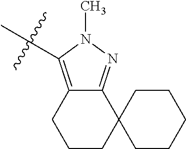 |
| 192 | CF₃ | 2-trifluoromethyl | 1-methyl-3-tert-butyl-(1,2,4- |
| | | | triazol-5-yl) |
| 193 | CF₃ | 2-fluoro | 4-tert-butyl-5-methyl-oxazol- |
| | | | 2-yl |
| 194 | CF₃ | 2-trifluoromethyl | 4-tert-butyl-5-methyl-oxazol- |
| | | | 2-yl |
| 195 | CF₃ | 2-trifluoromethoxy | 4-tert-butyl-5-methyl-oxazol- |
| | | | 2-yl |
| 196 | CF₃ | 2-chloro | 4-tert-butyl-5-methyl-oxazol- |
| | | | 2-yl |

In another embodiment, the present invention is directed to a compound of formula (I) that exhibits a % Inhibition at 0.2 μM of greater than or equal to about 10% (preferably greater than or equal to about 25%, more preferably greater than or equal to about 80%, more preferably greater than or equal to about 80%), also preferred are greater than or equal to 20% at 0.5 μM, and further preferred are greater than or equal to 30% at 1 μM, as measured according to the procedure described in Biological Example 1, which follows herein.

In an embodiment, the present invention is directed to a compound of formula (I) which exhibits an IC₅₀ of less than or 0.100 μM, preferably less than or equal to about 0.05 μM, more preferably less than or equal to about 0.025 μM, more preferably less than or equal to about 0.01 μM, more preferably less than or equal to about 0.005 μM, as measured according to the procedure described in Biological Example 1, which follows herein.

As used herein, the term “alkyl” whether used alone or as part of a substituent group, include straight and branched chains. For example, alkyl radicals include methyl, ethyl, propyl, isopropyl, butyl, isobutyl, sec-butyl, tert-butyl, pentyl and the like. Unless otherwise noted, the notation “C_{X-Y}alkyl” wherein X and Y are integers shall indicate an alkyl group as herein define containing between X and Y carbon atoms. For example, the term “C₁₋₄alkyl” shall include straight and branched alkyl chains containing between one to four carbon atoms.

As used herein, unless otherwise noted, “alkoxy” shall denote an oxygen ether radical of the above described straight or branched chain alkyl groups. For example, methoxy, ethoxy, n-propoxy, sec-butoxy, tert-butoxy, n-hexyloxy and the like. Similarly, the term “C_{X-Y}alkoxy” wherein X and Y are integers shall indicate an alkoxy group as herein define containing between X and Y carbon atoms. For example, the term “C₁₋₄alkoxy” shall include straight and branched alkoxy groups containing one to four carbon atoms, more particularly, methoxy and ethoxy.

As used herein, unless otherwise noted, the term “fluorinated C₁₋₄alkyl” shall mean any C₁₋₄alkyl group as defined above substituted with at least one fluoro atom. Suitable examples include but are not limited to —CF₃, —CHF₂, CH₂F, —CH₂—CF₃, —CF₂—CF₂—CF₂—CF₃, and the like. Similarly, as used herein, unless otherwise noted, the term “fluorinated C₁₋₄alkoxy” shall mean any C₁₋₄alkyl group as defined above substituted with at least one fluoro atom. Suitable examples include but are not limited to —OCF₃, —OCHF₂, —OCH₂F, —OCH₂—CF₃, —OCF₂—CF₂—CF₂—CF₃, and the like.

As used herein, unless otherwise noted, the term “hydroxy substituted C₁₋₄alkyl” shall mean a C₁₋₄alkyl group as defined above, substituted with at least one hydroxy group. Preferably, the C₁₋₄alkyl group is substituted with one hydroxy group. Preferably, the C₁₋₄alkyl group is substituted with one hydroxy group wherein the hydroxy group is bound at a terminal carbon. Suitable examples include, but are not limited to, —CH₂(OH), —CH₂—CH₂(OH), —CH(OH)—CH₃, —CH₂—CH(OH)—CH₃, —CH₂—CH₂—CH₂—OH, —CH₂—CH₂—CH(OH)—CH₃, CH₂—CH(OH)—CH₂—CH₃, —CH₂—CH₂—CH₂—CH₂(OH), and the like.

As used herein, unless otherwise noted, the term “C₃₋₆cycloalkyl” shall mean any stable 3-6 membered monocyclic, saturated ring system, for example cyclopropyl, cyclobutyl, cyclopentyl and cyclohexyl.

When a particular group is “substituted”, that group may have one or more substituents, preferably from one to five substituents, more preferably from one to three substituents, most preferably from one to two substituents, independently selected from the list of substituents. With reference to substituents, the term “independently” means that when more than one of such substituents is possible, such substituents may be the same or different from each other.

As used herein, the notation “*” shall denote the presence of a stereogenic center. Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

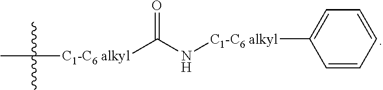

One skilled in the art will recognize that in the listing of substituent groups, as in for example Table 1, the substituent positions on the Q rings are defined as if the Q ring were unattached to any other portion of the molecule and therefore the convention for numbering of the Q ring group defines the position of the substituent. One skilled in the art will further recognize that wherein a complete compound name is listed (for examples in the Examples which follow herein), the compounds were named using standard conventions for assigning priorities and substitution positions. As such substituent groups and substituent bonding positions as listed in a complete compound name may be different from those listed for the corresponding unbound substituent groups, for example as listed in for example Table 1.

Abbreviations used in the specification, particularly the Schemes and Examples, are as follows:

| | |
|---|---|
| AcOH or HOAc = | Acetic Acid |
| ACN = | Acetonitrile |
| Boc = | tert-Butoxy-carbonyl |
| | (i.e. —C(O)O—C(CH₃)₃) |
| Boc₂O = | Di-tert-butyl dicarbonate |
| BOP = | Benzotriazole-1-yl-oxy- |
| | tris(dimethylamino)phosphonium |
| | hexafluorophosphate. |
| Bu₄NBr = | Tetrabutylammonium bromide |
| Chloramine-T = | N-Chloro-p-tosylamide sodium salt |
| CSA = | 10-Camphorsulfonic Acid |
| DAST = | (Diethylamino)sulfur trifluoride |
| DBU = | 1,8-Diazabicyclo[5.4.0]undec-7-ene |
| DCC = | N,N′-Dicyclohexylcarbodiimide |
| DCM = | Dichloromethane |
| Dess-Martin Periodinane = | 1,1,1-Tris(acetoxy)-1,1-dihydro-1,2- |
| | benziodoxol-3-(1H)-one] |
| DIEA or DIPEA = | N,N-Diisopropylethylamine |
| DME = | 1,2-Dimethoxyethane |
| DMF = | N,N-Dimethylformamide |
| DMSO = | Dimethylsulfoxide |
| Et₂O = | Diethyl Ether |
| EtOAc = | Ethyl Acetate |
| EtOH = | Ethanol |
| FBS = | Fetal Bovine Serum |
| HBTU = | O-(Benzotriazole-1-yl)-1,1,3,3- |
| | tetramethyluronium hexafluorophosphate |
| HOAc = | Acetic Acid |
| HPLC = | High Pressure Liquid Chromatography |
| KOAc = | Potassium Acetate |
| MeOH = | Methanol |
| NaOMe = | Sodium Methoxide |
| NBS = | N-Bromosuccinimide |
| NCS = | N-Chlorosuccinimide |
| NH₄OAc = | Ammonium Acetate |
| NMP = | N-Methyl-2-pyrrolidone |
| ¹H-NMR = | Proton Nuclear Magnetic Resonance |
| Pd/C = | Palladium on Carbon Catalyst |
| Pd(OAc)₂ = | Palladium (II) Acetate |
| PdCl₂dppf or (dppf)PdCl₂ = | [1,1′-Bis(diphenylphosphino)ferrocene] |
| | dichloropalladium(II). |
| PdCl₂dppf•DCM or | [1,1′-Bis(diphenylphosphino) ferrocene] |
| (dppf)PdCl₂•DCM = | dichloropalladium(II) dichloromethane (1:1) |
| | adduct (or complex) |
| Pd₂(dba)₃ = | Tris(dibenzylidene acetone)dipalladium(0) |
| Pd(PPh₃)₄ = | Tetrakis(triphenylphosphine) palladium (0) |
| Pt(Sulfided)/C = | Sulfided Platinum on Carbon Catalyst |
| PyBroP = | Bromo-tris(pyrrolidino)-phosphonium |
| | hexafluorophosphate |
| TEA = | Triethylamine |
| TFA = | Trifluoroacetic Acid |
| THF = | Tetrahydrofuran |
| TLC = | Thin Layer Chromatography |
| TRPM8 or TRP M8 = | Transient Receptor Potential M8 channel |
| p-TsOH = | p-Toluenesulfonic Acid |

As used herein, unless otherwise noted, the terms “treating”, “treatment” and the like, shall include the management and care of a subject or patient (preferably mammal, more preferably human) for the purpose of combating a disease, condition or disorder and includes the administration of a compound of the present invention to prevent the onset of the symptoms or complications, alleviate the symptoms or complications or eliminate the disease, condition or disorder.

As used herein, unless otherwise noted, the term “prevention” shall include (a) reduction in the frequency of one or more symptoms; (b) reduction in the severity of one or more symptoms; (c) delay or avoidance of the development of additional symptoms; and/or (d) delay or avoidance of the development of the disorder or condition.

One skilled in the art will recognize that wherein the present invention is directed to methods of prevention, a subject in need thereof (i.e., a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical professional to be at risk of developing said disorder, disease or condition. For example, the subject may be deemed at risk of developing a disorder, disease or condition (and therefore in need of prevention or preventive treatment) as a consequence of the subject's medical history, including but not limited to family history, pre-disposition, co-existing (comorbid) disorders or conditions, genetic testing and the like.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

For the purposes of the present invention, the term “antagonist” is used to refer to a compound capable of producing, depending on the circumstance, a functional antagonism of an ion channel, including but not limited to competitive antagonists, non-competitive antagonists, desensitizing agonists and partial agonists.

For purposes of the present invention, the term “TRPM8-modulated” is used to refer to the condition of being affected by the modulation of the TRPM8 channel, including but not limited to the state of being mediated by the TRPM8 channel.

As antagonists of the TRPM8 channel, the compounds of formula (I) are useful in methods for treating and preventing a disease, a syndrome, a condition or a disorder in a subject, including an animal, a mammal and a human in which the disease, the syndrome, the condition or the disorder is affected by the modulation of TRPM8 channels. Such methods comprise, consist of and consist essentially of administering to a subject, including an animal, a mammal and a human in need of such treatment or prevention, a therapeutically effective amount of a compound, salt or solvate of formula (I). In particular, the compounds of formula (I) are useful for preventing or treating pain or diseases, syndromes, conditions or disorders causing such pain or pulmonary or vascular dysfunction. More particularly, the compounds of formula (I) are useful for preventing or treating inflammatory pain, inflammatory hypersensitivity conditions, neuropathic pain, anxiety, depression, and cardiovascular disease aggravated by cold, including peripheral vascular disease, vascular hypertension, pulmonary hypertension, Raynaud's disease, and coronary artery disease, by administering to a subject in need thereof a therapeutically effective amount of a compound of formula (I).

Examples of inflammatory pain include pain due to a disease, condition, syndrome or disorder, including inflammatory bowel disease, visceral pain, migraine, post operative pain, osteoarthritis, rheumatoid arthritis, back pain, lower back pain, joint pain, abdominal pain, chest pain, labor, musculoskeletal diseases, skin diseases, toothache, pyresis, burn, sunburn, snake bite, venomous snake bite, spider bite, insect sting, neurogenic bladder, interstitial cystitis, urinary tract infection, rhinitis, contact dermatitis/hypersensitivity, itch, eczema, pharyngitis, mucositis, enteritis, irritable bowel syndrome, cholecystitis, pancreatitis, postmastectomy pain syndrome, menstrual pain, endometriosis, sinus headache, tension headache, or arachnoiditis.

One type of inflammatory pain is inflammatory hyperalgesia, which can be further distinguished as inflammatory somatic hyperalgesia or inflammatory visceral hyperalgesia. Inflammatory somatic hyperalgesia can be characterized by the presence of an inflammatory hyperalgesic state in which a hypersensitivity to thermal, mechanical and/or chemical stimuli exists. Inflammatory visceral hyperalgesia can also be characterized by the presence of an inflammatory hyperalgesic state, in which an enhanced visceral irritability exists. Examples of inflammatory hyperalgesia include a disease, syndrome, condition, disorder, or pain state including inflammation, osteoarthritis, rheumatoid arthritis, back pain, joint pain, abdominal pain, musculoskeletal diseases, skin diseases, post operative pain, headaches, toothache, burn, sunburn, insect sting, neurogenic bladder, urinary incontinence, interstitial cystitis, urinary tract infection, cough, asthma, chronic obstructive pulmonary disease, rhinitis, contact dermatitis/hypersensitivity, itch, eczema, pharyngitis, enteritis, irritable bowel syndrome, inflammatory bowel diseases including Crohn's Disease or ulcerative colitis.

One embodiment of the present invention is directed to a method for treating inflammatory somatic hyperalgesia in which a hypersensitivity to thermal, mechanical and/or chemical stimuli exists, comprising the step of administering to a mammal in need of such treatment a therapeutically effective amount of a compound, salt or solvate of formula (I).

A further embodiment of the present invention is directed to a method for treating inflammatory visceral hyperalgesia in which an enhanced visceral irritability exists, comprising, consisting of, and/or consisting essentially of the step of administering to a subject in need of such treatment a therapeutically effective amount of a compound, salt or solvate of formula (I).

A further embodiment of the present invention is directed to a method for treating neuropathic cold allodynia in which a hypersensitivity to a cooling stimuli exists, comprising, consisting of, and/or consisting essentially of the step of administering to a subject in need of such treatment a therapeutically effective amount of a compound, salt or solvate of formula (I).

Examples of a neuropathic pain include pain due to a disease, syndrome, condition or disorder, including cancer, neurological disorders, spine and peripheral nerve surgery, brain tumor, traumatic brain injury (TBI), spinal cord trauma, chronic pain syndrome, fibromyalgia, chronic fatigue syndrome, neuralgias (e.g., trigeminal neuralgia, glossopharyngeal neuralgia, postherpetic neuralgia and causalgia), lupus, sarcoidosis, peripheral neuropathy, bilateral peripheral neuropathy, diabetic neuropathy, central pain, neuropathies associated with spinal cord injury, stroke, amyotrophic lateral sclerosis (ALS), Parkinson's disease, multiple sclerosis, sciatic neuritis, mandibular joint neuralgia, peripheral neuritis, polyneuritis, stump pain, phantom limb pain, bony fractures, oral neuropathic pain, Charcot's pain, complex regional pain syndrome I and II (CRPS I/II), radiculopathy, Guillain-Barre syndrome, meralgia paresthetica, burning-mouth syndrome, optic neuritis, postfebrile neuritis, migrating neuritis, segmental neuritis, Gombault's neuritis, neuronitis, cervicobrachial neuralgia, cranial neuralgia, geniculate neuralgia, glossopharyngial neuralgia, migrainous neuralgia, idiopathic neuralgia, intercostals neuralgia, mammary neuralgia, Morton's neuralgia, nasociliary neuralgia, occipital neuralgia, red neuralgia, Sluder's neuralgia, splenopalatine neuralgia, supraorbital neuralgia, vulvodynia, or vidian neuralgia.

One type of neuropathic pain is neuropathic cold allodynia, which can be characterized by the presence of a neuropathy-associated allodynic state in which a hypersensitivity to cooling stimuli exists. Examples of neuropathic cold allodynia include allodynia due to a disease, condition, syndrome, disorder or pain state including neuropathic pain (neuralgia), pain arising from spine and peripheral nerve surgery or trauma, traumatic brain injury (TBI), trigeminal neuralgia, postherpetic neuralgia, causalgia, peripheral neuropathy, diabetic neuropathy, central pain, stroke, peripheral neuritis, polyneuritis, complex regional pain syndrome I and II (CRPS I/II) and radiculopathy.

As used herein, unless otherwise noted, the term “cardiovascular disease aggravated by cold” shall include peripheral vascular disease, vascular hypertension, pulmonary hypertension, Raynaud's disease and coronary artery disease.

In an embodiment, the present invention is directed to methods for the treatment of inflammatory pain, inflammatory hypersensitivity condition or neuropathic pain, comprising administering to a subject in need thereof a therapeutically effective amount of a compound of formula (I).

In an embodiment of the present invention, the inflammatory pain is pain due to inflammatory bowel disease, visceral pain, migraine, post operative pain, osteoarthritis, rheumatoid arthritis, back pain, lower back pain, joint pain, abdominal pain, chest pain, labor, musculoskeletal diseases, skin diseases, toothache, pyresis, burn, sunburn, snake bite, venomous snake bite, spider bite, insect sting, neurogenic bladder, interstitial cystitis, urinary tract infection, rhinitis, contact dermatitis/hypersensitivity, itch, eczema, pharyngitis, mucositis, enteritis, irritable bowel syndrome, cholecystitis, pancreatitis, postmastectomy pain syndrome, menstrual pain, endometriosis, sinus headache, tension headache, or arachnoiditis. Preferably, the inflammatory pain is inflammatory hyperalgesia.

In another embodiment of the present invention, the inflammatory hyperalgesia is inflammatory somatic hyperalgesia or inflammatory visceral hyperalgesia.

In another embodiment, the present invention is directed to methods for the treatment of inflammatory hyperplasia, wherein the inflammatory hyperalgesia is due to inflammation, osteoarthritis, rheumatoid arthritis, back pain, joint pain, abdominal pain, musculoskeletal diseases, skin diseases, post operative pain, headaches, fibromyalgia, toothache, burn, sunburn, insect sting, neurogenic bladder, urinary incontinence, interstitial cystitis, urinary tract infection, cough, asthma, chronic obstructive pulmonary disease, rhinitis, contact dermatitis/hypersensitivity, itch, eczema, pharyngitis, enteritis, irritable bowel syndrome, Crohn's Disease, or ulcerative colitis.

In another embodiment, the present invention is directed to methods of treating inflammatory hypersensitivity conditions, wherein the inflammatory hypersensitivity condition is urinary incontinence, benign prostatic hypertrophy, cough, asthma, rhinitis, nasal hypersensitivity, itch, contact dermatitis, dermal allergy, or chronic obstructive pulmonary disease.

In another embodiment, the present invention is directed to methods for the treatment of neuropathic pain, wherein the neuropathic pain is due to cancer, a neurological disorder, spine or peripheral nerve surgery, a brain tumor, traumatic brain injury (TBI), spinal cord trauma, a chronic pain syndrome, fibromyalgia, chronic fatigue syndrome, a neuralgia, lupus, sarcoidosis, peripheral neuropathy, bilateral peripheral neuropathy, diabetic neuropathy, central pain, neuropathies associated with spinal cord injury, stroke, ALS, Parkinson's disease, multiple sclerosis, sciatic neuritis, mandibular joint neuralgia, peripheral neuritis, polyneuritis, stump pain, phantom limb pain, a bony fracture, oral neuropathic pain, Charcot's pain, complex regional pain syndrome I and II (CRPS I/II), radiculopathy, Guillain-barre syndrome, meralgia paresthetica, burning-mouth syndrome, optic neuritis, postfebrile neuritis, migrating neuritis, segmental neuritis, Gombault's neuritis, neuronitis, cervicobrachial neuralgia, cranial neuralgia, geniculate neuralgia, glossopharyngial neuralgia, migrainous neuralgia, idiopathic neuralgia, intercostals neuralgia, mammary neuralgia, Morton's neuralgia, nasociliary neuralgia, occipital neuralgia, red neuralgia, Sluder's neuralgia, splenopalatine neuralgia, supraorbital neuralgia, vulvodynia or vidian neuralgia. Preferably, the neuropathic pain is neuropathic cold allodynia or neuralgia. Preferably, the neuralgia is trigeminal neuralgia, glossopharyngeal neuralgia, postherpetic neuralgia, or causalgia.

In another embodiment, the present invention is directed to methods for the treatment of neuropathic cold allodynia, wherein the neuropathic cold allodynia is pain arising from spine and peripheral nerve surgery or trauma, traumatic brain injury (TBI), trigeminal neuralgia, postherpetic neuralgia, causalgia, peripheral neuropathy, diabetic neuropathy, central pain, stroke, peripheral neuritis, polyneuritis, complex regional pain syndrome I and II (CRPS I/II), or radiculopathy.

In another embodiment, the present invention is directed to methods for the treatment of anxiety, wherein the anxiety is social anxiety, post traumatic stress disorder, phobias, social phobia, special phobias, panic disorder, obsessive compulsive disorder, acute stress disorder, separation anxiety disorder, or generalized anxiety disorder.

In another embodiment, the present invention is directed to methods for the treatment of depression wherein the depression is major depression, bipolar disorder, seasonal affective disorder, post natal depression, manic depression, or bipolar depression.

In another embodiment, the present invention is directed to a method for the treatment of inflammatory somatic hyperalgesia in which a hypersensitivity to thermal stimuli exists. In another embodiment, the present invention is directed to a method for the treatment of inflammatory visceral hyperalgesia in which an enhanced visceral irritability exists. In another embodiment, the present invention is directed to a method for the treatment of neuropathic cold allodynia in which a hypersensitivity to cooling stimuli exists.

In another embodiment, the present invention is directed to a method for the treatment of cardiovascular disease aggravated by cold, including peripheral vascular disease, vascular hypertension, pulmonary hypertension, Raynaud's disease and coronary artery disease.

In another embodiment, the present invention is directed to methods for the treatment and/or prevention of migraine, post herpetic neuralgia, post traumatic neuralgia, post chemotherapy neuralgia, complex regional pain syndrome I and II (CRPS I/II), fibromyalgia, inflammatory bowel disease, pruritis, asthma, chronic obstructive pulmonary disease, toothache, bone pain or pyresis in a mammal, which method comprises administering to a mammal in need of such treatment or prevention a therapeutically effective amount of a TRPM8 antagonist.

In another embodiment, the present invention is directed to methods for the treatment and/or prevention of hypertension, peripheral vascular disease,

Raynaud's disease, reperfusion injury or frostbite in a mammal, which method comprises administering to a mammal in need of such treatment or prevention a therapeutically effective amount of a TRPM8 antagonist.

In yet another embodiment, the present invention is directed to methods for accelerating postert-anesthetic recovery or post hypothermia recovery in a mammal, which method comprises administering to a mammal in need of such treatment a therapeutically effective amount of a TRPM8 antagonist.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same or different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems. One skilled in the art will further recognize that wherein two consecutive reaction or process steps are run without isolation of the intermediate product (i.e. the product of the first of the two consecutive reaction or process steps), then the first and second reaction or process steps may be run in the same solvent or solvent system; or alternatively may be run in different solvents or solvent systems following solvent exchange, which may be completed according to known methods.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any range therein.

Examples of suitable solvents, bases, reaction temperatures, and other reaction parameters and components are provided in the detailed descriptions which follow herein. One skilled in the art will recognize that the listing of said examples is not intended, and should not be construed, as limiting in any way the invention set forth in the claims which follow thereafter.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, triflate and the like.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art. As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, tert-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like; and benzylic groups such as benzyl, 4-methoxybenzyl, 2,4-dimethoxybenzyl, and the like. Other suitable nitrogen protecting groups may be found in texts such as T. W. Greene & P. G. M. Wuts, Protective Groups in Organic Synthesis, John Wiley & Sons, 1991.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α−obs]/[α−max])×100.

One embodiment of the present invention is directed to a composition comprising the dextrorotatory enantiomer of a compound of formula (I) wherein said composition is substantially free from the levorotatory isomer of said compound. In the present context, substantially free means less than 25%, preferably less than 10%, more preferably less than 5%, even more preferably less than 2% and even more preferably less than 1% of the levorotatory isomer calculated as.

$$\% dextrorotatory = \frac{( mass dextrorotatory )}{( mass dextrorotatory ) + ( mass levorotatory )} \times 100$$

Another embodiment of the present invention is a composition comprising the levorotatory enantiomer of a compound of formula (I) wherein said composition is substantially free from the dextrorotatory isomer of said compound. In the present context, substantially free from means less than 25%, preferably less than 10%, more preferably less than 5%, even more preferably less than 2% and even more preferably less than 1% of the dextrorotatory isomer calculated as

$$\% levorotatory = \frac{( mass levorotatory )}{( mass dextrorotatory ) + ( mass levorotatory )} \times 100.$$

### General Synthesis Schemes

Compounds of formula (I) of the present invention may be prepared according to the process outlined in Scheme 1, below.

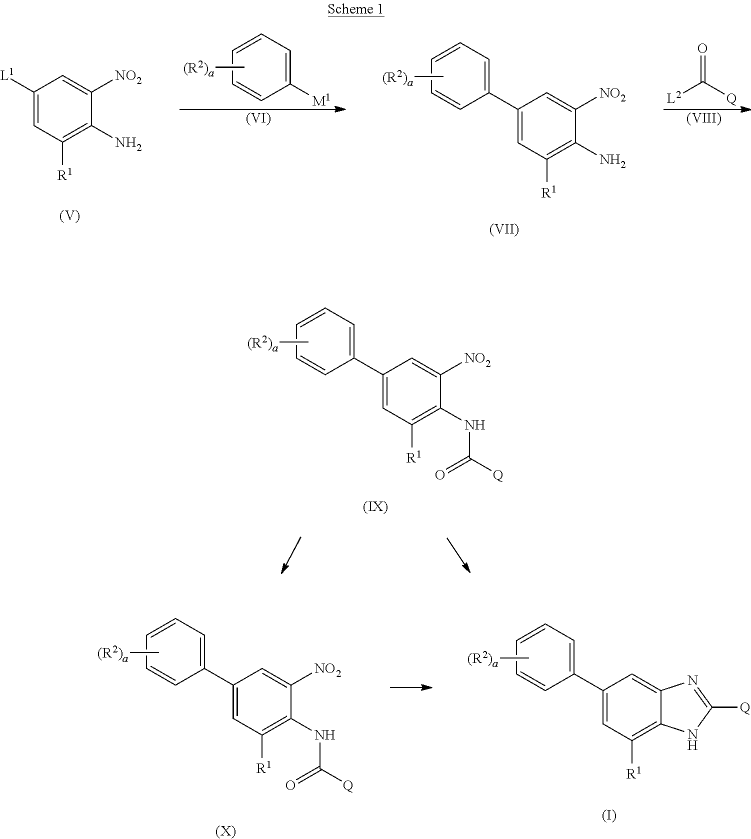

Accordingly, a suitably substituted compound of formula (V), wherein L¹ is a suitably selected leaving group such as chloro, bromo, and the like, a known compound or compound prepared by known methods, is reacted with a suitably substituted compound of formula (VI), wherein M¹ is a suitably selected activating group such as (a) boronic acid (—B(OH)₂), (b) a suitably selected boronic ester such as pinacolatoboryl, neopentylglycolatoboryl, and the like, (c) a suitably selected trialkylstannyl such as tri(n-butyl)tin, and the like, (d) a suitably selected trialkylsilyl such as triallylsilyl, and the like or (e) a suitably selected aryldialkylsilyl such as 2-(hydroxymethyl)phenyl-dimethylsilyl, and the like, a known compound or compound prepared by known methods, under suitable coupling conditions, to yield the corresponding compound of formula (VII).

For example, wherein compound of formula (VI), where M¹ is —B(OH)₂ or a suitably selected boronic ester, the compound of formula (V) is reacted with the compound of formula (VI) under Suzuki coupling conditions, more particularly in the presence of a suitably selected palladium catalyst such as palladium (II) acetate, palladium (II) chloride, bis(acetonitrile)-dichloro-palladium(II), allylpalladium (II) chloride dimer, tris(dibenzylidineacetone) dipalladium (0) (Pd₂(dba)₃), dichloro-bis(di-tert-butylphenylphosphine)-palladium (II), [1,1′-bis-(diphenylphosphino)-ferrocene]-palladium (II) dichloride dichloromethane adduct ((dppf)PdCl₂.DCM), tetrakis(triphenylphosphine) palladium(0) (Pd(PPh₃)₄), (1,1′-bis(di-tert-butylphosphino)ferrocene palladium (II) chloride, and the like; optionally in the presence of a suitably selected added ligand such as triphenylphosphine, tri-o-tolylphosphine, tri(tert-butyl)-phosphine, tricyclohexylphosphine, 1,1′-bis(diphenylphosphino)-ferrocene, bis[2-(diphenyl-phosphino)phenyl]ether, 2-dicyclohexylphosphino-2′,6′-dimethoxybiphenyl, tris(2-furyl)phosphine, 1-butyl-3-methylimidazolium hexafluorophosphate, and the like; in the presence of a suitably selected base such as cesium carbonate, potassium carbonate, sodium carbonate, cesium fluoride, potassium fluoride, tetrabutylammonium fluoride, potassium tert-butoxide, sodium tert-butoxide, aqueous sodium hydroxide, aqueous sodium bicarbonate; potassium phosphate or preferably aqueous sodium carbonate; in a suitably selected organic solvent such as ethanol, THF, DMF, toluene, benzene, DME, 1,4-dioxane, and the like; preferably at a suitable temperature in the range of from about room temperature to about 180° C.

One skilled in the art will recognize that compounds of formula (VII) wherein R¹ is chloro may alternatively be prepared by reacting a suitably substituted compound of formula (V) wherein R^{al} is hydrogen, with a suitably substituted compound of formula (VI), as described above, to yield the corresponding compound of formula (VII) wherein R^{al} is hydrogen. The compound of formula (VII) wherein R¹ is hydrogen is then chlorinated, according to known methods, for example, by reacting with a suitably selected chlorinating agent such as sulfuryl chloride, and the like; in a suitably selected solvent such as DCM, tetrachloromethane, and the like; to yield the corresponding compound of formula (VII), wherein R₁ is chloro.

One skilled in the art will further recognize that the compound of formula (VII) may alternatively be prepared as follows:

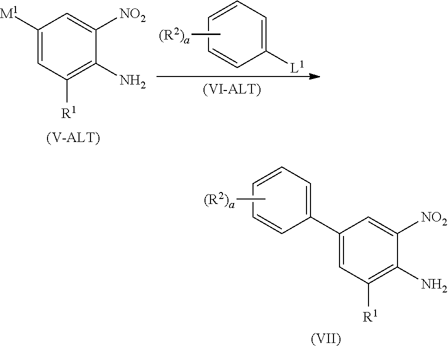

by reacting a suitably substituted compound of formula (V-ALT) wherein M¹ with a suitably selected activating group such as (a) boronic acid (—B(OH)₂), (b) a suitably selected boronic ester such as pinacolatoboryl, neopentylglycolatoboryl, and the like, (c) a suitably selected trialkylstannyl such as tri(n-butyl)tin, and the like, (d) a suitably selected trialkylsilyl such as triallylsilyl, and the like or (e) a suitably selected aryldialkylsilyl such as 2-(hydroxymethyl)phenyl-dimethylsilyl, and the like; with a suitably substituted compound of formula (VI-ALT), wherein L¹ is a suitably selected leaving group such as chloro, bromo, and the like, preferably bromo; as described in more detail above; to yield the corresponding compound of formula (VII).

The compound of formula (VII) is reacted with a suitably substituted compound of formula (VIII), wherein L² is a suitably selected leaving group such as chloro, bromo, fluoro, and the like, preferably chloro, a known compound or compound prepared by known methods; in the presence of a suitably selected base such as sodium hydride, potassium hydride, potassium tert-butoxide, n-butyllithium, and the like, preferably sodium hydride; in a suitably selected organic solvent such as DMF, THF, and the like, to yield the corresponding compound of formula (IX).

The compound of formula (IX) is reacted with a suitably selected reducing agent such as hydrogen in the presence of a catalyst such as palladium on carbon, hydrogen in the presence of a catalyst such as platinum on carbon doped with vanadium, Pt (Sulfided)/C, tin (II) chloride, Fe/NH₄Cl, and the like; in a suitably selected organic solvent such as methanol, ethanol, THF, and the like, to yield the corresponding compound of formula (X).

The compound of formula (X) is reacted with POCl₃ or a suitably selected acid catalyst such as (1S)-(+)-10-camphorsulfonic acid, p-toluenesulfonic acid, acetic acid, and the like; neat or in a suitably selected organic solvent such as 1,4-dioxane, toluene, and the like; to yield the corresponding compound of formula (I).

Alternatively, the compound of formula (IX) is reacted with a suitably selected reducing agent such as iron powder, and the like; in the presence of a suitably selected acid catalyst such as acetic acid, p-toluenesulfonic acid, camphorsulfonic acid, and the like; neat or in a suitably selected organic solvent such as acetic acid, 1,4-dioxane, toluene, and the like; preferably at a temperature in the range of from about 80° C. to about 100° C., to yield the corresponding compound of formula (I).

Compounds of formula (I) may alternatively be prepared according to the process outlined in Scheme 2, below.

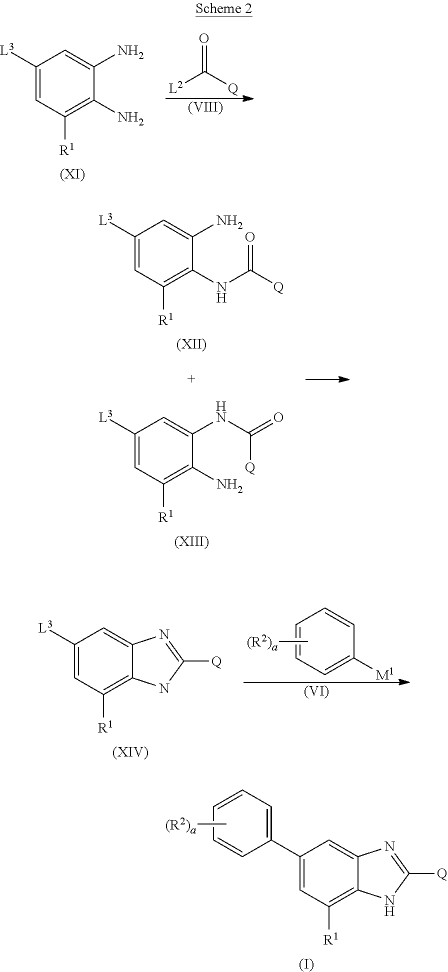

Accordingly, a suitably selected compound of formula (XI), wherein L³ is a suitably selected leaving group such as chloro, bromo, and the like, preferably bromo, is reacted with a suitably substituted compound of formula (VIII), wherein L² is a suitably selected leaving group such as chloro, bromo, fluoro, and the like, preferably chloro, a known compound or compound prepared by known methods; in the presence of a suitably selected base such as DIPEA, TEA, pyridine, and the like, preferably DIPEA, in a suitably selected organic solvent such as DMF, THF, and the like; to yield a mixture of the corresponding compound of formula (XII) and the corresponding compound of formula (XIII).

The mixture of the compound of formula (XII) and the compound of formula (XIII) is reacted with POCl₃ or a suitably selected acid catalyst such as (1S)-(+)-10-camphorsulfonic acid, p-toluenesulfonic acid, acetic acid, and the like; neat or in a suitably selected organic solvent such as 1,4-dioxane, toluene, and the like; to yield the corresponding compound of formula (XIV).

The compound of formula (XIV) is reacted with a suitably substituted compound of formula (VI), wherein M¹ is a suitably selected activating group such as (a) boronic acid (—B(OH)₂), (b) a suitably selected boronic ester such as pinacolatoboryl, neopentylglycolatoboryl, and the like, (c) a suitably selected trialkylstannyl such as tri(n-butyl)tin, and the like, (d) a suitably selected trialkylsilyl such as triallylsilyl, and the like or (e) a suitably selected aryldialkylsilyl such as 2-(hydroxymethyl)phenyl-dimethylsilyl, and the like, a known compound or compound prepared by known methods, under suitable coupling conditions, for example, as described in more detail in Scheme 1, above; to yield the corresponding compound of formula (I).

One skilled in the art will further recognize that the compound of formula (I) may alternatively be prepared as follows:

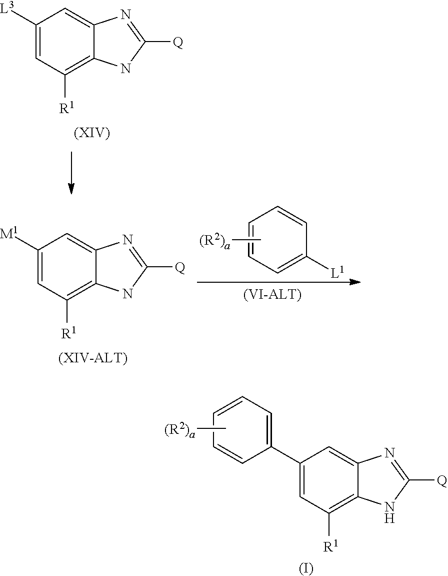

Compounds of formula (XIV) where L³ is a suitably selected leaving group such as bromo, and the like, may be reacted to yield the corresponding compound of formula (XIV-ALT), wherein M¹ is a suitably selected activating group such as (a) boronic acid (—B(OH)₂), (b) a suitably selected boronic ester such as pinacolatoboryl, neopentylglycolatoboryl, and the like, according to known methods. The compound of formula (XIV-ALT) may then be reacted with a suitably substituted compound of formula (VI-ALT), wherein L¹ is a suitably selected leaving group such as chloro, bromo, and the like, preferably bromo; as described in more detail above; to yield the corresponding compound of formula (I).

Compounds of formula (I) may alternatively be prepared according to the process outlined in Scheme 3, below.

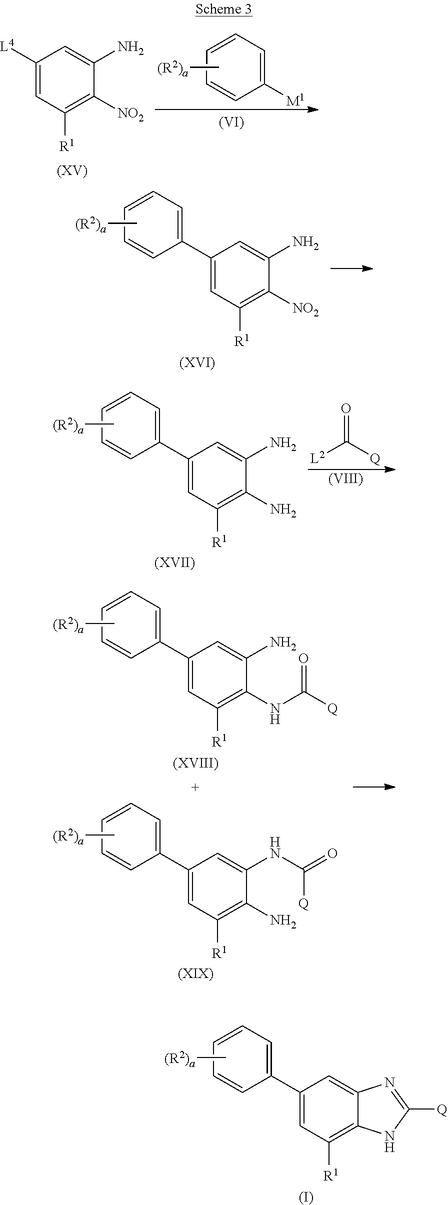

Accordingly, a suitably substituted compound of formula (XV), wherein L⁴ is a suitably selected leaving group such as chloro, bromo, and the like, a known compound or compound prepared by known methods, is reacted with a suitably substituted compound of formula (VI), wherein M¹ is a suitably selected activating group such as (a) boronic acid (—B(OH)₂), (b) a suitably selected boronic ester such as pinacolatoboryl, neopentylglycolatoboryl, and the like, (c) a suitably selected trialkylstannyl such as tri(n-butyl)tin, and the like, (d) a suitably selected trialkylsilyl such as triallylsilyl, and the like or (e) a suitably selected aryldialkylsilyl such as 2-(hydroxymethyl)phenyl-dimethylsilyl, and the like, a known compound or compound prepared by known methods, under suitable coupling conditions, to yield the corresponding compound of formula (XVI).

For example, wherein compound of formula (VI), where M¹ is —B(OH)₂ or a suitably selected boronic ester, the compound of formula (V) is reacted with the compound of formula (VI) under Suzuki coupling conditions, more particularly in the presence of a suitably selected palladium catalyst such as palladium (II) acetate, palladium (II) chloride, bis(acetonitrile)-dichloro-palladium(II), allylpalladium (II) chloride dimer, tris(dibenzylidineacetone) dipalladium (0) (Pd₂(dba)₃), dichloro-bis(di-tert-butylphenylphosphine)-palladium (II), [1,1′-bis-(diphenylphosphino)-ferrocene]-palladium (II) dichloride dichloromethane adduct ((dppf)PdCl₂.DCM), tetrakis(triphenylphosphine) palladium(0) (Pd(PPh₃)₄), (1,1′-bis(di-tert-butylphosphino)ferrocene palladium (II) chloride, and the like; optionally in the presence of a suitably selected added ligand such as triphenylphosphine, tri-o-tolylphosphine, tri(tert-butyl)-phosphine, tricyclohexylphosphine, 1,1′-bis(diphenylphosphino)-ferrocene, bis[2-(diphenyl-phosphino)phenyl]ether, 2-dicyclohexylphosphino-2′,6′-dimethoxybiphenyl, tris(2-furyl)phosphine, 1-butyl-3-methylimidazolium hexafluorophosphate, and the like; in the presence of a suitably selected base such as cesium carbonate, potassium carbonate, sodium carbonate, cesium fluoride, potassium fluoride, tetrabutylammonium fluoride, potassium tert-butoxide, sodium tert-butoxide, aqueous sodium hydroxide, aqueous sodium bicarbonate; potassium phosphate or preferably aqueous sodium carbonate; in a suitably selected organic solvent such as ethanol, THF, DMF, toluene, benzene, DME, 1,4-dioxane, and the like; preferably at a suitable temperature in the range of from about room temperature to about 180° C.

The compound of formula (XVI) is reacted with a suitably selected reducing agent, as hydrogen in the presence of a catalyst such as palladium on carbon, hydrogen in the presence of a catalyst such as platinum on carbon doped with vanadium, Pt (Sulfided)/C, tin (II) chloride, Fe/NH₄Cl, and the like; in a suitably selected organic solvent such as methanol, ethanol, THF, and the like, to yield the corresponding compound of formula (XVII).

The compound of formula (XVII) is reacted with a suitably substituted compound of formula (VIII), wherein L² is a suitably selected leaving group such as chloro, bromo, fluoro, and the like, preferably chloro, a known compound or compound prepared by known methods; in the presence of a suitably selected base such as DIPEA, TEA, pyridine, and the like, preferably DIPEA, in a suitably selected organic solvent such as DMF, THF, and the like; to yield a mixture of the corresponding compound of formula (XVIII) and the corresponding compound of formula (XIX).

The mixture of the compound of formula (XVIII) and the compound of formula (XIX) is reacted with POCl₃ or a suitably selected acid catalyst such as (1S)-(+)-10-camphorsulfonic acid, p-toluenesulfonic acid, acetic acid, and the like; neat or in a suitably selected organic solvent such as 1,4-dioxane, toluene, and the like; to yield the corresponding compound of formula (I).

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, esultin, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 mg to about 1,000 mg or any amount or range therein, and may be given at a dosage of from about 0.01 mg/kg/day to about 300 mg/kg/day, or any amount or range therein, preferably from about 0.1 mg/kg/day to about 50 mg/kg/day, or any amount or range therein, more preferably from about 0.1 mg/kg/day to about 10 mg/kg/day, or any amount or range therein, more preferably from about 0.1 mg/kg/day to about 5 mg/kg/day, or any amount or range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.01 mg to about 1,000 mg, or any amount or range therein, of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating TRP M8 mediated disorders described in the present invention may also be carried out using a pharmaceutical composition comprising any of the compounds as defined herein and a pharmaceutically acceptable carrier. The pharmaceutical composition may contain between about 0.01 mg and about 1,000 mg of the compound, or any amount or range therein; preferably about 0.1 mg to about 500 mg of the compound, or any amount or range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I), as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of the present invention may be administered in any of the foregoing compositions and according to dosage regimens established in the art whenever treatment of disorders mediated by TRPM8 is required.

The daily dosage of the products may be varied over a wide range from about 0.01 mg to about 1,000 mg per adult human per day, or any amount or range therein. For oral administration, the compositions are preferably provided in the form of tablets containing about 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.01 mg/kg to about 300 mg/kg of body weight per day, or any amount or range therein. Preferably, the range is from about 0.1 mg/kg/day to about 50.0 mg/kg of body weight per day, or any amount or range therein. More preferably, the range is from about 0.1 mg/kg/day to about 10 mg/kg/day, or any amount or range therein. More preferably, the range is from about 0.1 mg/kg/day to about 5 mg/kg/day, or any amount or range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder. One skilled in the art will further recognize that human clinical trials including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

In the Examples which follow herein, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

Examples A through AA, which follow herein, describe the synthesis of intermediates used in the synthesis of representative compounds of formula (I).

### Example A

### 5-tert-Butyl-2-methyl-2H-pyrazole-3-carboxylic acid

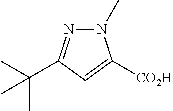

### STEP A. 5-tert-Butyl-2-methyl-2H-pyrazole-3-carboxylic acid ethyl ester

Ethyl 5,5-dimethyl-2,4-dioxo-hexanoate (1.02 g, 5.09 mmol) was dissolved in absolute EtOH (20 mL). CH₃NHNH₂ (0.270 mL, 5.09 mmol) was added dropwise and the resulting mixture was stirred at room temperature for 2 h. The resulting mixture was warmed to 80° C. for 4 h, and then cooled to room temperature. The solvent was removed under reduced pressure, and the resulting residue was chromatographed using a 70-g pre-packed SiO₂ column eluting with 1:19 EtOAc-hexanes to yield a residue. ¹H-NMR (400 MHz, CDCl₃) δ: 6.68 (s, 1H), 4.33 (q, J=7.2 Hz, 2H), 4.12 (s, 3H), 1.38 (t, J=7.1 Hz, 3H), 1.30 (s, 9H).

### STEP B. 5-tert-Butyl-2-methyl-2H-pyrazole-3-carboxylic acid

5-tert-Butyl-2-methyl-2H-pyrazole-3-carboxylic acid ethyl ester (1.08 g, 5.14 mmol, prepared as described in STEP A above) was dissolved in MeOH (15 mL), and H₂O (15 mL) and 2.5 M aqueous NaOH (5.00 mL, 12.5 mmol) was added. The resulting mixture was stirred at room temperature for 72 h, and then extracted with Et₂O (2×10 mL). The aqueous layer was acidified to ca. pH 2 using 3 M aqueous HCl and extracted with DCM (3×20 mL). The combined organic extracts were dried over anhydrous MgSO₄ and filtered, then the solvent was removed under reduced pressure to yield the title compound as a white solid.

¹H-NMR (400 MHz, CDCl₃) δ: 6.80 (s, 1H), 4.15 (s, 3H), 1.32 (s, 9H).

### Example B

### 5-tert-Butyl-2-ethyl-2H-pyrazole-3-carboxylic acid

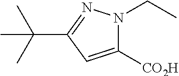

The title compound was prepared following the procedure described in Example A, and selecting and substituting reagents, starting materials, and conditions as would be known to those skilled in the art.

### Example C

### 5-tert-Butyl-2-isopropyl-2H-pyrazole-3-carboxylic acid

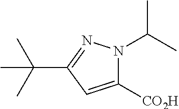

The title compound was prepared following the procedure described in Example A, and selecting and substituting reagents, starting materials, and conditions as would be known to those skilled in the art.

### Example D

### 5-tert-Butyl-2-benzyl-2H-pyrazole-3-carboxylic acid

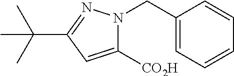

The title compound was prepared following the procedure described in Example A, and selecting and substituting reagents, starting materials, and conditions as would be known to those skilled in the art.

### Example E

### 3-tert-Butyl-1,4-dimethyl-1H-pyrazole-5-carboxylic acid

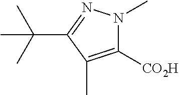

### STEP A. 3-tert-Butyl-1,4-dimethyl-1H-pyrazole-5-carboxylic acid ethyl ester

To a solution of 1.0 M lithium hexamethyldisilazide in hexanes (11.1 mL, 11.1 mmol) in THF (50 mL) at −78° C., 2,2-dimethylpentan-3-one (1.06 g, 9.25 mmol) was added dropwise. The reaction was stirred at −78° C. for 1 hour, then diethyl oxalate (1.2 eq., 1.5 mL, 11.1 mmol) was added dropwise to the resulting mixture. The resulting mixture was stirred at −78° C. for 10 minutes, then warmed to room temperature and stirred for 1 hour. The solvent was removed under reduced pressure. The resulting residue was dissolved in EtOAc (50 mL) and acidified with 10% citric acid solution (50 mL). The aqueous layer was extracted with EtOAc (3×10 mL), then the combined organic extracts were dried over MgSO₄, filtered, and the solvent removed under reduced pressure to yield 3,5,5-trimethyl-2,4-dioxo-hexanoic acid ethyl ester as a residue.

To a solution of the 3,5,5-trimethyl-2,4-dioxo-hexanoic acid ethyl ester (1.04 g, 4.87 mmol, prepared as described above) in anhydrous EtOH (20 mL) was added methylhydrazine (0.26 mL, 4.87 mmol). The resulting mixture was stirred at 80° C. for 12 hours, then cooled to room temperature. The solvent was removed under reduced pressure and the resulting residue was purified on silica eluting with 0:1 EtOAc/hexanes to 1:5 EtOAc/hexanes over 30 minutes to yield a residue. ¹H NMR (400 MHz, CDCl₃) δ: 4.36 (q, J=7.1 Hz, 2H), 4.05 (s, 3H), 2.37 (s, 3H), 1.39 (t, J=7.2 Hz, 3H), 1.35 (s, 9H).

### STEP B. 3-tert-Butyl-1,4-dimethyl-1H-pyrazole-5-carboxylic acid

To a solution of 3-tert-butyl-1,4-dimethyl-1H-pyrazole-5-carboxylic acid ethyl ester (401 mg, 1.79 mmol, as prepared in step A) in MeOH (5 mL) and 1,4-dioxane (5 mL), was added 2.5 M NaOH solution (2 mL, 5.0 mmol). The resulting mixture was then stirred at room temperature for 72 hours, then extracted with Et₂O (2×10 mL). The aqueous layer was acidified ca. to pH 2 using 3 M HCl and extracted with DCM (3×20 mL) and the combined organic extracts were dried over anhydrous MgSO₄, filtered. The solvent was removed under reduced pressure to yield the title compound.

¹H NMR (400 MHz, CDCl₃) δ: 11.7 (br s, 1H), 4.10 (s, 3H), 2.43 (s, 3H), 1.37 (s, 9H).

### Example F

### 3-tert-Butylisoxazole-5-carboxylic acid

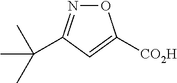

### STEP A. 3-tert-Butylisoxazole-5-carboxylic acid methyl ester

Pivaldehyde (1.10 mL, 10.0 mmol) was dissolved in dry DMF (10 mL), and NH₂OH.H₂O (0.590 mL of 55 wt % aqueous solution, 10.5 mmol) was added. The resulting mixture was stirred at room temperature for 4 h, then NCS (1.40 g, 10.5 mmol) was added in small portions, and the resulting mixture was stirred at room temperature for 1 h. CuSO₄.5H₂O (75.0 mg, 0.300 mmol), methyl propiolate (1.07 mL, 12.0 mmol), and H₂O (5 mL) were added, followed by Cu powder (25.0 mg, 0.393 mmol). The resulting mixture was stirred at room temperature for 16 h, then quenched with dilute aqueous NH₄OH (2 mL). The aqueous solution was extracted with hexanes (3×30 mL), and the combined organic extracts were dried over MgSO₄ and filtered. The solvent was removed under reduced pressure to yield a residue. ¹H-NMR (400 MHz, CDCl₃) δ: 6.88 (s, 1H), 3.96 (s, 3H), 1.36 (s, 9H).

### STEP B. 3-tert-Butylisoxazole-5-carboxylic acid

Following the procedure described in Example A, Step B, the title compound was prepared from 3-tert-butylisoxazole-5-carboxylic acid methyl ester (1.68 g, 9.19 mmol, prepared as described in the previous step) and 2.5 M aqueous NaOH (5.00 mL, 12.5 mmol).

¹H-NMR (400 MHz, CDCl₃) δ: 6.99 (s, 1H), 1.38 (s, 9H).

### Example G

### 3-Isobutylisoxazole-5-carboxylic acid

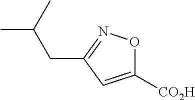

### STEP A. 3-Isobutylisoxazole-5-carboxylic acid methyl ester

To a solution of hydroxylamine hydrate (1.05 eq., 0.64 mL of 50 wt % aqueous solution, 10.5 mmol) in t-butanol (15 mL and H₂O (15 mL) was added 3-methylbutyraldehyde (1.07 mL, 10 mmol). The resulting mixture was stirred at room temperature for 2 hours, then Chloramine-T (also known as N-chloro tosylamide sodium salt) (2.96 g, 10.5 mmol) was added as a solid in small portions over 5 minutes. CuSO₄.5H₂O (0.3 eq., 75 mg, 0.3 mmol) and Cu powder (25 mg) were added as solids, followed by addition of methyl propiolate (1.05 eq., 0.93 mL, 10.5 mmol). The pH of the resulting mixture was adjusted ca. to pH 6 using 1 M NaOH. The resulting mixture was stirred at room temperature for 18 hours, then poured into ice water (550 mL) containing dilute NH₄OH solution (10 mL). The aqueous solution was extracted with DCM (3×30 mL), the combine organic extracts were dried over MgSO₄, filtered, and the solvent removed under reduced pressure. The resulting residue was purified using a 2000μ SiO₂ Prep plate developed with 1:9 EtOAc/hexanes to yield a residue. ¹H NMR (400 MHz, CDCl₃) δ: 6.80 (s, 1H), 3.96 (s, 3H), 2.61 (d, J=7.1 Hz, 2H), 1.92-2.04 (m, 1H), 0.97 (d, J=6.8 Hz, 6H).

### STEP B: 3-Isobutylisoxazole-5-carboxylic acid

The title compound was prepared following the procedure as described in Example A, Step B, reacting the 3-Isobutylisoxazole-5-carboxylic acid methyl ester prepared in STEP A above.

¹H NMR (400 MHz, CDCl₃) δ: 6.88 (s, 1H), 2.63 (d, J=7.1 Hz, 2H), 1.92-2.08 (m, 1H), 0.98 (d, J=6.6 Hz, 6H).

### Example H

### 3-n-Propyl-isoxazole-5-carboxylic acid

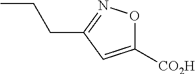

The title compound was prepared following the procedure described in Example G, and selecting and substituting reagents, starting materials, and conditions as would be known to those skilled in the art.

### Example I

### 3-(2,2-Dimethyl-propyl)-isoxazole-5-carboxylic acid

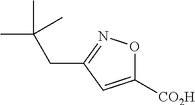

The title compound was prepared following the procedure described in Example G, and selecting and substituting reagents, starting materials, and conditions as would be known to those skilled in the art.

### Example J

### 3-Cyclohexyl-isoxazole-5-carboxylic acid

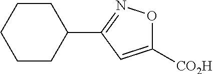

The title compound was prepared following the procedure described in Example G, and selecting and substituting reagents, starting materials, and conditions as would be known to those skilled in the art.

### Example K

### 3-Cyclohexyl-isoxazole-5-carboxylic acid

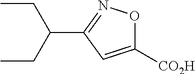

The title compound was prepared following the procedure described in Example G, and selecting and substituting reagents, starting materials, and conditions as would be known to those skilled in the art.

### Example L

### 3-Cyclopropyl-isoxazole-5-carboxylic acid

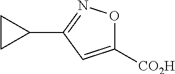

The title compound was prepared following the procedure described in Example G, and selecting and substituting reagents, starting materials, and conditions as would be known to those skilled in the art.

### Example M

### 3-Cyclopentyl-isoxazole-5-carboxylic acid

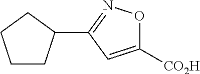

The title compound was prepared following the procedure described in Example G, and selecting and substituting reagents, starting materials, and conditions as would be known to those skilled in the art, the compound in Example M was prepared.

### Example N

### 2-Methyl-6,6-spirocyclohexyl-2,4,5,6-tetrahydro-cyclopentapyrazole-3-carboxylic acid

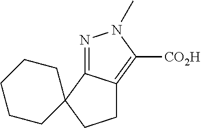

### STEP A. 2-Methyl-2H-6,6-spirocyclohexyl-2,4,5,6-tetrahydro-cyclopentapyrazole-3-carboxylic acid ethyl ester

To a solution of NaOCH₂CH₃ in EtOH (2.5 mL, 6.5 mmol, 21% in EtOH) at −10° C. was added a mixture of spiro[4.5]decan-1-one (432 mg, 2.83 mmol, prepared according to the general procedure described on page 7225 of MOLANDER, G. A., et al., “Intramolecular Nucleophilic Acyl Substitution Reactions of Halo-Substituted Esters and Lactones. New Applications of Organosamarium Reagents”, J. Org. Chem., 1993, pp 7216-7227, Vol. 58) and diethyloxalate (0.85 mL, 6.2 mmol) in EtOH (5 mL). After 15 min, the resulting mixture was allowed to warm to room temperature and then stirred for 6 h. The resulting mixture was treated with 1 N aqueous HCl (10 mL), and then extracted thrice with 20 mL of DCM. The organic layers were combined, dried (Na₂SO₄), and concentrated. The resulting residue was dissolved in EtOH (10 mL) and HOAc (2 mL). To this mixture was added, dropwise, anhydrous hydrazine (0.46 mL, 14 mmol). The resulting mixture was stirred at room temperature overnight. Water (20 mL) was added, and the resulting mixture was extracted twice with 20 mL of EtOAc. The organic layers were combined, dried (Na₂SO₄), and concentrated. The resulting residue was purified on silica (0:100-100:0 EtOAc-hexanes) to yield 6,6-spirocyclohexyl-2,4,5,6-tetrahydro-cyclopentapyrazole-3-carboxylic acid ethyl ester. ¹H-NMR (400 MHz, CDCl₃) δ: 4.34 (q, J=7.2 Hz, 2H), 2.72-2.79 (m, 2H), 2.22-2.29 (m, 2H), 1.40-1.80 (m, 13H), 1.35 (t, J=7.2 Hz, 3H).

To a solution of 6,6-spirocyclohexyl-2,4,5,6-tetrahydro-cyclopentapyrazole-3-carboxylic acid ethyl ester (185 mg, 0.747 mmol, as prepared above) in DMF (10 mL) was added K₂CO₃ (206 mg, 1.49 mmol). The resulting mixture was stirred for 10 min and then treated with CH₃I (0.046 mL, 1.49 mmol). The resulting mixture was stirred overnight, then poured into water (10 mL). The resulting mixture was extracted thrice with EtOAc (20 mL). The organic layers were combined, dried (Na₂SO₄), and concentrated. The resulting residue was purified on silica gel (0:100-100:0 EtOAc-hexanes) to yield a residue. ¹H-NMR (400 MHz; CDCl₃) δ: 4.22 (q, J=7.1 Hz, 2H), 4.05 (s, 3H), 2.66 (t, J=7.1 Hz, 2H), 2.12-2.17 (m, 2H), 1.59-1.72 (m, 4H), 1.36-1.48 (m, 6H), 1.27 (t, J=7.1 Hz, 3H).

### STEP B: 2-Methyl-2H-6,6-spirocyclohexyl-2,4,5,6-tetrahydro-cyclopentapyrazole-3-carboxylic acid

To a solution of 2-methyl-2H-6,6-spirocyclohexyl-2,4,5,6-tetrahydro-cyclopentapyrazole-3-carboxylic acid ethyl ester (prepared as described in the previous step, 124 mg, 0.472 mmol) in MeOH (6 mL) was added LiOH (56.5 mg, 2.36 mmol) followed by water (2 mL). The resulting mixture was stirred at reflux overnight. The resulting mixture was then allowed to cool to room temperature, and MeOH was removed in vacuo. The resulting mixture was acidified with 1 N aqueous HCl, and then extracted thrice with DCM (30 mL). The combined organic layers were dried (Na₂SO₄) and concentrated to yield the title compound as a white solid.

¹H-NMR (400 MHz, CDCl₃) δ: 10.77 (br s, 1H), 4.17 (s, 3H), 2.81 (t, J=7.1 Hz, 2H), 2.13-2.35 (m, 2H), 1.64-1.83 (m, 4H), 1.39-1.62 (m, 6H).

### Example O

### 1-Methyl-3-(1-methyl-cyclopropyl)-1H-pyrazole-5-carboxylic acid

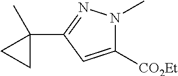

To a solution of 21 wt % sodium ethoxide in EtOH (4.85 mL, 13.0 mmol) in anhydrous toluene (13.3 mL) was added diethyl oxalate (1.22 mL, 10.0 mmol). Methyl 1-methylcyclopropyl ketone (1.10 mL, 10.0 mmol) was subsequently added and the resulting mixture was stirred at room temperature for 24 hours. The resulting mixture was acidified to pH 5 using 6 M HCl then the aqueous phase was extracted with EtOAc (3×40 mL). The combined organic extracts were dried over Na₂SO₄, filtered, and the solvent removed under reduced pressure.

The resulting residue was dissolved in absolute ethanol (100 mL) and then methylhydrazine (0.74 mL, 14.0 mmol) was added dropwise. The resulting mixture was heated to reflux for 16 hours, then cooled to room temperature. The resulting mixture was concentrated under reduced pressure. The resulting residue was purified on silica eluting with 1:4 EtOAc/hexanes to yield the title compound.

¹H NMR (400 MHz, CDCl₃) δ: 6.58 (s, 1H), 4.32 (q, J=7.1 Hz, 2H), 4.10 (s, 3H), 1.44 (s, 3H), 1.37 (t, J=7.1 Hz, 3H), 0.91-1.02 (m, 2H), 0.69-0.77 (m, 2H).

### Example P

### 5-Cyclobutyl-1-Methyl-3-(1-methyl-cyclopropyl)-1H-pyrazole-5-carboxylic acid and 3-cyclobutyl-1-methyl-1H-pyrazole-5-carboxylic acid

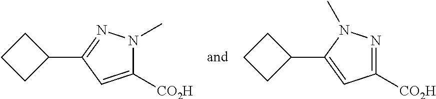

The title compounds were prepared following the procedure described in Example O, and selecting and substituting reagents, starting materials, and conditions as would be known to those skilled in the art. The compounds were prepared as a mixture, then separated by chromatography prior to ester hydrolysis, as would be readily understood by one skilled in the art.

### Example Q

### 2,6,6-Trimethyl-2,4,5,6-tetrahydro-cyclopentapyrazole-3-carboxylic acid and 1,6,6-Trimethyl-1,4,5,6-tetrahydro-cyclopentapyrazole-3-carboxylic acid

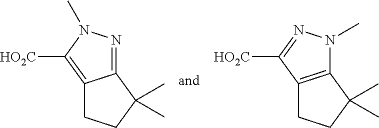

### STEP A. 2,6,6-Trimethyl-2,4,5,6-tetrahydro-cyclopentapyrazole-3-carboxylic acid ethyl ester (X) and 1,6,6-trimethyl-1,4,5,6-tetrahydro-cyclopentapyrazole-3-carboxylic acid ethyl ester (Y)

A solution of 6,6-dimethyl-2,4,5,6-tetrahydro-cyclopentapyrazole-3-carboxylic acid ethyl ester (prepared as described on page 12, Example 1, in FANG, Q. K., et al., US Patent Publication 2005/143443 A1, Published Jun. 30, 2005, 7.65 g, 10.9 mmol), Bu₄NBr (30 mg) and K₂CO₃ (2.29 g, 16.5 mmol) in DMF (20 mL) was treated with CH₃I (0.95 mL, 15 mmol). The resulting mixture was stirred at room temperature overnight, then poured on to water (50 mL). The aqueous mixture was extracted with thrice with diethyl ether (100 mL). The combined organic layers were dried over MgSO₄ and concentrated in vacuo. The resulting residue was purified on silica (0:100-100:0 EtOAc:hexane over 20 min) to yield 2,6,6-trimethyl-2,4,5,6-tetrahydro-cyclopentapyrazole-3-carboxylic acid ethyl ester ¹H-NMR (400 MHz, CDCl₃) δ: 4.31 (q, J=7.1 Hz, 2H), 4.12 (s, 3H), 2.76 (t, J=6.9 Hz, 2H), 2.22 (t, J=6.9 Hz, 2H), 1.36 (t, J=7.1 Hz, 3H), 1.31 (s, 6H) and 1,6,6-trimethyl-1,4,5,6-tetrahydro-cyclopentapyrazole-3-carboxylic acid ethyl ester ¹H-NMR (400 MHz; CDCl₃) δ: 4.36 (q, J=7.2 Hz, 2H), 3.86 (s, 3H), 2.70-2.77 (m, 2H), 2.38 (t, J=6.8 Hz, 2H), 1.37 (t, J=7.1 Hz, 3H), 1.34 (s, 6H).

### STEP B. 2,6,6-Trimethyl-2,4,5,6-tetrahydro-cyclopentapyrazole-3-carboxylic acid and 1,6,6-trimethyl-1,4,5,6-tetrahydro-cyclopentapyrazole-3-carboxylic acid

Following the procedure described in Example A, Step B, the title compounds were individually prepared from 2,6,6-trimethyl-2,4,5,6-tetrahydro-cyclopentapyrazole-3-carboxylic acid ethyl ester and 1,6,6-trimethyl-1,4,5,6-tetrahydro-cyclopentapyrazole-3-carboxylic acid ethyl ester (as prepared in the previous step) and isolated as off-white solids.

### 2,6,6-Trimethyl-2,4,5,6-tetrahydro-cyclopentapyrazole-3-carboxylic acid

¹H-NMR (400 MHz; CD₃OD) δ (ppm): 4.06 (s, 3H), 2.77 (t, J=7.1 Hz, 2H), 2.24 (t, J=6.9 Hz, 2H), 1.28 (s, 6H)

### 1,6,6-trimethyl-1,4,5,6-tetrahydro-cyclopentapyrazole-3-carboxylic acid

¹H-NMR (400 MHz; CD₃OD) δ (ppm): 3.84 (s, 3H), 2.71 (d, J=7.1 Hz, 2H), 2.39 (d, J=7.1 Hz, 2H), 1.36 (s, 6H).

### Example R

### 2,7,7-trimethyl-4,5,6,7-tetrahydro-2H-indazole-3-carboxylic acid

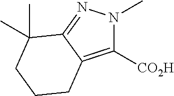

The title compound was prepared following the procedure described in Example O, and selecting and substituting reagents, starting materials, and conditions as would be known to those skilled in the art.

### Example S

### 2′-Methyl-2′,4′,5′,6′-tetrahydrospiro[cyclohexane-1,7′-indazole]-3′-carboxylic acid

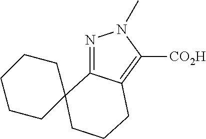

The title compound was prepared following the procedure described in Example N, and selecting and substituting reagents, starting materials and conditions, as would be known to those skilled in the art.

### Example T

### 5-tert-Butyl-4-chloro-2-methyl-2H-pyrazole-3-carboxylic acid

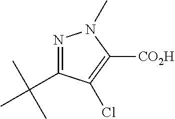

### STEP A. 5-tert-Butyl-4-chloro-2-methyl-2H-pyrazole-3-carboxylic acid ethyl ester

To a solution of 5-tert-butyl-2-methyl-pyrazole-3-carboxylic acid ethyl ester (2.10 g, 10.0 mmol) in DCM (25 mL) was added sulfuryl chloride (1.05 mL, 13.0 mmol) slowly under Ar. After stirring at room temperature for 3 h under Ar, the resulting mixture was treated with DCM (30 mL), washed with ice H₂O, saturated aqueous NaHCO₃ and brine, then dried with Na₂SO₄. The solvent was then removed under reduced pressure to yield a white solid. ¹H-NMR (400 MHz, CDCl₃) δ: 4.40 (q, J=7.2 Hz, 2H), 4.07 (s, 3H), 1.42 (t, J=7.2 Hz, 3H), 1.40 (s, 9H). Mass Spectrum (LCMS, ESI pos.) Calculated for C₁₁H₁₇ClN₂O₂: 245.1 (M+H), Measured: 245.1.

### STEP B. 5-tert-Butyl-4-chloro-2-methyl-2H-pyrazole-3-carboxylic acid

A mixture of 5-tert-butyl-4-chloro-2-methyl-2H-pyrazole-3-carboxylic acid ethyl ester (prepared as described in the previous step, 2.20 g, 9.00 mmol) and 3 N aqueous NaOH (7.50 mL, 22.5 mmol) in MeOH (40 mL) was stirred at room temperature for 4 h. The solvent was removed under reduced pressure, and the resulting residue was treated with H₂O (30 mL) and washed with Et₂O. The aqueous layer was then acidified to pH 7 by 2 N aqueous HCl and then extracted with DCM. The combined organic layers were washed with brine and dried with Na₂SO₄. The solvent was removed in vacuo to yield the title compound as a white solid.

¹H-NMR (400 MHz, CDCl₃) δ: 10.41 (br s, 1H), 4.12 (s, 3H), 1.42 (s, 9H). Mass Spectrum (LCMS, ESI pos.) Calculated for C₉H₁₃ClN₂O₂: 217.1 (M+H), Measured: 217.1.

### Example U

### 3-(tert-butyl)-4-methylisoxazole-5-carboxylic acid

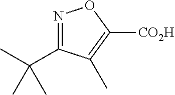

### STEP A. 4-Bromo-3-tert-butyl-isoxazole-5-carboxylic acid methyl ester

A mixture of 3-tert-butyl-isoxazole-5-carboxylic acid methyl ester (1.00 g, 5.46 mmol, as prepared in example F, step A) and NBS (1.46 g, 8.19 mmol) was stirred at 150° C. for 0.5 h under microwave radiation. After cooling to room temperature, the resulting mixture was concentrated in vacuo and the residue was purified by silica chromatography (0-5% EtOAc/hexanes) to yield a colorless oil (1.09 g, 76%). ¹H-NMR (400 MHz, CDCl₃) δ: 3.98 (s, 3H), 1.47 (s, 9H). Mass Spectrum (LCMS, ESI pos.) Calcd. For C₉H₁₂BrNO₃: 262.0 (M+H), Found 262.0.

### STEP B. 3-tert-Butyl-4-methyl-isoxazole-5-carboxylic acid methyl ester

To a solution of 4-bromo-3-tert-butyl-isoxazole-5-carboxylic acid methyl ester (as prepared in the previous step, 865 mg, 3.30 mmol) in THF (30 mL) at −78° C. under Ar was added n-butyllithium (3.09 mL, 4.95 mmol, 1.6 M in THF) and the resulting mixture was stirred at −78° C. for 0.5 h. CH₃I (937 mg, 6.60 mmol) was added, and the resulting mixture was warmed to room temperature and then stirred for 1 h under Ar. The resulting mixture was then treated with saturated NH₄Cl (10 mL) followed by H₂O (50 mL). The resulting mixture was then extracted with EtOAc (3×50 mL). The combined organic layers were washed with H₂O (30 mL), brine (30 mL) and dried (Na₂SO₄). The solvent was removed under reduced pressure followed by flash chromatography of the resulting residue on silica gel (0-4% EtOAc/hexane) to yield a residue. ¹H-NMR (400 MHz, CDCl₃) δ: 3.95 (s, 3H), 2.43 (s, 3H), 1.41 (s, 9H). Mass Spectrum (LCMS, ESI pos.) Calculated for C₁₀H₁₅NO₃: 198.1.0 (M+H), Measured: 198.1.

### STEP C. 3-tert-Butyl-4-methyl-isoxazole-5-carboxylic acid

Following the procedure as described in Example A, Step B, the title compound was prepared by reacting 3-tert-butyl-4-methyl-isoxazole-5-carboxylic acid methyl ester (as prepared in the previous step, 260 mg, 1.32 mmol) and 1.0 N NaOH (1.58 mL, 1.58 mmol) in MeOH (5 mL).

¹H-NMR (400 MHz, CDCl₃) δ: 9.88 (none, 1H), 2.46 (s, 3H), 1.42 (s, 9H). Mass Spectrum (LCMS, ESI pos.) Calculated for C₉H₁₃NO₃: 184.1 (M+H), Measured: 184.1.

### Example V

### 5-tert-Butyl-4-cyano-2-methyl-2H-pyrazole-3-carboxylic acid

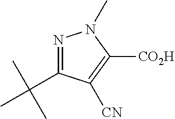

### STEP A. 4-Bromo-5-tert-butyl-2-methyl-2H-pyrazole-3-carboxylic acid ethyl ester

To a mixture of 5-tert-butyl-2-methyl-pyrazole-3-carboxylic acid ethyl ester (2.00 g, 9.51 mmol) and K₂CO₃ (3.94 g, 28.5 mmol) in DCM (120 mL), in the dark, was added Br₂ (1.46 mL, 28.5 mmol) slowly under Ar. After stirring at room temperature for 3 h under Ar, the resulting mixture was quenched with saturated aqueous Na₂S₂O₃ (50 mL). The organic layer was separated and washed with H₂O (50 mL) and brine (50 mL), then dried with Na₂SO₄. The solvent was removed under reduced pressure to yield a residue. ¹H-NMR (400 MHz, CDCl₃) δ: 4.40 (q, J=7.1 Hz, 2H), 4.08 (s, 3H), 1.43 (t, J=7.1 Hz, 3H), 1.42 (m, 9H). Mass Spectrum (LCMS, ESI pos.) Calculated For C₁₁H₁₇BrN₂O₂: 289.1 (M+H), Measured: 289.1.

### STEP B. 5-tert-Butyl-4-cyano-2-methyl-2H-pyrazole-3-carboxylic acid ethyl ester

A mixture of 4-bromo-5-tert-butyl-2-methyl-2H-pyrazole-3-carboxylic acid ethyl ester (1.00 g, 3.46 mmol, as prepared in the previous step) and CuCN (372 mg, 4.15 mmol) in NMP (10 mL) was stirred at 200° C. under microwave irradiation for 1 h. After cooling to room temperature, the resulting mixture was treated with DCM (100 mL) and filtered through diatomaceous earth. The filtrate was concentrated in vacuo, and the residue was purified by flash chromatography on silica gel (0:100-10:90 EtOAc-hexanes) to yield a residue. ¹H-NMR (400 MHz, CDCl₃) δ: 4.45 (q, J=7.2 Hz, 2H), 4.14 (s, 3H), 1.45 (t, J=7.2 Hz, 3H), 1.43 (s, 9H). Mass Spectrum (LCMS, ESI pos.) Calculated for C₁₂H₁₇N₃O₂: 236.1 (M+H), Measured: 236.1.

### STEP C: 5-tert-Butyl-4-cyano-2-methyl-2H-pyrazole-3-carboxylic acid

Following the procedure described in Example A, Step B, the title compound was prepared by reacting 5-tert-butyl-4-cyano-2-methyl-2H-pyrazole-3-carboxylic acid ethyl ester (prepared as described in the previous step, 610 mg, 2.59 mmol) and 1.0 N aqueous NaOH (4.00 mL, 4.00 mmol) in MeOH (10 mL).

¹H-NMR (400 MHz, CDCl₃) δ: 4.17 (s, 3H), 1.45 (s, 9H).

### Example W

### 3-(tert-Butyl)-1-methyl-4-(trifluoromethyl)-1H-pyrazole-5-carboxylic acid

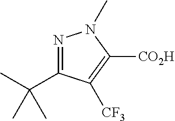

### STEP A. 5-tert-Butyl-2-methyl-4-trifluoromethyl-2H-pyrazole-3-carboxylic acid ethyl ester

A mixture of 4-bromo-5-tert-butyl-2-methyl-2H-pyrazole-3-carboxylic acid ethyl ester (as prepared in Example U, Step A, 1.00 g, 3.46 mmol), CF₃CO₂Na (941 mg, 6.92 mmol) and CuI (1.32 g, 6.92 mmol) in 2:1 DMF/toluene (15 mL) was stirred at 170° C. for 16 h under Ar. After cooling to room temperature, the resulting mixture was treated with EtOAc (100 mL) and filtered through a pad of diatomaceous earth. The filtrate was concentrated in vacuo and the resulting residue was purified by flash chromatography on silica gel (0-5% EtOAc/hexane) to yield a residue. ¹H-NMR (400 MHz, CDCl₃) δ: 4.41 (q, J=7.1 Hz, 2H), 3.95 (s, 3H), 1.39 (t, J=7.1 Hz, 3H), 1.36 (s, 9H). Mass Spectrum (LCMS, APCI pos.) Calculated for C₁₂H₁₇F₃N₂O₂: 279.1 (M+H), Measured: 279.1.

### STEP B. 5-tert-Butyl-2-methyl-4-trifluoromethyl-2H-pyrazole-3-carboxylic acid

Following the procedure as described in Example A, Step B, the title compound was prepared by reacting 5-tert-butyl-2-methyl-4-trifluoromethyl-2H-pyrazole-3-carboxylic acid ethyl ester (as prepared in the previous step, 278 mg, 1.00 mmol) and 1.0 N NaOH (1.05 mL, 1.05 mmol) in MeOH/THF (3 mL).

¹H-NMR (400 MHz, CDCl₃) δ: 4.05 (s, 3H), 1.38 (s, 9H).

### Example X

### 2-(tert-Butyl)thiazole-5-carboxylic acid

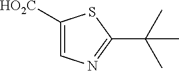

### STEP A. 2-tert-Butyl-thiazole-5-carbaldehyde

2,2-Dimethyl-thiopropionamide (1.17 g, 10.0 mmol, prepared as described on page 297, in BOYS, M, et. al., “Preparation of Primary Thioamides From Nitriles Using Sodium Hydrogen Sulfide and Diethylamine Hydrochloride”, Synth. Commun., 200, pp 295-298) and bromomalonaldehyde (1.51 g, 10.0 mmol) in EtOH (7.0 mL) was stirred at 60° C. for 3 h. The resulting mixture was concentrated and chromatographed on an 80-g silica gel column eluting with a gradient of 0-30% EtOAc-hexane, then held at 30% EtOAc-hexane yield a residue. ¹H-NMR (400 MHz, CDCl₃) δ (ppm): 10.00 (s, 1H), 8.30 (s, 1H), 1.48 (s, 9H). Mass Spectrum (LCMS, ESI pos.) Calcd. for C₈H₁₁NOS: 170.1 (M+H), Found 170.1.

### STEP B. 2-tert-Butyl-thiazole-5-carboxylic acid

A vigorously stirred two-phase mixture of 2-tert-butyl-thiazole-5-carbaldehyde (400 mg, 2.36 mmol, as prepared in the previous step), resorcinol (800 mg, 7.08 mmol), and 20% (w/v) aq NaH₂PO₄ monohydrate (8.14 mL, 11.8 mmol) in tert-butanol (10 mL) was treated with a solution of sodium chlorite (667 mg, 5.90 mmol, technical grade, 80%) in water (2 mL), dropwise over 1.5 min. (3.45 mL, 3.45 mmol) and then stirred at room temperature for 30 min. The resulting mixture was extracted with EtOAc (3×15 mL) and the combined extracts were washed with brine (10 mL), dried (Na₂SO₄), then concentrated. The resulting amber-colored syrup was suspended in brine (20 mL), adjusted to pH 8.5 with saturated aqueous NaHCO₃, and washed with Et₂O (3×20 mL). The aqueous layer was then adjusted to pH 2 with 1M HCl and extracted with Et₂O (3×20 mL). The combined extracts were washed with brine (25 mL), dried (Na₂SO₄), and concentrated to yield the title compound as a residue.

¹H-NMR (400 MHz, CDCl₃) δ (ppm): 8.38 (s, 1H), 1.48 (s, 9H). Mass Spectrum (LCMS, ESI pos.) Calculated for C₈H₁₁NO₂S: 186.1 (M+H), Measured: 186.1.

### Example Y

### 5-tert-Butyl-4-cyano-2-methyl-furan-3-carboxylic acid

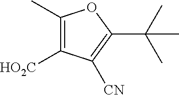

### STEP A. 5-tert-Butyl-4-formyl-2-methyl-furan-3-carboxylic acid

To a solution of 5-tert-butyl-4-hydroxymethyl-2-methyl-furan-3-carboxylic acid (212 mg, 1.00 mmol) in DCM (10 mL), Dess-Martin periodinane (508 mg, 1.20 mmol) was added. The resulting mixture was stirred at room temperature overnight, then diluted with saturated brine (10 mL). The organic layer was separated and the aqueous layer was extracted twice with CH₂Cl₂ (20 mL). The combined organic layers were dried (Na₂SO₄) and concentrated in vacuo. The resulting residue was purified on silica (0:100 to 100:0 EtOAc:hexane over 20 min), then re-purified on silica (0:100-100:0 DCM/hexane over 20 min) to yield a residue. ¹H-NMR (400 MHz; CDCl₃) δ: 10.21 (s, 1H), 2.68 (s, 3H), 1.54 (s, 3H).

### STEP B. 5-tert-Butyl-4-cyano-2-methyl-furan-3-carboxylic acid

To a solution of 5-tert-butyl-4-formyl-2-methyl-furan-3-carboxylic acid (as prepared in the previous step, 650 mg, 3.09 mmol) in formic acid (15 mL). NH₂OH.HCl (429.6 mg, 6.18 mmol) was added. The resulting mixture was heated at reflux overnight, then allowed to cool to room temperature and concentrated in vacuo. The resulting residue was partitioned between water and DCM. The organic layer was separated, dried (Na₂SO₄), and concentrated in vacuo. The resulting residue was purified on silica (0:100-100:0 DCM:hexane) to yield the title compound.

¹H-NMR (400 MHz, CDCl₃) δ: 2.60 (s, 3H), 1.45 (s, 3H).

### Example Z

### 2-(tert-butyl)-5-methyloxazole-4-carboxylic acid

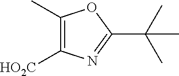

### STEP A. 2-(2,2-Dimethyl-propionylamino)-3-hydroxy-butyric acid methyl ester

To a solution of threonine methyl ester hydrochloride salt (10.0 g, 59 mmol) and TEA (16 mL, 110 mmol) in THF (150 mL) was added 2,2-dimethyl-propionyl chloride (8.0 mL, 65 mmol) dropwise at 0° C. and the resulting mixture stirred for 30 min. The resulting mixture was then allowed to warm up to room temperature. After 18 h, the resulting solution was poured into a mixture of EtOAc/H₂O 1:1 (100 mL). The layers were separated. The organic layer was washed successively with 1N HCl (2×50 mL), water (50 mL), aqueous NaHCO₃ (2×50 mL), H₂O (50 mL), brine (50 mL). The organic layer was dried over Na₂SO₄, filtered, and concentrated to yield a residue. ¹H-NMR (400 MHz, CDCl₃) δ (ppm) 6.3-6.5 (m, 1H), 4.52-4.61 (dd, J=2.5, 8.6 Hz, 1H), 4.32-4.38 (m, 1H), 3.76 (s, 3H), 2.38-2.42 (d, J=4.5 Hz, 1H), 1.24 (s, 9H), 1.18-1.21 (d, J=6.5 Hz, 3H).

### STEP B. 2-tert-Butyl-5-methyl-4,5-dihydro-oxazole-4-carboxylic acid methyl ester

A solution of 2-(2,2-dimethyl-propionylamino)-3-hydroxy-butyric acid methyl ester (5.2 g, 24 mmol, as prepared in previous step) in DCM (75 mL) was cooled to −78° C. under Ar and treated with DAST (3.8 mL, 29 mmol) over a period of 15 min. The solution was stirred at −78° C. for 2 h. To the resulting solution was then added K₂CO₃ (10 g) The resulting solution was stirred at −78° C. for 30 min, then allowed to warm up to room temperature. A solution of aqueous NaHCO₃ (20 mL) was then added slowly followed by H₂O (50 mL). The layers were separated and the organic layer was washed with aqueous NaHCO₃ (50 mL), H₂O (50 mL), brine (50 mL), dried over Na₂SO₄, filtered and concentrated to yield a residue. ¹H-NMR (400 MHz, CDCl₃) δ: (ppm) 4.8-4.9 (m, 1H), 4.7-4.76 (d, J=10.1 Hz, 1H), 3.70 (s, 3H), 1.2-1.3 (m, 12H).

### STEP C. 2-tert-Butyl-5-methyl-oxazole-4-carboxylic acid methyl ester

To a solution of 2-tert-butyl-5-methyl-4,5-dihydro-oxazole-4-carboxylic acid methyl ester (4.6 g, 23 mmol, as prepared in the previous step) and DBU (17 mL, 110 mmol) in DCM (330 mL) was added bromotrichloromethane (23 mL, 230 mmol). The resulting solution was stirred at room temperature for 18 h. The resulting solution was then concentrated and resulting residue was purified on silica EtOAc-hexanes (2:8) to yield a residue. ¹H-NMR (400 MHz, CDCl₃) δ (ppm) 3.86 (s, 3H) 2.60 (s, 3H) 1.38 (s, 9H).

### STEP D: 2-tert-Butyl-5-methyl-oxazole-4-carboxylic acid

To a solution of 2-tert-butyl-5-methyl-oxazole-4-carboxylic acid methyl ester (0.46 g, 2.3 mmol, as prepared in previous step) in MeOH-THF (1:1, 5 mL) was added 1N NaOH (5 mL, 5 mmol). The resulting solution was stirred at room temperature for 18 h. The resulting solution was concentrated and the residue was taken up in H₂O (25 mL), the resulting mixture cooled and the pH adjusted to pH 2 with 1N HCl. The aqueous layer was extracted with EtOAc (2×25 mL). The EtOAc layers were combined and washed with H₂O (25 mL), brine (25 mL), dried over Na₂SO₄, filtered, and concentrated and dried in vacuo to yield the title compound.

¹H-NMR (400 MHz, d6-DMSO) δ: 2.60 (s, 3H), 1.38 (s, 9H).

### Example AA

### 2-(tert-Butyl)-4-methyloxazole-5-carboxylic acid

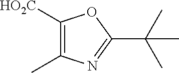

### STEP A. 2-(2,2-Dimethyl-propionyloxy)-3-oxo-butyric acid methyl ester

A solution of pivalic acid (5 g, 50 mmol), methyl-2-chloroacetoacetate (6.0 mL, 49 mmol) and TEA (21 mL, 150 mmol) in EtOAc (200 mL) was stirred at 65° C. for 18 h. The resulting solution was allowed to cool to room temperature and directly purified on silica to yield a residue. ¹H-NMR (400 MHz, CDCl₃) δ: 5.45 (s, 1H), 3.80 (s, 3H), 2.30 (s, 3H), 1.30 (s, 9H).

### STEP B. 2-tert-Butyl-4-methyl-oxazole-5-carboxylic acid methyl ester

A solution of 2-(2,2-dimethyl-propionyloxy)-3-oxo-butyric acid methyl ester (8.8 g, 40.7 mmol, as prepared in previous step) and ammonium trifluoroacetate (54.0 g, 407 mmol) was stirred at 150° C. for 5 min. The resulting mixture was cool to room temperature, then partitioned between EtOAc (50 mL) and H₂O (50 mL). The layers were separated and the organic layer was washed with H₂O (50 mL), concentrated, and dried in vacuo to yield a residue. ¹H-NMR (400 MHz, CDCl₃) δ: 3.90 (s, 3H), 2.44 (s, 3H), 1.40 (s, 9H).

### STEP C. 2-tert-Butyl-4-methyl-oxazole-5-carboxylic acid

A solution of 2-tert-butyl-4-methyl-oxazole-5-carboxylic acid methyl ester (8.0 g, 40.7 mmol, as prepared in previous step) in MeOH (100 mL) was treated with NaOH solution (1 N NaOH, 80 ml, 80 mmol). The resulting solution was stirred at room temperature for 18 h and concentrated. The resulting residue was partitioned between EtOAc and H₂O. The aqueous layer was acidified with 1N HCl to pH 2 and extracted with EtOAc (2×25 mL). The EtOAc layers were combined, washed with H₂O, dried over Na₂SO₄, filtered and concentrated to yield the title compound.

¹H-NMR (400 MHz, CDCl₃) δ: 2.48 (s, 3H), 1.40 (s, 9H).

Examples 1 through 29, which follow herein, describe the synthesis of representative compounds of formula (I) of the present invention.

### Example 1

### 2-(5-tert-Butyl-2-methyl-2H-pyrazol-3-yl)-5-(2-trifluoromethyl-phenyl)-1H-benzimidazole sodium salt (Compound #13)

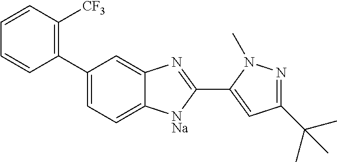

### STEP A. 5-Bromo-2-(5-tert-butyl-2-methyl-2H-pyrazol-3-yl)-1H-benzimidazole

A solution of 5-tert-butyl-2-methyl-2H-pyrazole-3-carboxylic acid (364 mg, 2.00 mmol, as prepared in Example A) was treated with oxalyl chloride (0.260 mL, 3.00 mmol) dropwise via syringe. The resulting mixture was stirred at room temperature for 3 h, and the solvent was removed under reduced pressure. The resulting residue was dissolved in anhydrous THF (5 mL).

The THF solution was added dropwise to a stirred solution of 4-bromo-1,2-diaminobenzene (561 mg, 3.00 mmol) and DIPEA (0.520 mL, 3.00 mmol) in anhydrous THF (10 mL) and the resulting mixture was stirred at room temperature for 16 h. The solvent was removed under reduced pressure. The residue was dissolved in 1,4-dioxane (10 mL), and p-TsOH.H₂O (61.0 mg, 4.00 mmol) was added as a solid. The resulting mixture was stirred at 100° C. for 18 h, cooled to room temperature, and basified to ca. pH 10 using 2.5 M aqueous NaOH. The aqueous layer was extracted with EtOAc (3×10 mL), and then the combined organic extracts were dried over MgSO₄ and filtered. The solvent was removed under reduced pressure, and the resulting residue was chromatographed using a 70-g pre-packed column eluting with 2:3 EtOAc-hexanes to yield a white solid. 1H-NMR (400 MHz, CD₃OD) δ: 7.65 (d, J=1.5 Hz, 1H), 7.39 (d, J=8.6 Hz, 1H), 7.25 (dd, J=8.5, 1.9 Hz, 1H), 6.68 (s, 1H), 4.19 (s, 3H), 1.31 (s, 9H).

### STEP B. 2-(5-tert-Butyl-2-methyl-2H-pyrazol-3-yl)-5-(2-trifluoromethylphenyl)-1H-benzimidazole

To a mixture of 5-bromo-2-(5-tert-butyl-2-methyl-2H-pyrazol-3-yl)-1H-benzimidazole (1.21 g, 3.63 mmol), 2-trifluoromethylphenylboronic acid (1.03 g, 5.45 mmol), and (dppf)PdCl₂.DCM (266 mg, 0.363 mmol) under Ar were added DME (16 mL) and 2 M aqueous Na₂CO₃ (4.00 mL, 8.00 mmol). The resulting mixture was stirred at 90° C. for 12 h and cooled to room temperature, and then the solvent was removed under reduced pressure. The resulting residue was chromatographed using a 90-g SiO₂ pre-packed column eluting with 0:1 to 2:3 EtOAc-hexanes to yield a white solid. ¹H-NMR (400 MHz, CD₃OD) δ: 7.75 (d, J=7.6 Hz, 1H), 7.58-7.71 (m, 2H), 7.55 (t, J=7.2 Hz, 1H), 7.46 (t, J=7.6 Hz, 1H), 7.38 (d, J=7.6 Hz, 1H), 7.22 (d, J=8.3 Hz, 1H), 6.80 (s, 1H), 4.29 (s, 3H), 1.36 (s, 9H).

### STEP C: 2-(5-tert-Butyl-2-methyl-2H-pyrazol-3-yl)-5-(2-trifluoromethylphenyl)-1H-benzimidazole sodium salt

To a solution of 2-(5-tert-butyl-2-methyl-2H-pyrazol-3-yl)-5-(2-trifluoromethylphenyl)-1H-benzimidazole (994 mg, 2.49 mmol) in anhydrous MeOH (10 mL) was added 0.5 M NaOMe in MeOH (5.00 mL, 2.49 mmol). The resulting mixture was shaken for 5 min, and then the solvent was removed under reduced pressure. The residue was triturated with Et₂O (5 mL), and the supernatant was removed by pipette. The solvent was removed under high vacuum to yield the title compound as a white powder.

¹H-NMR (400 MHz, d6-DMSO) δ 7.77 (d, J=7.9 Hz, 1H), 7.65 (t, J=7.4 Hz, 1H), 7.51 (t, J=7.7 Hz, 1H), 7.44 (d, J=7.6 Hz, 1H), 7.37 (d, J=8.1 Hz, 1H), 7.30 (s, 1H), 6.75 (d, J=8.1 Hz, 1H), 6.54 (s, 1H), 4.29 (s, 3H), 1.28 (s, 9H). Mass Spectrum (LCMS, ESI pos.): Calculated for C₂₂H₂₁F₃N₄: 399.2 (M+H); Measured: 399.3.

Following the procedure described in Example 1, and selecting and substituting reagents, starting materials, and conditions as would be known to those skilled in the art, the following compounds of formula (I) of the present invention were prepared:

| ID | |
|---|---|
| No. | Name and Measured Physical Property |
| 12 | 2-(5-tert-Butyl-2-methyl-2H-pyrazol-3-yl)-5-(2-methoxyphenyl)- |
| | 1H-benzimidazole sodium salt |
| | ¹H-NMR (400 MHz, d6-DMSO) δ: 7.45 (d, J = 1.4 Hz, 1H), 7.31 |
| | (d, J = 8.1 Hz, 1H), 7.29 (dd, J = 7.4, 1.7 Hz, 1H), 7.21 (td, J = |
| | 8.2, 1.7 Hz, 1H), 7.04 (d, J = 7.6 Hz, 1H), 6.98 (td, J = 7.4, 0.9 |
| | Hz, 1H), 6.88 (dd, J = 8.2, 1.7 Hz, 1H), 6.49 (s, 1H), 4.29 (s, |
| | 3H), 3.75 (s, 3H), 1.28 (s, 9H) |
| | Mass Spectrum (LCMS, ESI pos.): Calculated for C₂₂H₂₄N₄O: |
| | 361.2 (M + H); Measured: 361.3. |
| 14 | 2-(5-tert-Butyl-2-methyl-2H-pyrazol-3-yl)-5-(2- |
| | trifluoromethoxyphenyl)-1H-benzimidazole sodium salt |
| | ¹H-NMR (400 MHz, d6-DMSO) δ: 7.55 (dd, J = 7.4, 1.4 Hz, 1H), |
| | 7.47 (d, J = 1.6 Hz, 1H), 7.46-7.34 (m, 4H), 6.91 (dd, J = 8.2, 1.7 |
| | Hz, 1H), 6.54 (s, 1H), 4.29 (s, 3H), 1.28 (s, 9H) |
| | Mass Spectrum (LCMS, ESI pos.): Calculated for C₂₂H₂₁F₃N₄O: |
| | 415.2 (M + H); Measured: 415.3. |
| 40 | 2-(5-Trifluoromethyl-2-methyl-2H-pyrazol-3-yl)-5-(2- |
| | trifluoromethylphenyl)-1H-benzimidazole sodium salt |
| | ¹H-NMR (400 MHz, d6-DMSO) δ: 7.78 (d, J = 7.8 Hz, 1H), 7.66 |
| | (t, J = 7.3 Hz, 1H), 7.52 (t, J = 7.6 Hz, 1H), 7.44 (d, J = 8.1 Hz, |
| | 1H), 7.42 (d, J = 8.1 Hz, 1H), 7.35 (s, 1H), 7.01 (s, 1H), 6.78 (d, |
| | J = 8.1 Hz, 1H), 4.46 (s, 3H) |
| | Mass Spectrum (LCMS, ESI pos.): Calculated for C₁₉H₁₂F₆N₄: |
| | 411.1 (M + H); Measured: 411.3. |
| 41 | 2-(5-Trifluoromethyl-2-methyl-2H-pyrazol-3-yl)-5-(2- |
| | trifluoromethoxyphenyl)-1H-benzimidazole sodium salt |
| | ¹H-NMR (400 MHz, d6-DMSO) δ: 7.35-7.59 (m, 6H), 7.02 (s, |
| | 1H), 6.95 (dd, J = 8.2, 1.6 Hz, 1H), 4.47 (s, 3H) |
| | Mass Spectrum (LCMS, ESI pos.): Calculated for C₁₉H₁₂F₆N₄O: |
| | 427.1 (M + H); Measured: 427.3. |
| 50 | 2-(5-tert-Butyl-4-chloro-2-methyl-2H-pyrazol-3-yl)-5-(2- |
| | trifluoromethylphenyl)-1H-benzimidazole sodium salt |
| | ¹H-NMR (400 MHz, d6-DMSO) δ: 7.78 (d, J = 7.8 Hz, 1H), 7.66 |
| | (t, J = 7.3 Hz, 1H), 7.52 (t, J = 7.6 Hz, 1H), 7.44 (d, J = 7.8 Hz, |
| | 2H), 7.36 (s, 1H), 6.79 (d, J = 8.1 Hz, 1H), 4.04 (s, 3H), 1.39 (s, |
| | 9H) |
| | Mass Spectrum (LCMS, ESI pos.): Calculated for C₂₂H₂₀ClF₃N₄: |
| | 433.1 (M + H); Measured: 433.2. |
| 51 | 2-(5-tert-Butyl-4-chloro-2-methyl-2H-pyrazol-3-yl)-5-(2- |
| | trifluoromethoxyphenyl)-1H-benzimidazole sodium salt |
| | ¹H-NMR (400 MHz, d6-DMSO) δ: 7.55 (dd, J = 7.5, 1.1 Hz, 1H), |
| | 7.53 (d, J = 1.3 Hz, 1H), 7.48 (d, J = 8.3 Hz, 1H), 7.36-7.46 (m, |
| | 3H), 6.96 (dd, J = 8.1, 1.8 Hz, 1H), 4.05 (s, 3H), 1.39 (s, 9H) |
| | Mass Spectrum (LCMS, ESI pos.): Calculated for |
| | C₂₂H₂₀ClF₃N₄O: 449.1 (M + H); Measured: 449.2. |
| 70 | 2-(2-tert-Butyl-3-methyl-3H-imidazol-4-yl)-5-(2-fluoro-6-methoxy- |
| | phenyl)-1H-benzimidazole trifluoroacetic acid salt |
| | ¹H-NMR (400 MHz, d6-DMSO) δ: 8.12 (s, 1H), 7.72 (d, 1H, J = |
| | 8.4), 7.61 (s, 1H), 7.44-7.36 (m, 1H), 7.24 (d, 1H, J = 8.7), 7.00 |
| | (d, 1H, J = 8.5), 6.97-6.90 (m, 1H), 4.39 (s, 3H), 3.75 (s, 3H), |
| | 1.55 (s, 9H) |
| | Mass Spectrum (loop, ESI pos.): Calculated for C₂₂H₂₄FN₄O: |
| | 379.2 (M + H); found 379.2. |
| 63 | 2-(3-tert-Butyl-isoxazol-5-yl)-5-(2,6-dimethoxy-phenyl)-1H- |
| | benzimidazole trifluoroacetic acid salt |
| | ¹H-NMR (400 MHz, d6-DMSO) δ: 7.62 (d, 1H, J = 8.4), 7.43 (s, |
| | 1H), 7.32 (t, 1H, J = 8.4), 7.26 (s, 1H), 7.12 (dd, 1H, J = 1.5, |
| | 8.5), 6.77 (d, 2H, J = 8.4), 3.66 (s, 6H), 1.37 (s, 9H) |
| | Mass Spectrum (loop, ESI pos.): Calculated for C₂₂H₂₄N₃O₃: |
| | 378.2 (M + H); found 378.3. |
| 64 | 2-(3-tert-Butyl-isoxazol-5-yl)-5-(2-fluoro-6-methoxy-phenyl)-1H- |
| | benzimidazole trifluoroacetic acid salt |
| | ¹H-NMR (400 MHz, d6-DMSO) δ: 7.69 (d, 1H, J = 8.4), 7.58 (s, |
| | 1H), 7.43-7.36 (m, 1H), 7.28 (s, 1H), 7.26-7.22 (m, 1H), 7.00 (d, |
| | 1H, J = 8.5), 6.93 (t, 1H, J = 8.9), 3.75 (s, 3H), 1.37 (s, 9H) |
| | Mass Spectrum (loop, ESI pos.): Calculated for C₂₁H₂₁FN₃O₂: |
| | 366.2(M + H); found 366.3. |
| 65 | 2-(3-tert-Butyl-isoxazol-5-yl)-5-o-tolyl-1H-benzimidazole |
| | trifluoroacetic acid salt |
| | ¹H-NMR (400 MHz, d6-DMSO) δ: 7.70 (d, 1H, J = 8.3), 7.55 (s, |
| | 1H), 7.35-7.24 (m, 6H), 2.26 (s, 3H), 1.37 (s, 9H) |
| | Mass Spectrum (loop, ESI pos.): Calculated for C₂₁H₂₂N₃O: |
| | 332.2 (M + H); found 332.3. |
| 66 | 2-(3-tert-Butyl-isoxazol-5-yl)-5-(2-fluoro-phenyl)-1H- |
| | benzimidazole trifluoroacetic acid salt |
| | ¹H-NMR (400 MHz, d6-DMSO) δ: 7.79 (s, 1H), 7.75 (d, 1H, J = |
| | 8.5), 7.61 (dt, 1H, J = 1.7, 7.9), 7.50-7.46 (m, 1H), 7.45-7.40 (m, |
| | 1H), 7.37-7.31 (m, 2H), 7.30 (s, 1H), 1.37 (s, 9H) |
| | Mass Spectrum (loop, ESI pos.): Calculated for C₂₀H₁₉FN₃O: |
| | 336.2 (M + H); found 336.2. |
| 67 | 2-(3-tert-Butyl-isoxazol-5-yl)-5-(2,6-difluoro-phenyl)-1H- |
| | benzimidazole trifluoroacetic acid salt |
| | ¹H-NMR (400 MHz, d6-DMSO) δ: 7.76 (d, 1H, J = 8.8), 7.73 (s, |
| | 1H), 7.54-7.45 (m, 1H), 7.38-7.34 (m, 1H), 7.31 (s, 1H), 7.29- |
| | 7.21 (m, 2H), 1.37 (s, 9H) |
| | Mass Spectrum (loop, ESI pos.): Calculated for C₂₀H₁₈F₂N₃O: |
| | 354.1 (M + H); found 354.2. |
| 68 | 2-(3-tert-Butyl-isoxazol-5-yl)-5-(2-chloro-phenyl)-1H- |
| | benzimidazole trifluoroacetic acid salt |
| | ¹H-NMR (400 MHz, d6-DMSO) δ: 7.73 (d, 1H, J = 8.3), 7.68 (d, |
| | 1H, J = 0.9), 7.60 (dd, 1H, J = 1.9, 7.4), 7.52-7.40 (m, 3H), 7.36 |
| | (dd, 1H, J = 1.6, 8.3), 7.30 (s, 1H), 1.37 (s, 9H) |
| | Mass Spectrum (loop, ESI pos.): Calculated for C₂₀H₁₉ClN₃O: |
| | 352.1 (M + H); found 352.2. |
| 69 | 2-[2-(3-tert-Butyl-isoxazol-5-yl)-1H-benzimidazol-5-yl]- |
| | benzonitrile trifluoroacetic acid salt |
| | ¹H-NMR (400 MHz, d6-DMSO) δ: 7.98 (dd, 1H, J = 1.0, 7.8), |
| | 7.86 (s, 1H), 7.84-7.78 (m, 2H), 7.71 (d, 1H, J = 7.8), 7.60 (dt, |
| | 1H, J = 1.0, 7.6), 7.50 (dd, 1H, J = 1.7, 8.4), 7.32 (s, 1H), 1.37 |
| | (s, 9H) |
| | Mass Spectrum (loop, ESI pos.): Calculated for C₂₁H₁₉N₄O: |
| | 343.2 (M + H); found 343.2. |

### Example 2

### 2-(5-tert-Butyl-2-ethyl-2H-pyrazol-3-yl)-5-(2-trifluoromethylphenyl)-1H-benzimidazole sodium salt (Compound #44)

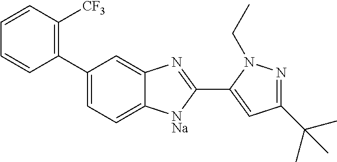

### STEP A. 5-Bromo-2-(5-tert-butyl-2-ethyl-2H-pyrazol-3-yl)-1H-benzimidazole

To a solution of 5-tert-butyl-2-ethyl-2H-pyrazole-3-carboxylic acid (196 mg, 1.00 mmol, prepared as described in example B) and 4-bromo-1,2-diaminobenzene (210 mg, 1.10 mmol) in dry 1,4-dioxane (5 mL) were added DIPEA (0.34 mL, 2.0 mmol) and PyBroP (466 mg, 1.00 mmol). The resulting mixture was stirred at room temperature for 16 h, p-TsOH.H₂O (190 mg, 1.00 mmol) was added as a solid, and the resulting mixture was stirred at 100° C. for 16 h. The resulting mixture was then cooled to room temperature and basified to ca. pH 10 using 2.5 M aqueous NaOH. The aqueous layer was extracted with EtOAc (3×20 mL), and the combined organic extracts were dried over MgSO₄ and filtered. The solvent was removed under reduced pressure, and the resulting residue was chromatographed using a 12-g SiO₂ pre-packed column eluting with 0:1 to 1:4 EtOAc-hexanes to yield a white solid. ¹H-NMR (400 MHz, CD₃OD) δ: 7.74 (br. s., 1H), 7.48 (br. s., 1H), 7.34 (dd, J=8.5, 1.9 Hz, 1H), 6.74 (s, 1H), 4.74 (q, J=7.2 Hz, 4H), 1.42 (t, J=7.1 Hz, 3H), 1.36 (s, 9H).

### STEP B. 2-(5-tert-Butyl-2-ethyl-2H-pyrazol-3-yl)-5-(2-trifluoromethylphenyl)-1H-benzimidazole

Following the procedure described in Step B of Example 1, the title compound was prepared from 5-bromo-2-(5-tert-butyl-2-ethyl-2H-pyrazol-3-yl)-1H-benzimidazole (80.8 mg, 0.233 mmol) and 2-trifluoromethylphenylboronic acid (66.0 mg, 0.350 mmol) to yield a white solid. ¹H-NMR (400 MHz, CD₃OD) δ: 7.78 (d, J=7.8 Hz, 1H), 7.47-7.73 (m, 4H), 7.42 (d, J=7.6 Hz, 1H), 7.22 (d, J=8.3 Hz, 1H), 6.77 (s, 1H), 4.76 (q, J=7.2 Hz, 2H), 1.43 (t, J=7.2 Hz, 3H), 1.37 (s, 9H). Mass Spectrum (LCMS, ESI pos.): Calculated for C₂₃H₂₃F₃N₄: 413.2 (M+H); Measured: 413.3.

### STEP C. 2-(5-tert-Butyl-2-ethyl-2H-pyrazol-3-yl)-5-(2-trifluoromethylphenyl)-1H-benzimidazole sodium salt

To a solution of 2-(5-tert-butyl-2-ethyl-2H-pyrazol-3-yl)-5-(2-trifluoromethylphenyl)-1H-benzimidazole (68.8 mg, 0.167 mmol) in anhydrous MeOH (1 mL) was added 0.5 M NaOMe in MeOH (0.330 mL, 0.167 mmol). The resulting mixture was shaken for 5 min, and the solvent was removed under reduced pressure. The residue was dissolved in Et₂O (2 mL), and then the solvent was removed under high vacuum to yield the title compound as a colorless foam.

¹H-NMR (400 MHz, d6-DMSO) δ: 7.79 (d, J=7.3 Hz, 1H), 7.67 (t, J=7.3 Hz, 1H), 7.54 (t, J=7.7 Hz, 1H), 7.45 (d, J=8.1 Hz, 2H), 7.36 (s, 1H), 6.86 (d, J=8.1 Hz, 1H), 6.64 (s, 1H), 4.85 (q, J=7.1 Hz, 2H), 1.35 (t, J=7.1 Hz, 3H), 1.28 (s, 9H). Mass Spectrum (LCMS, ESI pos.): Calculated for C₂₃H₂₃F₃N₄: 413.2 (M+H); Measured: 413.3.

Following the procedure described in Example 1, and selecting and substituting reagents, starting materials, and conditions as would be known to those skilled in the art, the following compounds of formula (I) of the present invention were prepared:

| ID | |
|---|---|
| No. | Name and Measured Physical Property |
| 45 | 2-(5-tert-butyl-2-ethyl-2H-pyrazol-3-yl)-5-(2- |
| | trifluoromethoxyphenyl)-1H-benzimidazole sodium salt |
| | ¹H-NMR (400 MHz, d6-DMSO) δ: 7.56 (d, J = 6.8 Hz, 1H), 7.50 |
| | (d, J = 1.3 Hz, 1H), 7.35-7.49 (m, 4H), 6.99 (dd, J = 8.1, 1.8 Hz, |
| | 1H), 6.61 (s, 1H), 4.86 (q, J = 7.1 Hz, 2H), 1.35 (t, J = 7.1 Hz, |
| | 3H), 1.28 (s, 9H) |
| | Mass Spectrum (LCMS, ESI pos.): Calculated for C₂₃H₂₃F₃N₄O: |
| | 429.2 (M + H); Measured: 429.3. |

### Example 3

### 2-(5-tert-Butyl-2-isopropyl-2H-pyrazol-3-yl)-5-(2-trifluoromethylphenyl)-1H-benzimidazole sodium salt (Compound #46)

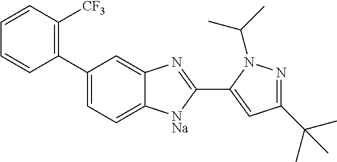

### STEP A. 5-Bromo-2-(5-tert-butyl-2-isopropyl-2H-pyrazol-3-yl)-1H-benzimidazole

To a solution of 5-tert-butyl-2-isopropyl-2H-pyrazole-3-carboxylic acid (210 mg, 1.0 mmol, as prepared in Example C) and 4-bromo-1,2-diaminobenzene (200 mg, 1.07 mmol) in dry 1,4-dioxane (20 mL) was added DCC (227 mg, 1.10 mmol), and the resulting mixture was stirred at room temperature for 16 h. p-TsOH.H₂O (190 mg, 1.00 mmol) was added, and the resulting mixture was stirred at 100° C. for 6 h, cooled to room temperature, and basified ca. to pH 10 using 2.5 M aqueous NaOH. The aqueous layer was extracted with EtOAc (3×20 mL), and the combined organic extracts were dried over MgSO₄ and filtered. The solvent was removed under reduced pressure, and the resulting residue was chromatographed using a 12-g SiO₂ pre-packed column eluting with 0:1 to 2:3 EtOAc-hexanes to yield a white solid. ¹H-NMR (400 MHz, CD₃OD) δ: 7.76 (br. s., 1H), 7.50 (br. s., 1H), 7.36 (dd, J=8.6, 1.8 Hz, 1H), 6.67 (s, 1H), 5.71 (spt, J=6.6 Hz, 1H), 1.49 (d, J=6.6 Hz, 6H), 1.35 (s, 9H).

### STEP B. 2-(5-tert-Butyl-2-isopropyl-2H-pyrazol-3-yl)-5-(2-trifluoromethylphenyl)-1H-benzimidazole

Following the procedure described in Step B of Example 1, the title compound was prepared from 5-bromo-2-(5-tert-butyl-2-isopropyl-2H-pyrazol-3-yl)-1H-benzimidazole (60.7 mg, 0.168 mmol) and 2-trifluoromethylphenylboronic acid (48.0 mg, 0.252 mmol) to yield a white solid. ¹H-NMR (400 MHz, CD₃OD) δ: 7.79 (d, J=7.8 Hz, 1H), 7.64 (t, J=7.2 Hz, 1H), 7.60 (br. s., 2H), 7.55 (t, J=7.7 Hz, 1H), 7.44 (d, J=7.6 Hz, 1H), 7.22 (d, J=8.3 Hz, 1H), 6.69 (s, 1H), 5.69 (spt, J=6.6 Hz, 1H), 1.51 (d, J=6.6 Hz, 6H), 1.36 (s, 9H). Mass Spectrum (LCMS, ESI pos.): Calculated for C₂₄H₂₅F₃N₄: 427.2 (M+H); Measured: 427.2.

### STEP C. 2-(5-tert-Butyl-2-isopropyl-2H-pyrazol-3-yl)-5-(2-trifluoromethylphenyl)-1H-benzimidazole sodium salt

Following the procedure described in Step C of Example 2, the title compound was prepared from 2-(5-tert-butyl-2-isopropyl-2H-pyrazol-3-yl)-5-(2-trifluoromethylphenyl)-1H-benzimidazole (19.1 mg, 45.0 μmol, as prepared in the previous step) and 0.5 M NaOMe in MeOH (90.0 μL, 45.0 μmol) to yield a colorless foam.

¹H-NMR (400 MHz, d6-DMSO) δ: 7.82 (d, J=7.3 Hz, 1H), 7.66-7.75 (m, 1H), 7.56-7.60 (m, 1H), 7.55 (d, J=8.3 Hz, 1H), 7.46 (d, J=7.6 Hz, 1H), 7.43 (s, 1H), 7.00 (d, J=8.1 Hz, 1H), 6.75 (s, 1H), 6.16 (spt, J=6.5 Hz, 1H), 1.43 (d, J=6.6 Hz, 6H), 1.30 (s, 9H). Mass Spectrum (LCMS, ESI pos.): Calculated for C₂₄H₂₅F₃N₄: 427.2 (M+H); Measured: 427.2.

Following the procedure described in Example 3, and selecting and substituting reagents, starting materials and conditions as would be known to those skilled in the art, the following compounds of formula (I) of the present invention were prepared:

| ID | |
|---|---|
| No. | Name and Measured Physical Property |
| 47 | 2-(5-tert-Butyl-2-isopropyl-2H-pyrazol-3-yl)-5-(2- |
| | trifluoromethoxyphenyl)-1H-benzimidazole sodium salt |
| | ¹H-NMR (400 MHz, d6-DMSO) δ: 7.53-7.66 (m, 3H), 7.41-7.53 |
| | (m, 3H), 7.18 (dd, J = 8.5, 0.9 Hz, 1H), 6.77 (s, 1H), 6.15 (spt, J = |
| | 6.6 Hz, 1H), 1.43 (d, J = 6.6 Hz, 6H), 1.30 (s, 9H) |
| | Mass Spectrum (LCMS, ESI pos.): Calculated for C₂₄H₂₅F₃N₄O: |
| | 443.2 (M + H); Measured: 443.2. |
| 48 | 2-(5-tert-Butyl-2-benzyl-2H-pyrazol-3-yl)-5-(2- |
| | trifluoromethylphenyl)-1H-benzimidazole sodium salt |
| | ¹H-NMR (400 MHz, d6-DMSO) δ: 7.77 (d, J = 7.3 Hz, 1H), 7.65 |
| | (t, J = 7.3 Hz, 1H), 7.52 (t, J = 7.6 Hz, 1H), 7.43 (d, J = 7.6 Hz, |
| | 1H), 7.39 (d, J = 8.1 Hz, 1H), 7.31 (s, 1H), 7.20-7.27 (m, 2H), |
| | 7.11-7.20 (m, 3H), 6.79 (d, J = 8.1 Hz, 1H), 6.70 (s, 1H), 6.24 (s, |
| | 2H), 1.28 (s, 9H) |
| | Mass Spectrum (LCMS, ESI pos.): Calculated for C₂₈H₂₅F₃N₄: |
| | 475.2 (M + H); Measured: 475.3. |
| 49 | 2-(5-tert-Butyl-2-benzyl-2H-pyrazol-3-yl)-5-(2- |
| | trifluoromethoxyphenyl)-1H-benzimidazole sodium salt |
| | ¹H-NMR (400 MHz, d6-DMSO) δ: 7.51-7.62 (m, 3H), 7.40-7.51 |
| | (m, 3H), 7.21-7.30 (m, 2H), 7.15-7.21 (m, 3H), 7.09-7.15 (m, |
| | 1H), 6.89 (s, 1H), 6.17 (s, 2H), 1.29 (s, 9H) |
| | Mass Spectrum (LCMS, ESI pos.): Calculated for C₂₈H₂₅F₃N₄O: |
| | 491.2 (M + H); Measured: 491.3. |

### Example 4

### 2-(5-tert-Butyl-2-methyl-2H-pyrazol-3-yl)-5-(2-fluoro-phenyl)-1H-benzimidazole trifluoroacetic acid salt (Compound #57)

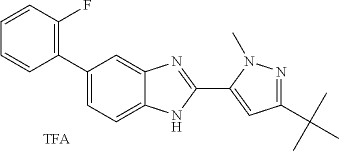

### STEP A. 5-Bromo-2-(5-tert-butyl-2-methyl-2H-pyrazol-3-yl)-1H-benzimidazole

To a solution of 3-tert-butyl-1-methyl-1H-pyrazole-5-carboxylic acid (2.68 g, 14.7 mmol, as prepared in Example A) in acetonitrile (150 mL) was added phosphoryl chloride (7.00 mL, 75.3 mmol). The resulting mixture was heated at reflux for 30 min, 4-bromobenzene-1,2-diamine (2.75 g, 14.7 mmol) was added and refluxing continued for an additional 4.5 h; a second portion of 4-bromobenzene-1,2-diamine (1.39 g, 7.40 mmol) was added. After 17 h additional heating at reflux, the resulting mixture was cooled and diluted into EtOAc (200 mL) and washed with saturated NaHCO₃/brine (1:1, 200 mL) and brine (100 mL). The organics were dried over MgSO₄, filtered and evaporated under vacuum. The residue was chromatographed on a pre-packed 150-g silica gel column eluting with 10:90-25:75 EtOAc-heptane. The product fractions were isolated and evaporated under vacuum. The resulting residue was triturated with 25:75 EtOAc-hexane (20 mL), the solid isolated by filtration and rinsed with 25:75 EtOAc-hexane (7 mL). The filtrate was evaporated under vacuum and the residue was triturated with 25:75 EtOAc-hexane (10 mL). The resulting solid was isolated by filtration, rinsed with 25:75 EtOAc-hexane (2 mL), and combined with the first batch of solid obtained. The combined solid was dried under vacuum at 50° C. for 1.5 h to yield an off-white powder. ¹H-NMR (300 MHz, d6-DMSO) δ: 13.05 (s, 1H), 7.81 (br s, 1H), 7.57 (br s, 1H), 7.37 (dd, J=1.9, 8.7 Hz, 1H), 6.88 (s, 1H), 4.24 (s, 3H), 1.30 (s, 9H). Mass Spectrum (loop, ESI pos.): Calculated for C₁₅H₁₈BrN₄: 333.1 (M+H); Measured: 333.1.

### STEP B. 2-(5-tert-Butyl-2-methyl-2H-pyrazol-3-yl)-5-(2-fluoro-phenyl)-1H-benzimidazole trifluoroacetic acid salt

Following the procedure described in Step B of Example 1, the title compound was prepared from 5-bromo-2-(3-tert-butyl-1-methyl-1H-pyrazol-5-yl)-1H-benzo[d]imidazole (as prepared in the previous step) and 2-fluorophenylboronic acid followed by purification by reverse-phase HPLC (Supelcosil ABZ+Plus column, 5 μm particle size, 25 cm×21.2 mm, at a 15 mL/min flow rate and using a gradient of 25-95% of acetonitrile/0.1% TFA in water/0.1% TFA over 30 minutes).

¹H-NMR (400 MHz, d6-DMSO) δ: 7.76 (s, 1H), 7.71 (d, J=8.4 Hz, 1H), 7.67 (dt, J=1.7, 8.0 Hz, 1H), 7.45-7.39 (m, 2H), 7.37-7.30 (m, 2H), 6.90 (s, 1H), 7.27 (s, 3H), 1.31 (s, 9H). Mass Spectrum (loop, ESI pos.): Calculated for C₂₁H₂₂FN₄: 349.2 (M+H); Measured: 349.3.

Following the procedure described in Example 4, and selecting and substituting reagents, starting materials, and conditions as would be known to those skilled in the art, the following compounds of formula (I) of the present invention were prepared:

| ID | |
|---|---|
| No. | Name and Measured Physical Property |
| 54 | 2-(5-tert-Butyl-2-methyl-2H-pyrazol-3-yl)-5-(2, 6-dimethoxy- |
| | phenyl)-1H-benzimidazole trifluoroacetic acid salt |
| | ¹H-NMR (400 MHz, d6-DMSO) δ: 7.59 (d, J = 8.1 Hz, 1H), |
| | 7.40 (s, 1H), 7.32 (d, J = 8.4 Hz, 1H), 7.08 (dd, J = 1.4, 8.4 |
| | Hz, 1H), 6.87 (s, 1H), 6.76 (d, J = 8.5 Hz, 1H), 4.25 (s, 3H), |
| | 3.66 (s, 6H), 1.31 (s, 9H) |
| | Mass Spectrum (loop, ESI pos.): Calculated for C₂₃H₂₇N₄O₂: |
| | 391.2 (M + H); Measured: 391.3. |
| 55 | 2-(5-tert-Butyl-2-methyl-2H-pyrazol-3-yl)-5-o-tolyl-1H- |
| | benzimidazole trifluoroacetic acid salt |
| | ¹H-NMR (400 MHz, d6-DMSO) δ: 7.66 (d J = 8.3 Hz, 1H), |
| | 7.52 (s, 1H), 7.34-7.25 (m, 4H), 7.20 (s J = 1.5, 8.3 Hz, 1H), |
| | 6.89 (s, 1H), 4.27 (s, 3H), 2.27 (s, 3H), 1.31 (s, 9H) |
| | Mass Spectrum (loop, ESI pos.): Calculated for C₂₂H₂₅N₄: |
| | 345.2 (M + H); Measured: 345.3. |
| 56 | 2-(5-tert-Butyl-2-methyl-2H-pyrazol-3-yl)-5-(2-ethyl-phenyl)- |
| | 1H-benzimidazole trifluoroacetic acid salt |
| | ¹H-NMR (400 MHz, d6-DMSO) δ: 7.67 (d J = 8.4 Hz, 1H), |
| | 7.49 (s, 1H), 7.37-7.30 (m, 2H), 7.29-7.20 (m, 2H), 7.17 (dd, J = |
| | 1.6, 8.3 Hz, 1H), 6.88 (s, 1H), 4.27 (s, 3H), 2.59 (q, J = 7.6 |
| | Hz, 2H), 1.31 (s, 9H), 1.05 (t, J = 7.5 Hz, 3H) |
| | Mass Spectrum (loop, ESI pos.): Calculated for C₂₃H₂₇N₄: |
| | 359.2 (M + H); Measured: 359.3. |
| 58 | 2-(5-tert-Butyl-2-methyl-2H-pyrazol-3-yl)-5-(2,6-difluoro- |
| | phenyl)-1H-benzimidazole trifluoroacetic acid salt |
| | ¹H-NMR (400 MHz, d6-DMSO) δ: 7.75-7.66 (m, 2H), 7.53- |
| | 7.44 (m, 1H), 7.32-7.20 (m, 3H), 6.90 (s, 1H), 4.27 (s, 3H), |
| | 1.31 (s, 9H) |
| | Mass Spectrum (loop, ESI pos.): Calculated for C₂₁H₂₁F₂N₄: |
| | 367.2 (M + H); Measured: 367.2. |
| 59 | 2-(5-tert-Butyl-2-methyl-2H-pyrazol-3-yl)-5-(2-chloro-phenyl)- |
| | 1H-benzimidazole trifluoroacetic acid salt |
| | ¹H-NMR (400 MHz, d6-DMSO) δ: 7.69 (d, J = 8.3 Hz, 1H), |
| | 7.64 (s, 1H), 7.59 (dd, J = 1.7, 7.4 Hz, 1H), 7.52-7.38 (m, 3H), |
| | 7.30 (dd J = 1.4, 8.4 Hz, 1H), 6.90 (s, 1H), 4.27 (s, 3H), 1.31 |
| | (s, 9H) |
| | Mass Spectrum (loop, ESI pos.): Calculated for C₂₁H₂₂ClN₄: |
| | 365.2 (M + H); Measured: 365.2. |
| 60 | 2-[2-(5-tert-Butyl-2-methyl-2H-pyrazol-3-yl)-1H-benzimidazol- |
| | 5-yl]-benzonitrile trifluoroacetic acid salt |
| | ¹H-NMR (400 MHz, d6-DMSO) δ: 7.97 (dd, 1H, J = 1.1, 7.8), |
| | 7.85-7.73 (m, 3H), 7.71 (dd, J = 0.8, 7.8 Hz, 1H), 7.58 (dt, J = |
| | 1.2, 7.6 Hz, 1H), 7.44 (dd, J = 1.7, 8.3 Hz, 1H), 6.93 (s, 1H), |
| | 4.28 (s, 3H), 1.32 (s, 9H) |
| | Mass Spectrum (loop, ESI pos.): Calculated for C₂₂H₂₂N₅: |
| | 356.2 (M + H); Measured: 356.3. |

### Example 5

### 2-(5-tert-Butyl-2-methyl-2H-pyrazol-3-yl)-5-(2-fluoro-6-trifluoromethylphenyl)-1H-benzimidazole sodium salt (Compound #61)

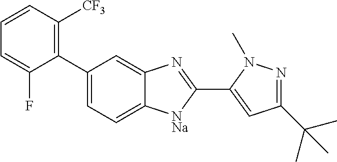

### STEP A. 6′-Fluoro-3-nitro-2′-trifluoromethyl-biphenyl-4-ylamine

To a mixture of 1-bromo-2-fluoro-6-trifluoromethylbenzene (0.850 mL, 6.09 mmol), 4-amino-3-nitrophenylboronic acid pinacol ester (2.09 g, 7.92 mmol), and (dppf)PdCl₂.DCM (249 mg, 0.300 mmol) under Ar was added DME (24 mL) and 2 M aqueous Na₂CO₃ (8.00 mL, 16.0 mmol). The resulting mixture was stirred at 90° C. for 24 h, cooled to room temperature, diluted with EtOAc (25 mL), and washed sequentially with water (10 mL) and brine (10 mL). The resulting solution was dried over MgSO₄ and filtered. The solvent was removed under reduced pressure, and the resulting residue was chromatographed using a 80-g SiO₂ pre-packed column eluting with 0:1 to 2:3 EtOAc-hexanes to yield a yellow solid. ¹H-NMR (400 MHz, CDCl₃) δ: 8.10 (s, 1H), 7.53-7.61 (m, 1H), 7.43-7.53 (m, 1H), 7.35 (t, J=8.5 Hz, 1H), 7.30 (d, J=7.3 Hz, 1H), 6.89 (d, J=8.3 Hz, 1H), 5.47 (br. s., 2H).

### STEP B. 2-(5-tert-Butyl-2-methyl-2H-pyrazol-3-yl)-5-(2-fluoro-6-trifluoromethylphenyl)-1H-benzimidazole

6′-Fluoro-3-nitro-2′-trifluoromethyl-biphenyl-4-ylamine (184 mg, 0.613 mmol) was placed under Ar, and anhydrous THF (3 mL) was added via syringe. NaH (74.0 mg of 60% mineral oil dispersion, 1.84 mmol) was added in small portions to the stirred solution, and the resulting mixture was stirred at room temperature for 15 min. 5-tert-butyl-2-methyl-2H-pyrazole-3-carbonyl chloride (123 mg, 0.613 mmol) in anhydrous THF (2 mL) was added dropwise to the stirred mixture over a period of 2 min. The resulting mixture was then stirred at room temperature for 1 h, quenched with H₂O (2 mL), and extracted with EtOAc (3×10 mL). The combined organic extracts were dried over anhydrous MgSO₄ and filtered, and then the solvent was removed under reduced pressure.

The residue was dissolved in absolute EtOH (10 mL), and then H₂O (2.5 mL), NH₄Cl (328 mg, 6.13 mmol), and Fe powder (171 mg, 3.07 mmol) were added to the solution. The resulting mixture was stirred at 80° C. for 16 h, cooled to room temperature, poured into H₂O (ca. 20 mL), and extracted with EtOAc (3×20 mL). The combined organic extracts were dried over MgSO₄ and filtered. The solvent was removed under reduced pressure.

The resulting residue was dissolved in anhydrous 1,4-dioxane (10 mL). CSA (28.5 mg, 0.123 mmol) was added as a solid, and the mixture was stirred at 100° C. for 12 h. The resulting mixture was cooled to room temperature, poured into H₂O (10 mL), and basified to ca. pH 10 with 2 M aqueous NaOH. The aqueous layer was extracted with EtOAc (3×20 mL), and then the combined organic extracts were dried over MgSO₄ and filtered. The solvent was removed under reduced pressure, and the residue was chromatographed using a 40-g SiO₂ pre-packed column eluting with 0:1 to 3:7 EtOAc-hexanes to yield a white solid. ¹H-NMR (400 MHz, CD₃OD) δ: 7.34-7.91 (m, 5H), 7.19 (d, J=7.6 Hz, 1H), 6.79 (s, 1H), 4.26 (s, 3H), 1.37 (s, 9H). Mass Spectrum (LCMS, ESI pos.): Calculated for C₂₂H₂₀F₄N₄: 417.2 (M+H); Measured: 417.3.

### STEP C. 2-(5-tert-Butyl-2-methyl-2H-pyrazol-3-yl)-5-(2-fluoro-6-trifluoromethylphenyl)-1H-benzimidazole sodium salt

2-(5-tert-Butyl-2-methyl-2H-pyrazol-3-yl)-5-(2-fluoro-6-trifluoromethylphenyl)-1H-benzimidazole (135 mg, 0.323 mmol) was suspended in anhydrous MeOH (4 mL), and 0.5 M NaOMe in MeOH (0.650 mL, 0.323 mmol) was added via syringe. The resulting mixture was sonicated until complete dissolution occurred. The solvent was removed under reduced pressure to yield the title compound as a pale yellow foam.

¹H-NMR (400 MHz, d6-DMSO) δ: 7.63-7.71 (m, 1H), 7.53-7.63 (m, 2H), 7.42 (d, J=8.3 Hz, 1H), 7.28 (s, 1H), 6.72 (d, J=8.1 Hz, 1H), 6.58 (s, 1H), 4.28 (s, 3H), 1.28 (s, 9H). Mass Spectrum (LCMS, ESI pos.): Calculated for C₂₂H₂₀F₄N₄: 417.2 (M+H); Measured: 417.3.

### Example 6

### 2-(5-tert-Butyl-2,4-dimethyl-2H-pyrazol-3-yl)-5-(2-trifluoromethylphenyl)-1H-benzimidazole sodium salt (Compound #76)

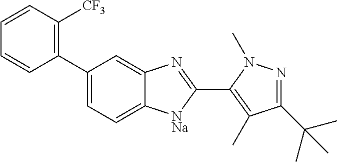

### STEP A. 3-Nitro-2′-trifluoromethyl-biphenyl-4-ylamine

To a mixture of 4-bromo-2-nitroaniline (1.30 g, 6.00 mmol), 2-trifluoromethylphenylboronic acid (1.48 g, 7.80 mmol), and (dppf)PdCl₂.DCM (245 mg, 0.300 mmol) under Ar was added DME (24 mL) and 2 M aqueous Na₂CO₃ (8.00 mL, 16.0 mmol). The resulting mixture was stirred at 90° C. for 16 h, cooled to room temperature, diluted with EtOAc (25 mL), and washed sequentially with H₂O (10 mL) and brine (10 mL). The resulting solution was dried over MgSO₄ and filtered. The solvent was removed under reduced pressure, and the residue was chromatographed using a 80-g SiO₂ pre-packed column eluting with 0:1 to 2:3 EtOAc-hexanes to yield an orange solid. ¹H-NMR (400 MHz, CDCl₃) δ: 8.11 (d, J=2.0 Hz, 1H), 7.75 (d, J=7.8 Hz, 1H), 7.54-7.62 (m, J=7.6 Hz, 1H), 7.44-7.53 (m, J=7.8 Hz, 1H), 7.36 (dd, J=8.6, 1.3 Hz, 1H), 7.33 (d, J=7.6 Hz, 1H), 6.84 (d, J=8.6 Hz, 1H).

### STEP B. 2-(5-tert-Butyl-2,4-dimethyl-2H-pyrazol-3-yl)-5-(2-trifluoromethylphenyl)-1H-benzimidazole

Oxalyl chloride (76.0 μL, 0.867 mmol) was added dropwise to a solution of 5-tert-butyl-2,4-dimethyl-2H-pyrazole-3-carboxylic acid (113 mg, 0.578 mmol, as prepared in Example E) in DCM (2 mL) and DMF (ca. 10 μL). The resulting mixture was stirred at room temperature for 1 h, the solvent was removed under reduced pressure, and resulting residue was dissolved in anhydrous THF (2 mL).

3-Nitro-2′-trifluoromethyl-biphenyl-4-ylamine (163 mg, 0.578 mmol, as prepared in the previous step) was placed under Ar, and anhydrous THF (3 mL) was added via syringe. NaH (77.0 mg of 60% mineral oil dispersion, 1.93 mmol) was added in small portions to the stirred solution, and the resulting mixture was stirred at room temperature for 15 min. The THF solution of 5-tert-butyl-2,4-dimethyl-2H-pyrazole-3-carbonyl chloride was added dropwise to the stirred resulting mixture over 2 min. The resulting mixture was stirred at room temperature for 1 h, quenched with H₂O (1 mL), and extracted with EtOAc (3×15 mL). The combined organic extracts were dried over MgSO₄ and filtered, and then the solvent was removed under reduced pressure.

The residue was dissolved in absolute EtOH (10 mL), and NH₄Cl (31.0 mg, 0.578 mmol), Fe powder (161 mg, 2.89 mmol), and glacial AcOH (2 mL) were added. The resulting mixture was stirred at 80° C. for 16 h, cooled to room temperature, poured into H₂O (ca. 20 mL), and extracted with EtOAc (3×10 mL). The combined organic extracts were dried over MgSO₄ and filtered. The solvent was removed under reduced pressure, and the residue was chromatographed using a 24-g SiO₂ pre-packed column eluting with 0:1 to 2:5 EtOAc-hexanes to yield the title compound as a white solid. ¹H-NMR (400 MHz, CD₃OD) δ: 7.79 (d, J=7.8 Hz, 1H), 7.69 (d, J=8.3 Hz, 1H), 7.58-7.65 (m, 2H), 7.53 (t, J=7.6 Hz, 1H), 7.43 (d, J=7.6 Hz, 1H), 7.31 (dd, J=8.3, 1.0 Hz, 1H), 3.94 (s, 3H), 2.30 (s, 3H), 1.41 (s, 9H). Mass Spectrum (LCMS, ESI pos.): Calculated for C₂₃H₂₃F₃N, 413.2 (M+H); Measured: 413.3.

### STEP C. 2-(5-tert-Butyl-2,4-dimethyl-2H-pyrazol-3-yl)-5-(2-trifluoromethylphenyl)-1H-benzimidazole sodium salt

Following the procedure described in Step C of Example 1, the title compound was prepared from 2-(5-tert-butyl-2,4-dimethyl-2H-pyrazol-3-yl)-5-(2-trifluoromethylphenyl)-1H-benzimidazole (136 mg, 0.330 mmol, as prepared in the previous step) and 0.5 M NaOMe in MeOH (0.660 mL, 0.330 mmol) as a white powder.

¹H-NMR (400 MHz, d6-DMSO) δ: 7.78 (d, J=7.6 Hz, 1H), 7.66 (t, J=7.5 Hz, 1H), 7.51 (t, J=7.6 Hz, 1H), 7.43 (d, J=7.8 Hz, 1H), 7.40 (d, J=8.3 Hz, 1H), 7.33 (s, 1H), 6.78 (d, J=8.1 Hz, 1H), 4.03 (s, 3H), 2.40 (s, 3H), 1.33 (s, 9H). Mass Spectrum (LCMS, ESI pos.): Calculated for C₂₃H₂₃F₃N₄: 413.2 (M+H); Measured: 413.2.

### Example 7

### 2-(5-tert-Butyl-2,4-dimethyl-2H-pyrazol-3-yl)-5-(2-fluoro-6-trifluoromethyl-phenyl)-1H-benzimidazole sodium salt (Compound #78)

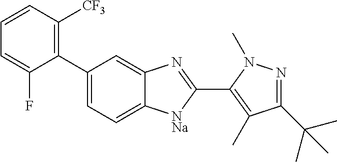

### STEP A. 2-(5-tert-Butyl-2,4-dimethyl-2H-pyrazol-3-yl)-5-(2-fluoro-6-trifluoromethyl-phenyl)-1H-benzimidazole

To 5-tert-butyl-2,4-dimethyl-2H-pyrazole-3-carboxylic acid (118 mg, 0.601 mmol, as prepared in Example E) were added DCM (2 mL) and DMF (ca. 10 μL), and then oxalyl chloride (79.0 μL, 0.902 mmol) was added dropwise. The resulting mixture was stirred at room temperature for 1 h, and the solvent was removed under reduced pressure. The residue was dissolved in anhydrous THF (2 mL).

To 2′-fluoro-3-nitro-6′-trifluoromethyl-biphenyl-4-ylamine (180 mg, 0.601 mmol, as prepared in Example 5, Step A) was under Ar was added anhydrous THF (3 mL), and then NaH (72.0 mg of 60% mineral oil dispersion, 1.80 mmol) was added in small portions. The resulting mixture was stirred at room temperature for 15 min, and the THF solution of 5-tert-butyl-2,4-dimethyl-2H-pyrazole-3-carbonyl chloride was added dropwise to the stirred solution over 2 min. The resulting mixture was stirred at room temperature for 1 h, quenched with H₂O (1 mL), and extracted with EtOAc (3×10 mL). The combined organic extracts were dried over MgSO₄ and filtered, and the solvent was removed under reduced pressure.

The residue was dissolved in absolute EtOH (10 mL), and then H₂O (2.5 mL), NH₄Cl (321 mg, 6.01 mmol), and Fe powder (168 mg, 3.01 mmol) were added. The resulting mixture was stirred at 80° C. for 16 h, cooled to room temperature, poured into H₂O (ca. 20 mL), and extracted with EtOAc (3×20 mL). The combined organic extracts were dried over MgSO₄ and filtered. The solvent was removed under reduced pressure.

The residue was dissolved in anhydrous 1,4-dioxane (10 mL), p-TsOH.H₂O (23.0 mg, 0.121 mmol) was added, and the resulting mixture was stirred at 100° C. for 12 h. The resulting mixture was then cooled to room temperature, poured into H₂O (ca. 20 mL), and basified to ca. pH 10 using 2 M aqueous NaOH. The aqueous layer was extracted with EtOAc (3×20 mL), and the combined organic extracts were dried over MgSO₄ and filtered. The solvent was removed under reduced pressure, and the residue was chromatographed using a 40-g SiO₂ pre-packed column eluting with 0:1 to 3:7 EtOAc-hexanes to yield the title compound as a white solid. ¹H-NMR (400 MHz, CD₃OD) δ: 7.51-7.87 (m, 4H), 7.47 (t, J=8.6 Hz, 1H), 7.23 (d, J=8.3 Hz, 1H), 3.91 (s, 3H), 2.29 (s, 3H), 1.40 (s, 9H). Mass Spectrum (LCMS, ESI pos.): Calculated for C₂₃H₂₂F₄N₄: 431.2 (M+H); Measured: 431.2.

### STEP B. 2-(5-tert-Butyl-2,4-dimethyl-2H-pyrazol-3-yl)-5-(2-fluoro-6-trifluoromethyl-phenyl)-1H-benzimidazole sodium salt

Following the procedure described in Step C of Example 5, the title compound was prepared from 2-(5-tert-butyl-2,4-dimethyl-2H-pyrazol-3-yl)-5-(2-fluoro-6-trifluoromethyl-phenyl)-1H-benzimidazole (159 mg, 0.369 mmol, as prepared in the previous step) and 0.5 M NaOMe in MeOH (0.740 mL, 0.369 mmol as a white foam.

¹H-NMR (400 MHz, d6-DMSO) δ: 7.63-7.71 (m, 1H), 7.53-7.63 (m, 2H), 7.42 (d, J=8.1 Hz, 1H), 7.27 (s, 1H), 6.70 (d, J=8.3 Hz, 1H), 4.03 (s, 3H), 2.39 (s, 3H), 1.33 (s, 9H). Mass Spectrum (LCMS, ESI pos.): Calculated for C₂₃H₂₂F₄N₄: 431.2 (M+H); Measured: 431.3.

### Example 8

### 2-(5-tert-Butyl-2-methyl-2H-pyrazol-3-yl)-7-methyl-5-(2-trifluoromethylphenyl)-1H-benzimidazole hydrochloride (Compound #79)

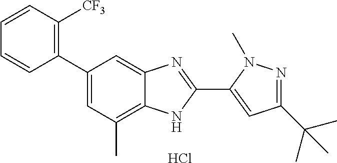

### STEP A. 3-Methyl-5-nitro-2′-trifluoromethyl-biphenyl-4-ylamine

Following the procedure described in Step A of Example 6, the title compound was prepared from 4-bromo-6-methyl-2-nitroaniline (242 mg, 1.05 mmol) and 2-trifluoromethylphenylboronic acid (298 mg, 1.57 mmol. ¹H-NMR (400 MHz, CDCl₃) δ: 8.03 (d, J=1.8 Hz, 1H), 7.73 (d, J=7.6 Hz, 1H), 7.56 (t, J=7.5 Hz, 1H), 7.46 (t, J=7.7 Hz, 1H), 7.31 (d, J=7.6 Hz, 1H), 7.32 (s, 1H), 6.27 (br. s., 2H), 2.28 (s, 3H).

### STEP B. 2-(5-tert-Butyl-2-methyl-2H-pyrazol-3-yl)-7-methyl-5-(2-trifluoromethylphenyl)-1H-benzimidazole

Following the procedure described in Step B of Example 5, the title compound was prepared from 3-methyl-5-nitro-2′-trifluoromethyl-biphenyl-4-ylamine (125 mg, 0.422 mmol, as prepared in the previous step) and 5-tert-butyl-2-methyl-2H-pyrazole-3-carbonyl chloride (85.0 mg, 0.422 mmol). ¹H-NMR (400 MHz, CD₃OD) δ: 7.77 (d, J=7.8 Hz, 1H), 7.62 (t, J=7.2 Hz, 1H), 7.52 (t, J=7.6 Hz, 1H), 7.48 (br. s., 0.5H), 7.41 (d, J=7.6 Hz, 1H), 7.28 (br. s., 0.5H), 7.03 (br. s., 1H), 6.87 (br. s., 0.5H), 6.74 (br. s., 0.5H), 4.25 (br. s., 3H), 2.63 (br. s., 3H), 1.37 (s, 9H). Mass Spectrum (LCMS, ESI pos.): Calculated for C₂₃H₂₃F₃N₄: 413.2 (M+H); Measured: 413.3.

### STEP C. 2-(5-tert-Butyl-2-methyl-2H-pyrazol-3-yl)-7-methyl-5-(2-trifluoromethylphenyl)-1H-benzimidazole hydrochloride

2-(5-tert-Butyl-2-methyl-2H-pyrazol-3-yl)-7-methyl-5-(2-trifluoromethylphenyl)-1H-benzimidazole (64.9 mg, 0.157 mmol, as prepared in the previous step) was dissolved in EtOAc (1 mL), and 1 M HCl in Et₂O (157 μL, 0.157 mmol) was added via syringe. The solution was thoroughly mixed, and the solvent was removed under reduced pressure. The residue was triturated with Et₂O, generating a white powder. The supernatant was removed via pipette, and the solid was rinsed once more with Et₂O. The residual solvent was removed under vacuum to yield the title compound as a white powder.

¹H-NMR (400 MHz, d6-DMSO) δ: 7.84 (d, J=7.6 Hz, 1H), 7.72 (t, J=7.3 Hz, 1H), 7.62 (t, J=7.6 Hz, 1H), 7.46 (d, J=7.6 Hz, 1H), 7.37 (s, 1H), 7.03 (s, 1H), 6.95 (s, 1H), 4.26 (s, 3H), 2.61 (s, 3H), 1.32 (s, 9H). Mass Spectrum (LCMS, ESI pos.): Calculated for C₂₃H₂₃F₃N₄: 413.2 (M+H); Measured: 413.3.

### Example 9

### 2-(5-tert-Butyl-2-methyl-2H-pyrazol-3-yl)-5-(2-difluoromethoxyphenyl)-1H-benzimidazole hydrochloride (Compound #81)

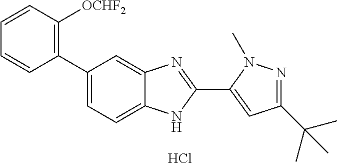

### STEP A. 3-Nitro-2′-difluoromethoxy-biphenyl-4-ylamine

Following the procedure described in Step A of Example 5, the title compound was prepared from 1-bromo-2-difluoromethoxybenzene (223 mg, 1.00 mmol) and 4-amino-3-nitrophenylboronic acid (273 mg, 1.50 mmol). ¹H-NMR (400 MHz, CDCl₃) δ: 7.58 (dd, J=8.6, 2.0 Hz, 1H), 7.42 (dd, J=7.5, 1.9

Hz, 1H), 7.35 (dd, J=7.7, 1.9 Hz, 1H), 7.30 (dd, J=7.5, 1.4 Hz, 1H), 7.22 (dd, J=8.1, 1.0 Hz, 1H), 6.87 (d, J=8.6 Hz, 1H), 6.39 (t, J=74 Hz, 1H), 6.16 (br. s., 2H).

### STEP B. 3-tert-Butyl-1-methyl-1H-pyrazole-5-carboxylic acid (3-nitro-2′-difluoromethoxy-biphenyl-4-yl)-amide

3-Nitro-2′-difluoromethoxy-biphenyl-4-ylamine (122 mg, 0.670 mmol as prepared in the previous step), 5-tert-butyl-2-methyl-2H-pyrazole-3-carboxylic acid (159 mg, 0.871 mmol, as prepared in Example A), and BOP (356 mg, 0.804 mmol) were dissolved in anhydrous pyridine (5 mL). NaH (165 mg of a 60% mineral oil dispersion, 4.13 mmol) was added as a solid in small portions to the stirred resulting mixture. After completion of the addition, the resulting mixture was stirred at room temperature for 12 h, diluted with DCM (2 mL), and quenched with AcOH (0.5 mL). The solvent was removed under reduced pressure, and the residue was dissolved in EtOAc (20 mL) and washed with saturated aqueous NaHCO₃ (20 mL). The aqueous layer was extracted with EtOAc (2×10 mL), and the combined organic extracts were dried over MgSO₄ and filtered. The solvent was removed under reduced pressure, and the residue was chromatographed using a 12-g SiO₂ pre-packed column eluting with 0:1 to 3:7 EtOAc-hexanes to yield a residue. ¹H-NMR (400 MHz, CDCl₃) δ: 11.19 (s, 1H), 8.95 (d, J=8.8 Hz, 1H), 8.44 (d, J=2.0 Hz, 1H), 7.86 (dd, J=8.7, 2.1 Hz, 1H), 7.40-7.51 (m, 2H), 7.34-7.37 (m, J=7.6 Hz, 1H), 7.27-7.30 (m, 1H), 6.68 (s, 1H), 6.45 (t, J=73 Hz, 1H), 4.21 (s, 3H), 1.36 (s, 9H).

### STEP C. 2-(5-tert-Butyl-2-methyl-2H-pyrazol-3-yl)-5-(2-difluoromethoxyphenyl)-1H-benzimidazole

3-tert-Butyl-1-methyl-1H-pyrazole-5-carboxylic acid (3-nitro-2′-difluoromethoxy-biphenyl-4-yl)-amide (103 mg, 0.231 mmol, as prepared in previous step) was dissolved in absolute EtOH (5 mL), and then H₂O (1.25 mL), NH₄Cl (124 mg, 2.31 mmol), and Fe powder (65.0 mg, 1.16 mmol) were added. The resulting mixture was stirred at 80° C. for 12 h, cooled to room temperature, and filtered. The solvent was removed under reduced pressure, the residue was dissolved in anhydrous 1,4-dioxane (6 mL), and CSA (11.0 mg, 0.0460 mmol) was added. The resulting mixture was stirred at 100° C. for 12 h, cooled to room temperature, and poured into sat aqueous NaHCO₃. The aqueous phase was extracted with EtOAc (3×10 mL), and the combined organic extracts were dried over MgSO₄ and filtered. The solvent was removed under reduced pressure, and the residue was chromatographed using a 40-g SiO₂ pre-packed column eluting with 0:1 to 3/7 EtOAc-hexanes to yield a residue. ¹H-NMR (400 MHz, d6-DMSO) δ: 12.95 (br. s., 1H), 7.67-7.86 (m, 1H), 7.55-7.67 (m, 1H), 7.49-7.55 (m, 1H), 7.40-7.49 (m, J=7.6 Hz, 1H), 7.24-7.40 (m, 3H), 7.07 (t, J=74 Hz, 1H), 6.89 (s, 1H), 4.28 (s, 3H), 1.31 (s, 9H). Mass Spectrum (LCMS, ESI pos.): Calculated for C₂₂H₂₂F₂N₄O: 397.2 (M+H); Measured: 397.3.

### STEP D. 2-(5-tert-Butyl-2-methyl-2H-pyrazol-3-yl)-5-(2-difluoromethoxyphenyl)-1H-benzimidazole hydrochloride

Following the procedure described in Step C of Example 8, the title compound was prepared from 2-(5-tert-butyl-2-methyl-2H-pyrazol-3-yl)-5-(2-difluoromethoxyphenyl)-1H-benzimidazole (26.8 mg, 0.0680 mmol, as prepared in the previous step) and 1 M HCl in Et₂O (68.0 μL, 0.0680 mmol).

¹H-NMR (400 MHz, d6-DMSO) δ: 7.67-7.77 (m, 2H), 7.53 (dd, J=7.5, 1.6 Hz, 1H), 7.42-7.50 (m, 1H), 7.34-7.42 (m, 2H), 7.33 (d, J=8.1 Hz, 1H), 7.17 (t, J=74 Hz, 1H), 6.93 (s, 1H), 4.26 (s, 3H), 1.32 (s, 9H). Mass Spectrum (LCMS, ESI pos.): Calculated for C₂₂H₂₂F₂N₄O: 397.2 (M+H); Measured: 397.3.

### Example 10

### 2-(5-tert-Butyl-2-ethyl-2H-pyrazol-3-yl)-7-methyl-5-(2-trifluoromethylphenyl)-1H-benzimidazole sodium salt (Compound #98)

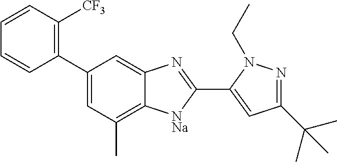

### STEP A. 5-Methyl-2′-trifluoromethyl-biphenyl-3,4-diamine

3-Methyl-5-nitro-2′-trifluoromethyl-biphenyl-4-ylamine (145 mg, 0.490 mmol, as prepared in Step A of Example 8) was dissolved in absolute EtOH (3 mL), and then 3 M HCl (0.6 mL) and Fe powder (137 mg, 2.45 mmol)) were added. The resulting mixture was stirred at 80° C. for 4 h, cooled to room temperature, and filtered, and the solvent was removed under reduced pressure. The residue was washed with 2 M aqueous NaOH (10 mL), and the aqueous phase was extracted with EtOAc (3×15 mL). The combined organic extracts were dried over MgSO₄ and filtered. The solvent was removed under reduced pressure to yield a residue. ¹H-NMR (400 MHz, CDCl₃) δ: 7.69 (d, J=7.6 Hz, 1H), 7.49 (t, J=7.2 Hz, 1H), 7.38 (t, J=7.7 Hz, 1H), 7.30 (d, J=7.6 Hz, 1H), 6.63 (s, 2H), 3.83 (br. s., 4H), 2.22 (s, 3H).

### STEP B. 2-(5-tert-Butyl-2-ethyl-2H-pyrazol-3-yl)-7-methyl-5-(2-trifluoromethylphenyl)-1H-benzimidazole

5-tert-Butyl-2-ethyl-2H-pyrazole-3-carboxylic acid (87 mg, 0.44 mmol, as prepared in Example B) was dissolved in DCM (1 mL), and then DMF (ca. 10 μL) and oxalyl chloride (58 μL, 0.67 mmol) were added. The resulting mixture was stirred at room temperature for 2 h, and the solvent was removed under reduced pressure. The resulting residue was dissolved in anhydrous DCM (15 mL).

5-Methyl-2′-trifluoromethyl-biphenyl-3,4-diamine (130 mg, 0.488 mmol, as prepared in the previous step) was dissolved in DCM (25 mL), and TEA (0.190 mL, 1.33 mmol) was added. The DCM solution of the residue prepared above was placed in a dropping funnel and added dropwise to the stirred resulting mixture. After completion of the addition, the resulting mixture was stirred at room temperature for 1 h. The solvent was removed under reduced pressure, and the residue was chromatographed using a 24-g SiO₂ pre-packed column eluting with 0:1 to 2:3 EtOAc-hexanes, to yield a residue as a mixture of the mono-amide isomers.

The residue (mono-amide mixture) was dissolved in anhydrous 1,4-dioxane (1 mL), and CSA (17.0 mg, 0.0720 mmol) was added. The resulting mixture was stirred at 100° C. for 3 h, cooled to room temperature, and quenched with TEA (0.1 mL). The solvent was removed under reduced pressure, and the residue was chromatographed using a 24-g SiO₂ pre-packed column eluting with 0:1 to 1:3 EtOAc-heptane, to yield a residue. ¹H-NMR (400 MHz, CD₃OD) δ: 7.77 (d, J=7.8 Hz, 1H), 7.61 (t, J=7.2 Hz, 1H), 7.52 (t, J=7.6 Hz, 1H), 7.41 (d, J=7.6 Hz, 1H), 7.38 (br. s., 1H), 7.02 (s, 1H), 6.80 (br. s., 1H), 4.76 (q, J=7.1 Hz, 2H), 2.64 (s, 3H), 1.43 (t, J=7.1 Hz, 3H), 1.37 (s, 9H).

### STEP C. 2-(5-tert-Butyl-2-ethyl-2H-pyrazol-3-yl)-7-methyl-5-(2-trifluoromethylphenyl)-1H-benzimidazole sodium salt

Following the procedure described in Step C of Example 5, the title compound was prepared from 2-(5-tert-butyl-2-ethyl-2H-pyrazol-3-yl)-7-methyl-5-(2-trifluoromethylphenyl)-1H-benzimidazole (139 mg, 0.326 mmol, as prepared in the previous step) and 0.5 M NaOMe in MeOH (0.650 mL, 0.326 mmol) as a white foam.

¹H-NMR (400 MHz, d6-DMSO) δ: 7.76 (d, J=7.6 Hz, 1H), 7.63 (t, J=7.5 Hz, 1H), 7.49 (t, J=7.7 Hz, 1H), 7.42 (d, J=7.6 Hz, 1H), 7.13 (s, 1H), 6.54 (s, 1H), 6.51 (s, 1H), 4.89 (q, J=7.1 Hz, 2H), 1.34 (t, J=7.1 Hz, 3H), 1.28 (s, 9H). Mass Spectrum (LCMS, ESI pos.): Calculated for C₂₄H₂₅F₃N₄: 427.2 (M+H); Measured: 427.4.

### Example 11

### 2-{3-tert-Butyl-5-[5-(2-trifluoromethylphenyl)-1H-benzimidazol-2-yl]-pyrazol-1-yl}-ethanol hydrochloride (Compound #102)

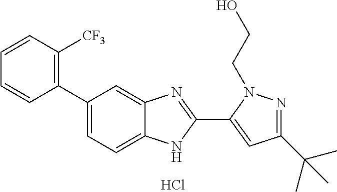

### STEP A. 2-(2-Benzyloxy-ethyl)-5-tert-butyl-2H-pyrazole-3-carboxylic acid ethyl ester

5-tert-Butyl-2H-pyrazole-3-carboxylic acid ethyl ester (108 mg, 0.550 mmol) was dissolved in anhydrous ACN (2 mL), and then K₂CO₃ (114 mg, 0.825 mmol) and 1-bromo-2-benzyloxyethane (0.100 mL, 0.605 mmol) were added. The resulting mixture was stirred at 80° C. for 3 days, cooled to room temperature, poured into EtOAc (10 mL), and filtered. The solvent was removed under reduced pressure, and the residue was chromatographed using a 12-g SiO₂ pre-packed column eluting with 0:1 to 3:7 EtOAc-hexanes to yield a residue. ¹H-NMR (400 MHz, CDCl₃) δ: 7.17-7.33 (m, 5H), 6.69 (s, 1H), 4.77 (t, J=5.8 Hz, 2H), 4.47 (s, 2H), 4.29 (q, J=7.1 Hz, 2H), 3.83 (t, J=5.9 Hz, 2H), 1.34 (t, J=7.1 Hz, 3H), 1.31 (s, 9H).

### STEP B. 1-(2-Benzyloxy-ethyl)-3-tert-butyl-1H-pyrazole-5-carboxylic acid

2-(2-Benzyloxy-ethyl)-5-tert-butyl-2H-pyrazole-3-carboxylic acid ethyl ester (134 mg, 0.406 mmol, as prepared in the previous step) was dissolved in a mixture of 1,4-dioxane (2 mL) and MeOH (2 mL), and then 2.5 M aqueous NaOH (1 mL, 2.50 mmol) was added. The resulting mixture was stirred at room temperature for 16 h, and the solvent was removed under reduced pressure. The residue was dissolved in H₂O (4 mL) and acidified to ca. pH 2 using 3 M aqueous HCl. The aqueous solution was extracted with DCM (3×20 mL), and the combined organic extracts were dried over MgSO₄ and filtered. The solvent was removed under reduced pressure to yield a residue. ¹H-NMR (400 MHz, CDCl₃) δ: 11.39 (br. s., 1H), 7.15-7.30 (m, 5H), 6.81 (s, 1H), 4.79 (t, J=5.7 Hz, 2H), 4.47 (s, 2H), 3.84 (t, J=5.8 Hz, 2H), 1.33 (s, 9H).

### STEP C. 1-[2-(2-Benzyloxy-ethyl)-3-tert-butyl-1H-pyrazol-5-yl]-5-(2-trifluoromethylphenyl)-1H-benzimidazole

Oxalyl chloride (28.0 μL, 0.320 mmol) was added dropwise to a solution of 1-(2-benzyloxy-ethyl)-3-tert-butyl-1H-pyrazole-5-carboxylic acid (64.3 mg, 0.213 mmol, as prepared in the previous step) in DCM (2 mL) and DMF (ca. 10 μL), and then the resulting mixture was stirred at room temperature for 1 h. The solvent was removed under reduced pressure, and the resulting residue was dissolved in anhydrous THF (3 mL).

3-Nitro-2′-trifluoromethyl-biphenyl-4-ylamine (60.1 mg, 0.213 mmol, as prepared in Step A of Example 6) was placed under Ar, and anhydrous THF (1 mL) was added via syringe. NaH (26.0 mg of 60% mineral oil dispersion, 0.639 mmol) was added in small portions to the stirred solution, and the resulting mixture was stirred at room temperature for 15 min. The THF solution of the residue prepared as described above was added dropwise to the stirred resulting mixture over 2 min. The resulting mixture was stirred at room temperature for 1 h, quenched with H₂O (1 mL), and extracted with EtOAc (3×15 mL). The combined organic extracts were dried over anhydrous MgSO₄ and filtered, and the solvent was removed under reduced pressure.

The residue was dissolved in absolute EtOH (6 mL), and then glacial AcOH (1 mL) and Fe powder (59.0 mg, 1.07 mmol) were added. The resulting mixture was stirred at 80° C. for 10 h, cooled to room temperature, and filtered. The filtrate was concentrated under reduced pressure, and the residue was dissolved in EtOAc (15 mL) and washed with H₂O (ca. 20 mL). The aqueous layer was extracted with EtOAc (2×15 mL), and the combined organic extracts were dried over MgSO₄ and filtered. The solvent was removed under reduced pressure, and the residue was chromatographed using a 24-g SiO₂ pre-packed column eluting with 0:1 to 2:3 EtOAc-hexanes to yield a white solid. ¹H-NMR (400 MHz, CD₃OD) δ: 7.79 (d, J=7.8 Hz, 1H), 7.71 (br. s., 0.5H), 7.64 (br. s., 0.5H), 7.64 (t, J=7.3 Hz, 1H), 7.54 (t, J=7.6 Hz, 1H), 7.45 (br. s., 0.5H), 7.42 (d, J=7.6 Hz, 1H), 7.33 (br. s., 0.5H), 7.21 (d, J=8.1 Hz, 1H), 7.08-7.17 (m, 3H), 6.96-7.08 (m, 2H), 6.75 (s, 1H), 4.92 (t, J=5.3 Hz, 2H), 4.38 (s, 2H), 3.89 (t, J=5.2 Hz, 2H), 1.37 (s, 9H). Mass Spectrum (LCMS, ESI pos.): Calculated for C₃₀H₂₉F₃N₄O: 519.2 (M+H); Measured: 519.2.

### STEP D. 2-{3-tert-Butyl-5-[5-(2-trifluoromethylphenyl)-1H-benzimidazol-2-yl]-pyrazol-1-yl}-ethanol

1-[2-(2-Benzyloxy-ethyl)-3-tert-butyl-1H-pyrazol-5-yl]-5-(2-trifluoromethyl-phenyl)-1H-benzimidazole (59.3 mg, 0.114 mmol, as prepared in the previous step) was dissolved in absolute EtOH (1.5 mL), and 10% Pd on charcoal (20 mg) was added. The resulting mixture was purged with H₂ for 1 min and stirred at room temperature under an H₂ atmosphere for 24 h. The H₂ was vented, the suspension was filtered, and the solvent was removed under reduced pressure. The residue was chromatographed using a 24 g SiO₂ pre-packed column eluting with 0:1 to 3:2 EtOAc-hexanes to yield a residue. ¹H-NMR (400 MHz, CD₃OD) δ: 7.79 (d, J=7.8 Hz, 1H), 7.65 (t, J=7.6 Hz, 1H), 7.60 (br. s., 2H), 7.55 (t, J=7.6 Hz, 1H), 7.43 (d, J=7.6 Hz, 1H), 7.23 (d, J=8.3 Hz, 1H), 6.77 (s, 1H), 4.78 (t, J=5.6 Hz, 2H), 4.00 (t, J=5.6 Hz, 2H), 1.37 (s, 9H). Mass Spectrum (LCMS, ESI pos.): Calculated for C₂₃H₂₃F₃N₄O: 429.2 (M+H); Measured: 429.2.

### STEP E. 2-{3-tert-Butyl-5-[5-(2-trifluoromethylphenyl)-1H-benzimidazol-2-yl]-pyrazol-1-yl}-ethanol hydrochloride

Following the procedure described in Step C of Example 8, the title compound was prepared 2-{3-tert-butyl-5-[5-(2-trifluoromethylphenyl)-1H-benzimidazol-2-yl]-pyrazol-1-yl}-ethanol (29.3 mg, 0.0680 mmol, as prepared in the previous step) and 1 M HCl in Et₂O (68.0 μL, 0.0680 mmol).

¹H-NMR (400 MHz, d6-DMSO) δ: 7.86 (d, J=8.1 Hz, 1H), 7.71-7.77 (m, 1H), 7.69 (d, J=8.3 Hz, 1H), 7.59-7.67 (m, 1H), 7.55 (s, 1H), 7.48 (d, J=7.6 Hz, 1H), 7.22 (d, J=8.3 Hz, 1H), 6.89 (s, 1H), 4.77 (t, J=6.1 Hz, 2H), 3.79 (t, J=6.2 Hz, 2H), 1.32 (s, 9H). Mass Spectrum (LCMS, ESI pos.): Calculated for C₂₃H₂₃F₃N₄O: 429.2 (M+H); Measured: 429.2.

Following the procedure described in Example 11, and selecting and substituting reagents, starting materials, and conditions as would be known to those skilled in the art, the following compounds of formula (I) of the present invention were prepared:

| ID | |
|---|---|
| No. | Name and Measured Physical Property |
| 101 | 2-{3-tert-Butyl-5-[5-(2-trifluoromethylphenyl)-1H-benzimidazol- |
| | 2-yl]-pyrazol-1-yl}-propan-1-ol hydrochloride |
| | ¹H-NMR (400 MHz, d6-DMSO) δ: 7.86 (d, J = 7.6 Hz, 1H), |
| | 7.70-7.78 (m, 1H), 7.67 (d, J = 8.3 Hz, 1H), 7.59-7.66 (m, 1H), |
| | 7.53 (s, 1H), 7.49 (d, J = 7.6 Hz, 1H), 7.20 (d, J = 8.6 Hz, 1H), |
| | 6.90 (s, 1H), 4.75 (t, J = 6.9 Hz, 2H), 3.42 (t, J = 6.3 Hz, 2H), |
| | 1.89-2.04 (m, 2H), 1.32 (s, 9H) |
| | Mass Spectrum (LCMS, ESI pos.): Calculated for |
| | C₂₄H₂₅F₃N₄O: 443.2 (M + H); Measured: 443.3. |

### Example 12

### 2-(3-Isobutylisoxazol-5-yl)-5-(2-trifluoromethylphenyl)-1H-benzimidazole hydrochloride (Compound #22)

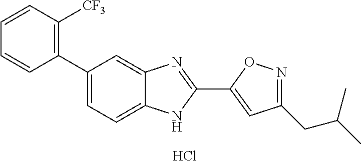

### STEP A. 5-Bromo-2-(3-isobutylisoxazol-5-yl)-1H-benzimidazole

3-Isobutylisoxazole-5-carboxylic acid (257 mg, 1.52 mmol, as prepared in Example G) was dissolved in DCM (3 mL), and DMF (ca. 10 μL) was added. Oxalyl chloride (0.200 mL, 2.28 mmol) was added dropwise to the stirred resulting mixture, and the resulting mixture was stirred at room temperature for 3 h. The solvent was removed under reduced pressure, and the crude acid chloride was dissolved in anhydrous 1,4-dioxane (15 mL).

The 1,4-dioxane solution of the acid chloride was added dropwise to a stirred solution of 4-bromo-1,2-diaminobenzene (426 mg, 2.28 mmol) and DIPEA (0.400 mL, 2.28 mmol) in anhydrous 1,4-dioxane (20 mL). After completion of the addition, the resulting mixture was stirred at room temperature for 16 h and p-TsOH.H₂O (578 mg, 3.04 mmol) was added. The resulting mixture was stirred at 100° C. for 72 h, cooled to room temperature, and basified to ca. pH 10 using 2.5 M aqueous NaOH. The aqueous layer was extracted with EtOAc (3×10 mL), and then the combined organic extracts were dried over MgSO₄ and filtered. The solvent was removed under reduced pressure, and the residue was chromatographed using a 70-g pre-packed column eluting with 1:9 EtOAc-hexanes to yield a residue. ¹H-NMR (400 MHz, CD₃OD) δ: 7.82 (d, J=1.8 Hz, 1H), 7.58 (d, J=8.6 Hz, 1H), 7.46 (dd, J=8.6, 1.8 Hz, 1H), 6.99 (s, 1H), 2.66 (d, J=7.1 Hz, 2H), 1.97-2.16 (m, 1H), 1.02 (d, J=6.6 Hz, 6H).

### STEP B. 2-(3-Isobutylisoxazol-5-yl)-6-(2-trifluoromethylphenyl)-1H-benzimidazole

Following the procedure described in Step B of Example 1, the title compound was prepared from 5-bromo-2-(3-isobutylisoxazol-5-yl)-1H-benzimidazole (68.0 mg, 0.213 mmol, as prepared in the previous step) and 2-trifluoromethylphenylboronic acid (61.0 mg, 0.320 mmol). ¹H-NMR (400 MHz, CD₃OD) δ: 7.80 (d, J=8.1 Hz, 1H), 7.69 (d, J=8.3 Hz, 1H), 7.62-7.68 (m, 1H), 7.59 (s, 1H), 7.52-7.58 (m, 1H), 7.43 (d, J=7.6 Hz, 1H), 7.25-7.33 (m, 1H), 7.00 (s, 1H), 2.66 (d, J=7.1 Hz, 2H), 1.99-2.15 (m, 1H), 1.02 (d, J=6.8 Hz, 6H).

### STEP C. 2-(3-Isobutylisoxazol-5-yl)-6-(2-trifluoromethylphenyl)-1H-benzimidazole hydrochloride

Following the procedure described in Step C of Example 8, the title compound was prepared from 2-(3-isobutylisoxazol-5-yl)-6-(2-trifluoromethylphenyl)-1H-benzimidazole (30.3 mg, 0.0790 mmol, as prepared in the previous step) and 1 M HCl in Et₂O (87.0 μL, 0.0870 mmol) as a white powder.

¹H-NMR (400 MHz, d6-DMSO) δ: 7.86 (d, J=7.1 Hz, 1H), 7.75 (t, J=7.6 Hz, 1H), 7.73 (d, J=8.8 Hz, 1H), 7.64 (t, J=7.6 Hz, 1H), 7.59 (s, 1H), 7.49 (d, J=7.6 Hz, 1H), 7.27 (dd, J=8.3, 1.0 Hz, 1H), 7.18 (s, 1H), 2.64 (d, J=7.1 Hz, 2H), 1.97-2.11 (m, 1H), 0.97 (d, J=6.8 Hz, 6H). Mass Spectrum (LCMS, ESI pos.): Calculated for C₂₁H₁₈F₃N₃O: 386.1 (M+H); Measured: 386.2.

Following the procedure described in Example 12, and selecting and substituting reagents, starting materials, and conditions as would be known to those skilled in the art, the following compounds of formula (I) of the present invention were prepared:

| ID | |
|---|---|
| No. | Name and Measured Physical Property |
| 23 | 2-(3-Isobutylisoxazol-5-yl)-6-(2-trifluoromethoxyphenyl)-1H- |
| | benzimidazole hydrochloride |
| | ¹H-NMR (400 MHz, d6-DMSO) δ: 7.76 (d, J = 8.6 Hz, 1H), |
| | 7.74 (s, 1H), 7.59-7.66 (m, 1H), 7.48-7.58 (m, 3H), 7.41 (dd, J = |
| | 8.3, 1.8 Hz, 1H), 7.17 (s, 1H), 2.64 (d, J = 7.1 Hz, 2H), 2.04 |
| | (spt, J = 6.8 Hz, 1H), 0.97 (d, J = 6.8 Hz, 6H) |
| | Mass Spectrum (LCMS, ESI pos.): Calculated for |
| | C₂₁H₁₈F₃N₃O₂: 402.1 (M + H); Measured: 402.2. |
| 18 | 2-(3-Propylisoxazol-5-yl)-6-(2-trifluoromethylphenyl)-1H- |
| | benzimidazole hydrochloride |
| | ¹H-NMR (400 MHz, d6-DMSO) δ: 7.86 (d, J = 7.1 Hz, 1H), |
| | 7.74 (dd, J = 8.3, 0.8 Hz, 1H), 7.64 (t, J = 7.6 Hz, 1H), 7.60 |
| | (s, 1H), 7.49 (d, J = 7.6 Hz, 1H), 7.75 (t, J = 7.9 Hz, 1H), 7.28 |
| | (dd, J = 8.5, 1.1 Hz, 1H), 7.23 (s, 1H), 2.74 (t, J = 7.5 Hz, 2H), |
| | 1.73 (tq, J = 7.5, 7.5 Hz, 2H), 0.97 (t, J = 7.5 Hz, 3H) |
| | Mass Spectrum (LCMS, ESI pos.): Calculated for |
| | C₂₀H₁₆F₃N₃O: 372.1 (M + H); Measured: 372.2. |
| 19 | 2-(3-Propylisoxazol-5-yl)-6-(2-trifluoromethoxyphenyl)-1H- |
| | benzimidazole hydrochloride |
| | ¹H-NMR (400 MHz, d6-DMSO) δ: 7.77 (dd, J = 8.6, 0.6 Hz, |
| | 1H), 7.74 (d, J = 1.0 Hz, 1H), 7.58-7.66 (m, 1H), 7.49-7.57 |
| | (m, 3H), 7.42 (dd, J = 8.3, 1.8 Hz, 1H), 7.20 (s, 1H), 2.73 (t, J = |
| | 7.5 Hz, 2H), 1.73 (tq, J = 7.5, 7.5 Hz, 2H), 0.97 (t, J = 7.3 |
| | Hz, 3H) |
| | Mass Spectrum (LCMS, ESI pos.): Calculated for |
| | C₂₀H₁₆F₃N₃O₂: 388.1 (M + H); Measured: 388.1. |
| 20 | 2-[3-(2,2-Dimethylpropyl)isoxazol-5-yl]-6-(2- |
| | trifluoromethylphenyl)-1H-benzimidazole hydrochloride |
| | ¹H-NMR (400 MHz, d6-DMSO) δδ: 7.86 (d, J = 7.8 Hz, 1H), |
| | 7.75 (t, J = 7.3 Hz, 1H), 7.73 (d, J = 8.3 Hz, 1H), 7.64 (t, J = |
| | 7.4, 1H), 7.59 (s, 1H), 7.50 (d, J = 7.1 Hz, 1H), 7.26 (d, J = |
| | 8.3 Hz, 1H), 7.14 (s, 1H), 2.66 (s, 2H), 1.00 (s, 9H) |
| | Mass Spectrum (LCMS, ESI pos.): Calculated for |
| | C₂₂H₂₀F₃N₃O: 400.2 (M + H); Measured: 400.2. |
| 21 | 2-[3-(2,2-Dimethylpropyl)-isoxazol-5-yl]-6-(2- |
| | trifluoromethoxyphenyl)-1H-benzimidazole hydrochloride |
| | ¹H-NMR (400 MHz, d6-DMSO) δ: 7.77 (d, J = 8.6 Hz, 1H), |
| | 7.75 (d, J = 1.0 Hz, 1H), 7.59-7.65 (m, 1H), 7.48-7.57 (m, |
| | 3H), 7.42 (dd, J = 8.5, 1.6 Hz, 1H), 7.16 (s, 1H), 2.66 (s, 2H), |
| | 1.00 (s, 9H) |
| | Mass Spectrum (LCMS, ESI pos.): Calculated for |
| | C₂₂H₂₀F₃N₃O₂: 416.2 (M + H); Measured: 416.2. |
| 24 | 2-(3-Cyclohexylisoxazol-5-yl)-6-(2-trifluoromethylphenyl)-1H- |
| | benzimidazole hydrochloride |
| | ¹H-NMR (400 MHz, d6-DMSO) δ: 7.86 (d, J = 7.8 Hz, 1H), |
| | 7.74 (t, J = 7.6 Hz, 1H), 7.72 (d, J = 8.3 Hz, 1H), 7.64 (t, J = |
| | 7.6 Hz, 1H), 7.58 (s, 1H), 7.49 (d, J = 7.6 Hz, 1H), 7.25 (d, J = |
| | 8.6 Hz, 1H), 7.21 (s, 1H), 2.78-2.91 (m, 1H), 1.93-2.04 (m, |
| | 2H), 1.75-1.84 (m, 2H), 1.65-1.75 (m, 1H), 1.35-1.57 (m, 4H), |
| | 1.22-1.34 (m, 1H) |
| | Mass Spectrum (LCMS, ESI pos.): Calculated for |
| | C₂₃H₂₀F₃N₃O: 412.2 (M + H); Measured: 412.2. |
| 25 | 2-(3-Cyclohexylisoxazol-5-yl)-6-(2-trifluoromethoxyphenyl)- |
| | 1H-benzimidazole hydrochloride |
| | ¹H-NMR (400 MHz, d6-DMSO) δ: 7.76 (d, J = 8.3 Hz, 1H), |
| | 7.73 (d, J = 1.0 Hz, 1H), 7.58-7.65 (m, 1H), 7.47-7.57 (m, |
| | 3H), 7.41 (dd, J = 8.3, 1.5 Hz, 1H), 7.22 (s, 1H), 2.79-2.90 (m, |
| | 1H), 1.93-2.03 (m, 2H), 1.75-1.84 (m, 2H), 1.66-1.75 (m, 1H), |
| | 1.35-1.58 (m, 4H), 1.21-1.34 (m, 1H) |
| | Mass Spectrum (LCMS, ESI pos.): Calculated for |
| | C₂₃H₂₀F₃N₃O₂: 428.2 (M + H); Measured: 428.2. |
| 26 | 2-[3-(1-Ethylpropyl)-isoxazol-5-yl]-6-(2-trifluoromethylphenyl)- |
| | 1H-benzimidazole hydrochloride |
| | ¹H-NMR (400 MHz, d6-DMSO) δ: 7.86 (d, J = 7.1 Hz, 1H), |
| | 7.71-7.79 (m, 2H), 7.61-7.68 (m, 1H), 7.60 (s, 1H), 7.50 (d, J = |
| | 7.6 Hz, 1H), 7.27 (dd, J = 8.3, 1.0 Hz, 1H), 7.23 (s, 1H), |
| | 2.66-2.77 (m, 1H), 1.56-1.81 (m, 4H), 0.84 (t, J = 7.3 Hz, 6H) |
| | Mass Spectrum (LCMS, ESI pos.): Calculated for |
| | C₂₂H₂₀F₃N₃O: 400.2 (M + H); Measured: 400.2. |
| 27 | 2-[3-(1-Ethylpropyl)-isoxazol-5-yl]-6-(2- |
| | trifluoromethoxyphenyl)-1H-benzimidazole hydrochloride |
| | ¹H-NMR (400 MHz, d6-DMSO) δ: 7.77 (d, J = 8.3 Hz, 1H), |
| | 7.74 (d, J = 1.0 Hz, 1H), 7.59-7.66 (m, 1H), 7.48-7.56 (m, |
| | 3H), 7.42 (dd, J = 8.5, 1.6 Hz, 1H), 7.22 (s, 1H), 2.66-2.77 (m, |
| | 1H), 1.57-1.82 (m, 4H), 0.84 (t, J = 7.5 Hz, 6H) |
| | Mass Spectrum (LCMS, ESI pos.): Calculated for |
| | C₂₂H₂₀F₃N₃O₂: 416.2 (M + H); Measured: 416.2. |
| 16 | 2-(3-cyclopropylisoxazol-5-yl)-5-(2-trifluoromethylphenyl)-1H- |
| | benzimidazole sodium salt |
| | ¹H-NMR (400 MHz, d6-DMSO) δ: 7.81 (d, 1H, J = 7.8), 7.70 |
| | (t, 1H, J = 7.5), 7.57 (t, J = 7.7 Hz, 1H), 7.55 (d, J = 8.2 Hz, |
| | 1H), 7.46 (d, J = 8.2 Hz, 1H), 7.44 (s, 1H), 7.00 (d, J = 8.3 Hz, |
| | 1H), 6.72 (s, 1H), 2.13-2.02 (m, 1H), 1.08-1.03 (m, 2H), 0.87- |
| | 0.83 (m, 2H) |
| | Mass Spectrum (LCMS, ESI pos.): Calculated for |
| | C₂₀H₁₄F₃N₃O: 370.1 (M + H); Measured: 370.2. |
| 15 | 2-(3-cyclopropylisoxazol-5-yl)-5-(2-methoxyphenyl)-1H- |
| | benzimidazole sodium salt |
| | ¹H-NMR (400 MHz, d6-DMSO) δ: 7.47 (d, J = 1.2 Hz, 1H), |
| | 7.35 (d, J = 8.4 Hz, 1H), 7.29 (dd, J = 7.5, 1.7 Hz, 1H), 7.23 |
| | (ddd, J = 8.2, 7.4, 1.8 Hz, 1H), 7.05 (dd, J = 8.2, 0.8 Hz, 1H), |
| | 6.99 (td, J = 7.4, 1.1 Hz, 1H), 6.93 (dd, J = 8.3, 1.7 Hz, 1H), |
| | 6.39 (s, 1H), 3.74 (s, 3H), 2.01 (tt, J = 8.4, 5.0 Hz, 1H), 1.05- |
| | 0.98 (m, 2H), 0.85-0.79 (m, 2H) |
| | Mass Spectrum (LCMS, ESI pos.): Calculated for |
| | C₂₀H₁₇N₃O₂: 332.1 (M + H); Measured: 332.2. |
| 17 | 2-(3-cyclopropylisoxazol-5-yl)-5-(2-trifluoromethoxyphenyl)- |
| | 1H-benzimidazole sodium salt |
| | ¹H-NMR (400 MHz, d6-DMSO) δ: 7.59-7.53 (m, 3H), 7.50- |
| | 7.43 (m, 3H), 7.11 (dd, J = 8.3, 1.7 Hz, 1H), 6.65 (s, 1H), |
| | 2.11-2.01 (m, 1H), 1.08-1.01 (m, 2H), 0.89-0.81 (m, 2H) |
| | Mass Spectrum (LCMS, ESI pos.): Calculated for |
| | C₂₀H₁₄F₃N₃O₂: 386.1 (M + H); Measured: 386.2. |
| 1 | 2-(3-Isopropylisoxazol-5-yl)-5-phenyl-1H-benzimidazole |
| | sodium salt |
| | ¹H-NMR (400 MHz, d6-DMSO) δ: 7.67-7.62 (m, 3H), 7.44 (d, |
| | J = 8.3 Hz, 1H), 7.40 (t, J = 7.7 Hz, 2H), 7.22 (t, J = 7.3 Hz, |
| | 1H), 7.13 (d, J = 8.3 Hz, 1H), 6.63 (s, 1H), 3.02 (spt, J = 6.9 |
| | Hz, 1H), 1.28 (d, J = 6.9 Hz, 6H) |
| | Mass Spectrum (LCMS, ESI pos.): Calculated for C₁₉H₁₇N₃O: |
| | 304.1 (M + H); Measured: 304.1. |
| 2 | 2-(3-Isopropylisoxazol-5-yl)-5-(2-methoxyphenyl)-1H- |
| | benzimidazole sodium salt |
| | ¹H-NMR (400 MHz, d6-DMSO) δ: 7.48 (d, J = 1.3 Hz, 1H), |
| | 7.36 (d, J = 8.2 Hz, 1H), 7.29 (dd, J = 7.5, 1.7 Hz, 1H), 7.23 |
| | (dt, J = 7.8, 1.3 Hz, 1H), 7.05 (d, J = 8.1 Hz, 1H), 6.99 (dt, J = |
| | 7.4, 1.0 Hz, 1H), 6.93 (dd, J = 8.2, 1.7 Hz, 1H), 6.60 (s, 1H), |
| | 3.75 (s, 3H), 3.02 (spt, J = 6.9 Hz, 1H), 1.28 (d, J = 6.9 Hz, |
| | 6H) |
| | Mass Spectrum (LCMS, ESI pos.): Calculated for |
| | C₂₀H₁₉N₃O₂: 334.2 (M + H); Measured: 334.2. |
| 3 | 2-(3-Isopropylisoxazol-5-yl)-5-(2-trifluoromethylphenyl)-1H- |
| | benzimidazole sodium salt |
| | ¹H-NMR (400 MHz, d6-DMSO) δ: 7.77 (d, J = 8.0 Hz, 1H), |
| | 7.65 (t, J = 7.3 Hz, 1H), 7.51 (t, J = 7.6 Hz, 1H), 7.44 (d, J = |
| | 7.6 Hz, 1H), 7.39 (d, J = 8.2 Hz, 1H), 7.32 (s, 1H), 6.76 (d, J = |
| | 8.2 Hz, 1H), 6.62 (s, 1H), 3.02 (spt, J = 6.9 Hz, 1H), 1.28 (d, J = |
| | 6.9 Hz, 6H) |
| | Mass Spectrum (LCMS, ESI pos.): Calculated for |
| | C₂₀H₁₆F₃N₃O: 372.1 (M + H); Measured: 372.2. |
| 5 | 2-(3-Isopropylisoxazol-5-yl)-5-(2-trifluoromethoxyphenyl)-1H- |
| | benzimidazole sodium salt |
| | ¹H-NMR (400 MHz, d6-DMSO) δ: 7.54 (dd, J = 7.7, 1.2 Hz, |
| | 1H) 7.49 (d, J = 1.3 Hz, 1H), 7.47-7.36 (m, 4H), 6.93 (dd, J = |
| | 8.3, 1.7 Hz, 1H), 6.64 (s, 1H), 3.03 (spt, J = 6.9 Hz, 1H), 1.28 |
| | (d, J = 6.9 Hz, 6H) |
| | Mass Spectrum (LCMS, ESI pos.): Calculated for |
| | C₂₀H₁₆F₃N₃O₂: 388.1 (M + H); Measured: 388.1. |
| 6 | 2-(3-Isopropylisoxazol-5-yl)-5-(2-ethoxyphenyl)-1H- |
| | benzimidazole sodium salt |
| | ¹H-NMR (400 MHz, d6-DMSO) δ: 7.54 (d, J = 1.2 Hz, 1H), |
| | 7.37 (d, J = 8.2 Hz, 1H), 7.30 (dd, J = 7.5, 1.7 Hz, 1H), 7.20 |
| | (dt, J = 7.7, 1.3 Hz, 1H), 7.04 (d, J = 8.0 Hz, 1H), 7.00 (dd, J = |
| | 8.3, 1.8 Hz, 1H), 6.98 (dt, J = 7.5, 1.0 Hz, 1H), 6.62 (s, 1H), |
| | 4.02 (q, J = 6.9 Hz, 2H), 3.02 (spt, J = 6.9 Hz, 1H), 1.28 (d, J = |
| | 6.9 Hz, 6H), 1.27 (t, J = 7.0 Hz, 3H) |
| | Mass Spectrum (LCMS, ESI pos.): Calculated for |
| | C₂₁H₂₁N₃O₂: 348.2 (M + H); Measured: 348.1. |
| 7 | 2-(3-Isopropylisoxazol-5-yl)-5-(3-trifluoromethylphenyl)-1H- |
| | benzimidazole sodium salt |
| | ¹H-NMR (400 MHz, d6-DMSO) δ: 7.96 (d, J = 7.8 Hz, 1H), |
| | 7.89 (s, 1H), 7.73 (d, J = 1.5 Hz, 1H), 7.64 (t, J = 7.8 Hz, 1H), |
| | 7.57 (d, J = 7.9 Hz, 1H), 7.47 (d, J = 8.3 Hz, 1H), 7.18 (dd, J = |
| | 8.3, 1.9 Hz, 1H), 6.64 (s, 1H), 3.03 (spt, J = 6.9 Hz, 1H), |
| | 1.28 (d, J = 6.9 Hz, 6H) |
| | Mass Spectrum (LCMS, ESI pos.): Calculated for |
| | C₂₀H₁₆F₃N₃O: 372.1 (M + H); Measured: 372.1. |
| 10 | 2-(3-tert-Butylisoxazol-5-yl)-5-(2-trifluoromethoxyphenyl)-1H- |
| | benzimidazole sodium salt |
| | ¹H-NMR (400 MHz, d6-DMSO) δ: 7.55 (dd, J = 7.4, 1.0 Hz, |
| | 1H), 7.51 (d, J = 1.3 Hz, 1H), 7.47-7.36 (m, 4H), 6.95 (dd, J = |
| | 8.3, 1.8 Hz, 1H), 6.72 (s, 1H), 1.35 (s, 9H) |
| | Mass Spectrum (LCMS, ESI pos.): Calculated for |
| | C₂₁H₁₈F₃N₃O₂: 402.1 (M + H); Measured: 402.1. |
| 9 | 2-(3-tert-Butylisoxazol-5-yl)-5-(2-trifluoromethylphenyl)-1H- |
| | benzimidazole sodium salt |
| | ¹H-NMR (400 MHz, d6-DMSO) δ: 7.78 (d, J = 7.9 Hz, 1H), |
| | 7.66 (t, J = 7.4 Hz, 1H), 7.51 (t, J = 7.6 Hz, 1H), 7.44 (d, J = |
| | 7.6 Hz, 1H), 7.40 (d, J = 8.2 Hz, 1H), 7.33 (s, 1H), 6.77 (dd, J = |
| | 8.2, 1.0 Hz, 1H), 6.70 (s, 1H), 1.34 (s, 9H) |
| | Mass Spectrum (LCMS, ESI pos.): Calculated for |
| | C₂₁H₁₈F₃N₃O: 386.1 (M + H); Measured: 386.2. |
| 8 | 2-(3-tert-Butylisoxazol-5-yl)-5-(2-methoxyphenyl)-1H- |
| | benzimidazole sodium salt |
| | ¹H-NMR (400 MHz, d6-DMSO) δ: 7.48 (d, J = 1.2 Hz, 1H), |
| | 7.36 (d, J = 8.2 Hz, 1H), 7.29 (dd, J = 7.5, 1.7 Hz, 1H), 7.23 |
| | (ddd, J = 8.2, 7.5, 1.8 Hz, 1H), 7.05 (dd, J = 7.5, 0.7 Hz, 1H), |
| | 7.05 (m, 1H), 6.99 (dt, J = 7.4, 1.1 Hz, 1H), 6.94 (dd, J = 8.2, |
| | 1.7 Hz, 1H), 6.66 (s, 1H), 3.75 (s, 3H), 1.33 (s, 9H) |
| | Mass Spectrum (LCMS, ESI pos.): Calculated for |
| | C₂₁H₂₁N₃O₂: 348.2 (M + H); Measured: 348.1. |
| 92 | 2-(3-Cyclopentylisoxazol-5-yl)-5-(2-trifluoromethylphenyl)-1H- |
| | benzimidazole |
| | ¹H-NMR (400 MHz, CD3OD) δ: 7.80 (d, J = 7.8 Hz, 1H), 7.70 |
| | (br. s., 1H), 7.65 (t, J = 7.6 Hz, 1H), 7.59 (br. s., 1H), 7.56 (t, J = |
| | 7.6 Hz, 1H), 7.43 (d, J = 7.6 Hz, 1H), 7.29 (d, J = 8.3 Hz, |
| | 1H), 7.01 (s, 1H), 3.27 (p, J = 7.8 Hz, 1H), 2.06-2.22 (m, 2H), |
| | 1.70-1.90 (m, 6H) |
| | Mass Spectrum (LCMS, ESI pos.): Calculated for |
| | C₂₂H₁₈F₃N₃O: 398.1 (M + H); Measured: 398.1. |
| 52 | 2-[2-Methyl-5-(1-methylcyclopropyl)-2H-pyrazol-3-yl]-5-(2- |
| | trifluoromethylphenyl)-1H-benzimidazole sodium salt |
| | ¹H-NMR (400 MHz, d6-DMSO) δ: 7.78 (d, J = 7.3 Hz, 1H), |
| | 7.66 (t, J = 7.6 Hz, 1H), 7.52 (t, J = 7.7 Hz, 1H), 7.44 (d, J = |
| | 7.6 Hz, 1H), 7.41 (d, J = 8.3 Hz, 1H), 7.32 (s, 1H), 6.80 (d, J = |
| | 8.1 Hz, 1H), 6.47 (s, 1H), 4.26 (s, 3H), 1.41 (s, 3H), 0.85-0.93 |
| | (m, 2H), 0.64-0.71 (m, 2H) |
| | Mass Spectrum (LCMS, ESI pos.): Calculated for C₂₂H₁₉F₃N₄: |
| | 397.2 (M + H); Measured: 397.3. |
| 53 | 2-[2-Methyl-5-(1-methylcyclopropyl)-2H-pyrazol-3-yl]-5-(2- |
| | trifluoromethoxyphenyl)-1H-benzimidazole sodium salt |
| | ¹H-NMR (400 MHz, d6-DMSO) δ: 7.52-7.59 (m, 1H), 7.49 (d, |
| | J = 1.5 Hz, 1H), 7.35-7.47 (m, 4H), 6.97 (dd, J = 8.1, 1.5 Hz, |
| | 1H), 6.48 (s, 1H), 4.27 (s, 3H), 1.41 (s, 3H), 0.86-0.93 (m, |
| | 2H), 0.63-0.72 (m, 2H) |
| | Mass Spectrum (LCMS, ESI pos.): Calculated for |
| | C₂₂H₁₉F₃N₄O: 413.2 (M + H); Measured: 413.3. |
| 91 | 2-(5-Cyclobutyl-1-methyl-1H-pyrazol-3-yl)-5-(2-trifluoromethyl- |
| | phenyl)-1H-benzimidazole |
| | ¹H-NMR (400 MHz, d6-DMSO + d1-TFA) δ: 7.90 (d, J = 7.8 |
| | Hz, 1H), 7.85 (d, J = 8.6 Hz, 1H), 7.75-7.82 (m, 1H), 7.71 (s, |
| | 1H), 7.65-7.73 (m, 1H), 7.50 (s, 1H), 7.48 (s, 1H), 7.12 (s, |
| | 1H), 3.90 (s, 3H), 3.67-3.82 (m, 1H), 2.43-2.50 (m, 2H), 2.01- |
| | 2.21 (m, 3H), 1.86-1.99 (m, 1H) |
| | Mass Spectrum (LCMS, ESI pos.): Calculated for C₂₂H₁₉F₃N₄: |
| | 397.2 (M + H); Measured: 397.2. |
| 37 | 2-(5-Cyclobutyl-2-methyl-2H-pyrazol-3-yl)-5-(2-trifluoromethyl- |
| | phenyl)-1H-benzimidazole sodium salt |
| | ¹H-NMR (400 MHz, CD₃OD) δ: 7.77 (d, J = 7.8 Hz, 1H), 7.58- |
| | 7.68 (m, 2H), 7.47-7.57 (m, 2H), 7.44 (d, J = 7.6 Hz, 1H), |
| | 7.04-7.11 (m, 1H), 6.73 (s, 1H), 4.21 (s, 3H), 3.57 (quin, J = |
| | 8.7 Hz, 1H), 2.20-2.43 (m, 4H), 1.87-2.14 (m, 2H) |
| | Mass Spectrum (LCMS, ESI pos.): Calculated for C₂₂H₁₉F₃N₄: |
| | 397.2 (M + H); Measured: 397.2. |
| 38 | 2-(5-Cyclobutyl-2-methyl-2H-pyrazol-3-yl)-5-(2- |
| | trifluoromethoxy-phenyl)-1H-benzimidazole sodium salt |
| | ¹H-NMR (400 MHz, CD₃OD) δ: 7.68 (d, J = 1.0 Hz, 1H), 7.62 |
| | (d, J = 8.3 Hz, 1H), 7.52-7.57 (m, 1H), 7.33-7.44 (m, 3H), |
| | 7.17 (dd, J = 8.3, 1.8 Hz, 1H), 6.71 (s, 1H), 4.20 (s, 3H), 3.56 |
| | (quin, J = 8.7 Hz, 1H), 2.20-2.43 (m, 4H), 1.86-2.13 (m, 2H) |
| | Mass Spectrum (LCMS, ESI pos.): Calculated for |
| | C₂₂H₁₉F₃N₄O: 413.2 (M + H); Measured: 413.2. |
| 39 | 2-(5-Cyclobutyl-2-methyl-2H-pyrazol-3-yl)-5-(2-chlorophenyl)- |
| | 1H-benzimidazole sodium salt |
| | ¹H-NMR (400 MHz, CD₃OD) δ: 7.59-7.67 (m, 2H), 7.48 (dd, J = |
| | 7.8, 1.3 Hz, 1H), 7.43 (dd, J = 7.6, 1.8 Hz, 1H), 7.24-7.38 |
| | (m, 2H), 7.18 (dd, J = 8.2, 1.6 Hz, 1H), 6.73 (s, 1H), 4.21 (s, |
| | 3H), 3.57 (quin, J = 8.6 Hz, 1H), 2.18-2.44 (m, 4H), 1.88-2.15 |
| | (m, 2H) |
| | Mass Spectrum (LCMS, ESI pos.): Calculated for C₂₂H₁₉ClN₄: |
| | 363.1 (M + H); Measured: 363.2. |
| 30 | 2,7,7-Trimethyl-3-[5-(2-trifluoromethylphenyl)-1H- |
| | benzimidazol-2-yl]-4,5,6,7-tetrahydro-2H-indazole sodium salt |
| | ¹H-NMR (400 MHz, CD₃OD) δ: 7.75 (d, J = 7.6 Hz, 1H), 7.61 |
| | (t, J = 7.6 Hz, 1H), 7.58 (dd, J = 8.1, 0.5 Hz, 1H), 7.52 (s, 1H), |
| | 7.49 (t, J = 7.7 Hz, 1H), 7.44 (d, J = 7.6 Hz, 1H), 7.01 (dd, J = |
| | 8.1, 1.0 Hz, 1H), 4.02 (s, 3H), 2.72 (t, J = 6.3 Hz, 2H), 1.76- |
| | 1.87 (m, 2H), 1.63-1.72 (m, 2H), 1.32 (s, 6H) |
| | Mass Spectrum (LCMS, ESI pos.): Calculated for C₂₄H₂₃F₃N₄: |
| | 425.2 (M + H); Measured: 425.3. |
| 31 | 2,7,7-Trimethyl-3-[5-(2-trifluoromethoxyphenyl)-1H- |
| | benzimidazol-2-yl]-4,5,6,7-tetrahydro-2H-indazole sodium salt |
| | ¹H-NMR (400 MHz, CD₃OD) δ: 7.68 (d, J = 1.3 Hz, 1H), 7.63 |
| | (d, J = 8.3 Hz, 1H), 7.52-7.58 (m, 1H), 7.34-7.44 (m, 3H), |
| | 7.18 (dd, J = 8.3, 1.8 Hz, 1H), 4.03 (s, 3H), 2.72 (t, J = 6.2 |
| | Hz, 2H), 1.76-1.86 (m, 2H), 1.64-1.73 (m, 2H), 1.32 (s, 6H) |
| | Mass Spectrum (LCMS, ESI pos.): Calculated for |
| | C₂₄H₂₃F₃N₄O: 441.2 (M + H); Measured: 441.3. |
| 32 | 3-[5-(2-Chlorophenyl)-1H-benzimidazol-2-yl]-2,7,7-trimethyl- |
| | 4,5,6,7-tetrahydro-2H-indazole sodium salt |
| | ¹H-NMR (400 MHz, CD₃OD) δ: 7.59-7.64 (m, 2H), 7.48 (dd, J = |
| | 7.8, 1.3 Hz, 1H), 7.44 (dd, J = 7.6, 1.5 Hz, 1H), 7.23-7.37 |
| | (m, 2H), 7.14 (dd, J = 8.1, 1.8 Hz, 1H), 4.03 (s, 3H), 2.72 (t, J = |
| | 6.3 Hz, 2H), 1.76-1.86 (m, 2H), 1.64-1.73 (m, 2H), 1.32 (s, |
| | 6H) |
| | Mass Spectrum (LCMS, ESI pos.): Calculated for C₂₄H₂₃ClN₄: |
| | 391.2 (M + H); Measured: 391.3. |

### Example 13

### 2-(3-Chloroisoxazol-5-yl)-5-(2-trifluoromethylphenyl)-1H-benzimidazole sodium salt (Compound #33)

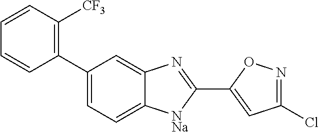

### STEP A. 2-Methyl-3-oxo-2,3-dihydro-isoxazole-5-carboxylic acid methyl ester

3-Hydroxy-isoxazole-5-carboxylic acid methyl ester (716 mg, 5.00 mmol) and K₂CO₃ (1.73 g, 12.5 mmol) were suspended in acetone (25 mL), and CH₃I (0.800 mL, 12.5 mmol) was added. The resulting mixture was stirred at room temperature for 16 h, diluted with EtOAc (10 mL), and filtered. The solvent was removed under reduced pressure, and the residue was chromatographed using a 40-g SiO₂ pre-packed column eluting with 0:1 to 1:4 EtOAc-hexanes to yield a residue. ¹H-NMR (400 MHz, CDCl₃) δ: 6.40 (s, 1H), 3.96 (s, 3H), 3.62 (s, 3H).

### STEP B. 3-Chloroisoxazole-5-carboxylic acid methyl ester

2-Methyl-3-oxo-2,3-dihydro-isoxazole-5-carboxylic acid methyl ester (121 mg, 0.769 mmol, as prepared in the previous step) was dissolved in POCl₃ (1.5 mL), and the resulting mixture was stirred at 90° C. for 12 h. The solvent was removed under reduced pressure, and the residue was dissolved in DCM (20 mL) and washed with H₂O (10 mL). The aqueous phase was extracted with DCM (2×20 mL), and then the combined organic extracts were washed with sat aqueous NaHCO₃ (10 mL), dried over MgSO₄, and filtered. The solvent was removed under reduced pressure to yield a residue. ¹H-NMR (400 MHz, CDCl₃) δ: 6.96 (s, 1H), 4.00 (s, 3H).

### STEP C. (4-Bromo-2-tert-butoxycarbonylaminophenyl)-carbamic acid tert-butyl ester

4-Bromo-1,2-diaminobenzene (1.87 g, 10.0 mmol) was dissolved in DCM (50 mL), and Boc₂O (5.46 g, 25.0 mmol) was added. 2.5 M aqueous NaOH (10.0 mL, 25.0 mmol) was added then the resulting mixture was stirred at room temperature for 72 h. Additional Boc₂O (2.50 g) was added, and the resulting mixture was stirred at room temperature for an additional 72 h. The resulting mixture was diluted with H₂O (10 mL) and extracted with DCM (3×20 mL). The combined organic extracts were dried over MgSO₄ and filtered. The solvent was removed under reduced pressure, and the residue was chromatographed using a 50-g SiO₂ pre-packed column eluting with 1:9 EtOAc-hexanes, to yield a residue, which was used directly in the next step.

### STEP D. (3-tert-Butoxycarbonylamino-2′-trifluoromethylbiphenyl-4-yl)-carbamic acid tert-butyl ester

Following the procedure described in Step B of Example 1, the title compound was prepared from (4-bromo-2-tert-butoxycarbonylaminophenyl)-carbamic acid tert-butyl ester (775 mg, 2.00 mmol, as prepared in the previous step) and 2-trifluoromethylphenylboronic acid (570 mg, 3.00 mmol). ¹H-NMR (400 MHz, CDCl₃) δ: 7.72 (d, J=7.3 Hz, 1H), 7.50-7.57 (m, 2H), 7.41-7.48 (m, 2H), 7.33 (d, J=7.6 Hz, 1H), 7.11 (d, J=7.1 Hz, 1H), 6.77 (br. s., 2H), 1.54 (s, 9H), 1.50 (s, 9H).

### STEP E. 2′-Trifluoromethyl-biphenyl-3,4-diamine

(3-tert-Butoxycarbonylamino-2′-trifluoromethyl-biphenyl-4-yl)-carbamic acid tert-butyl ester (226 mg, 0.500 mmol, as prepared in the previous step) was dissolved in DCM (3 mL), and TFA (3 mL) was added dropwise over 2 min. The resulting mixture was stirred at room temperature for 12 h, and the solvent was removed under reduced pressure. The residue was dissolved in DCM (10 mL), washed with sat aqueous NaHCO₃ (10 mL), dried over MgSO₄, and filtered. The solvent was removed under reduced pressure to yield a residue. ¹H-NMR (400 MHz, CDCl₃) δ: 7.69 (d, J=8.1 Hz, 1H), 7.49 (t, J=7.1 Hz, 1H), 7.39 (t, J=7.5 Hz, 1H), 7.31 (d, J=7.6 Hz, 1H), 6.64-6.72 (m, 3H), 3.41 (br. s., 4H).

### STEP F. 2-(3-Chloroisoxazol-5-yl)-5-(2-trifluoromethylphenyl)-1H-benzimidazole

3-Chloroisoxazole-5-carboxylic acid methyl ester (95.9 mg, 0.594 mmol, as prepared in Step B of Example 13) was dissolved in a mixture of 1,4-dioxane (2 mL) and MeOH (2 mL), and 2.5 M aqueous NaOH (0.50 mL, 1.25 mmol) was added. The resulting mixture was stirred at room temperature for 72 h and extracted with Et₂O (2×10 mL). The aqueous layer was acidified to ca. pH 2 with 3 M aqueous HCl and extracted with DCM (3×20 mL). The combined organic extracts were dried over MgSO₄ and filtered. The solvent was removed under reduced pressure, to yield a residue, which was used directly in the next step.

A solution of 3-chloroisoxazole-5-carboxylic acid (35.1 mg, 0.238 mmol, as prepared above) in DCM (1 mL) was treated with oxalyl chloride (31.0 μL, 0.357 mmol), and the resulting mixture was stirred at room temperature for 3 h. The solvent was removed under reduced pressure, and the resulting residue was dissolved in anhydrous THF (3 mL).

To a solution of 2′-trifluoromethyl-biphenyl-3,4-diamine (126 mg, 0.500 mmol, as prepared in the previous step) in dry THF (5 mL) was added DIPEA (87.0 μL, 0.500 mmol). The THF solution of the residue prepared as described above was added dropwise over 5 min. The resulting mixture was stirred at room temperature for 16 h, then the solvent was removed under reduced pressure. The residue was chromatographed using a 12-g SiO₂ pre-packed column eluting with 0:1 to 2:3 EtOAc-hexanes, to yield a residue as a mixture of the mono-amide isomers.

The residue (the mixture of mono-amide isomers) was dissolved in anhydrous 1,4-dioxane (2 mL), p-TsOH.H₂O (24.0 mg, 0.124 mmol) was added, and the resulting mixture was stirred at 100° C. for 6 h. The resulting mixture was cooled to room temperature and basified to ca. pH 10 using 2.5 M NaOH. The aqueous layer was extracted with EtOAc (3×20 mL), and the combined organic extracts were dried over MgSO₄ and filtered. The solvent was removed under reduced pressure, and the residue was chromatographed using a 12-g SiO₂ pre-packed column eluting with 0:1 to 2:3 EtOAc-hexanes to yield a white solid. ¹H-NMR (400 MHz, CD₃OD) δ: 7.81 (d, J=7.8 Hz, 1H), 7.72 (d, J=8.3 Hz, 1H), 7.64-7.70 (m, 1H), 7.61 (s, 1H), 7.54-7.60 (m, 1H), 7.44 (d, J=7.6 Hz, 1H), 7.33 (d, J=8.3 Hz, 1H), 7.20 (s, 1H). Mass Spectrum (LCMS, ESI pos.): Calculated for C₁₇H₉ClF₃N₃O: 364.0 (M+H); Measured: 364.1.

### STEP G. 2-(3-Chloroisoxazol-5-yl)-5-(2-trifluoromethylphenyl)-1H-benzimidazole sodium salt

Following the procedure described in Step C of Example 2, the title compound was prepared from 2-(3-chloroisoxazol-5-yl)-5-(2-trifluoromethylphenyl)-1H-benzimidazole (38.5 mg, 0.106 mmol, as prepared in the previous step) and 0.5 M NaOMe in MeOH (212 μL, 0.106 mmol) as a white foam.

¹H-NMR (400 MHz, d6-DMSO) δ: 7.78 (d, J=7.1 Hz, 1H), 7.67 (t, J=7.6 Hz, 1H), 7.53 (t, J=7.7 Hz, 1H), 7.44 (d, J=7.6 Hz, 1H), 7.44 (d, J=7.6 Hz, 1H), 7.35 (s, 1H), 6.91 (s, 1H), 6.80 (dd, J=8.2, 1.1 Hz, 1H). Mass Spectrum (LCMS, ESI pos.): Calculated for C₁₇H₉ClF₃N₃O: 364.0 (M+H); Measured: 364.1.

Following the procedure described in Example 13, and selecting and substituting reagents, starting materials, and conditions as would be known to those skilled in the art, the following compounds of formula (I) of the present invention were prepared:

| ID | |
|---|---|
| No. | Name and Measured Physical Property |
| 34 | 2-(3-Chloroisoxazol-5-yl)-5-(2-trifluoromethoxyphenyl)-1H- |
| | benzimidazole sodium salt |
| | ¹H-NMR (400 MHz, d6-DMSO) δ: 7.53-7.57 (m, 1H), 7.53 (d, J = |
| | 1.0 Hz, 1H), 7.47 (d, J = 8.8 Hz, 1H), 7.37-7.46 (m, 3H), 6.97 |
| | (dd, J = 8.2, 1.9 Hz, 1H), 6.91 (s, 1H) |
| | Mass Spectrum (LCMS, ESI pos.): Calculated for |
| | C₁₇H₉ClF₃N₃O₂: 380.0 (M + H); Measured: 380.1. |

### Example 14

### 2-(3-Bromoisoxazol-5-yl)-5-(2-trifluoromethylphenyl)-1H-benzimidazole sodium salt (Compound #42)

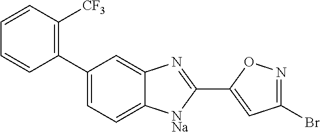

### STEP A. 3-Bromoisoxazole-5-carboxylic acid tert-butyl ester

Dibromoformaldoxime (1.05 g, 5.18 mmol) was dissolved in EtOAc (25 mL), and tert-butyl propiolate (1.40 mL, 10.4 mmol) was added. Saturated aqueous NaHCO₃ (5 mL) was added, and the resulting biphasic mixture was stirred at room temperature for 16 h. The organic layer was separated, and the aqueous phase was extracted with EtOAc (2×10 mL). The combined organic extracts were dried over MgSO₄ and filtered. The solvent was removed under reduced pressure, and the residue was chromatographed using a 40-g SiO₂ pre-packed column eluting with 0:1 to 3:97 EtOAc-hexanes to yield a residue. ¹H-NMR (400 MHz, CDCl₃) δ: 6.90 (s, 1H), 1.60 (s, 9H).

### STEP B. 2-(3-Bromoisoxazol-5-yl)-5-(2-trifluoromethylphenyl)-1H-benzimidazole

3-Bromoisoxazole-5-carboxylic acid tert-butyl ester (143 mg, 0.574 mmol, as prepared in the previous step) was dissolved in DCM (1 mL), and TFA (1 mL) was added dropwise. The resulting mixture was stirred at room temperature for 12 h, and the solvent was removed under reduced pressure, to yield a residue, which was used directly in the next step.

To a solution of 3-bromoisoxazole-5-carboxylic acid (19.0 mg, 0.100 mmol, as prepared above) and 2′-trifluoromethyl-biphenyl-3,4-diamine (25.1 mg, 1.00 mmol, as prepared in Step E of Example 13) in dry 1,4-dioxane (2 mL) was added DCC (23.0 mg, 0.110 mmol). The resulting mixture was stirred at room temperature for 16 h, and p-TsOH.H₂O (19.0 mg, 0.100 mmol) was added. The resulting mixture was stirred at 100° C. for 6 h, cooled to room temperature, and basified ca. to pH 10 using 2.5 aqueous M NaOH. The aqueous layer was extracted with EtOAc (3×20 mL), and the combined organic extracts were dried over MgSO₄ and filtered. The solvent was removed under reduced pressure, and the residue was chromatographed using a 12-g SiO₂ pre-packed column eluting with 0:1 to 2:3 EtOAc-hexanes, to yield a white solid. ¹H-NMR (400 MHz, CD₃OD) δ: 7.80 (d, J=7.8 Hz, 1H), 7.72 (d, J=8.3 Hz, 1H), 7.63-7.70 (m, 1H), 7.61 (s, 1H), 7.54-7.60 (m, 1H), 7.44 (d, J=7.6 Hz, 1H), 7.28-7.36 (m, 1H), 7.24 (s, 1H). Mass Spectrum (LCMS, ESI pos.): Calculated for C₁₇H₉BrF₃N₃O: 408.0 (M+H); Measured: 408.0.

### STEP C. 2-(3-Bromoisoxazol-5-yl)-5-(2-trifluoromethylphenyl)-1H-benzimidazole sodium salt

Following the procedure described in Step C of Example 2, the title compound was prepared from 2-(3-bromoisoxazol-5-yl)-5-(2-trifluoromethylphenyl)-1H-benzimidazole (17.9 mg, 0.0420 mmol, as prepared in the previous step) and 0.5 M NaOMe in MeOH (84.0 μL, 0.0420 mmol), as a white foam.

¹H-NMR (400 MHz, d6-DMSO) δ: 7.78 (d, J=7.8 Hz, 1H), 7.66 (t, J=7.2 Hz, 1H), 7.52 (t, J=7.7 Hz, 1H), 7.44 (d, J=7.6 Hz, 1H), 7.43 (d, J=7.8 Hz, 1H), 7.35 (s, 1H), 6.90 (s, 1H), 6.80 (dd, J=8.3, 1.0 Hz, 1H). Mass Spectrum (LCMS, ESI pos.): Calculated for C₁₇H₉BrF₃N₃O: 408.0 (M+H); Measured: 408.0.

Following the procedure described in Example 14, and selecting and substituting reagents, starting materials, and conditions as would be known to those skilled in the art, the following compounds of formula (I) of the present invention were prepared:

| ID | |
|---|---|
| No. | Name and Measured Physical Property |
| 43 | 2-(3-Bromoisoxazol-5-yl)-5-(2-trifluoromethoxyphenyl)-1H- |
| | benzimidazole sodium salt |
| | ¹H-NMR (400 MHz, d6-DMSO) δ: 7.54-7.59 (m, 1H), 7.53 (d, J = |
| | 1.3 Hz, 1H), 7.48 (d, J = 8.8 Hz, 1H), 7.37-7.47 (m, 3H), 6.98 |
| | (dd, J = 8.3, 1.8 Hz, 1H), 6.93 (s, 1H) |
| | Mass Spectrum (LCMS, ESI pos.): Calculated for |
| | C₁₇H₉BrF₃N₃O₂: 424.0 (M + H); Measured: 424.0. |

### Example 15

### 2-(5-tert-Butyl-4-chloro-2-methyl-2H-pyrazol-3-yl)-5-(2,6-difluoro-phenyl)-1H-benzimidazole hydrochloride (Compound #85)

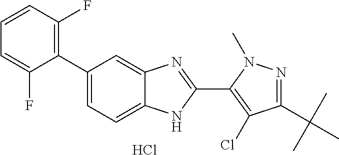

### STEP A. 2′,6′-Difluoro-3-nitro-biphenyl-4-ylamine

To a mixture of 4-amino-2-nitrophenylboronic acid (606 mg, 3.30 mmol, 90%), 2-bromo-1,3-difluoro-benzene (579 mg, 3.00 mmol) and Pd(PPh₃)₄ (347 mg, 0.300 mmol) in 1,4-dioxane (9 mL) was added 2.0 M aqueous Na₂CO₃ (6.00 mL, 12.0 mmol). The resulting mixture was stirred at 110° C. under microwave irradiation for 1 h and then cooled to room temperature. The resulting mixture was treated with EtOAc (50 mL), then was washed with H₂O and brine and was dried (Na₂SO₄). The solvent was removed under reduced pressure followed by flash chromatography of the residue on silica gel (5-40% EtOAc-hexanes) to yield a yellow solid. ¹H-NMR (400 MHz, CDCl₃) δ: 8.30 (s, 1H), 7.50 (dd, J=8.6, 1.8 Hz, 1H), 7.23-7.34 (m, 1H), 6.95-7.05 (m, 1H), 6.90 (d, J=8.6 Hz, 1H), 6.19 (br. s., 2H). Mass Spectrum (LCMS, ESI pos.) Calculated For C₁₂H₈F₂N₂O₂: 251.1 (M+H), Measured: 251.0.

### STEP B. 5-tert-Butyl-4-chloro-2-methyl-2H-pyrazole-3-carboxylic acid (2′,6′-difluoro-3-nitro-biphenyl-4-yl)-amide

To a mixture of NaH (30.3 mg, 1.20 mmol, 95%) in 3 mL of DMF was added 2′,6′-difluoro-3-nitro-biphenyl-4-ylamine (75.0 mg, 0.300 mmol). The resulting mixture was stirred at room temperature for 15 min under Ar.

To a mixture of 5-tert-butyl-4-chloro-2-methyl-2H-pyrazole-3-carboxylic acid (as prepared in Example T, 68.3 mg, 0.315 mmol) in DCM (5 mL) (containing 50 μL of DMF) was added oxalyl chloride (28.0 μL, 0.322 mmol). After stirring at room temperature for 1 h under Ar, the mixture was concentrated in vacuo, and the solid residue was added to the sodium salt solution prepared above. The resulting mixture was stirred at room temperature for 2 h under Ar and then quenched with 5 mL of saturated aqueous NH₄Cl. The resulting mixture was treated with EtOAc (50 mL), then was washed with H₂O and brine and was dried with Na₂SO₄. The solvent was removed under reduced pressure followed by flash chromatography of the residue on silica gel (0-5% EtOAc-hexanes) to yield a light yellow solid. ¹H-NMR (400 MHz, CDCl₃) δ: 11.14 (s, 1H), 8.88 (d, J=8.8 Hz, 1H), 8.40 (s, 1H), 7.82 (dd, J=8.8, 1.5 Hz, 1H), 7.38 (tt, J=8.5, 6.3 Hz, 1H), 6.95-7.13 (m, 2H), 4.13 (s, 3H), 1.44 (s, 9H). Mass Spectrum (LCMS, ESI pos.) Calculated For C₂₁H₁₉ClF₂N₄O₃: 449.1 (M+H), Measured: 449.1.

### STEP C. 2-(5-tert-Butyl-4-chloro-2-methyl-2H-pyrazol-3-yl)-5-(2,6-difluoro-phenyl)-1H-benzimidazole

A mixture of 5-tert-butyl-4-chloro-2-methyl-2H-pyrazole-3-carboxylic acid (2′,6′-difluoro-3-nitro-biphenyl-4-yl)-amide (as prepared in the previous step, 80.0 mg, 0.178 mmol) and iron powder (80.0 mg, 1.43 mmol) in 1:1 HOAc/EtOH (2 mL) was stirred at 110° C. under microwave irradiation for 1 h, then cooled to room temperature. The solid was removed by filtration through a pad of diatomaceous earth and washed with DCM (20 mL). The filtrate was concentrated under reduced pressure, and the residue was purified by flash chromatography on silica gel (5-20% EtOAc-hexanes) to yield a white solid. ¹H-NMR (400 MHz, CDCl₃) δ: 7.61-7.90 (m, 2H), 7.32-7.48 (m, 2H), 7.03-7.15 (m, 2H), 4.04 (s, 3H), 1.45 (s, 9H). Mass Spectrum (LCMS, ESI pos.) Calculated For C₂₁H₁₉ClF₂N₄: 401.1 (M+H), Measured: 401.1.

### STEP D. 2-(5-tert-Butyl-4-chloro-2-methyl-2H-pyrazol-3-yl)-5-(2,6-difluoro-phenyl)-1H-benzimidazole hydrochloride

To a solution of 2-(5-tert-butyl-4-chloro-2-methyl-2H-pyrazol-3-yl)-5-(2,6-difluoro-phenyl)-1H-benzimidazole (as prepared in the previous step, 49.7 mg, 0.124 mmol) in 2 mL of 1:1 MeOH/DCM was added 1.0 M HCl in ethyl ether (124 μL, 0.124 mmol). After stirring at room temperature for 0.5 h, the mixture was concentrated in vacuo to yield the title compound as a white solid.

¹H-NMR (400 MHz, CD₃OD) δ: 8.03 (d, J=8.6 Hz, 1H), 8.00 (s, 1H), 7.78 (d, J=8.6 Hz, 1H), 7.46-7.55 (m, 1H), 7.11-7.21 (m, 2H), 4.08 (s, 3H), 1.46 (s, 9H). Mass Spectrum (LCMS, ESI pos.) Calculated For C₂₁H₁₉ClF₂N₄: 401.1 (M+H), Measured: 401.2.

Following the procedure described in Example 15, and selecting and substituting reagents, starting materials, and conditions as would be known to those skilled in the art, the following compounds of formula (I) of the present invention were prepared:

| ID | |
|---|---|
| No. | Name and Measured Physical Property |
| 86 | 2-(5-tert-Butyl-4-chloro-2-methyl-2H-pyrazol-3-yl)-5-(2-fluoro-6- |
| | trifluoromethyl-phenyl)-1H-benzimidazole hydrochloride |
| | ¹H-NMR (400 MHz, CD₃OD) δ: 8.04 (d, J = 8.6 Hz, 1H), 7.90 |
| | (s, 1H), 7.68-7.76 (m, 2H), 7.66 (d, J = 8.6 Hz, 1H), 7.53-7.61 |
| | (m, 1H), 4.09 (s, 3H), 1.47 (s, 9H) |
| | Mass Spectrum (LCMS, ESI pos.) Calculated For |
| | C₂₂H₁₉ClF₄N₄: 451.1 (M + H), Measured: 451.2 |
| 88 | 2-(5-tert-Butyl-4-chloro-2-methyl-2H-pyrazol-3-yl)-5-(2-fluoro-6- |
| | trifluoromethoxy-phenyl)-1H-benzimidazole hydrochloride |
| | ¹H-NMR (400 MHz, CD3OD) δ: 7.90 (d, J = 8.3 Hz, 1H), 7.81 |
| | (s, 1H), 7.47-7.60 (m, 2H), 7.27-7.37 (m, 2H), 4.06 (s, 3H), |
| | 1.46 (s, 9H) |
| | Mass Spectrum (LCMS, ESI pos.) Calculated For |
| | C22H19ClF4N4O: 467.1 (M + H), Measured: 467.2 |
| 87 | 5-(2,6-Bis-trifluoromethyl-phenyl)-2-(5-tert-butyl-4-chloro-2- |
| | methyl-2H-pyrazol-3-yl)-1H-benzimidazole hydrochloride |
| | ¹H-NMR (400 MHz, CD₃OD) δ: 8.15 (d, J = 8.1 Hz, 2H), 7.91 |
| | (d, J = 8.6 Hz, 1H), 7.86 (t, J = 8.1 Hz, 1H), 7.80 (s, 1H), 7.54 |
| | (d, J = 8.6 Hz, 1H), 4.08 (s, 3H), 1.46 (s, 9H) |
| | Mass Spectrum (LCMS, ESI pos.) Calculated For |
| | C₂₃H₁₉ClF₆N₄: 501.1 (M + H), Measured: 501.3 |
| 95 | 2-(5-tert-Butyl-2,4-dimethyl-2H-pyrazol-3-yl)-5-(2,6-difluoro- |
| | phenyl)-1H-benzimidazole hydrochloride |
| | ¹H-NMR (400 MHz, CD₃OD) δ: 8.00 (d, J = 8.6 Hz, 1H), 7.97 |
| | (s, 1H), 7.75 (dd, J = 8.6, 1.3 Hz, 1H), 7.50 (tt, J = 8.4, 6.3 Hz, |
| | 1H), 7.12-7.21 (m, 3H), 3.98 (s, 3H), 2.35 (s, 3H), 1.42 (s, 9H) |
| | Mass Spectrum (LCMS, ESI pos.) Calculated For C₂₂H₂₂F₂N₄: |
| | 381.2 (M + H), Measured: 381.2. |
| 97 | 5-(2,6-Bis-trifluoromethyl-phenyl)-2-(5-tert-butyl-2,4-dimethyl- |
| | 2H-pyrazol-3-yl)-1H-benzimidazole hydrochloride |
| | ¹H-NMR (400 MHz, CD₃OD) δ: 8.16 (d, J = 8.1 Hz, 2H), 7.95 |
| | (d, J = 8.6 Hz, 1H), 7.88 (t, J = 8.1 Hz, 1H), 7.83 (s, 1H), 7.60 |
| | (d, J = 8.6 Hz, 1H), 4.00 (s, 3H), 2.36 (s, 3H), 1.42 (s, 9H) |
| | Mass Spectrum (LCMS, ESI pos.) Calculated For C₂₄H₂₂F₆N₄: |
| | 481.2 (M + H), Measured: 481.2. |
| 103 | 2-(3-tert-Butyl-isoxazol-5-yl)-5-(2-fluoro-6-trifluoromethoxy- |
| | phenyl)-1H-benzimidazole hydrochloride |
| | ¹H-NMR (400 MHz, CD₃OD) δ: 7.96 (d, J = 8.6 Hz, 1H), 7.86 |
| | (s, 1H), 7.55-7.67 (m, 2H), 7.42 (br. s., 1H), 7.30-7.38 (m, 2H), |
| | 1.45 (s, 9H) |
| | Mass Spectrum (LCMS, APCI pos.) Calculated For |
| | C₂₁H₁₇F₄N₃O₂: 420.1 (M + H), Measured: 420.1. |
| 104 | 5-(2,6-Bis-trifluoromethyl-phenyl)-2-(3-tert-butyl-isoxazol-5-yl)- |
| | 1H-benzimidazole hydrochloride |
| | ¹H-NMR (400 MHz, CD₃OD) δ: 8.14 (d, J = 8.1 Hz, 2H), 7.84- |
| | 7.93 (m, 2H), 7.79 (s, 1H), 7.56 (d, J = 8.6 Hz, 1H), 7.45 (s, |
| | 1H), 1.45 (s, 9H) |
| | Mass Spectrum (LCMS, APCI pos.) Calculated For |
| | C₂₂H₁₇F₆N₃O: 454.1 (M + H), Measured: 454.1. |
| 109 | 2-(3-tert-Butyl-4-methyl-isoxazol-5-yl)-5-(2,6-difluoro-phenyl)- |
| | 1H-benzimidazole hydrochloride |
| | ¹H-NMR (400 MHz, CD₃OD) δ: 8.01 (d, J = 8.6 Hz, 1H), 7.98 |
| | (s, 1H), 7.76 (dd, J = 8.6, 1.3 Hz, 1H), 7.50 (tt, J = 8.4, 6.3 Hz, |
| | 1H), 7.10-7.20 (m, 2H), 2.63 (s, 3H), 1.49 (s, 9H) |
| | Mass Spectrum (LCMS, ESI pos.) Calculated For |
| | C₂₁H₁₉F₂N₃O: 368.2 (M + H), Measured: 368.2. |
| 110 | 2-(3-tert-Butyl-4-methyl-isoxazol-5-yl)-5-(2-fluoro-6- |
| | trifluoromethoxy-phenyl)-1H-benzimidazole hydrochloride |
| | ¹H-NMR (400 MHz, CD₃OD) δ: 8.02 (d, J = 8.6 Hz, 1H), 7.92 |
| | (s, 1H), 7.69 (d, J = 8.6 Hz, 1H), 7.61 (td, J = 8.5, 6.3 Hz, 1H), |
| | 7.30-7.41 (m, 2H), 2.62 (s, 3H), 1.50 (s, 9H) |
| | Mass Spectrum (LCMS, ESI pos.) Calculated For |
| | C₂₂H₁₉F₄N₃O₂: 434.1 (M + H), Measured: 434.1. |
| 111 | 2-(3-tert-Butyl-4-methyl-isoxazol-5-yl)-5-(2-fluoro-6- |
| | trifluoromethyl-phenyl)-1H-benzimidazole hydrochloride |
| | ¹H-NMR (400 MHz, CD₃OD) δ: 8.00 (d, J = 8.6 Hz, 1H), 7.86 |
| | (s, 1H), 7.66-7.75 (m, 2H), 7.61 (d, J = 8.6 Hz, 1H), 7.53-7.59 |
| | (m, 1H), 2.63 (s, 3H), 1.49 (s, 9H) |
| | Mass Spectrum (LCMS, ESI pos.) Calculated For |
| | C₂₂H₁₉F₄N₃O: 418.2 (M + H), Measured: 418.2. |
| 122 | 2-(5-tert-Butyl-2,4-dimethyl-2H-pyrazol-3-yl)-5-(2,6-difluoro- |
| | phenyl)-7-trifluoromethyl-1H-benzimidazole hydrochloride |
| | ¹H-NMR (400 MHz, CD₃OD) δ: 8.12 (s, 1H), 7.88 (s, 1H), 7.48- |
| | 7.56 (m, 1H), 7.14-7.22 (m, 2H), 3.95 (s, 3H), 2.31 (s, 3H), |
| | 1.43 (s, 9H) |
| | Mass Spectrum (LCMS, ESI pos.) Calculated for C₂₄H₂₁F₇N₄: |
| | 449.2 (M + H), Measured: 449.2. |
| 119 | 2-(5-tert-Butyl-2,4-dimethyl-2H-pyrazol-3-yl)-5-(2-fluoro-6- |
| | trifluoromethyl-phenyl)-7-trifluoromethyl-1H-benzimidazole |
| | hydrochloride |
| | ¹H-NMR (400 MHz, CD₃OD) δ: 7.96 (s, 1H), 7.70 (s, 1H), 7.67- |
| | 7.75 (m, 2H), 7.54-7.60 (m, 1H), 3.94 (s, 3H), 2.31 (s, 3H), |
| | 1.42 (s, 9H) |
| | Mass Spectrum (LCMS, ESI pos.) Calculated for C₂₄H₂₁F₇N₄: |
| | 499.2 (M + H), Measured: 499.3. |
| 123 | 2-(5-tert-Butyl-2,4-dimethyl-2H-pyrazol-3-yl)-5-(2-fluoro-6- |
| | trifluoromethoxy-phenyl)-7-trifluoromethyl-1H-benzimidazole |
| | hydrochloride |
| | ¹H-NMR (400 MHz, CD₃OD) δ: 8.09 (s, 1H), 7.84 (s, 1H), 7.62 |
| | (td, J = 8.5, 6.3 Hz, 1H), 7.34-7.42 (m, 2H), 3.96 (s, 3H), 2.32 |
| | (s, 3H), 1.43 (s, 9H) |
| | Mass Spectrum (LCMS, ESI pos.) Calculated for C₂₄H₂₁F₇N₄O: |
| | 515.2 (M + H), Measured: 515.2. |
| 136 | 2-(5-tert-Butyl-2,4-dimethyl-2H-pyrazol-3-yl)-7-chloro-5-(2,6- |
| | difluoro-phenyl)-1H-benzimidazole hydrochloride |
| | ¹H-NMR (400 MHz, CD₃OD) δ: 7.81 (s, 1H), 7.65 (s, 1H), 7.45- |
| | 7.54 (m, 1H), 7.11-7.20 (m, 2H), 3.94 (s, 3H), 2.31 (s, 3H), |
| | 1.42 (s, 9H) |
| | Mass Spectrum (LCMS, ESI pos.) Calculated for |
| | C₂₂H₂₁ClF₂N₄: 415.1 (M + H), Measured: 415.2. |
| 136 | 2-(5-tert-Butyl-2,4-dimethyl-2H-pyrazol-3-yl)-7-chloro-5-(2,6- |
| | difluoro-phenyl)-1H-benzimidazole sodium salt |
| | ¹H-NMR (400 MHz, CD₃OD) δ: 7.60 (d, J = 1.3 Hz, 1H), 7.35- |
| | 7.44 (m, 1H), 7.26 (d, J = 1.5 Hz, 1H), 7.04-7.13 (m, 2H), 3.88 |
| | (s, 3H), 2.27 (s, 3H), 1.40 (s, 9H) |
| | Mass Spectrum (LCMS, ESI pos.) Calculated for |
| | C₂₂H₂₁ClF₂N₄: 415.1 (M + H), Measured: 415.3. |
| 137 | 2-(5-tert-Butyl-2,4-dimethyl-2H-pyrazol-3-yl)-7-chloro-5-(2- |
| | fluoro-6-trifluoromethyl-phenyl)-1H-benzimidazole |
| | hydrochloride |
| | ¹H-NMR (400 MHz, CD₃OD) δ: 7.69 (s, 1H), 7.66-7.74 (m, 2H), |
| | 7.53-7.59 (m, 1H), 7.52 (s, 1H), 3.95 (s, 3H), 2.32 (s, 3H), 1.42 |
| | (s, 9H) |
| | Mass Spectrum (LCMS, ESI pos.) Calculated for |
| | C₂₃H₂₁ClF₄N₄: 465.1 (M + H), Measured: 465.3. |
| 137 | 2-(5-tert-Butyl-2,4-dimethyl-2H-pyrazol-3-yl)-7-chloro-5-(2- |
| | fluoro-6-trifluoromethyl-phenyl)-1H-benzimidazole sodium salt |
| | ¹H-NMR (400 MHz, CD₃OD) δ: 7.55-7.68 (m, 2H), 7.48 (t, J = |
| | 8.6 Hz, 1H), 7.43 (s, 1H), 7.05 (s, 1H), 3.88 (s, 3H), 2.27 (s, |
| | 3H), 1.40 (s, 9H) |
| | Mass Spectrum (LCMS, ESI pos.) Calculated for |
| | C₂₃H₂₁ClF₄N₄: 465.1 (M + H), Measured: 465.3. |
| 138 | 2-(5-tert-Butyl-2,4-dimethyl-2H-pyrazol-3-yl)-7-chloro-5-(2- |
| | fluoro-6-trifluoromethoxy-phenyl)-1H-benzimidazole |
| | hydrochloride |
| | ¹H-NMR (400 MHz, CD₃OD) δ: 7.72 (s, 1H), 7.59 (td, J = 8.5, |
| | 6.3 Hz, 1H), 7.53 (s, 1H), 7.30-7.39 (m, 2H), 3.94 (s, 3H), 2.31 |
| | (s, 3H), 1.42 (s, 9H) |
| | Mass Spectrum (LCMS, ESI pos.) Calculated for |
| | C₂₃H₂₁ClF₃N₄O: 481.1 (M + H), Measured: 481.3. |
| 138 | 2-(5-tert-Butyl-2,4-dimethyl-2H-pyrazol-3-yl)-7-chloro-5-(2- |
| | fluoro-6-trifluoromethoxy-phenyl)-1H-benzimidazole sodium |
| | salt |
| | ¹H-NMR (400 MHz, CD₃OD) δ: 7.50 (s, 1H), 7.44-7.49 (m, 1H), |
| | 7.23-7.30 (m, 2H), 7.08 (s, 1H), 3.87 (s, 3H), 2.27 (s, 3H), 1.40 |
| | (s, 9H) |
| | Mass Spectrum (LCMS, ESI pos.) Calculated for |
| | C₂₃H₂₁ClF₃N₄O: 481.1 (M + H), Measured: 481.3. |
| 146 | 2-[2-(5-tert-Butyl-4-chloro-2-methyl-2H-pyrazol-3-yl)-7- |
| | trifluoromethyl-1H-benzimidazol-5-yl]-3-fluoro-benzonitrile |
| | hydrochloride |
| | ¹H-NMR (400 MHz, CD₃OD) δ: 8.07 (s, 1H), 7.73-7.78 (m, 2H), |
| | 7.60-7.68 (m, 2H), 4.10 (s, 3H), 1.46 (s, 9H) |
| | Mass Spectrum (LCMS, ESI pos.) Calculated for |
| | C₂₃H₁₈ClF₄N₅: 476.1 (M + H), Measured: 476.3. |
| 58 | 2-(5-tert-Butyl-2-methyl-2H-pyrazol-3-yl)-5-(2,6-difluoro- |
| | phenyl)-1H-benzimidazole hydrochloride |
| | ¹H-NMR (400 MHz; CD₃OD) δ: 7.54-7.86 (m, 2H), 7.29-7.45 |
| | (m, 2H), 7.01-7.17 (m, 2H), 6.78 (s, 1H), 4.26 (s, 3H), 1.32- |
| | 1.42 (m, 9H) |
| | Mass Spectrum (LCMS, ESI pos.) Calculated For C₂₁H₂₀F₄O₂: |
| | 367.1 (M + H), Measured: 367.2. |

### Example 16

### 2-(5-tert-Butyl-4-chloro-2-methyl-2H-pyrazol-3-yl)-5-(2-fluoro-phenyl)-1H-benzimidazole hydrochloride (Compound #90)

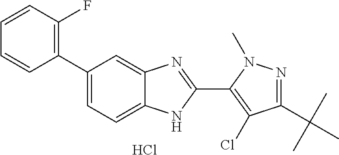

### STEP A. 2′-Fluoro-3-nitro-biphenyl-4-ylamine

To a mixture of 4-bromo-2-nitro-phenylamine (1.00 g, 4.61 mmol), 2-fluoro-phenylboronic acid (710 mg, 5.07 mmol) and Pd(PPh₃)₄ (533 mg, 0.461 mmol) in 1,4-dioxane (12 mL) was added 2.0 M aqueous Na₂CO₃ (9.2 mL, 18.4 mmol). The resulting mixture was stirred at 110° C. under microwave irradiation for 1 h and then cooled to room temperature. The resulting mixture was treated with EtOAc (50 mL), then washed with H₂O and brine and was dried (Na₂SO₄).

The solvent was removed under reduced pressure followed by flash chromatography of the residue on silica gel (10-40% EtOAc-hexanes) to yield an orange solid. ¹H-NMR (400 MHz, CDCl₃) δ: 8.35 (d, J=1.8 Hz, 1H), 7.63 (dt, J=8.6, 2.0 Hz, 1H), 7.44 (td, J=7.8, 1.8 Hz, 1H), 7.28-7.36 (m, 1H), 7.11-7.25 (m, 2H), 6.90 (d, J=8.6 Hz, 1H), 6.17 (br. s., 2H). Mass Spectrum (LCMS, ESI pos.) Calculated For C₁₂H₉FN₂O₂: 233.1 (M+H), Measured: 233.1.

### STEP B. 5-tert-Butyl-4-chloro-2-methyl-2H-pyrazole-3-carboxylic acid (2′-fluoro-3-nitro-biphenyl-4-yl)-amide

Following the procedure for Step B in Example 15, the title compound was prepared from 2′-fluoro-3-nitro-biphenyl-4-ylamine (as prepared in the previous step, 70.0 mg, 0.300 mmol), NaH (30.3 mg, 1.20 mmol, 95%), 5-tert-butyl-4-chloro-2-methyl-2H-pyrazole-3-carboxylic acid (68.3 mg, 0.315 mmol, as prepared in Example T), and oxalyl chloride (28.0 μL, 0.322 mmol) as a light yellow solid. ¹H-NMR (400 MHz, CDCl₃) δ: 11.11 (s, 1H), 8.85 (d, J=8.8 Hz, 1H), 8.46 (s, 1H), 7.91 (dt, J=8.8, 1.6 Hz, 1H), 7.48 (td, J=7.7, 1.8 Hz, 1H), 7.37-7.44 (m, 1H), 7.25-7.31 (m, 1H), 7.18-7.25 (m, 1H), 4.14 (s, 3H), 1.44 (s, 9H). Mass Spectrum (LCMS, ESI pos.) Calculated For C₂₁H₂₀ClFN₄O₃: 431.1 (M+H), Measured: 431.1.

### STEP C. 2-(5-tert-Butyl-4-chloro-2-methyl-2H-pyrazol-3-yl)-5-(2-fluoro-phenyl)-1H-benzimidazole

Following the procedure for Step C in Example 15, the title compound was prepared from 5-tert-butyl-4-chloro-2-methyl-2H-pyrazole-3-carboxylic acid (2′-fluoro-3-nitro-biphenyl-4-yl)-amide (as prepared in the previous step, 75.0 mg, 0.174 mmol) and iron powder (77.6 mg, 1.39 mmol) in 2 mL of 1:1 AcOH/EtOH as a white solid. ¹H-NMR (400 MHz, CDCl₃) δ: 7.84 (s, 1H), 7.75 (d, J=8.6 Hz, 1H), 7.49-7.57 (m, 2H), 7.32-7.41 (m, 1H), 7.15-7.30 (m, 2H), 4.03 (s, 3H), 1.44 (s, 9H). Mass Spectrum (LCMS, ESI pos.) Calculated For C₂₁H₂₀ClFN₄O₃: 383.1 (M+H), Measured: 383.2.

### STEP D. 2-(5-tert-Butyl-4-chloro-2-methyl-2H-pyrazol-3-yl)-5-(2-fluoro-phenyl)-1H-benzimidazole hydrochloride

Following the procedure for Step D in Example 15, the title compound was prepared from 2-(5-tert-butyl-4-chloro-2-methyl-2H-pyrazol-3-yl)-5-(2-fluoro-phenyl)-1H-benzimidazole (as prepared in the previous step, 45.0 mg, 0.118 mmol) and 1.0 M HCl (118 μL, 0.118 mmol) in 2 mL of 1:1 MeOH/DCM as a white solid.

¹H-NMR (400 MHz, CD₃OD) δ: 8.07 (s, 1H), 8.01 (d, J=8.6 Hz, 1H), 7.89 (d, J=8.8 Hz, 1H), 7.58-7.66 (m, 1H), 7.43-7.51 (m, 1H), 7.32-7.38 (m, 1H), 7.28 (dd, J=10.9, 8.3 Hz, 1H), 4.09 (s, 3H), 1.47 (s, 9H). Mass Spectrum (LCMS, ESI pos.) Calculated For C₂₁H₂₀ClFN₄: 383.1 (M+H), Measured: 383.2.

Following the procedure described in Example 16, and selecting and substituting reagents, starting materials, and conditions as would be known to those skilled in the art, the following compounds of formula (I) of the present invention were prepared:

| ID | |
|---|---|
| No. | Name and Measured Physical Property |
| 89 | 2-(5-tert-Butyl-4-chloro-2-methyl-2H-pyrazol-3-yl)-5-(2-chloro- |
| | phenyl)-1H-benzimidazole hydrochloride |
| | ¹H-NMR (400 MHz, CD₃OD) δ: 8.01 (d, J = 8.6 Hz, 1H), 7.96 |
| | (s, 1H), 7.77 (dd, J = 8.6, 1.5 Hz, 1H), 7.56-7.61 (m, 1H), 7.41- |
| | 7.53 (m, 3H), 4.09 (s, 3H), 1.47 (s, 9H) |
| | Mass Spectrum (LCMS, ESI pos.) Calculated For |
| | C₂₁H₂₀Cl₂N₄: 399.1 (M + H), Measured: 399.2. |
| 50 | 2-(5-tert-Butyl-4-chloro-2-methyl-2H-pyrazol-3-yl)-5-(2- |
| | trifluoromethyl-phenyl)-1H-benzimidazole hydrochloride |
| | ¹H-NMR (400 MHz, CDCl₃) δ: 9.99-10.16 (m, 1H), 7.75-7.88 |
| | (m, 2H), 7.53-7.63 (m, 2H), 7.51 (s, 2H), 7.42 (d, J = 7.6 Hz, |
| | 1H), 7.30 (s, 1H), 4.38 (d, J = 3.3 Hz, 3H), 1.46 (d, 9H) |
| | Mass Spectrum (LCMS, ESI pos.) Calculated For |
| | C₂₂H₂₀ClF₃N₄: 433.1 (M + H), Measured: 433.2. |
| 94 | 2-(5-tert-Butyl-2,4-dimethyl-2H-pyrazol-3-yl)-5-(2-chloro- |
| | phenyl)-1H-benzimidazole hydrochloride |
| | ¹H-NMR (400 MHz, CD₃OD) δ: 7.97 (d, J = 8.6 Hz, 1H), 7.91 |
| | (s, 1H), 7.74 (dd, J = 8.6, 1.5 Hz, 1H), 7.56-7.60 (m, 1H), 7.43- |
| | 7.51 (m, 3H), 3.98 (s, 3H), 2.35 (s, 3H), 1.42 (s, 9H) |
| | Mass Spectrum (LCMS, ESI pos.) Calculated For C₂₂H₂₃ClN₄: |
| | 379.2 (M + H), Measured: 379.2. |
| 93 | 2-(5-tert-Butyl-2,4-dimethyl-2H-pyrazol-3-yl)-5-(2-fluoro- |
| | phenyl)-1H-benzimidazole hydrochloride |
| | ¹H-NMR (400 MHz, CD₃OD) δ: 8.04 (s, 1H), 7.99 (d, J = 8.6 |
| | Hz, 1H), 7.83-7.90 (m, 1H), 7.62 (td, J = 7.8, 1.6 Hz, 1H), |
| | 7.43-7.51 (m, 1H), 7.31-7.38 (m, 1H), 7.28 (dd, J = 11.1, 8.3 |
| | Hz, 1H), 3.99 (s, 3H), 2.35 (s, 3H), 1.42 (s, 9H) |
| | Mass Spectrum (LCMS, ESI pos.) Calculated For C₂₂H₂₃FN₄: |
| | 363.2 (M + H), Measured: 363.2. |
| 105 | 2-(3-tert-Butyl-isoxazol-5-yl)-5-(5-fluoro-2-methoxy-phenyl)- |
| | 1H-benzimidazole hydrochloride |
| | ¹H-NMR (400 MHz, CD₃OD) δ: 7.95 (s, 1H), 7.84-7.89 (m, |
| | 1H), 7.78 (dd, J = 8.8, 1.5 Hz, 1H), 7.43 (s, 1H), 7.17 (dt, J = |
| | 8.8, 1.9 Hz, 1H), 7.10-7.15 (m, 2H), 3.81 (s, 3H), 1.45 (s, 9H) |
| | Mass Spectrum (LCMS, ESI pos.) Calculated For |
| | C₂₁H₂₀FN₃O₂: 366.2 (M + H), Measured: 366.1. |
| 106 | 2-(3-tert-Butyl-4-methyl-isoxazol-5-yl)-5-(2-fluoro-phenyl)-1H- |
| | benzimidazole hydrochloride |
| | ¹H-NMR (400 MHz, CD₃OD) δ: 8.04 (s, 1H), 7.98 (d, J = 8.8 |
| | Hz, 1H), 7.84-7.91 (m, 1H), 7.61 (td, J = 7.8, 1.6 Hz, 1H), |
| | 7.42-7.50 (m, 1H), 7.34 (td, J = 7.5, 1.1 Hz, 1H), 7.27 (dd, J = |
| | 10.5, 8.7 Hz, 1H), 7.23-7.30 (m, 1H), 2.62 (s, 3H), 2.62 (s, |
| | 3H), 2.62 (s, 3H), 1.49 (s, 9H), 1.43-1.56 (m, 9H). |
| | Mass Spectrum (LCMS, ESI pos.) Calculated For |
| | C₂₁H₂₀FN₃O: 350.2 (M + H), Measured: 350.2. |
| 107 | 2-(3-tert-Butyl-4-methyl-isoxazol-5-yl)-5-(2-chloro-phenyl)-1H- |
| | benzimidazole hydrochloride |
| | ¹H-NMR (400 MHz, CD₃OD) δ: 7.97 (dd, J = 8.6, 0.8 Hz, 1H), |
| | 7.92 (dd, J = 1.4, 0.6 Hz, 1H), 7.73 (dd, J = 8.5, 1.6 Hz, 1H), |
| | 7.55-7.59 (m, 1H), 7.40-7.50 (m, 3H), 2.63 (s, 3H), 1.50 (s, |
| | 9H) |
| | Mass Spectrum (LCMS, ESI pos.) Calculated For |
| | C₂₁H₂₀ClN₃O: 366.1 (M + H), Measured: 366.1. |
| 108 | 2-(3-tert-Butyl-4-methyl-isoxazol-5-yl)-5-(2-trifluoromethyl- |
| | phenyl)-1H-benzimidazole hydrochloride |
| | ¹H-NMR (400 MHz, CD₃OD) δ: 7.97 (dd, J = 8.6, 0.8 Hz, 1H), |
| | 7.92 (dd, J = 1.4, 0.6 Hz, 1H), 7.73 (dd, J = 8.5, 1.6 Hz, 1H), |
| | 7.54-7.59 (m, 1H), 7.39-7.51 (m, 3H), 2.63 (s, 3H), 1.50 (s, |
| | 9H) |
| | Mass Spectrum (LCMS, ESI pos.) Calculated For |
| | C₂₂H₂₀F₃N₃O: 400.2 (M + H), Measured: 400.2. |
| 120 | 2-(5-tert-Butyl-2,4-dimethyl-2H-pyrazol-3-yl)-7-trifluoromethyl- |
| | 5-(2-trifluoromethyl-phenyl)-1H-benzimidazole hydrochloride |
| | ¹H-NMR (400 MHz, CD₃OD) δ: 7.96 (s, 1H), 7.88 (d, J = 7.8 |
| | Hz, 1H), 7.74-7.75 (m, 1H), 7.63-7.69 (m, 1H), 7.51 (d, J = 7.3 |
| | Hz, 1H), 3.95 (s, 3H), 2.31 (s, 3H), 1.42 (s, 9H) |
| | Mass Spectrum (LCMS, ESI pos.) Calculated for C₂₄H₂₂F₆N₄: |
| | 481.2 (M + H), Measured: 481.3. |
| 118 | 2-(5-tert-Butyl-2,4-dimethyl-2H-pyrazol-3-yl)-5-(2-fluoro- |
| | phenyl)-7-trifluoromethyl-1H-benzimidazole hydrochloride |
| | ¹H-NMR (400 MHz, CD₃OD) δ: 8.14 (s, 1H), 7.92 (s, 1H), |
| | 7.60-7.66 (m, 1H), 7.44-7.51 (m, 1H), 7.33-7.38 (m, 1H), 7.26- |
| | 7.32 (m, 1H), 3.93 (s, 3H), 2.30 (s, 3H), 1.42 (s, 9H) |
| | Mass Spectrum (LCMS, ESI pos.) Calculated for C₂₃H₂₂F₄N₄: |
| | 431.2 (M + H), Measured: 431.3. |
| 121 | 2-(5-tert-Butyl-2,4-dimethyl-2H-pyrazol-3-yl)-5-(2-chloro- |
| | phenyl)-7-trifluoromethyl-1H-benzimidazole hydrochloride |
| | ¹H-NMR (400 MHz, CD₃OD) δ: 8.03 (s, 1H), 7.81 (s, 1H), |
| | 7.56-7.61 (m, 1H), 7.49-7.53 (m, 1H), 7.41-7.48 (m, 2H), 3.94 |
| | (s, 3H), 2.30 (s, 3H), 1.42 (s, 9H) |
| | Mass Spectrum (LCMS, ESI pos.) Calculated for C₂₃H₂₁F₅N₄: |
| | 447.2 (M + H), Measured: 447.2. |
| 135 | 2-(5-tert-Butyl-2,4-dimethyl-2H-pyrazol-3-yl)-7-chloro-5-(2- |
| | trifluoromethyl-phenyl)-1H-benzimidazole hydrochloride |
| | ¹H-NMR (400 MHz, CD₃OD) δ: 7.85 (d, J = 7.8 Hz, 1H), 7.68- |
| | 7.75 (m, 1H), 7.66 (s, 1H), 7.61-7.68 (m, 1H), 7.51 (s, 1H), |
| | 7.48 (d, J = 7.3 Hz, 1H), 3.94 (s, 3H), 2.31 (s, 3H), 1.41 (s, 9H) |
| | Mass Spectrum (LCMS, ESI pos.) Calculated for |
| | C₂₃H₂₂ClF₃N₄: 477.2 (M + H), Measured: 477.3. |
| 135 | 2-(5-tert-Butyl-2,4-dimethyl-2H-pyrazol-3-yl)-7-chloro-5-(2- |
| | trifluoromethyl-phenyl)-1H-benzimidazole sodium salt |
| | ¹H-NMR (400 MHz, CD₃OD) δ: 7.80 (d, J = 7.8 Hz, 1H), 7.63- |
| | 7.69 (m, 1H), 7.53-7.59 (m, 1H), 7.47 (s, 1H), 7.46-7.46 (m, |
| | 1H), 7.16 (s, 1H), 3.88 (s, 3H), 2.27 (s, 3H), 1.40 (s, 9H) |
| | Mass Spectrum (LCMS, ESI pos.) Calculated for |
| | C₂₃H₂₂ClF₃N₄: 477.2 (M + H), Measured: 477.3. |
| 133 | 2-(5-tert-Butyl-2,4-dimethyl-2H-pyrazol-3-yl)-7-chloro-5-(2- |
| | fluoro-phenyl)-1H-benzimidazole hydrochloride |
| | ¹H-NMR (400 MHz, CD₃OD) δ: 7.85 (s, 1H), 7.72 (s, 1H), |
| | 7.56-7.63 (m, 1H), 7.41-7.49 (m, 1H), 7.30-7.36 (m, 1H), 7.23- |
| | 7.30 (m, 1H), 3.93 (s, 3H), 2.30 (s, 3H), 1.41 (s, 9H) |
| | Mass Spectrum (LCMS, ESI pos.) Calculated for C₂₂H₂₂ClFN₄: |
| | 397.2 (M + H), Measured: 397.3. |
| 133 | 2-(5-tert-Butyl-2,4-dimethyl-2H-pyrazol-3-yl)-7-chloro-5-(2- |
| | fluoro-phenyl)-1H-benzimidazole sodium salt |
| | ¹H-NMR (400 MHz, CD₃OD) δ: 7.71 (t, J = 1.4 Hz, 1H), 7.55 |
| | (td, J = 7.7, 1.8 Hz, 1H), 7.42 (t, J = 1.4 Hz, 1H), 7.34-7.41 (m, |
| | 1H), 7.27 (td, J = 7.6, 1.3 Hz, 1H), 7.18-7.24 (m, 1H), 3.88 (s, |
| | 3H), 2.27 (s, 3H), 1.40 (s, 9H) |
| | Mass Spectrum (LCMS, ESI pos.) Calculated for C₂₂H₂₂ClFN₄: |
| | 397.2 (M + H), Measured: 397.3. |
| 134 | 2-(5-tert-Butyl-2,4-dimethyl-2H-pyrazol-3-yl)-7-chloro-5-(2- |
| | chloro-phenyl)-1H-benzimidazole hydrochloride |
| | ¹H-NMR (400 MHz, CD₃OD) δ: 7.72 (d, J = 1.3 Hz, 1H), 7.58 |
| | (d, J = 1.3 Hz, 1H), 7.55-7.58 (m, 1H), 7.46-7.50 (m, 1H), |
| | 7.41-7.45 (m, 2H), 3.93 (s, 3H), 2.30 (s, 3H), 1.42 (s, 9H) |
| | Mass Spectrum (LCMS, ESI pos.) Calculated for C₂₂H₂₂Cl₂N₄: |
| | 413.1 (M + H), Measured: 413.3. |
| 134 | 2-(5-tert-Butyl-2,4-dimethyl-2H-pyrazol-3-yl)-7-chloro-5-(2- |
| | chloro-phenyl)-1H-benzimidazole sodium salt |
| | ¹H-NMR (400 MHz, CD₃OD) δ: 7.57 (d, J = 1.3 Hz, 1H), 7.49- |
| | 7.52 (m, 1H), 7.42-7.45 (m, 1H), 7.31-7.40 (m, J = 7.5, 7.5, |
| | 7.5, 7.5, 1.8 Hz, 2H), 7.30 (d, J = 1.5 Hz, 1H), 3.89 (s, 3H), |
| | 2.26 (s, 3H), 1.40 (s, 9H) |
| | Mass Spectrum (LCMS, ESI pos.) Calculated for C₂₂H₂₂Cl₂N₄: |
| | 413.1 (M + H), Measured: 413.3. |
| 184 | 2-tert-Butyl-5-methyl-4-[7-trifluoromethyl-5-(2-trifluoromethyl- |
| | phenyl)-1H-benzimidazol-2-yl]-furan-3-carbonitrile sodium salt |
| | ¹H-NMR (400 MHz; CD₃OD) δ: 7.78 (d, J = 7.8 Hz, 1H), 7.60- |
| | 7.71 (m, 2 H), 7.43-7.56 (m, 2 H), 7.20 (s, 1 H), 2.50 (s, 3 H), |
| | 1.49, (s, 9 H) |
| | Mass Spectrum (LCMS, ESI pos.) Calculated For |
| | C₂₃H₁₉F₆N₃O: 492.1 (M + H), Measured: 492.2. |
| 174 | 2-(5-tert-Butyl-2-methyl-furan-3-yl)-5-(2-fluoro-phenyl)-1H- |
| | benzimidazole sodium salt |
| | ¹H-NMR (400 MHz; CD₃OD) δ: 7.72 (s, 1H), 7.62 (d, J = 8.3 |
| | Hz, 1 H), 7.54 (td, J = 7.7, 1.8 Hz, 1 H), 7.41 (dt, J = 8.4, 1.6 Hz, |
| | 1 H), 7.31-7.39 (m, 1 H), 7.23-7.30 (m, 1 H), 7.16-7.23 (m, 1 |
| | H), 6.49 (s, 1 H), 1.89 (s, 3 H), 1.34, (s, 9 H) |
| | Mass Spectrum (LCMS, ESI pos.) Calculated For |
| | C₂₂H₂₁FN₂O: 349.1 (M + H), Measured: 349.3. |
| 131 | 5-(2-Fluoro-phenyl)-2-(2,6,6-trimethyl-2,4,5,6-tetrahydro- |
| | cyclopentapyrazol-3-yl)-1H-benzimidazole hydrochloride |
| | ¹H-NMR (400 MHz; CD₃OD) δ: 7.79 (s, 1H), 7.69 (d, J = 8.3 |
| | Hz, 1H), 7.52 (td, J = 7.8, 1.8 Hz, 1H), 7.47 (dt, J = 8.5, 1.5 |
| | Hz, 1H), 7.31-7.38 (m, 1H), 7.15-7.27 (m, 2H), 4.17 (s, 3H), |
| | 2.90 (t, J = 6.9 Hz, 2H), 2.32 (d, J = 6.9 Hz, 2H), 1.33 (s, 6H) |
| | Mass Spectrum (LCMS, ESI pos.) Calculated For C₂₂H₂₁FN₄: |
| | 361.2 (M + H), Measured: 361.3. |
| 132 | 5-(2-fluoro-phenyl)-2-(1,6,6-trimethyl-1,4,5,6-tetrahydro- |
| | cyclopentapyrazol-3-yl)-1H-benzimidazole hydrochloride |
| | ¹H-NMR (400 MHz; CD₃OD) δ: 7.74 (s, 1H), 7.63 (d, J = 8.3 |
| | Hz, 1H), 7.47-7.52 (m, 1H), 7.38-7.42 (m, 1H), 7.28-7.35 (m, |
| | 1H), 7.13-7.25 (m, 2H), 3.86 (s, 3H), 2.89 (d, J = 7.1 Hz, 2H), |
| | 2.43 (t, J = 7.1 Hz, 2H), 1.37 (s, 6H) |
| | Mass Spectrum (LCMS, ESI pos.) Calculated For C₂₂H₂₁FN₄: |
| | 361.2 (M + H), Measured: 361.3. |

### Example 17

### 2-(5-tert-Butyl-2,4-dimethyl-2H-pyrazol-3-yl)-5-(2-fluoro-6-trifluoromethoxy-phenyl)-1H-benzimidazole hydrochloride (Compound #96)

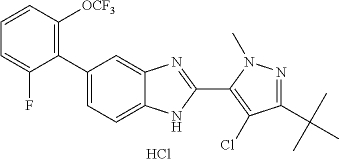

### STEP A. 6′-Fluoro-3-nitro-2′-trifluoromethoxy-biphenyl-4-ylamine

Following the procedure for Step A in Example 15, the title compound was prepared from 4-amino-2-nitrophenylboronic acid (450 mg, 2.23 mmol, 90%), 1-fluoro-2-iodo-3-trifluoromethoxy-benzene (745 mg, 2.45 mmol), Pd(PPh₃)₄ (258 mg, 0.223 mmol) and 2.0 M aqueous Na₂CO₃ (4.46 mL, 8.92 mmol) in 8 mL of 1,4-dioxane, as a yellow solid. ¹H-NMR (400 MHz, CDCl₃) δ: 8.22 (s, 1H), 7.33-7.44 (m, 2H), 7.12-7.22 (m, 2H), 6.90 (d, J=8.6 Hz, 1H), 6.20 (br. s., 2H). Mass Spectrum (LCMS, ESI pos.) Calculated For C₁₃H₈F₄N₂O₃: 317.1 (M+H), Measured: 317.0.

### STEP B. 5-tert-Butyl-2,4-dimethyl-2H-pyrazole-3-carboxylic acid (6′-fluoro-3-nitro-2′-trifluoromethoxy-biphenyl-4-yl)-amide

Following the procedure for Step B in Example 15, the title compound was prepared from 6′-fluoro-3-nitro-2′-trifluoromethoxy-biphenyl-4-ylamine (as prepared in the previous step, 126 mg, 0.400 mmol), dry NaH (40.0 mg, 1.60 mmol, 95%), 5-tert-butyl-2,4-dimethyl-2H-pyrazole-3-carboxylic acid (82.4 mg, 0.420 mmol, as prepared in Example E) and oxalyl chloride (214 μL, 0.428 mmol, 2.0 M in DCM), as a yellow solid. ¹H-NMR (400 MHz, CDCl₃) δ: 10.78 (s, 1H), 9.03 (d, J=8.8 Hz, 1H), 8.35 (s, 1H), 7.75 (d, J=8.8 Hz, 1H), 7.46 (td, J=8.4, 6.2 Hz, 1H), 7.13-7.26 (m, 2H), 4.04 (s, 3H), 2.49 (s, 3H), 1.41 (s, 9H). Mass Spectrum (LCMS, ESI pos.) Calculated For C₂₃H₂₂F₄N₄O₄: 495.2 (M+H), Measured: 495.2.

### STEP C. 2-(5-tert-Butyl-2,4-dimethyl-2H-pyrazol-3-yl)-5-(2-fluoro-6-trifluoromethoxy-phenyl)-1H-benzimidazole hydrochloride

Following the procedure for Step C in Example 15, 2-(5-tert-butyl-2,4-dimethyl-2H-pyrazol-3-yl)-5-(2-fluoro-6-trifluoromethoxy-phenyl)-1H-benzimidazole was prepared from 5-tert-butyl-2,4-dimethyl-2H-pyrazole-3-carboxylic acid (6′-fluoro-3-nitro-2′-trifluoromethoxy-biphenyl-4-yl)-amide (as prepared in the previous step, 121 mg, 0.245 mmol) and iron powder (109 mg, 1.96 mmol) in 3 mL of 1:1 AcOH/EtOH, as a white solid.

Following the procedure for Step D in Example 15, the title compound was prepared from 2-(5-tert-butyl-2,4-dimethyl-2H-pyrazol-3-yl)-5-(2-fluoro-6-trifluoromethoxy-phenyl)-1H-benzimidazole (as prepared above, 90.0 mg, 0.202 mmol) and 1.0 M HCl in Et₂O (202 μL, 0.202 mmol), as a white solid.

¹H-NMR (400 MHz, CD₃OD) δ: 8.02 (d, J=8.6 Hz, 1H), 7.91 (s, 1H), 7.68 (d, J=8.6 Hz, 1H), 7.61 (td, J=8.5, 6.3 Hz, 1H), 7.32-7.40 (m, 2H), 3.99 (s, 3H), 2.35 (s, 3H), 1.42 (s, 9H). Mass Spectrum (LCMS, ESI pos.) Calculated For C₂₃H₂₂F₄N₄O: 447.2 (M+H), Measured: 447.2.

### Example 18

### 2-(5-tert-Butyl-4-chloro-2-methyl-2H-pyrazol-3-yl)-5-(2-fluoro-phenyl)-1H-benzimidazole hydrochloride (Compound #114)

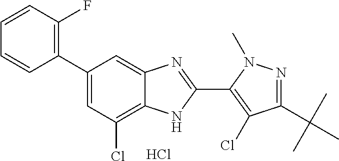

### STEP A. 4-Bromo-2-chloro-6-nitro-phenylamine

A mixture of 4-bromo-2-nitro-phenylamine (3.00 g, 13.8 mmol) and NCS (1.84 g, 13.8 mmol) in DMF (10 mL) was stirred at 80° C. for 3 h. After cooling to room temperature, the mixture was treated with EtOAc (100 mL), washed with H₂O and brine, then dried with Na₂SO₄. The solvent was removed under reduced pressure followed by flash chromatography of the residue on silica gel (0:100-5:95 EtOAc:hexanes) to yield a bright yellow solid. ¹H-NMR (400 MHz, CDCl₃) δ: 8.26 (d, J=2.3 Hz, 1H), 7.66 (d, J=2.3 Hz, 1H), 6.59 (br. s., 2H).

### STEP B. 3-Chloro-5-nitro-2′-fluoro-biphenyl-4-ylamine

Following the procedure for Step A in Example 15, the title compound was prepared from 4-bromo-2-chloro-6-nitro-phenylamine (as prepared in the previous step, 500 mg, 1.99 mmol), 2-fluoro-phenylboronic acid (306 mg, 2.19 mmol), Pd(PPh₃)₄ (230 mg, 0.199 mmol) and 2.0 M aqueous Na₂CO₃ (8.0 mL, 16.0 mmol) in 1,4-dioxane (10 mL), as a red solid. ¹H-NMR (400 MHz, CDCl₃) δ: 8.32 (s, 1H), 7.81 (s, 1H), 7.42 (td, J=7.8, 1.8 Hz, 1H), 7.31-7.39 (m, 1H), 7.12-7.26 (m, 2H), 6.65 (br. s., 2H). Mass Spectrum (LCMS, ESI pos.) Calculated For C₁₂H₈ClFN₂O₂: 267.0 (M+H), Measured: 267.1.

### STEP C. 5-tert-Butyl-4-chloro-2-methyl-2H-pyrazole-3-carboxylic acid (3-chloro-2′-fluoro-5-nitro-biphenyl-4-yl)-amide

Following the procedure for Step B in Example 15, the title compound was prepared from 3-chloro-5-nitro-2′-fluoro-biphenyl-4-ylamine (as prepared in the previous step, 93.3 mg, 0.350 mmol), dry NaH (35.4 mg, 1.40 mmol, 95%), 5-tert-butyl-4-chloro-2-methyl-2H-pyrazole-3-carboxylic acid (80.0 mg, 0.368 mmol, as prepared in Example T) and oxalyl chloride (193 μL, 0.385 mmol, 2.0 M), as a yellow solid. ¹H-NMR (400 MHz, CDCl₃) δ: 9.16 (s, 1H), 8.12 (s, 1H), 7.97 (s, 1H), 7.40-7.51 (m, 2H), 7.17-7.34 (m, 2H), 4.13 (s, 3H), 1.44 (s, 9H). Mass Spectrum (LCMS, ESI pos.) Calculated For C₂₁H₁₉Cl₂FN₄O₃: 465.1 (M+H), Measured: 465.1.

### STEP D. 2-(5-tert-Butyl-4-chloro-2-methyl-2H-pyrazol-3-yl)-7-chloro-5-(2-fluoro-phenyl)-1H-benzimidazole

Following the procedure for Step C in Example 15, the title compound was prepared from 5-tert-butyl-4-chloro-2-methyl-2H-pyrazole-3-carboxylic acid (3-chloro-2′-fluoro-5-nitro-biphenyl-4-yl)-amide (as prepared in the previous step, 118 mg, 0.254 mmol) and iron powder (113 mg, 2.03 mmol) in 3 mL of 1:1 AcOH/EtOH, as a white solid. ¹H-NMR (400 MHz, CD₃OD) δ: 7.75 (s, 1H), 7.50-7.58 (m, 2H), 7.36-7.43 (m, 1H), 7.28 (td, J=7.6, 1.3 Hz, 1H), 7.23 (ddd, J=11.0, 8.2, 1.0 Hz, 1H), 4.05 (s, 3H), 1.44 (s, 9H). Mass Spectrum (LCMS, ESI pos.) Calculated For C₂₁H₁₉Cl₂FN₄: 417.1 (M+H), Measured: 417.3.

### STEP E. 2-(5-tert-Butyl-4-chloro-2-methyl-2H-pyrazol-3-yl)-5-(2-fluoro-phenyl)-1H-benzimidazole hydrochloride

Following the procedure for Step D in Example 15, the title compound was prepared from 2-(5-tert-butyl-4-chloro-2-methyl-2H-pyrazol-3-yl)-7-chloro-5-(2-fluoro-phenyl)-1H-benzimidazole (as prepared in the previous step, 102 mg, 0.244 mmol) and 1.0 M HCl (244 μL, 0.244 mmol) in 3 mL of 1:1 MeOH/DCM, as a white solid.

¹H-NMR (400 MHz, CD₃OD) δ: 7.93 (s, 1H), 7.81 (s, 1H), 7.61 (td, J=7.8, 1.6 Hz, 1H), 7.43-7.52 (m, 1H), 7.34 (td, J=7.6, 1.3 Hz, 1H), 7.28 (ddd, J=11.1, 8.2, 1.1 Hz, 1H), 4.05 (s, 3H), 1.46 (s, 9H). Mass Spectrum (LCMS, ESI pos.) Calculated For C₂₁H₁₉Cl₂FN₄: 417.1 (M+H), Measured: 417.3.

Following the procedure described in Example 18, and selecting and substituting reagents, starting materials, and conditions as would be known to those skilled in the art, the following compounds of formula (I) of the present invention were prepared:

| ID | |
|---|---|
| No. | Name and Measured Physical Property |
| 114 | 2-(5-tert-Butyl-4-chloro-2-methyl-2H-pyrazol-3-yl)-7-chloro-5- |
| | (2-fluoro-phenyl)-1H-benzimidazole sodium salt |
| | ¹H-NMR (400 MHz, CD₃OD) δ: 7.70 (t, J = 1.5 Hz, 1H), 7.53 |
| | (td, J = 7.8, 1.8 Hz, 1H), 7.27-7.36 (m, 2H), 7.21-7.26 (m, 1H), |
| | 7.17 (ddd, J = 11.1, 8.1, 1.3 Hz, 1H), 3.91 (s, 3H), 1.39-1.48 |
| | (m, 9H) |
| | Mass Spectrum (LCMS, ESI pos.) Calculated For |
| | C₂₁H₁₉Cl₂FN₄: 417.1 (M + H), Measured: 417.2. |
| 115 | 2-(5-tert-Butyl-4-chloro-2-methyl-2H-pyrazol-3-yl)-7-chloro-5- |
| | (2-chloro-phenyl)-1H-benzimidazole sodium salt |
| | ¹H-NMR (400 MHz, CD₃OD) δ: 7.55 (d, J = 1.5 Hz, 1H), 7.48 |
| | (dd, J = 7.8, 1.3 Hz, 1H), 7.42 (dd, J = 7.5, 1.6 Hz, 1H), 7.34 |
| | (td, J = 7.5, 1.3 Hz, 1H), 7.26-7.31 (m, 1H), 7.13 (d, J = 1.5 Hz, |
| | 1H), 3.89 (s, 3H), 1.44 (s, 9H) |
| | Mass Spectrum (LCMS, ESI pos.) Calculated For C₂₁H₁₉Cl₃N₄: |
| | 433.1 (M + H), Measured: 433.2. |
| 115 | 2-(5-tert-Butyl-4-chloro-2-methyl-2H-pyrazol-3-yl)-7-chloro-5- |
| | (2-chloro-phenyl)-1H-benzimidazole hydrochloride |
| | ¹H-NMR (400 MHz, CD₃OD) δ: 7.79 (d, J = 1.3 Hz, 1H), 7.67 |
| | (d, J = 1.3 Hz, 1H), 7.55-7.60 (m, 1H), 7.41-7.51 (m, 3H), 4.05 |
| | (s, 3H), 1.46 (s, 9H) |
| | Mass Spectrum (LCMS, ESI pos.) Calculated For C₂₁H₁₉Cl₃N₄: |
| | 433.1 (M + H), Measured: 433.2. |
| 116 | 2-(5-tert-Butyl-4-chloro-2-methyl-2H-pyrazol-3-yl)-7-chloro-5- |
| | (2-trifluoromethyl-phenyl)-1H-benzimidazole hydrochloride |
| | ¹H-NMR (400 MHz, CD₃OD) δ: 7.86 (d, J = 7.8 Hz, 1H), 7.69- |
| | 7.76 (m, 2H), 7.63-7.68 (m, 1H), 7.57 (s, 1H), 7.50 (d, J = 7.6 |
| | Hz, 1H), 4.05 (s, 3H), 1.46 (s, 9H) |
| | Mass Spectrum (LCMS, ESI pos.) Calculated For |
| | C₂₂H₁₉Cl₂F₃N₄: 467.1 (M + H), Measured: 467.3. |
| 116 | 2-(5-tert-Butyl-4-chloro-2-methyl-2H-pyrazol-3-yl)-7-chloro-5- |
| | (2-trifluoromethyl-phenyl)-1H-benzimidazole sodium salt |
| | ¹H-NMR (400 MHz, CD₃OD) δ: 7.77 (d, J = 7.8 Hz, 1H), 7.58- |
| | 7.65 (m, 1H), 7.48-7.54 (m, 1H), 7.46 (s, 1H), 7.43 (d, J = 7.6 |
| | Hz, 1H), 7.04 (s, 1H), 3.91 (s, 3H), 1.44 (s, 9H) |
| | Mass Spectrum (LCMS, ESI pos.) Calculated For |
| | C₂₂H₁₉Cl₂F₃N₄: 467.1 (M + H), Measured: 467.3. |
| 117 | 2-(5-tert-Butyl-4-chloro-2-methyl-2H-pyrazol-3-yl)-7-chloro-5- |
| | (2-trifluoromethoxy-phenyl)-1H-benzimidazole hydrochloride |
| | ¹H-NMR (400 MHz, CD₃OD) δ: 7.83 (d, J = 1.5 Hz, 1H), 7.70 |
| | (d, J = 1.5 Hz, 1H), 7.60-7.63 (m, 1H), 7.47-7.60 (m, 3H), 4.05 |
| | (s, 3H), 1.46 (s, 9H) |
| | Mass Spectrum (LCMS, ESI pos.) Calculated For |
| | C₂₂H₁₉Cl₂F₃N₄O: 483.1 (M + H), Measured: 483.2. |
| 117 | 2-(5-tert-Butyl-4-chloro-2-methyl-2H-pyrazol-3-yl)-7-chloro-5- |
| | (2-trifluoromethoxy-phenyl)-1H-benzimidazole sodium salt |
| | ¹H-NMR (400 MHz, CD₃OD) δ: 7.61 (d, J = 1.5 Hz, 1H), 7.52- |
| | 7.57 (m, 1H), 7.34-7.45 (m, 3H), 7.19 (d, J = 1.5 Hz, 1H), 3.90 |
| | (s, 3H), 1.44 (s, 9H) |
| | Mass Spectrum (LCMS, ESI pos.) Calculated For |
| | C₂₂H₁₉Cl₂F₃N₄O 483.1 (M + H), Measured: 483.3. |
| 167 | 3-tert-Butyl-5-[7-chloro-5-(2-fluoro-phenyl)-1H-benzimidazol-2- |
| | yl]-1-methyl-1H-pyrazole-4-carbonitrile sodium salt |
| | ¹H-NMR (400 MHz, CD₃OD) δ: 7.73 (t, J = 1.5 Hz, 1H), 7.54 |
| | (td, J = 7.8, 1.8 Hz, 1H), 7.30-7.37 (m, 2H), 7.22-7.28 (m, 1H), |
| | 7.19 (ddd, J = 11.1, 8.1, 1.3 Hz, 1H), 4.06 (s, 3H), 1.45-1.49 |
| | (m, 9H) |
| | Mass Spectrum (LCMS, ESI pos.) Calculated For C₂₂H₁₉ClFN₅: |
| | 408.1 (M + H), Measured: 408.3. |
| 168 | 3-tert-Butyl-5-[7-chloro-5-(2-chloro-phenyl)-1H-benzimidazol-2- |
| | yl]-1-methyl-1H-pyrazole-4-carbonitrile sodium salt |
| | ¹H-NMR (400 MHz, CD₃OD) δ: 7.56 (d, J = 1.5 Hz, 1H), 7.49 |
| | (dd, J = 7.8, 1.3 Hz, 1H), 7.42-7.46 (m, 1H), 7.36 (td, J = 7.5, |
| | 1.5 Hz, 1H), 7.28-7.33 (m, 1H), 7.13 (d, J = 1.5 Hz, 1H), 4.05 |
| | (s, 3H), 1.47 (s, 9H) |
| | Mass Spectrum (LCMS, ESI pos.) Calculated For C₂₂H₁₉Cl₂N₅: |
| | 424.1 (M + H), Measured: 424.3. |
| 169 | 3-tert-Butyl-5-[7-chloro-5-(2-trifluoromethyl-phenyl)-1H- |
| | benzimidazol-2-yl]-1-methyl-1H-pyrazole-4-carbonitrile sodium |
| | salt |
| | ¹H-NMR (400 MHz, CD₃OD) δ: 7.77 (d, J = 7.8 Hz, 1H), 7.59- |
| | 7.66 (m, 1H), 7.49-7.55 (m, 1H), 7.48 (s, 1H), 7.45 (d, J = 7.6 |
| | Hz, 1H), 7.06 (s, 1H), 4.06 (s, 3H), 1.47 (s, 9H) |
| | Mass Spectrum (LCMS, ESI pos.) Calculated For |
| | C₂₃H₁₉ClF₃N₅: 458.1 (M + H), Measured: 458.3. |
| 170 | 3-tert-Butyl-5-[7-chloro-5-(2-trifluoromethoxy-phenyl)-1H- |
| | benzimidazol-2-yl]-1-methyl-1H-pyrazole-4-carbonitrile sodium |
| | salt |
| | ¹H-NMR (400 MHz, CD₃OD) δ: 7.62 (d, J = 1.5 Hz, 1H), 7.53- |
| | 7.57 (m, 1H), 7.35-7.46 (m, 3H), 7.18 (d, J = 1.5 Hz, 1H), 4.05 |
| | (s, 3H), 1.47 (s, 9H) |
| | Mass Spectrum (LCMS, ESI pos.) Calculated For |
| | C₂₃H₁₉ClF₃N₅O: 474.1 (M + H), Measured: 474.2. |

### Example 19

### 2-(5-tert-Butyl-4-chloro-2-methyl-2H-pyrazol-3-yl)-7-chloro-5-(2,6-difluoro-phenyl)-1H-benzimidazole hydrochloride (Compound #112)

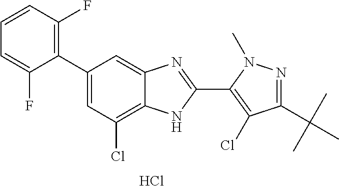

### STEP A. 2-Chloro-4-(5,5-dimethyl-[1,3,2]dioxaborinan-2-yl)-6-nitro-phenylamine

To a mixture of bis(neopentylglycolato)diboron (2.69 g, 11.9 mmol), 4-bromo-2-chloro-6-nitro-phenylamine (as prepared in Example 15, Step A, 2.00 g, 7.95 mmol), and Pd(dppf)Cl₂.DCM (581 mg, 0.795 mmol) in DMF (30 mL) was added KOAc (1.56 g, 15.9 mmol). The resulting mixture was stirred at 100° C. under microwave irradiation for 6 h and then cooled to room temperature. The mixture was treated with EtOAc (150 mL), then washed with H₂O (3×30 mL). The aqueous layers were extracted with EtOAc (2×30 mL). The combined organic layers were washed with H₂O and brine, then dried with Na₂SO₄. The solvent was removed under reduced pressure followed by flash chromatography of the residue on silica gel (5:95-20:80 EtOAc:hexanes) to yield a light gray solid. ¹H-NMR (400 MHz, CDCl₃) δ: 8.53 (d, J=1.3 Hz, 1H), 7.91 (d, J=1.3 Hz, 1H), 6.69 (br. s., 2H), 3.77 (s, 4H), 1.03 (s, 6H).

### STEP B. 3-Chloro-2′,6′-difluoro-5-nitro-biphenyl-4-ylamine

Following the procedures for Step A in Example 15, the title compound was prepared from 2-chloro-4-(5,5-dimethyl-[1,3,2]dioxaborinan-2-yl)-6-nitro-phenylamine (as prepared in the previous step, 500 mg, 1.76 mmol), 2-bromo-1,3-difluoro-benzene (340 mg, 1.76 mmol), Pd(PPh₃)₄ (203 mg, 0.176 mmol) and 2.0 M aqueous Na₂CO₃ (7.00 mL, 14.0 mmol) in 1,4-dioxane (10 mL), as a yellow solid. ¹H-NMR (400 MHz, CDCl₃) δ: 8.28 (s, 1H), 7.70 (s, 1H), 7.32 (tt, J=8.3, 6.3 Hz, 1H), 6.96-7.07 (m, 2H), 6.69 (br. s., 2H). Mass Spectrum (LCMS, ESI pos.) Calculated For C₁₂H₇CIF₂N₂O₂: 285.0 (M+H), Measured: 285.2.

### STEP C. 5-tert-Butyl-4-chloro-2-methyl-2H-pyrazole-3-carboxylic acid (3-chloro-2′,6′-difluoro-5-nitro-biphenyl-4-yl)-amide

Following the procedure for Step B in Example 16, the title compound was prepared from 3-chloro-2′,6′-difluoro-5-nitro-biphenyl-4-ylamine (as prepared in the previous step, 100 mg, 0.351 mmol), NaH (35.5 mg, 1.40 mmol, 95%), 5-tert-butyl-4-chloro-2-methyl-2H-pyrazole-3-carboxylic acid (80.0 mg, 0.368 mmol, as prepared in Example T), and oxalyl chloride (193 μL, 0.385 mmol, 2.0 M in DCM), as a light yellow solid. ¹H-NMR (400 MHz, CDCl₃) δ: 9.19 (s, 1H), 8.07 (s, 1H), 7.89 (d, J=1.8 Hz, 1H), 7.34-7.47 (m, 1H), 6.99-7.15 (m, 2H), 4.12 (s, 3H), 1.45 (s, 9H). Mass Spectrum (LCMS, ESI pos.) Calculated For C₂₁H₁₈O₂F₂N₄O₃: 483.1 (M+H), Measured: 483.2.

### STEP D. 2-(5-tert-Butyl-4-chloro-2-methyl-2H-pyrazol-3-yl)-7-chloro-5-(2,6-difluoro-phenyl)-1H-benzimidazole

Following the procedure for Step C in Example 15, the title compound was prepared from 5-tert-butyl-4-chloro-2-methyl-2H-pyrazole-3-carboxylic acid (3-chloro-2′,6′-difluoro-5-nitro-biphenyl-4-yl)-amide (as prepared in the previous step, 115 mg, 0.238 mmol) and iron powder (106 mg, 1.90 mmol), as a white solid. ¹H-NMR (400 MHz, CD₃OD) δ: 7.67 (s, 1H), 7.35-7.47 (m, 2H), 7.09 (t, J=8.1 Hz, 2H), 4.04 (s, 3H), 1.44 (s, 9H). Mass Spectrum (LCMS, ESI pos.) Calculated For C₂₁H₁₈Cl₂F₂N₄: 435.1 (M+H), Measured: 435.3.

### STEP E. 2-(5-tert-Butyl-4-chloro-2-methyl-2H-pyrazol-3-yl)-7-chloro-5-(2,6-difluoro-phenyl)-1H-benzimidazole hydrochloride

Following the procedure for Step D in Example 15, the title compound was prepared from 2-(5-tert-butyl-4-chloro-2-methyl-2H-pyrazol-3-yl)-7-chloro-5-(2,6-difluoro-phenyl)-1H-benzimidazole (as prepared in the previous step, 103 mg, 0.237 mmol) and 1.0 M HCl in Et₂O (244 μL, 0.244 mmol) in 3 mL of 1:1 MeOH/DCM, as a white solid.

¹H-NMR (400 MHz, CD₃OD) δ: 7.85 (d, J=1.3 Hz, 1H), 7.69 (d, J=1.3 Hz, 1H), 7.43-7.56 (m, 1H), 7.10-7.23 (m, 2H), 4.05 (s, 3H), 1.46 (s, 9H). Mass Spectrum (LCMS, ESI pos.) Calculated For C₂₁H₁₈O₂F₂N₄: 435.1 (M+H), Measured: 435.3.

Following the procedure described in Example 19, and selecting and substituting reagents, starting materials, and conditions as would be known to those skilled in the art, the following compounds of formula (I) of the present invention were prepared:

| ID | |
|---|---|
| No. | Name and Measured Physical Property |
| 112 | 2-(5-tert-Butyl-4-chloro-2-methyl-2H-pyrazol-3-yl)-7-chloro-5- |
| | (2,6-difluoro-phenyl)-1H-benzimidazole sodium salt |
| | ¹H-NMR (400 MHz, CD₃OD) δ: 7.60 (d, J = 1.0 Hz, 1H), 7.28- |
| | 7.39 (m, 1H), 7.17 (s, 1H), 6.99-7.09 (m, 2H), 3.92 (s, 3H), |
| | 1.44 (s, 9H) |
| | Mass Spectrum (LCMS, ESI pos.) Calculated For |
| | C₂₁H₁₈Cl₂F₂N₄: 435.1 (M + H), Measured: 435.2. |
| 113 | 2-(5-tert-Butyl-4-chloro-2-methyl-2H-pyrazol-3-yl)-7-chloro-5- |
| | (2-fluoro-6-trifluoromethyl-phenyl)-1H-benzimidazole sodium |
| | salt |
| | ¹H-NMR (400 MHz, CD₃OD) δ: 7.60-7.65 (m, 1H), 7.51-7.59 |
| | (m, 1H), 7.38-7.48 (m, 2H), 6.99 (s, 1H), 3.92 (s, 3H), 1.44 (s, |
| | 9H) |
| | Mass Spectrum (LCMS, ESI pos.) Calculated For |
| | C₂₂H₁₈Cl₂F₄N₄: 485.1 (M + H), Measured: 485.3. |
| 113 | 2-(5-tert-Butyl-4-chloro-2-methyl-2H-pyrazol-3-yl)-7-chloro-5- |
| | (2-fluoro-6-trifluoromethyl-phenyl)-1H-benzimidazole |
| | hydrochloride |
| | ¹H-NMR (400 MHz, CD₃OD) δ: 7.65-7.75 (m, 3H), 7.50-7.59 |
| | (m, 2H), 4.05 (s, 3H), 1.46 (s, 9H) |
| | Mass Spectrum (LCMS, ESI pos.) Calculated For |
| | C₂₂H₁₈Cl₂F₄N₄: 485.1 (M + H), Measured: 485.3. |
| 130 | 2-(5-tert-Butyl-4-chloro-2-methyl-2H-pyrazol-3-yl)-7-chloro-5- |
| | (2-fluoro-6-trifluoromethoxy-phenyl)-1H-benzimidazole |
| | hydrochloride |
| | ¹H-NMR (400 MHz, CDCl₃) δ: 7.74 (s, 1H), 7.58 (td, J = 8.4, |
| | 6.2 Hz, 1H), 7.53 (s, 1H), 7.29-7.39 (m, 2H), 4.05 (s, 3H), 1.46 |
| | (s, 9H) |
| | Mass Spectrum (LCMS, APCI pos.) Calculated For |
| | C₂₂H₁₈Cl₂F₄N₄O: 501.1 (M + H), Measured: 501.3. |
| 130 | 2-(5-tert-Butyl-4-chloro-2-methyl-2H-pyrazol-3-yl)-7-chloro-5- |
| | (2-fluoro-6-trifluoromethoxy-phenyl)-1H-benzimidazole sodium |
| | salt |
| | ¹H-NMR (400 MHz, CD₃OD) δ: 7.53 (s, 1H), 7.46 (td, J = 8.4, |
| | 6.2 Hz, 1H), 7.21-7.31 (m, 2H), 7.09 (s, 1H), 3.93 (s, 3H), 1.44 |
| | (s, 9H) |
| | Mass Spectrum (LCMS, ESI pos.) Calculated For |
| | C₂₂H₁₈Cl₂F₄N₄O 501.1 (M + H), Measured: 501.3. |
| 171 | 3-tert-Butyl-5-[7-chloro-5-(2,6-difluoro-phenyl)-1H- |
| | benzimidazol-2-yl]-1-methyl-1H-pyrazole-4-carbonitrile sodium |
| | salt |
| | ¹H-NMR (400 MHz, CD₃OD) δ: 7.62 (d, J = 1.5 Hz, 1H), 7.37 |
| | (tt, J = 8.3, 6.3 Hz, 1H), 7.18 (d, J = 1.5 Hz, 1H), 7.02-7.11 (m, |
| | 2H), 4.06 (s, 3H), 1.47 (s, 9H) |
| | Mass Spectrum (LCMS, ESI pos.) Calculated For |
| | C₂₂H₁₈ClF₂N₅: 426.1 (M + H), Measured: 426.3. |
| 172 | 3-tert-Butyl-5-[7-chloro-5-(2-fluoro-6-trifluoromethyl-phenyl)- |
| | 1H-benzimidazol-2-yl]-1-methyl-1H-pyrazole-4-carbonitrile |
| | sodium salt |
| | ¹H-NMR (400 MHz, CD₃OD) δ: 7.60-7.67 (m, 1H), 7.58 (dd, J = |
| | 8.1, 5.3 Hz, 1H), 7.40-7.49 (m, 2H), 6.97 (s, 1H), 4.06 (s, |
| | 3H), 1.47 (s, 9H) |
| | Mass Spectrum (LCMS, ESI pos.) Calculated For |
| | C₂₃H₁₈ClF₄N₅: 476.1 (M + H), Measured: 476.3. |
| 173 | 3-tert-Butyl-5-[7-chloro-5-(2-fluoro-6-trifluoromethoxy-phenyl)- |
| | 1H-benzimidazol-2-yl]-1-methyl-1H-pyrazole-4-carbonitrile |
| | sodium salt |
| | ¹H-NMR (400 MHz, CD₃OD) δ: 7.53 (s, 1H), 7.46 (td, J = 8.3, |
| | 6.1 Hz, 1H), 7.21-7.30 (m, 2H), 7.05 (s, 1H), 4.06 (s, 3H), 1.48 |
| | (s, 9H) |
| | Mass Spectrum (LCMS, ESI pos.) Calculated For |
| | C₂₃H₁₈ClF₄N₅O: 492.1 (M + H), Measured: 492.2. |

### Example 20

### 2-(5-tert-Butyl-4-chloro-2-methyl-2H-pyrazol-3-yl)-5-(2-fluoro-phenyl)-7-trifluoromethyl-1H-benzimidazole hydrochloride (Compound #127)

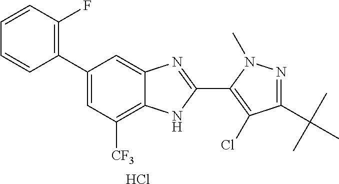

### STEP A. 2′-Fluoro-5-nitro-3-trifluoromethyl-biphenyl-4-ylamine

4-Bromo-2-nitro-6-trifluoromethylaniline (350 mg, 1.23 mmol), 2-fluorophenylboronic acid (258 mg, 1.85 mmol), and (dppf)PdCl₂.DCM (45.0 mg, 0.0615 mmol) under Ar were treated with DME (degassed, 6.0 mL) and 2.00 M aqueous Na₂CO₃ (degassed, 2.46 mL, 4.92 mmol). The mixture was stirred at 80° C. for 18 h, cooled to room temperature, and concentrated under reduced pressure to remove the DME. The resulting mixture was extracted with EtOAc (3×15 mL), and the combined extracts were dried (Na₂SO₄) and concentrated. The resulting dark oil was purified by chromatography using a 24-g SiO₂ pre-packed column and eluting with 0:100-60:40 DCM-hexanes to yield a yellow solid. ¹H-NMR (400 MHz, CDCl₃) δ: 8.57 (d, J=1.8 Hz, 1H), 7.98 (s, 1H), 7.42 (td, J=7.7, 1.8 Hz, 1H), 7.33-7.40 (m, 1H), 7.25 (dd, J=7.3, 1.3 Hz, 1H), 7.14-7.23 (m, 2H), 6.75 (br. s., 2H).

### STEP B. 5-tert-Butyl-4-chloro-2-methyl-2H-pyrazole-3-carboxylic acid (2′-fluoro-5-nitro-3-trifluoromethyl-biphenyl-4-yl)-amide

Following the procedure for Step B in Example 15, the title compound was prepared from 3-chloro-5-nitro-2′-fluoro-biphenyl-4-ylamine (94.3 mg, 0.314 mmol, as prepared in the previous step), dry NaH (32.0 mg, 1.26 mmol, 95%), 5-tert-butyl-4-chloro-2-methyl-2H-pyrazole-3-carboxylic acid (75.0 mg, 0.346 mmol, as prepared in Example T), and oxalyl chloride (188 μL, 0.377 mmol, 2.0 M in DCM), as a light yellow solid. ¹H-NMR (400 MHz, CDCl₃) δ: 8.97 (s, 1H), 8.35 (s, 1H), 8.16 (s, 1H), 7.42-7.54 (m, 2H), 7.29-7.38 (m, 1H), 7.19-7.26 (m, 1H), 4.10 (s, 3H), 1.44 (s, 9H). Mass Spectrum (LCMS, ESI pos.) Calculated For C₂₂H₁₉ClF₄N₄O₃: 499.1 (M+H), Measured: 499.0.

### STEP C. 2-(5-tert-Butyl-4-chloro-2-methyl-2H-pyrazol-3-yl)-5-(2-fluoro-phenyl)-7-trifluoromethyl-1H-benzimidazole

Following the procedure for Step C in Example 15, the title compound was prepared from 5-tert-butyl-4-chloro-2-methyl-2H-pyrazole-3-carboxylic acid (2′-fluoro-5-nitro-3-trifluoromethyl-biphenyl-4-yl)-amide (as prepared in the previous step, 97.0 mg, 0.194 mmol) and iron powder (87.1 mg, 1.56 mmol) in 3 mL of 1:1 AcOH/EtOH, as a white solid. ¹H-NMR (400 MHz, CD₃OD) δ: 8.03 (br. s., 1H), 7.75 (s, 1H), 7.52 (td, J=7.8, 1.8 Hz, 1H), 7.33-7.40 (m, 1H), 7.25 (td, J=7.5, 1.1 Hz, 1H), 7.15-7.23 (m, 1H), 4.07 (s, 3H), 1.42 (s, 9H). Mass Spectrum (LCMS, ESI pos.) Calculated For C₂₂H₁₉ClF₄N₄: 451.1 (M+H), Measured: 451.3.

### STEP D. 2-(5-tert-Butyl-4-chloro-2-methyl-2H-pyrazol-3-yl)-5-(2-fluoro-phenyl)-7-trifluoromethyl-1H-benzimidazole hydrochloride

Following the procedure for Step D in Example 15, the title compound was prepared from 2-(5-tert-butyl-4-chloro-2-methyl-2H-pyrazol-3-yl)-5-(2-fluoro-phenyl)-7-trifluoromethyl-1H-benzimidazole (as prepared in the previous step, 77.8 mg, 0.173 mmol) and 1.0 M HCl (173 μL, 0.173 mmol) in 3 mL of 1:1 MeOH/DCM, as a white solid.

¹H-NMR (400 MHz, CDCl₃) δ: 8.13 (s, 1H), 7.87 (s, 1H), 7.62 (td, J=7.8, 1.8 Hz, 1H), 7.41-7.50 (m, 1H), 7.31-7.37 (m, 1H), 7.24-7.31 (m, 1H), 4.06 (s, 3H), 1.46 (s, 9H). Mass Spectrum (LCMS, APCI pos.) Calculated For C₂₂H₁₉ClF₄N₄: 451.1 (M+H), Measured: 451.3.

Following the procedure described in Example 20, and selecting and substituting reagents, starting materials, and conditions as would be known to those skilled in the art, the following compounds of formula (I) of the present invention were prepared:

| ID | |
|---|---|
| No. | Name and Measured Physical Property |
| 127 | 2-(5-tert-Butyl-4-chloro-2-methyl-2H-pyrazol-3-yl)-5-(2- |
| | fluoro-phenyl)-7-trifluoromethyl-1H-benzimidazole sodium |
| | salt |
| | ¹H-NMR (400 MHz, CD₃OD) δ: 7.98 (s, 1H), 7.52-7.60 (m, |
| | 2H), 7.31-7.38 (m, 1H), 7.23-7.29 (m, 1H), 7.20 (ddd, J = |
| | 11.0, 8.2, 1.0 Hz, 1H), 1.44 (s, 9H) |
| | Mass Spectrum (LCMS, ESI pos.) Calculated For |
| | C₂₂H₁₉ClF₄N₄: 451.1 (M + H), Measured: 451.2. |
| 128 | 2-(5-tert-Butyl-4-chloro-2-methyl-2H-pyrazol-3-yl)-5-(2- |
| | chloro-phenyl)-7-trifluoromethyl-1H-benzimidazole sodium |
| | salt |
| | ¹H-NMR (400 MHz, CD₃OD) δ: 7.84 (s, 1H), 7.51 (dd, J = |
| | 7.8, 1.3 Hz, 1H), 7.46 (dd, J = 7.6, 1.8 Hz, 1H), 7.35-7.41 |
| | (m, 2H), 7.29-7.35 (m, 1H), 3.89 (s, 3H), 1.44 (s, 9H) |
| | Mass Spectrum (LCMS, ESI pos.) Calculated For |
| | C₂₂H₁₉Cl₂F₃N₄: 467.1 (M + H), Measured: 467.2. |
| 128 | 2-(5-tert-Butyl-4-chloro-2-methyl-2H-pyrazol-3-yl)-5-(2- |
| | chloro-phenyl)-7-trifluoromethyl-1H-benzimidazole |
| | hydrochloride |
| | ¹H-NMR (400 MHz, CDCl₃) δ: 8.01 (s, 1H), 7.75 (s, 1H), |
| | 7.55-7.61 (m, 1H), 7.40-7.53 (m, 3H), 4.06 (s, 3H), 1.46 (s, |
| | 9H) |
| | Mass Spectrum (LCMS, APCI pos.) Calculated For |
| | C₂₂H₁₉Cl₂F₃N₄: 467.1 (M + H), Measured: 467.3. |
| 129 | 2-(5-tert-Butyl-4-chloro-2-methyl-2H-pyrazol-3-yl)-7- |
| | trifluoromethyl-5-(2-trifluoromethyl-phenyl)-1H- |
| | benzimidazole hydrochloride |
| | ¹H-NMR (400 MHz, CDCl₃) δ: 7.92 (s, 1H), 7.86 (d, J = 7.8 |
| | Hz, 1H), 7.68-7.76 (m, 1H), 7.61-7.68 (m, 2H), 7.50 (d, J = |
| | 7.3 Hz, 1H), 4.07 (s, 3H), 1.46 (s, 9H) |
| | Mass Spectrum (LCMS, APCI pos.) Calculated For |
| | C₂₃H₁₉ClF₆N₄: 501.1 (M + H), Measured: 501.3. |
| 129 | 2-(5-tert-Butyl-4-chloro-2-methyl-2H-pyrazol-3-yl)-7- |
| | trifluoromethyl-5-(2-trifluoromethyl-phenyl)-1H- |
| | benzimidazole sodium salt |
| | ¹H-NMR (400 MHz, CD₃OD) δ: 7.80 (d, J = 7.8 Hz, 1H), 7.75 |
| | (s, 1H), 7.61-7.69 (m, 1H), 7.51-7.57 (m, 1H), 7.46 (d, J = |
| | 7.6 Hz, 1H), 7.30 (s, 1H), 3.90 (s, 3H), 1.44 (s, 9H) |
| | Mass Spectrum (LCMS, ESI pos.) Calculated For |
| | C₂₃H₁₉ClF₆N₄: 501.1 (M + H), Measured: 501.2. |

### Example 21

### 2-(5-tert-Butyl-4-chloro-2-methyl-2H-pyrazol-3-yl)-5-(2,6-difluoro-phenyl)-7-trifluoromethyl-1H-benzimidazole hydrochloride (Compound #124)

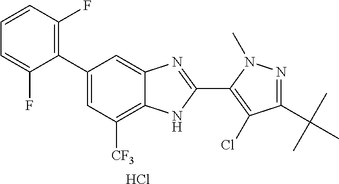

### STEP A. 4-(5,5-Dimethyl-[1,3,2]dioxaborinan-2-yl)-2-nitro-6-trifluoromethyl-phenylamine

4-Bromo-2-nitro-6-trifluoromethylaniline (500 mg, 1.75 mmol, Trylead Chemical Co.), bis(neopentylglycolato)diboron (712 mg, 3.15 mmol), KOAc (515 mg, 5.25 mmol), and (dppf)PdCl₂.DCM (128 mg, 0.175 mmol) under Ar were treated with anhydrous 1,4-dioxane (degassed, 5.0 mL). The mixture was stirred at 100° C. for 16 h, cooled to room temperature, and concentrated under reduced pressure. The resulting dark oil was purified by chromatography by applying to a 40-g SiO₂ pre-packed column in DCM-hexane (2:1) and eluting with 0:100-25:75 EtOAc-heptane to yield a crystalline yellow solid. ¹H-NMR (400 MHz, CDCl₃) δ: 8.75 (s, 1H), 8.11 (s, 1H), 6.78 (br. s., 2H), 3.76 (s, 4H), 1.02 (s, 6H).

### STEP B. 2′,6′-Difluoro-5-nitro-3-trifluoromethyl-biphenyl-4-ylamine

4-(5,5-Dimethyl-[1,3,2]dioxaborinan-2-yl)-2-nitro-6-trifluoromethyl-phenylamine (510 mg, 1.60 mmol, as prepared in the previous step), 3-bromo-2,6-difluorobenzene (463 mg, 2.40 mmol), and (PPh₃)₄Pd (92.5 mg, 0.0800 mmol) under Ar were treated with DME (degassed, 7.0 mL) and 2.00 M aqueous Na₂CO₃ (degassed, 3.20 mL, 6.40 mmol). The mixture was stirred at 85° C. for 18 h, cooled to room temperature, and concentrated under reduced pressure to remove the DME. The residue was extracted with EtOAc (3×15 mL), and the combined extracts were dried (Na₂SO₄) and concentrated. The resulting dark oil was purified by chromatography using a 24-g SiO₂ pre-packed column and eluting with 0:100-60:40 DCM-hexanes. Trituration of the resulting yellow resin with Et₂O-hexanes followed by concentration under vacuum yielded a crystalline yellow solid. ¹H-NMR (400 MHz, CDCl₃) δ: 8.51 (s, 1H), 7.87 (s, 1H), 7.28-7.39 (m, 1H), 6.97-7.07 (m, 2H), 6.79 (br. s., 2H).

### STEP C. 5-tert-Butyl-4-chloro-2-methyl-2H-pyrazole-3-carboxylic acid (2′,6′-difluoro-5-nitro-3-trifluoromethyl-biphenyl-4-yl)-amide

Following the procedure for Step B in Example 15, the title compound was prepared from 2′,6′-difluoro-5-nitro-3-trifluoromethyl-biphenyl-4-ylamine (as prepared in the previous step, 100 mg, 0.314 mmol), NaH (32.0 mg, 1.26 mmol, 95%), 5-tert-butyl-4-chloro-2-methyl-2H-pyrazole-3-carboxylic acid (75.0 mg, 0.346 mmol, as prepared in Example T) and oxalyl chloride (188 μL, 0.377 mmol) as a light yellow solid. ¹H-NMR (400 MHz, CDCl₃) δ: 9.00 (s, 1H), 8.31 (s, 1H), 8.10 (s, 1H), 7.44 (tt, J=8.5, 6.3 Hz, 1H), 7.02-7.15 (m, 2H), 4.10 (s, 3H), 1.44 (s, 9H). Mass Spectrum (LCMS, ESI pos.) Calculated For C₂₂H₁₈ClF₅N₄O₃: 517.1 (M+H), Measured: 517.2.

### STEP D. 2-(5-tert-Butyl-4-chloro-2-methyl-2H-pyrazol-3-yl)-5-(2,6-difluoro-phenyl)-7-trifluoromethyl-1H-benzimidazole

Following the procedure for Step C in Example 15, the title compound was prepared from 5-tert-butyl-4-chloro-2-methyl-2H-pyrazole-3-carboxylic acid (2′,6′-difluoro-5-nitro-3-trifluoromethyl-biphenyl-4-yl)-amide (as prepared in the previous step, 78.0 mg, 0.151 mmol) and iron powder (67.6 mg, 1.21 mmol) in 1:1 AcOH/EtOH (3 mL) as a white solid. ¹H-NMR (400 MHz, CD₃OD) δ: 7.98 (s, 1H), 7.66 (s, 1H), 7.43 (tt, J=8.5, 6.3 Hz, 1H), 7.05-7.16 (m, 2H), 4.08 (s, 3H), 1.43 (s, 9H). Mass Spectrum (LCMS, ESI pos.) Calculated For C₂₂H₁₈ClF₅N₄: 469.1 (M+H), Measured: 469.3.

### STEP E. 2-(5-tert-Butyl-4-chloro-2-methyl-2H-pyrazol-3-yl)-5-(2,6-difluoro-phenyl)-7-trifluoromethyl-1H-benzimidazole hydrochloride

Following the procedure for Step D in Example 15, the title compound was prepared from 2-(5-tert-butyl-4-chloro-2-methyl-2H-pyrazol-3-yl)-5-(2,6-difluoro-phenyl)-7-trifluoromethyl-1H-benzimidazole (as prepared in the previous step, 63.3 mg, 0.135 mmol) and 1.0 M HCl (135 μL, 0.135 mmol) in 1:1 MeOH/DCM (3 mL) as a white solid.

¹H-NMR (400 MHz, CD₃OD) δ: 8.07 (s, 1H), 7.78 (s, 1H), 7.49 (tt, J=8.5, 6.4 Hz, 1H), 7.10-7.21 (m, 2H), 4.07 (s, 3H), 1.46 (s, 9H). Mass Spectrum (LCMS, APCI pos.) Calculated For C₂₂H₁₈ClF₅N₄: 469.1 (M+H), Measured: 469.3.

Following the procedure described in Example 21, and selecting and substituting reagents, starting materials, and conditions as would be known to those skilled in the art, the following compounds of formula (I) of the present invention were prepared:

| ID | |
|---|---|
| No. | Name and Measured Physical Property |
| 124 | 2-(5-tert-Butyl-4-chloro-2-methyl-2H-pyrazol-3-yl)-5-(2,6- |
| | difluoro-phenyl)-7-trifluoromethyl-1H-benzimidazole sodium |
| | salt |
| | ¹H-NMR (400 MHz, CD₃OD) δ: 7.88 (s, 1H), 7.41 (s, 1H), |
| | 7.32-7.40 (m, 1H), 7.08 (t, J = 8.1 Hz, 2H), 3.90 (s, 3H), 1.44 |
| | (s, 9H) |
| | Mass Spectrum (LCMS, ESI pos.) Calculated For |
| | C₂₂H₁₈ClF₅N₄: 469.1 (M + H), Measured: 469.3. |
| 125 | 2-(5-tert-Butyl-4-chloro-2-methyl-2H-pyrazol-3-yl)-5-(2-fluoro- |
| | 6-trifluoromethyl-phenyl)-7-trifluoromethyl-1H-benzimidazole |
| | sodium salt |
| | ¹H-NMR (400 MHz, CD₃OD) δ: 7.73 (s, 1H), 7.63-7.69 (m, |
| | 1H), 7.59 (td, J = 7.8, 5.6 Hz, 1H), 7.45-7.51 (m, 1H), 7.26 (s, |
| | 1H), 3.92 (s, 3H), 1.44 (s, 9H) |
| | Mass Spectrum (LCMS, ESI pos.) Calculated For |
| | C₂₃H₁₈ClF₇N₄: 519.1 (M + H), Measured: 519.3. |
| 125 | 2-(5-tert-Butyl-4-chloro-2-methyl-2H-pyrazol-3-yl)-5-(2-fluoro- |
| | 6-trifluoromethyl-phenyl)-7-trifluoromethyl-1H-benzimidazole |
| | hydrochloride |
| | ¹H-NMR (400 MHz, CD₃OD) δ: 7.88 (s, 1H), 7.64-7.74 (m, |
| | 2H), 7.51-7.60 (m, 2H), 4.08 (s, 3H), 1.46 (s, 9H) |
| | Mass Spectrum (LCMS, APCI pos.) Calculated For |
| | C₂₃H₁₈ClF₇N₄: 519.1 (M + H), Measured: 519.4. |
| 126 | 2-(5-tert-Butyl-4-chloro-2-methyl-2H-pyrazol-3-yl)-5-(2-fluoro- |
| | 6-trifluoromethoxy-phenyl)-7-trifluoromethyl-1H- |
| | benzimidazole hydrochloride |
| | ¹H-NMR (400 MHz, CDCl₃) δ: 7.96 (s, 1H), 7.65 (s, 1H), 7.58 |
| | (td, J = 8.5, 6.3 Hz, 1H), 7.29-7.40 (m, 2H), 4.08 (s, 3H), 1.46 |
| | (s, 9H) |
| | Mass Spectrum (LCMS, APCI pos.) Calculated For |
| | C₂₃H₁₈ClF₇N₄O: 535.1 (M + H), Measured: 535.3. |
| 126 | 2-(5-tert-Butyl-4-chloro-2-methyl-2H-pyrazol-3-yl)-5-(2-fluoro- |
| | 6-trifluoromethoxy-phenyl)-7-trifluoromethyl-1H- |
| | benzimidazole sodium salt |
| | ¹H-NMR (400 MHz, CD₃OD) δ: 7.82 (s, 1H), 7.49 (td, J = 8.4, |
| | 6.2 Hz, 1H), 7.36 (s, 1H), 7.23-7.34 (m, 2H), 3.93 (s, 3H), |
| | 1.44 (s, 9H) |
| | Mass Spectrum (LCMS, ESI pos.) Calculated For |
| | C₂₃H₁₈ClF₇N₄O: 535.1 (M + H), Measured: 535.3. |

### Example 22

### 3-tert-Butyl-5-[(5-(2-fluoro-phenyl)-7-trifluoromethyl-1H-benzimidazol-2-yl]-1-methyl-1H-pyrazole-4-carbonitrile sodium salt (Compound #147)

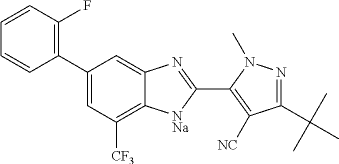

### STEP A. 5-tert-Butyl-4-cyano-2-methyl-2H-pyrazole-3-carboxylic acid (4-bromo-2-nitro-6-trifluoromethyl-phenyl)-amide

Following the procedure for Step B in Example 15, the title compound was prepared from 4-bromo-2-nitro-6-trifluoromethyl-phenylamine (713 mg, 2.50 mmol), NaH (253 mg, 10.0 mmol, 95%), 5-tert-butyl-4-cyano-2-methyl-2H-pyrazole-3-carboxylic acid (518 mg, 2.50 mmol, as prepared in Example V), and oxalyl chloride (1.50 mL, 3.00 mmol, 2.0 M in DCM), as a white solid. ¹H-NMR (400 MHz, CDCl₃) δ: 8.45 (s, 1H), 8.36 (d, J=2.0 Hz, 1H), 8.14 (d, J=2.3 Hz, 1H), 4.11 (s, 3H), 1.47 (s, 9H).

### STEP B. 5-(5-Bromo-7-trifluoromethyl-1H-benzimidazol-2-yl)-3-tert-butyl-1-methyl-1H-pyrazole-4-carbonitrile

A mixture of 5-tert-butyl-4-cyano-2-methyl-2H-pyrazole-3-carboxylic acid (4-bromo-2-nitro-6-trifluoromethyl-phenyl)-amide (as prepared in the previous step, 750 mg, 1.58 mmol) and iron powder (706 mg, 12.6 mmol) in 1:1 HOAc/EtOH (20 mL) was stirred at 100° C. for 1 h, then cooled to room temperature. The solid was removed by filtration through a pad of diatomaceous earth and washed with DCM (20 mL). The filtrate was concentrated under reduced pressure, and the residue was purified by flash chromatography on silica gel (0:1-3:17 EtOAc-hexanes) to yield as a white solid. ¹H-NMR (400 MHz, CD₃OD) δ: 8.12 (s, 1H), 7.77 (s, 1H), 4.12 (s, 3H), 1.47 (s, 9H). Mass Spectrum (LCMS, APCI pos.) Calculated For C₁₇H₁₅BrF₃N₅: 426.1 (M+H), Measured: 426.3.

### STEP C. 3-tert-Butyl-5-[5-(2-fluoro-phenyl)-7-trifluoromethyl-1H-benzimidazol-2-yl]-1-methyl-1H-pyrazole-4-carbonitrile

Following the procedure for Step A in Example 15, the title compound was prepared from 3-tert-butyl-5-[5-(2-fluoro-phenyl)-7-trifluoromethyl-1H-benzimidazol-2-yl]-1-methyl-1H-pyrazole-4-carbonitrile (as prepared in the previous step, 60.0 mg, 0.141 mmol), 2-fluoro-phenylboronic acid (23.6 mg, 0.169 mmol), Pd(PPh₃)₄ (16.3 mg, 0.0141 mmol) and 2.0 M aqueous Na₂CO₃ (564 μL, 1.13 mmol) in 1,4-dioxane (2 mL), as a white solid. ¹H-NMR (400 MHz, CD₃OD) δ: 8.10 (s, 1H), 7.82 (s, 1H), 7.61 (td, J=7.8, 1.8 Hz, 1H), 7.38-7.48 (m, 1H), 7.30-7.37 (m, 1H), 7.27 (dd, J=11.1, 8.3 Hz, 1H), 4.14 (s, 3H), 1.49 (s, 9H). Mass Spectrum (LCMS, ESI pos.) Calculated For C₂₃H₁₉F₄N₅: 442.2 (M+H), Measured: 442.2.

### STEP D. 3-tert-Butyl-5-[5-(2-fluoro-phenyl)-7-trifluoromethyl-1H-benzimidazol-2-yl]-1-methyl-1H-pyrazole-4-carbonitrile sodium salt

To a solution of 3-tert-butyl-5-[5-(2-fluoro-phenyl)-7-trifluoromethyl-1H-benzimidazol-2-yl]-1-methyl-1H-pyrazole-4-carbonitrile (as prepared in the previous step, 39.1 mg, 0.0886 mmol) in 1:1 MeOH/THF (1 mL) was added a solution of 0.50 M NaOMe in MeOH (177 μL, 0.0886 mmol). After stirring at room temperature for 0.5 h, the mixture was concentrated in vacuo to yield the title compound as a white solid.

¹H-NMR (400 MHz, CD₃OD) δ: 8.00 (s, 1H), 7.53-7.61 (m, 2H), 7.31-7.39 (m, 1H), 7.27 (td, J=7.5, 1.4 Hz, 1H), 7.21 (dd, J=12.3, 8.2 Hz, 1H), 4.02-4.07 (m, 3H), 1.48 (s, 9H). Mass Spectrum (LCMS, ESI pos.) Calculated For C₂₃H₁₉F₄N₅: 442.2 (M+H), Measured: 442.2.

Following the procedure described in Example 22, and selecting and substituting reagents, starting materials, and conditions as would be known to those skilled in the art, the following compounds of formula (I) of the present invention were prepared:

| ID | |
|---|---|
| No. | Name and Measured Physical Property |
| 82 | 2-(3-tert-Butyl-isoxazol-5-yl)-5-(2-fluoro-6-trifluoromethyl- |
| | phenyl)-1H-benzimidazole hydrochloride |
| | ¹H-NMR (400 MHz, CD₃OD) δ: 7.73 (d, J = 8.3 Hz, 1H), 7.55- |
| | 7.70 (m, 3H), 7.45-7.54 (m, 1H), 7.26 (d, J = 8.3 Hz, 1H), 7.11 (s, |
| | 1H), 1.43 (s, 9H) |
| | Mass Spectrum (LCMS, ESI pos.) Calculated For C₂₁H₁₇F₄N₃O: |
| | 404.0 (M + H), Measured: 404.1. |
| 147 | 3-tert-Butyl-5-[5-(2-fluoro-phenyl)-7-trifluoromethyl-1H- |
| | benzimidazol-2-yl]-1-methyl-1H-pyrazole-4-carbonitrile |
| | hydrochloride |
| | ¹H-NMR (400 MHz, CD₃OD) δ: 8.11 (s, 1H), 7.83 (s, 1H), 7.62 |
| | (td, J = 7.8, 1.6 Hz, 1H), 7.41-7.47 (m, 1H), 7.31-7.37 (m, 1H), |
| | 7.23-7.31 (m, 1H), 4.15 (s, 3H), 1.44-1.51 (m, 9H) |
| | Mass Spectrum (LCMS, ESI pos.) Calculated For C₂₃H₁₉F₄N₅: |
| | 442.2 (M + H), Measured: 442.2. |
| 148 | 3-tert-Butyl-5-[5-(2-chloro-phenyl)-7-trifluoromethyl-1H- |
| | benzimidazol-2-yl]-1-methyl-1H-pyrazole-4-carbonitrile |
| | hydrochloride |
| | ¹H-NMR (400 MHz, CD₃OD) δ: 7.98 (s, 1H), 7.70 (s, 1H), 7.55- |
| | 7.59 (m, 1H), 7.48-7.52 (m, 1H), 7.39-7.47 (m, 2H), 4.14 (s, 3H), |
| | 1.49 (m, 9H) |
| | Mass Spectrum (LCMS, ESI pos.) Calculated For C₂₃H₁₉ClF₃N₅: |
| | 458.1 (M + H), Measured: 458.2. |
| 148 | 3-tert-Butyl-5-[5-(2-chloro-phenyl)-7-trifluoromethyl-1H- |
| | benzimidazol-2-yl]-1-methyl-1H-pyrazole-4-carbonitrile sodium |
| | salt |
| | ¹H-NMR (400 MHz, CD₃OD) δ: 7.86 (s, 1H), 7.51 (dd, J = 7.8, |
| | 1.3 Hz, 1H), 7.47 (dd, J = 7.6, 1.8 Hz, 1H), 7.28-7.43 (m, 3H), |
| | 4.04 (s, 3H), 1.48 (s, 9H) |
| | Mass Spectrum (LCMS, ESI pos.) Calculated For C₂₃H₁₉ClF₃N₅: |
| | 458.1 (M + H), Measured: 458.2. |
| 149 | 3-tert-Butyl-1-methyl-5-[7-trifluoromethyl-5-(2-trifluoromethyl- |
| | phenyl)-1H-benzimidazol-2-yl]-1H-pyrazole-4-carbonitrile |
| | sodium salt |
| | ¹H-NMR (400 MHz, CD₃OD) δ: 7.81 (d, J = 7.8 Hz, 1H), 7.78 (s, |
| | 1H), 7.67 (t, J = 7.3 Hz, 1H), 7.56 (t, J = 7.6 Hz, 1H), 7.48 (d, J = |
| | 7.6 Hz, 1H), 7.35 (s, 1H), 4.06 (s, 3H), 1.48 (s, 9H) |
| | Mass Spectrum (LCMS, ESI pos.) Calculated For C₂₄H₁₉F₆N₅: |
| | 492.2 (M + H), Measured: 492.2. |
| 149 | 3-tert-Butyl-1-methyl-5-[7-trifluoromethyl-5-(2-trifluoromethyl- |
| | phenyl)-1H-benzimidazol-2-yl]-1H-pyrazole-4-carbonitrile |
| | hydrochloride |
| | ¹H-NMR (400 MHz, CD₃OD) δ: 7.89 (s, 1H), 7.86 (d, J = 7.8 Hz, |
| | 1H), 7.70-7.76 (m, 1H), 7.61-7.66 (m, 1H), 7.59 (s, 1H), 7.51 (d, |
| | J = 7.6 Hz, 1H), 4.15 (s, 3H), 1.49 (s, 9H) |
| | Mass Spectrum (LCMS, ESI pos.) Calculated For C₂₄H₁₉F₆N₅: |
| | 492.2 (M + H), Measured: 492.2. |
| 150 | 3-tert-Butyl-1-methyl-5-[5-(2-trifluoromethoxy-phenyl)-7- |
| | trifluoromethyl-1H-benzimidazol-2-yl]-1H-pyrazole-4-carbonitrile |
| | sodium salt |
| | ¹H-NMR (400 MHz, CD₃OD) δ: 7.94 (s, 1H), 7.57-7.64 (m, 1H), |
| | 7.53 (s, 1H), 7.39-7.51 (m, 3H), 4.07 (s, 3H), 1.48 (s, 9H) |
| | Mass Spectrum (LCMS, ESI pos.) Calculated For C₂₄H₁₉F₆N₅O: |
| | 508.2 (M + H), Measured: 508.2. |
| 150 | 3-tert-Butyl-1-methyl-5-[5-(2-trifluoromethoxy-phenyl)-7- |
| | trifluoromethyl-1H-benzimidazol-2-yl]-1H-pyrazole-4-carbonitrile |
| | hydrochloride |
| | ¹H-NMR (400 MHz, CD₃OD) δ: 8.03 (s, 1H), 7.75 (s, 1H), 7.61- |
| | 7.65 (m, 1H), 7.53 (ddd, J = 7.2, 4.9, 2.0 Hz, 2H), 7.46-7.51 (m, |
| | 1H), 4.15 (s, 3H), 1.49 (m, 9H) |
| | Mass Spectrum (LCMS, ESI pos.) Calculated For C₂₄H₁₉F₆N₅O: |
| | 508.2 (M + H), Measured: 508.2. |
| 185 | 2-(5-tert-Butyl-2-methyl-4-trifluoromethyl-2H-pyrazol-3-yl)-5-(2- |
| | fluoro-phenyl)-7-trifluoromethyl-1H-benzimidazole sodium salt |
| | ¹H-NMR (400 MHz, CD₃OD) δ: 7.97 (s, 1H), 7.52-7.60 (m, 2H), |
| | 7.31-7.39 (m, 1H), 7.27 (td, J = 7.5, 1.3 Hz, 1H), 7.17-7.24 (m, |
| | 1H), 3.67 (s, 3H), 1.42 (s, 9H) |
| | Mass Spectrum (LCMS, ESI pos.) Calculated For C₂₃H₁₉F₇N₄: |
| | 485.2 (M + H), Measured: 485.2. |
| 186 | 2-(5-tert-Butyl-2-methyl-4-trifluoromethyl-2H-pyrazol-3-yl)-5-(2- |
| | chloro-phenyl)-7-trifluoromethyl-1H-benzimidazole sodium salt |
| | ¹H-NMR (400 MHz, CD3OD) δ: 7.83 (s, 1H), 7.52 (dd, J = 7.7, |
| | 1.4 Hz, 1H), 7.45-7.49 (m, 1H), 7.43 (s, 1H), 7.31-7.41 (m, 2H), |
| | 3.61-3.72 (m, 3H), 1.37-1.46 (m, 9H) |
| | Mass Spectrum (LCMS, ESI pos.) Calculated For C₂₃H₁₉ClF₆N₄: |
| | 501.1 (M + H), Measured: 501.1. |
| 187 | 2-(5-tert-Butyl-2-methyl-4-trifluoromethyl-2H-pyrazol-3-yl)-7- |
| | trifluoromethyl-5-(2-trifluoromethyl-phenyl)-1H-benzimidazole |
| | sodium salt |
| | ¹H-NMR (400 MHz, CD₃OD) δ: 7.80 (d, J = 7.8 Hz, 1H), 7.74 (s, |
| | 1H), 7.66 (t, J = 7.5 Hz, 1H), 7.55 (t, J = 7.6 Hz, 3H), 7.48 (d, J = |
| | 7.6 Hz, 1H), 7.33 (s, 1H), 3.69 (s, 3H), 1.42 (s, 9H) |
| | Mass Spectrum (LCMS, ESI pos.) Calculated For C₂₄H₁₉F₉N₄O₃: |
| | 535.2 (M + H), Measured: 535.1. |
| 188 | 2-(5-tert-Butyl-2-methyl-4-trifluoromethyl-2H-pyrazol-3-yl)-5-(2- |
| | trifluoromethoxy-phenyl)-7-trifluoromethyl-1H-benzimidazole |
| | sodium salt |
| | ¹H-NMR (400 MHz, CD₃OD) δ: 7.94 (s, 1H), 7.57-7.64 (m, 1H), |
| | 7.53 (s, 1H), 7.39-7.51 (m, 3H), 4.07 (s, 3H), 1.48 (s, 9H) |
| | Mass Spectrum (LCMS, ESI pos.) Calculated For C₂₄H₁₉F₆N₅O: |
| | 508.2 (M + H), Measured: 508.2. |
| 139 | 2-(3-tert-Butyl-isoxazol-5-yl)-5-(2-fluoro-phenyl)-7- |
| | trifluoromethyl-1H-benzimidazole sodium salt |
| | ¹H-NMR (400 MHz, CD₃OD) δ: 7.97 (s, 1H), 7.54-7.60 (m, 1H), |
| | 7.53 (s, 1H), 7.29-7.38 (m, 1H), 7.23-7.29 (m, 1H), 7.14-7.23 |
| | (m, 1H), 7.09 (s, 1H), 1.39 (s, 9H) |
| | Mass Spectrum (LCMS, ESI pos.) Calculated For C₂₁H₁₇F₄N₃O: |
| | 404.1 (M + H), Measured: 404.1. |
| 139 | 2-(3-tert-Butyl-isoxazol-5-yl)-5-(2-fluoro-phenyl)-7- |
| | trifluoromethyl-1H-benzimidazole hydrochloride |
| | ¹H-NMR (400 MHz, CD₃OD) δ: 8.12 (s, 1H), 7.92 (s, 1H), 7.62 |
| | (td, J = 7.8, 1.8 Hz, 1H), 7.44-7.51 (m, 1H), 7.42 (s, 1H), 7.34 |
| | (td, J = 7.6, 1.3 Hz, 1H), 7.24-7.32 (m, 1H), 1.44 (s, 9H) |
| | Mass Spectrum (LCMS, APCI pos.) Calculated For |
| | C₂₁H₁₇F₄N₃O: 404.1 (M + H), Measured: 404.1. |
| 141 | 2-(3-tert-Butyl-isoxazol-5-yl)-5-(2-chloro-phenyl)-7- |
| | trifluoromethyl-1H-benzimidazole hydrochloride |
| | ¹H-NMR (400 MHz, CD₃OD) δ: 8.00 (s, 1H), 7.80 (s, 1H), 7.37- |
| | 7.59 (m, 4H), 7.43 (s, 1H), 1.43 (s, 9H) |
| | Mass Spectrum (LCMS, APCI pos.) Calculated For |
| | C₂₁H₁₇ClF₃N₃O: 420.1 (M + H), Measured: 420.1. |
| 141 | 2-(3-tert-Butyl-isoxazol-5-yl)-5-(2-chloro-phenyl)-7- |
| | trifluoromethyl-1H-benzimidazole sodium salt |
| | ¹H-NMR (400 MHz, CD₃OD) δ: 7.84 (s, 1H), 7.51 (dd, J = 7.8, |
| | 1.3 Hz, 1H), 7.44-7.48 (m, 1H), 7.43 (s, 1H), 7.38 (td, J = 7.5, |
| | 1.5 Hz, 1H), 7.30-7.35 (m, 1H), 1.37-1.46 (m, 9H) |
| | Mass Spectrum (LCMS, ESI pos.) Calculated For |
| | C₂₁H₁₇ClF₃N₃O: 420.1 (M + H), Measured: 420.1. |
| 142 | 2-(3-tert-Butyl-isoxazol-5-yl)-7-trifluoromethyl-5-(2- |
| | trifluoromethyl-phenyl)-1H-benzimidazole sodium salt |
| | ¹H-NMR (400 MHz, CD₃OD) δ: 7.79 (d, J = 7.6 Hz, 1H), 7.74 (s, |
| | 1H), 7.62-7.68 (m, 1H), 7.51-7.58 (m, 1H), 7.46 (d, J = 7.6 Hz, |
| | 1H), 7.30 (s, 1H), 7.11 (s, 1H), 1.42 (s, 9H) |
| | Mass Spectrum (LCMS, ESI pos.) Calculated For C₂₂H₁₇F₆N₃O: |
| | 454.1 (M + H), Measured: 454.1. |
| 142 | 2-(3-tert-Butyl-isoxazol-5-yl)-7-trifluoromethyl-5-(2- |
| | trifluoromethyl-phenyl)-1H-benzimidazole hydrochloride |
| | ¹H-NMR (400 MHz, CD₃OD) δ: 7.92 (s, 1H), 7.85 (d, J = 7.8 Hz, |
| | 1H), 7.68-7.76 (m, 2H), 7.61-7.67 (m, 1H), 7.50 (d, J = 7.6 Hz, |
| | 1H), 7.44 (s, 1H), 1.43 (s, 9H) |
| | Mass Spectrum (LCMS, APCI pos.) Calculated For |
| | C₂₂H₁₇F₆N₃O: 454.1 (M + H), Measured: 454.1. |
| 143 | 2-(3-tert-Butyl-isoxazol-5-yl)-5-(2-trifluoromethoxy-phenyl)-7- |
| | trifluoromethyl-1H-benzimidazole sodium salt |
| | ¹H-NMR (400 MHz, CD₃OD) δ: 7.89 (s, 1H), 7.55-7.61 (m, 1H), |
| | 7.48 (s, 1H), 7.37-7.46 (m, 3H), 7.11 (s, 1H), 1.42 (s, 9H) |
| | Mass Spectrum (LCMS, ESI pos.) Calculated For C₂₂H₁₇F₆N₃O₂: |
| | 470.1 (M + H), Measured: 470.1. |
| 143 | 2-(3-tert-Butyl-isoxazol-5-yl)-5-(2-trifluoromethoxy-phenyl)-7- |
| | trifluoromethyl-1H-benzimidazole hydrochloride |
| | ¹H-NMR (400 MHz, CD₃OD) δ: 8.05 (s, 1H), 7.84 (s, 1H), 7.62 |
| | (d, J = 6.8 Hz, 1H), 7.44-7.58 (m, 3H), 7.43 (s, 1H), 1.42 (s, 9H) |
| | Mass Spectrum (LCMS, APCI pos.) Calculated For |
| | C₂₂H₁₇F₆N₃O₂: 470.1 (M + H), Measured: 470.1. |

### Example 23

### 3-tert-Butyl-5-[5-(2,6-difluoro-phenyl)-7-trifluoromethyl-1H-benzimidazol-2-yl]-1-methyl-1H-pyrazole-4-carbonitrile sodium salt (Compound 151)

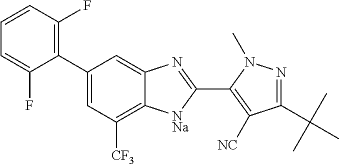

### STEP A. 3-tert-Butyl-5-[5-(5,5-dimethyl-[1,3,2]dioxaborinan-2-yl)-7-trifluoromethyl-1H-benzimidazol-2-yl]-1-methyl-1H-pyrazole-4-carbonitrile

To a mixture of bis(neopentylglycolato)diboron (152 mg, 0.600 mmol), 3-tert-butyl-5-(5-bromo-7-trifluoromethyl-1H-benzimidazol-2-yl)-1-methyl-1H-pyrazole-4-carbonitrile (as prepared in the Example 22, Step B, 213 mg, 0.500 mmol), and Pd(dppf)Cl₂ (36.6 mg, 0.0500 mmol) in 1,4-dioxane (6 mL) was added KOAc (98.2 mg, 1.00 mmol). The resulting mixture was stirred at 110° C. under microwave irradiation for 1.5 h and then cooled to room temperature. The resulting mixture was then treated with EtOAc (50 mL), then washed with H₂O and brine and was dried (Na₂SO₄). The solvent was removed under reduced pressure followed by flash chromatography of the residue on silica gel (0:1-1:3 EtOAc-hexanes) to yield a white solid. ¹H-NMR (400 MHz, CD₃OD) δ: 8.29 (br. s., 1H), 7.98 (s, 1H), 4.12 (s, 3H), 1.48 (s, 9H), 1.40 (s, 12H). Mass Spectrum (LCMS, ESI pos.) Calculated For C₂₃H₂₇BF₃N₅O₂: 474.2 (M+H), Measured: 474.3.

### STEP B. 3-tert-Butyl-5-[5-(2,6-difluoro-phenyl)-7-trifluoromethyl-1H-benzimidazol-2-yl]-1-methyl-1H-pyrazole-4-carbonitrile

To a mixture of 3-tert-butyl-5-[5-(5,5-dimethyl-[1,3,2]dioxaborinan-2-yl)-7-trifluoromethyl-1H-benzimidazol-2-yl]-1-methyl-1H-pyrazole-4-carbonitrile (as prepared in the previous step, 66.7 mg, 0.141 mmol), 2-bromo-1,3-difluoro-benzene (27.2 mg, 0.141 mmol) and Pd(PPh₃)₄ (16.3 mg, 0.0141 mmol) in 1,4-dioxane (2 mL) was added 2.0 M aqueous Na₂CO₃ (564 μL, 1.13 mmol). The resulting mixture was stirred at 110° C. under microwave irradiation for 1 h and then cooled to room temperature. The resulting mixture was treated with EtOAc (50 mL), then washed with H₂O and brine and then dried (Na₂SO₄). The solvent was removed under reduced pressure followed by flash chromatography of the residue on silica gel (1:19-2:3 EtOAc-hexanes) to yield as a white solid. ¹H-NMR (400 MHz, CD₃OD) δ: 8.03 (br. s., 1H), 7.71 (s, 1H), 7.45 (tt, J=8.4, 6.3 Hz, 1H), 7.07-7.20 (m, 2H), 4.15 (s, 3H), 1.48 (s, 9H). Mass Spectrum (LCMS, ESI pos.) Calculated For C₂₃H₁₈F₅N₅: 460.2 (M+H), Measured: 460.2.

### STEP C. 3-tert-Butyl-5-[5-(2,6-difluoro-phenyl)-7-trifluoromethyl-1H-benzimidazol-2-yl]-1-methyl-1H-pyrazole-4-carbonitrile sodium salt

To a solution of 3-tert-butyl-5-[5-(2,6-difluoro-phenyl)-7-trifluoromethyl-1H-benzimidazol-2-yl]-1-methyl-1H-pyrazole-4-carbonitrile (as prepared in the previous step, 28.4 mg, 0.0618 mmol) in 1:1 MeOH/THF (1 mL) was added a solution of 0.50 M NaOMe in MeOH (124 μL, 0.0618 mmol). After stirring at room temperature for 0.5 h, the mixture was concentrated in vacuo to yield the title compound as a white solid.

¹H-NMR (400 MHz, CD₃OD) δ: 7.90 (s, 1H), 7.42 (s, 1H), 7.32-7.41 (m, 1H), 7.09 (t, J=8.1 Hz, 2H), 4.05 (s, 3H), 1.48 (s, 9H). Mass Spectrum (LCMS, ESI pos.) Calculated For C₂₃H₁₈F₅N₅: 460.2 (M+H), Measured: 460.2.

Following the procedure described in Example 23, and selecting and substituting reagents, starting materials, and conditions as would be known to those skilled in the art, the following compounds of formula (I) of the present invention were prepared:

| ID | |
|---|---|
| No. | Name and Measured Physical Property |
| 151 | 3-tert-Butyl-5-[5-(2,6-difluoro-phenyl)-7-trifluoromethyl-1H- |
| | benzimidazol-2-yl]-1-methyl-1H-pyrazole-4-carbonitrile |
| | hydrochloride |
| | ¹H-NMR (400 MHz, CD₃OD) δ: 8.04 (s, 1H), 7.72 (s, 1H), 7.43- |
| | 7.52 (m, 1H), 7.11-7.20 (m, 2H), 4.15 (s, 3H), 1.49 (m, 9H) |
| | Mass Spectrum (LCMS, ESI pos.) Calculated For C₂₃H₁₈F₅N₅: |
| | 460.2 (M + H), Measured: 460.2. |
| 152 | 3-tert-Butyl-5-[5-(2-fluoro-6-trifluoromethyl-phenyl)-7- |
| | trifluoromethyl-1H-benzimidazol-2-yl]-1-methyl-1H-pyrazole-4- |
| | carbonitrile sodium salt |
| | ¹H-NMR (400 MHz, CD₃OD) δ: 7.73 (s, 1H), 7.63-7.69 (m, 1H), |
| | 7.56-7.63 (m, 1H), 7.45-7.51 (m, 1H), 7.24 (s, 1H), 4.03-4.07 |
| | (m, 3H), 1.48 (s, 9H) |
| | Mass Spectrum (LCMS, ESI pos.) Calculated For C₂₄H₁₈F₇N₅: |
| | 510.2 (M + H), Measured: 510.2. |
| 152 | 3-tert-Butyl-5-[5-(2-fluoro-6-trifluoromethyl-phenyl)-7- |
| | trifluoromethyl-1H-benzimidazol-2-yl]-1-methyl-1H-pyrazole-4- |
| | carbonitrile hydrochloride |
| | ¹H-NMR (400 MHz, CD₃OD) δ: 7.89 (s, 1H), 7.68-7.74 (m, 2H), |
| | 7.54-7.59 (m, 2H), 4.15 (s, 3H), 1.49 (s, 9H) |
| | Mass Spectrum (LCMS, ESI pos.) Calculated For C₂₄H₁₈F₇N₅: |
| | 510.2 (M + H), Measured: 510.2. |
| 140 | 2-(3-tert-Butyl-isoxazol-5-yl)-5-(2,6-difluoro-phenyl)-7- |
| | trifluoromethyl-1H-benzimidazole sodium salt |
| | ¹H-NMR (400 MHz, CD₃OD) δ: 7.87 (s, 1H), 7.31-7.44 (m, 2H), |
| | 7.02-7.14 (m, 3H), 1.42 (s, 9H) |
| | Mass Spectrum (LCMS, ESI pos.) Calculated For |
| | C₂₁H₁₆F₅N₃O: 422.1 (M + H), Measured: 422.1. |
| 140 | 2-(3-tert-Butyl-isoxazol-5-yl)-5-(2,6-difluoro-phenyl)-7- |
| | trifluoromethyl-1H-benzimidazole hydrochloride |
| | ¹H-NMR (400 MHz, CD₃OD) δ: 8.01 (s, 1H), 7.76 (s, 1H), 7.40- |
| | 7.52 (m, 1H), 7.38 (s, 1H), 7.13 (t, J = 8.0 Hz, 2H), 1.42 (s, 9H) |
| | Mass Spectrum (LCMS, APCI pos.) Calculated For |
| | C₂₁H₁₆F₅N₃O: 422.1 (M + H), Measured: 422.1. |
| 144 | 2-(3-tert-Butyl-isoxazol-5-yl)-5-(2-fluoro-6-trifluoromethyl- |
| | phenyl)-7-trifluoromethyl-1H-benzimidazole hydrochloride |
| | ¹H-NMR (400 MHz, CD₃OD) δ: 7.91 (s, 1H), 7.62-7.74 (m, 3H), |
| | 7.51-7.58 (m, 1H), 7.41 (s, 1H), 1.43 (s, 9H) |
| | Mass Spectrum (LCMS, APCI pos.) Calculated For |
| | C₂₂H₁₆F₇N₃O: 472.1 (M + H), Measured: 472.2. |
| 144 | 2-(3-tert-Butyl-isoxazol-5-yl)-5-(2-fluoro-6-trifluoromethyl- |
| | phenyl)-7-trifluoromethyl-1H-benzimidazole sodium salt |
| | ¹H-NMR (400 MHz, CD₃OD) δ: 7.70 (s, 1H), 7.63-7.68 (m, 1H), |
| | 7.54-7.62 (m, 1H), 7.44-7.51 (m, 1H), 7.23 (s, 1H), 7.11 (s, |
| | 1H), 1.37-1.45 (m, 9H) |
| | Mass Spectrum (LCMS, ESI pos.) Calculated For |
| | C₂₂H₁₆F₇N₃O: 472.1 (M + H), Measured: 472.1. |
| 145 | 2-(3-tert-Butyl-isoxazol-5-yl)-5-(2-fluoro-6-trifluoromethoxy- |
| | phenyl)-7-trifluoromethyl-1H-benzimidazole sodium salt |
| | ¹H-NMR (400 MHz, CD₃OD) δ: 7.80 (s, 1H), 7.42-7.52 (m, 1H), |
| | 7.32 (s, 1H), 7.22-7.31 (m, 2H), 7.10 (s, 1H), 1.42 (s, 9H) |
| | Mass Spectrum (LCMS, ESI pos.) Calculated For |
| | C₂₂H₁₆F₇N₃O₂: 488.1 (M + H), Measured: 488.1. |
| 145 | 2-(3-tert-Butyl-isoxazol-5-yl)-5-(2-fluoro-6-trifluoromethoxy- |
| | phenyl)-7-trifluoromethyl-1H-benzimidazole hydrochloride |
| | ¹H-NMR (400 MHz, CD₃OD) δ: 7.97 (s, 1H), 7.69 (s, 1H), 7.57 |
| | (td, J = 8.4, 6.2 Hz, 1H), 7.38 (s, 1H), 7.28-7.37 (m, 2H), 1.42 |
| | (s, 9H) |
| | Mass Spectrum (LCMS, APCI pos.) Calculated For |
| | C₂₂H₁₆F₇N₃O₂: 488.1 (M + H), Measured: 488.1. |

### Example 24

### 2-(2-tert-Butyl-thiazol-5-yl)-5-(2-trifluoromethyl-phenyl)-1H-benzimidazole hydrochloride (Compound #100)

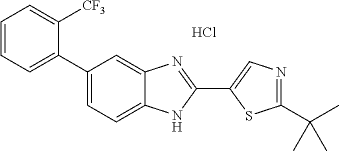

### STEP A. 2-tert-Butyl-thiazole-5-carboxylic acid (3-nitro-2′-trifluoromethyl-biphenyl-4-yl)-amide

To a mixture of 3-nitro-2′-trifluoromethyl-biphenyl-4-ylamine (108 mg, 0.383 mmol, as prepared in Example 6, Step A) and NaH (36.8 mg, 1.53 mmol, 95%) under Ar was added 3.0 mL of anhydrous THF, and the deep red solution stirred at room temperature for 1 h. Separately, 2-tert-butyl-thiazole-5-carboxylic acid (71.0 mg, 0.383 mmol, as prepared in Example X) in anhydrous DCM (3 mL) was treated with oxalyl chloride (48.6 μL, 0.575 mmol) and anhydrous DMF (10 μL). After stirring under a CaSO₄ drying tube for 1 h, the mixture was concentrated, dissolved in anhydrous THF-anhydrous DMF (1:1) (2 mL), and added dropwise over 1 min to the above-prepared solution. After stirring for 20 min, the mixture was quenched with saturated aqueous NH₄Cl (2 mL), concentrated to remove the THF, and treated with EtOAc (25 mL). The mixture was washed with water (3×10 mL) and brine (10 mL), dried (Na₂SO₄), and concentrated. The resulting residue was chromatographed on a 12-g silica gel column eluting with 0:1-1:3 EtOAc-hexanes to yield a yellow foam. ¹H-NMR (400 MHz, CDCl₃) δ: 11.26 (s, 1H), 8.94 (d, J=8.6 Hz, 1H), 8.32 (s, 1H), 8.27 (d, J=2.0 Hz, 1H), 7.80 (d, J=7.8 Hz, 1H), 7.68 (dd, J=8.8, 2.0 Hz, 1H), 7.63 (t, J=7.6 Hz, 1H), 7.55 (t, J=7.6 Hz, 1H), 7.35 (d, J=7.6 Hz, 1H), 1.50 (s, 9H). Mass Spectrum (LCMS, ESI pos.) Calculated for C₂₁H₁₈F₃N₃O₃S: 450.1 (M+H), Measured: 450.1.

### STEP B. 2-(2-tert-Butyl-thiazol-5-yl)-5-(2-trifluoromethyl-phenyl)-1H-benzimidazole

A solution of 2-tert-butyl-thiazole-5-carboxylic acid (3-nitro-2′-trifluoromethyl-biphenyl-4-yl)-amide (157 mg, 0.349 mmol, as prepared in the previous step) in HOAc-EtOH (1:1) (4 mL) under Ar was treated with iron powder (<10 μm, 97.7 mg, 1.75 mmol) and refluxed for 18 h. The mixture was concentrated to a dark solid, which was suspended in warm DCM (20 mL) and filtered (diatomaceous earth), washing the filter cake with DCM (3×10 mL). The filtrate was concentrated and chromatographed on a 12-g silica gel column eluting with a gradient of 1:9-3:1 EtOAc-hexanes to yield the title compound as a white crystalline solid.

¹H-NMR (400 MHz, CD₃OD) δ: 8.30 (s, 1H), 7.80 (d, J=7.8 Hz, 1H), 7.40-7.72 (m, 5H), 7.24 (m, 1H), 1.52 (s, 9H). Mass Spectrum (LCMS, ESI pos.) Calculated for C₂₁H₁₈F₃N₃S: 402.1 (M+H), Measured: 402.2.

### Example 25

### 2-(5-tert-Butyl-2-methyl-2H-[1,2,4]triazol-3-yl)-7-trifluoromethyl-5-(2-trifluoromethyl-phenyl)-1H-benzimidazole sodium salt (Compound #192)

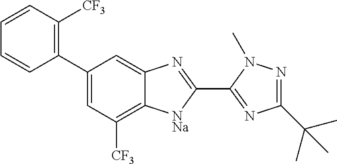

### STEP A. 2,2-Dimethylpropanimidic acid 2-methylhydrazide hydrochloride

2,2-Dimethylpropionimidic acid methyl ester hydrochloride (3.02 g, 19.9 mmol) in anhydrous MeOH (30 mL) was treated with methylhydrazine (1.15 mL, 21.9 mmol), stirred at room temperature for 8 h, and concentrated to a beige solid. The solid was dissolved in a minimum of hot MeOH, triturated with Et₂O to obtain a precipitate, and stirred vigorously overnight to obtain a fine suspension. The solid was filtered, washed with Et₂O, and dried under vacuum to yield a white powder. ¹H-NMR (400 MHz, CDCl₃) δ: 11.13 (br. s., 1H), 9.10 (br. s., 1H), 7.52 (br. s., 1H), 4.51 (br. s., 1H), 2.73 (s, 3H), 1.46 (s, 9H). Mass Spectrum (LCMS, ESI pos.) Calculated for C₆H₁₅N₃: 130.1 (M+H), Measured: 130.1.

### STEP B. 5-tert-Butyl-2-methyl-2H-[1,2,4]-triazole-3-carboxylic acid ethyl ester

A stirred suspension of 2,2-dimethylpropanimidic acid 2-methylhydrazide hydrochloride (802 mg, 484 mmol, as prepared in the previous step) in anhydrous toluene (30 mL) was treated with ethyl chlorooxoacetate (0.541 mL, 484 mmol), heated at reflux for 1 h, and concentrated to yield a colorless oily semisolid. Chromatography on an 80-g pre-packed silica gel column with 0:1-1:9 EtOAc-DCM yielded a colorless oil. ¹H-NMR (400 MHz, CDCl₃) δ: 4.48 (q, J=7.1 Hz, 2H), 4.17 (s, 3H), 1.44 (t, J=7.1 Hz, 3H), 1.39 (s, 9H). Mass Spectrum (LCMS, ESI pos.) Calculated for C₁₀H₁₇N₃O₂: 212.1 (M+H), Measured: 212.2.

### STEP C. 5-tert-Butyl-2-methyl-2H-[1,2,4]-triazole-3-carboxylic acid (4-bromo-2-nitro-6-trifluoromethyl-phenyl)-amide

A solution of 5-tert-butyl-2-methyl-2H-[1,2,4]triazole-3-carboxylic acid ethyl ester (300 mg, 1.42 mmol, as prepared in the previous step) in THF (6 mL) and MeOH (3 mL) was treated with 1.00 M aqueous NaOH (1.49 mL, 1.49 mmol) and stirred at room temperature for 1 h. Concentration to dryness yielded 5-tert-butyl-2-methyl-2H-[1,2,4]triazole-3-carboxylic acid sodium salt as a white solid that was suspended in anhydrous DCM (4 mL) under a CaSO₄ drying tube, treated with oxalyl chloride (0.184 mL, 2.17 mmol) followed by anhydrous DMF (2.2 μL (0.021 mmol), and stirred for 1 h at room temperature. Concentration in vacuo (normal rotovap vacuum only, room temperature) yielded a residue (the corresponding acid chloride and NaCl), which was used immediately in the following step.

At the same time, a mixture of 4-bromo-2-nitro-6-trifluoromethyl-phenylamine (386 mg, 1.86 mmol) and NaH (107 mg, 4.47 mmol) under Ar was treated with anhydrous THF (4 mL), and the dark purple solution was stirred at room temperature for 1 h. The residue prepared above, as a suspension in anhydrous THF (5 mL), was added to the prepared dark purple solution and the resulting mixture was stirred at room temperature for 16 h. The mixture was quenched with saturated aqueous NH₄Cl (3 mL), brine (20 mL) was added, and the mixture was extracted with EtOAc (3×20 mL). The combined organics were washed with brine (25 mL), dried (Na₂SO₄), and concentrated in vacuo to yield a brown resin. Chromatography on an 80-g pre-packed silica gel column with 0:100-30:70 EtOAc-hexanes yielded a pale yellow resin. ¹H-NMR (400 MHz, CDCl₃) δ: 9.60 (br. s., 1H), 8.31 (d, J=2.3 Hz, 1H), 8.09 (d, J=2.3 Hz, 1H), 4.17 (s, 3H), 1.38 (s, 9H). Mass Spectrum (LCMS, ESI pos.) Calculated for C₁₅H₁₅BrF₃N₅O₃: 450.0/452.0 (M+H), Measured: 450.1/452.0.

### STEP D. 5-Bromo-2-(5-tert-butyl-2-methyl-2H-[1,2,4]-triazol-3-yl)-7-trifluoromethyl-1H-benzimidazole

5-tert-Butyl-2-methyl-2H-[1,2,4]triazole-3-carboxylic acid (4-bromo-2-nitro-6-trifluoromethyl-phenyl)-amide (500 mg, 1.11 mmol, as prepared in the previous step) and iron powder (<10 um, 310 mg, 5.55 mmol) in acetic acid (8 mL) in a sealed vial were heated at 90° C. for 1.5 h. The mixture was filtered (diatomaceous earth), the filter cake was washed with DCM, and the filtrate was concentrated in vacuo to yield a brown solid. Extraction of the solid with DCM (4×15 mL) and filtration (diatomaceous earth) and concentration of the combined extracts yielded a brown solid. Chromatography on a 40-g pre-packed silica gel column with 0:100-20:80 EtOAc-DCM yielded a white solid. ¹H-NMR (400 MHz, CD₃OD) δ: 8.06 (s., 1H), 7.71 (s, 1H), 4.39 (s, 3H), 1.42 (s, 9H). Mass Spectrum (LCMS, ESI pos.) Calculated for C₁₅H₁₅BrF₃N₅: 402.1/404.1 (M+H), Measured: 402.2/404.1.

### STEP E. 2-(5-tert-Butyl-2-methyl-2H-[1,2,4]-triazol-3-yl)-7-trifluoromethyl-5-(2-trifluoromethyl-phenyl)-1H-benzimidazole sodium salt

To a mixture of 5-bromo-2-(5-tert-butyl-2-methyl-2H-[1,2,4]triazol-3-yl)-7-trifluoromethyl-1H-benzimidazole (80.0 mg, 0.199 mmol, as prepared in the previous step), PdCl₂(dppf).DCM (7.28 mg, 0.00995 mmol), and 2-trifluoromethylphenylboronic acid under Ar was added degassed (Ar) DME (3 mL) and degassed 2.00 M aqueous Na₂CO₃ (0.497 mL, 0.994 mmol). The mixture was heated with stirring at 85° C. for 18 h. After concentration in vacuo, the resulting residue was chromatographed on a 12-g pre-packed silica gel column eluting with 0:100-25:75 EtOAc-DCM to yield a colorless resin. This material in MeOH (3 mL) was treated with 1.0M NaOH (174 μL, 0.174 mmol) and concentrated to yield a semi-solid which, after trituration with Et₂O-hexanes and concentration, yielded the title compound as a white solid.

¹H-NMR (400 MHz, CD₃OD) δ: 7.80 (d, J=7.8 Hz, 1H), 7.76 (s, 1H), 7.63-7.69 (m, 1H), 7.52-7.58 (m, 1H), 7.47 (d, J=7.6 Hz, 1H), 7.31 (s, 1H), 4.16 (s, 3H), 1.42 (s, 9H). Mass Spectrum (LCMS, ESI pos.) Calculated for C₂₂H₁₉F₆N₅: 468.2 (M+H), Measured: 468.2.

### Example 26

### 2-(5-tert-Butyl-[1,3,4]oxadiazol-2-yl)-7-trifluoromethyl-5-(2-trifluoromethyl-phenyl)-1H-benzimidazole sodium salt (Compound #190)

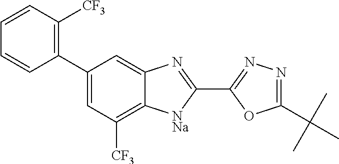

### STEP A. 5-tert-Butyl-[1,3,4]oxadiazole-2-carboxylic acid (4-bromo-2-nitro-6-trifluoromethyl-phenyl)-amide

A solution of 5-tert-butyl-[1,3,4]oxadiazole-2-carboxylic acid ethyl ester (500 mg, 2.52 mmol, as prepared according to the procedure described on page 7 in NEWTON, T. W., European Patent Application EP 0726263 A2, Published Aug. 14, 1996) in THF (10 mL) and MeOH (5 mL) was treated with 1.00 M aqueous NaOH (2.65 mL, 2.65 mmol) and stirred at room temperature for 1 h. Concentration to dryness yielded 5-tert-butyl-2-methyl-2H-[1,2,4]triazole-3-carboxylic acid sodium salt as a white solid that was suspended in anhydrous DCM (8 mL) under a CaSO₄ drying tube, treated with oxalyl chloride (0.319 mL, 3.77 mmol) followed by anhydrous DMF (4.0 μL, 0.052 mmol), and stirred for 1 h at room temperature. Concentration in vacuo (normal rotovap vacuum only, room temperature) yielded as residue (a mixture of the corresponding acid chloride and NaCl) that was used immediately in the following step.

At the same time, a mixture of 4-bromo-2-nitro-6-trifluoromethyl-phenylamine (751 mg, 2.64 mmol) and dry NaH (199 mg, 8.29 mmol) under Ar was treated with anhydrous THF (10 mL), and the dark purple solution was stirred at room temperature for 1 h. The residue prepared above, as a suspension in anhydrous THF (8 mL), was added to the dark purple solution and the resulting mixture was stirred at room temperature for 16 h. The mixture was quenched with 3 mL saturated aqueous NH₄Cl, 20 mL brine was added, and the mixture was extracted with EtOAc (3×25 mL). The combined organic extracts were washed with brine (25 mL), dried (Na₂SO₄), and concentrated in vacuo to yield a brown resin. Chromatography on an 80-g pre-packed silica gel column with 0:100-30:70 EtOAc:hexanes yielded a pale yellow resin. ¹H-NMR (400 MHz, CDCl₃) δ: 8.93 (br. s., 1H), 8.35 (d, J=2.0 Hz, 1H), 8.13 (d, J=2.0 Hz, 1H), 1.50 (s, 9H). Mass Spectrum (LCMS, ESI pos.) Calculated for C₁₄H₁₂BrF₃N₄O₄: 437.0/439.0 (M+H), Measured: 437.1/439.1.

### STEP B. 5-Bromo-2-(5-tert-butyl-[1,3,4]oxadiazol-2-yl)-7-trifluoromethyl-1H-benzimidazole

5-tert-Butyl-[1,3,4]oxadiazole-2-carboxylic acid (4-bromo-2-nitro-6-trifluoromethyl-phenyl)-amide (215 mg, 0.492 mmol, as prepared in the previous step) and iron powder (137 mg, 2.50 mmol, <10 um) in AcOH (5 mL) in a sealed vial were heated at 90° C. for 2 h. The mixture was filtered (diatomaceous earth), the filter cake was washed with DCM, and the filtrate was concentrated in vacuo to yield a brown solid. Chromatography on a 40-g pre-packed silica gel column with 0:100-30:70 EtOAc-DCM yielded a white solid. ¹H-NMR (400 MHz, CD₃OD/CDCl₃ (1:1)) δ: 8.09 (s, 1H), 7.77 (s, 1H), 1.56 (s, 9H). Mass Spectrum (LCMS, ESI pos.) Calculated for C₁₅H₁₅BrF₃N₅: 389.0/391.0 (M+H), Measured: 389.1/391.1.

### STEP C. 2-(5-tert-Butyl-[1,3,4]oxadiazol-2-yl)-7-trifluoromethyl-5-(2-trifluoromethyl-phenyl)-1H-benzimidazole sodium salt

To a mixture of 5-tert-butyl-[1,3,4]oxadiazole-2-carboxylic acid (4-bromo-2-nitro-6-trifluoromethyl-phenyl)-amide (12.6 mg 0.0.324 mmol, as prepared in the previous step), PdCl₂(dppf).DCM (2.37 mg, 0.00324 mmol), and 2-trifluoromethylphenylboronic acid (12.3 mg, 0.0648 mmol) under Ar was added degassed (Ar) DME (2 mL) and degassed 2.00 M aqueous Na₂CO₃ (0.0809 mL, 0.162 mmol). The mixture was capped and heated in a microwave reactor with stirring at 115° C. for 2 h. After concentration in vacuo, the resulting residue was chromatographed on a 12-g pre-packed silica gel column eluting with 0:100-12:88 EtOAc-DCM to yield a white solid. The white solid, combined with additional material prepared from a second batch (14.0 mg, 0.0308 mmol), in MeOH (1 mL) was treated with 1.0 M NaOH (30.8 μL, 0.0308 mmol) and concentrated to yield the title compound as a white solid.

¹H-NMR (400 MHz, CD₃OD/CDCl₃ (1:1)) δ: 7.81 (s, 1H), 7.77 (d, J=7.8 Hz, 1H), 7.59-7.64 (m, 1H), 7.48-7.54 (m, 1H), 7.46 (d, J=7.6 Hz, 1H), 7.36 (s, 1H), 1.55 (s, 9H). Mass Spectrum (LCMS, ESI pos.) Calculated for C₂₁H₁₆F₆N₄O: 455.1 (M+H), Measured: 455.2.

### Example 27

### 2-(2-tert-Butyl-5-methyl-oxazol-4-yl)-7-trifluoromethyl-5-(2-trifluoromethyl-phenyl)-1H-benzimidazole hydrochloride (Compound #153)

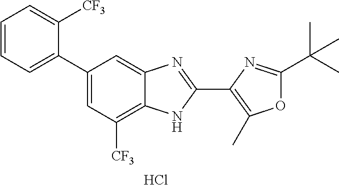

### STEP A. 2-tert-Butyl-5-methyl-oxazole-4-carboxylic acid (2-amino-5-bromo-3-trifluoromethyl-phenyl)-amide

A solution of 5-bromo-3-trifluoromethyl-benzene-1,2-diamine (0.255 g, 1.00 mmol), DIPEA (0.52 mL, 3.0 mmol), HBTU (0.38 g, 1.0 mmol), and 2-tert-butyl-5-methyl-oxazole-4-carboxylic acid (0.18 g, 1.0 mmol, as prepared in Example Z) in DMF (2 mL) was stirred at 35° C. for 18 h. The solution was cooled to room temperature, poured into H₂O, and extracted with EtOAc. The organic layer was separated, washed with H₂O (25 mL) and dried over Na₂SO₄. The solution was concentrated to yield a residue, which was used directly in the next step without further purification.

### STEP B. 5-Bromo-2-(2-tert-butyl-5-methyl-oxazol-4-yl)-7-trifluoromethyl-1H-benzimidazole

A solution of 2-tert-butyl-5-methyl-oxazole-4-carboxylic acid (2-amino-5-bromo-3-trifluoromethyl-phenyl)-amide (assumed 1 mmol, as prepared in the previous step) in AcOH (3 mL) was stirred at 90° C. for 6 h. The solution was cooled to room temperature, and toluene was added and concentrated to azeotrope off most of the AcOH. The residue was taken up in EtOAc and applied to a 2000-micron SiO₂ prep TLC plate, which was developed with EtOAc-hexanes (1:9). The desired band was isolated and extracted with EtOAc. The EtOAc was filtered, and the filtrate was concentrated in vacuo to yield a residue. Mass Spectrum (LCMS, ESI pos.) Calculated For C₁₆H₁₅BrF₃N₃O: 402.2 (M+H), Measured: 402.1.

### STEP C. 2-(2-tert-Butyl-5-methyl-oxazol-4-yl)-7-trifluoromethyl-5-(2-trifluoromethyl-phenyl)-1H-benzimidazole hydrochloride

A solution of 5-bromo-2-(2-tert-butyl-5-methyl-oxazol-4-yl)-7-trifluoromethyl-1H-benzimidazole (0.075 g, 0.19 mmol, as prepared in the previous step), 2-trifluoromethylphenylboronic acid (0.071 g, 0.37 mmol), and PdCl₂(dppf) (0.031 g, 0.037 mmol) in DME-2M aqueous Na₂CO₃ (2:1, 2.2 mL) was stirred at 90° C. for 3 h. The solution was cooled to room temperature, and the upper layer was applied to two 2000-micron SiO₂ prep TLC plates, which were developed with EtOAc-hexanes (1:9) to yield 2-(2-tert-butyl-5-methyl-oxazol-4-yl)-7-trifluoromethyl-5-(2-trifluoromethyl-phenyl)-1H-benzimidazole. 2-(2-tert-butyl-5-methyl-oxazol-4-yl)-7-trifluoromethyl-5-(2-trifluoromethyl-phenyl)-1H-benzimidazole (0.036 g, 0.077 mmol) was converted to the hydrochloride salt using 2 M HCl in diethyl ether (0.041 mL, 0.083 mmol). The mixture was concentrated in vacuo to yield the title compound.

¹H-NMR (400 MHz, CD₃OD) δ: 7.92 (s, 1H), 7.87 (d, J=7.8 Hz, 1H), 7.72-7.77 (m, 1H), 7.71 (s, 1H), 7.63-7.69 (m, 1H), 7.51 (d, J=7.6 Hz, 1H), 2.78 (s, 3H), 1.48 (s, 9H). Mass Spectrum (LCMS, ESI pos.) Calculated For C₂₃H₁₉F₆N₃O: 468.1 (M+H), Measured: 468.2.

Following the procedure described in Example 27, and selecting and substituting reagents, starting materials, and conditions as would be known to those skilled in the art, the following compounds of formula (I) of the present invention were prepared:

| ID | |
|---|---|
| No. | Name and Measured Physical Property |
| 154 | 2-(2-tert-Butyl-5-methyl-oxazol-4-yl)-5-(2-fluoro-phenyl)-7- |
| | trifluoromethyl-1H-benzimidazole hydrochloride |
| | ¹H-NMR (400 MHz, CD₃OD) δ: 8.14 (s, 1H), 7.96 (s, 1H), 7.63 |
| | (td, J = 7.8, 1.8 Hz, 1H), 7.44-7.53 (m, 1H), 7.33-7.39 (m, 1H), |
| | 7.30 (ddd, J = 11.1, 8.1, 1.3 Hz, 1H), 2.78 (s, 3H), 1.48 (s, 9H) |
| | Mass Spectrum (LCMS, ESI pos.) Calculated For C₂₂H₁₉F₄N₃O: |
| | 418.1 (M + H), Measured: 418.2. |
| 155 | 2-(2-tert-Butyl-5-methyl-oxazol-4-yl)-5-(2-chloro-phenyl)-7- |
| | trifluoromethyl-1H-benzimidazole hydrochloride |
| | ¹H-NMR (400 MHz, CD₃OD) δ: 8.03 (dd, J = 1.4, 0.6 Hz, 1H), |
| | 7.85 (dd, J = 1.5, 0.8 Hz, 1H), 7.58-7.61 (m, 1H), 7.49-7.53 |
| | (m, 1H), 7.44-7.49 (m, 2H), 2.78 (s, 3H), 1.48 (s, 9H) |
| | Mass Spectrum (LCMS, ESI pos.) Calculated For |
| | C₂₂H₁₉ClF₃N₃O: 434.1 (M + H), Measured: 434.2. |
| 156 | 2-(2-tert-Butyl-5-methyl-oxazol-4-yl)-5-(2-trifluoromethoxy- |
| | phenyl)-7-trifluoromethyl-1H-benzimidazole hydrochloride |
| | ¹H-NMR (400 MHz, CD₃OD) δ: 8.06 (s, 1H), 7.87 (s, 1H), 7.47- |
| | 7.67 (m, 4H), 2.78 (s, 3H), 1.48 (s, 9H) |
| | Mass Spectrum (LCMS, ESI pos.) Calculated For C₂₃H₁₉F₆N₃O₂: |
| | 484.1 (M + H), Measured: 484.2. |
| 157 | 2-(2-tert-Butyl-5-methyl-oxazol-4-yl)-5-phenyl-7-trifluoromethyl- |
| | 1H-benzimidazole hydrochloride |
| | ¹H-NMR (400 MHz, CD₃OD) δ: 8.17 (s, 1H), 8.03 (s, 1H), 7.71- |
| | 7.77 (m, 2H), 7.50-7.57 (m, 2H), 7.41-7.49 (m, 1H), 2.78 (s, |
| | 3H), 1.48 (s, 9H) |
| | Mass Spectrum (LCMS, ESI pos.) Calculated For C₂₂H₂₀F₃N₃O: |
| | 400.2 (M + H), Measured: 400.2. |
| 158 | 2-(2-tert-Butyl-5-methyl-oxazol-4-yl)-5-(2-fluoro-6-trifluoromethyl- |
| | phenyl)-7-trifluoromethyl-1H-benzimidazole hydrochloride |
| | ¹H-NMR (400 MHz, CD₃OD) δ: 7.92 (s, 1H), 7.66-7.76 (m, 3H), |
| | 7.54-7.61 (m, 1H), 2.78 (s, 3H), 1.48 (s, 9H) |
| | Mass Spectrum (LCMS, ESI pos.) Calculated For C₂₃H₁₈F₇N₃O: |
| | 486.1 (M + H), Measured: 486.2 |
| 159 | 2-(2-tert-Butyl-5-methyl-oxazol-4-yl)-7-chloro-5-(2- |
| | trifluoromethoxy-phenyl)-1H-benzimidazole hydrochloride |
| | ¹H-NMR (400 MHz, CD₃OD) δ: 7.81 (d, J = 1.3 Hz, 1H), 7.71 (d, |
| | J = 1.3 Hz, 1H), 7.46-7.64 (m, 4H), 2.79 (s, 3H), 1.48 (s, 9H) |
| | Mass Spectrum (LCMS, ESI pos.) Calculated For |
| | C₂₂H₁₉ClF₃N₃O₂: 450.1 (M + H), Measured: 450.1. |
| 161 | 2-(2-tert-Butyl-5-methyl-oxazol-4-yl)-7-chloro-5-(2-fluoro-phenyl)- |
| | 1H-benzimidazole hydrochloride |
| | ¹H-NMR (400 MHz, CD₃OD) δ: 7.87 (t, J = 1.3 Hz, 1H), 7.79 (t, J = |
| | 1.3 Hz, 1H), 7.60 (td, J = 7.8, 1.8 Hz, 1H), 7.44-7.51 (m, J = |
| | 10.3, 5.2, 5.2, 1.8 Hz, 1H), 7.24-7.37 (m, 2H), 2.79 (s, 3H), 1.48 |
| | (s, 9H) |
| | Mass Spectrum (LCMS, ESI pos.) Calculated For C₂₁H₁₉ClFN₃O: |
| | 384.1 (M + H), Measured: 384.2. |
| 163 | 2-(2-tert-Butyl-5-methyl-oxazol-4-yl)-7-chloro-5-(2-chloro-phenyl)- |
| | 1H-benzimidazole hydrochloride |
| | ¹H-NMR (400 MHz, CD₃OD) δ: 7.73 (d, J = 1.3 Hz, 1H), 7.64 (d, |
| | J = 1.3 Hz, 1H), 7.55-7.60 (m, 1H), 7.41-7.51 (m, 3H), 2.78 (s, |
| | 3H), 1.48 (s, 9H) |
| | Mass Spectrum (LCMS, ESI pos.) Calculated For C₂₁H₁₉Cl₂N₃O: |
| | 400.1 (M + H), Measured: 400.1. |
| 165 | 2-(2-tert-Butyl-5-methyl-oxazol-4-yl)-7-chloro-5-(2-trifluoromethyl- |
| | phenyl)-1H-benzimidazole hydrochloride |
| | ¹H-NMR (400 MHz, CD₃OD) δ: 7.86 (d, J = 7.8 Hz, 1H), 7.70- |
| | 7.76 (m, 1H), 7.62-7.68 (m, 2H), 7.55 (s, 1H), 7.49 (d, J = 7.3 |
| | Hz, 1H), 2.79 (s, 3H), 1.48 (s, 9H)Mass Spectrum (LCMS, ESI |
| | pos.) Calculated For C₂₂H₁₉ClF₃N₃O: 434.1 (M + H), Measured: |
| | 434.2. |

### Example 28

### 2-(2-tert-Butyl-4-methyl-oxazol-5-yl)-5-(2-fluoro-phenyl)-7-trifluoromethyl-1H-benzimidazole hydrochloride (Compound #175)

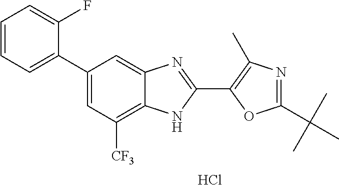

### STEP A. 2-tert-Butyl-4-methyl-oxazole-5-carboxylic acid (5-bromo-2-nitro-3-trifluoromethyl-phenyl)-amide

A solution of 2-tert-butyl-4-methyl-oxazole-5-carboxylic acid (0.72 g, 3.9 mmol, as prepared in Example AA), 5-bromo-2-nitro-3-trifluoromethyl-phenylamine (1.0 g, 3.9 mmol), DIEA (2.0 mL, 7.8 mmol) and HBTU (1.5 g, 3.9 mmol) in DMF (10 mL) was stirred at 35° C. for 18 h. The solution was cooled to room temperature and poured into EtOAc-H₂O (1:1). The organic layer was separated, washed with H₂O and brine, and dried over Na₂SO₄. The residue was taken up in EtOAc, applied to a silica gel plug (50 g), and eluted with EtOAc-hexanes (2:8) to yield a residue, which was used directly in the next step.

### STEP B. 5-Bromo-2-(2-tert-butyl-4-methyl-oxazol-5-yl)-7-trifluoromethyl-1H-benzimidazole

A solution of 2-tert-butyl-4-methyl-oxazole-5-carboxylic acid (5-bromo-2-nitro-3-trifluoromethyl-phenyl)-amide (1.6 g, 3.9 mmol, as prepared in the previous step) and iron powder (0.54 g, 9.6 mmol) in AcOH (10.0 mL) was stirred at 90° C. After 1 h, the resulting mixture was cooled to room temperature and concentrated. The residue was partitioned between EtOAc-saturated aqueous NaHCO₃ (25-25 mL), the layers were separated, and the organic layer was washed with saturated aqueous NaHCO₃ (20 mL), H₂O (20 mL), brine (20 mL) and dried over Na₂SO₄. The resulting residue was chromatographed on silica gel to yield a residue. ¹H-NMR (400 MHz, CDCl₃) δ: 8.08 (br.s., 1H), 7.62 (br.s., 1H), 2.63 (s, 3H), 1.44 (s, 9H).

### STEP C. 2-(2-tert-Butyl-4-methyl-oxazol-5-yl)-5-(2-fluoro-phenyl)-7-trifluoromethyl-1H-benzimidazole hydrochloride

A solution of 2-tert-butyl-4-methyl-oxazole-5-carboxylic acid (5-bromo-2-nitro-3-trifluoromethyl-phenyl)-amide (0.076 g, 0.19 mmol, as prepared in the previous step), 2-fluorophenylboronic acid (0.052 g, 0.38 mmol), PdCl₂(dppf) (0.030 g, 0.037 mmol) in 2 M aqueous Na₂CO₃/DME (1:2, 2.2 mL) was stirred at 90° C. for 18 h. The solution was cooled to room temperature. The upper layer was applied to five 2000-micron prep TLC plates, which were developed using EtOAc-hexanes (2:8) to yield 2-(2-tert-butyl-4-methyl-oxazol-5-yl)-5-(2-fluoro-phenyl)-7-trifluoromethyl-1H-benzimidazole. The HCl salt was prepared using 2 M HCl in ethyl ether to yield the title compound.

¹H-NMR (400 MHz, CDCl₃) δ: 8.12 (s, 1H), 7.93 (s, 1H), 7.63 (td, J=7.8, 1.6 Hz, 1H), 7.45-7.50 (m, 1H), 7.36 (td, J=7.6, 1.3 Hz, 1H), 7.29 (ddd, J=11.1, 8.3, 1.1 Hz, 1H), 2.62 (s, 3H), 1.51 (s, 9H). Mass Spectrum (LCMS, ESI pos.) Calculated For C₂₂H₁₉F₄N₃O: 418.1 (M+H), Measured: 418.2.

Following the procedure described in Example 28, and selecting and substituting reagents, starting materials and conditions as would be known to those skilled in the art, the following compounds of formula (I) of the present invention were prepared:

| ID | |
|---|---|
| No. | Name and Measured Physical Property |
| 179 | 2-(2-tert-Butyl-4-methyl-oxazol-5-yl)-5-(2-chloro-phenyl)-7- |
| | trifluoromethyl-1H-benzimidazole hydrochloride |
| | ¹H-NMR (400 MHz, CD₃OD) δ: 7.99 (dd, J = 1.5, 0.8 Hz, |
| | 1H), 7.79 (dd, J = 1.5, 0.8 Hz, 1H), 7.57-7.61 (m, 1H), 7.41- |
| | 7.53 (m, 3H), 2.62 (s, 3H), 1.51 (s, 9H) |
| | Mass Spectrum (LCMS, ESI pos.) Calculated For |
| | C₂₂H₁₉ClF₃N₃O: 434.1 (M + H), Measured: 434.2. |
| 176 | 2-(2-tert-Butyl-4-methyl-oxazol-5-yl)-5-(2-trifluoromethoxy- |
| | phenyl)-7-trifluoromethyl-1H-benzimidazole hydrochloride |
| | ¹H-NMR (400 MHz, CD₃OD) δ: 8.00 (s, 1H), 7.78 (s, 1H), |
| | 7.61-7.65 (m, 1H), 7.46-7.59 (m, 3H), 2.62 (s, 3H), 1.51 |
| | (s, 9H) |
| | Mass Spectrum (LCMS, ESI pos.) Calculated For |
| | C₂₃H₁₉F₆N₃O₂: 484.1 (M + H), Measured: 484.2. |
| 178 | 2-(2-tert-Butyl-4-methyl-oxazol-5-yl)-5-phenyl-7- |
| | trifluoromethyl-1H-benzimidazole hydrochloride |
| | ¹H-NMR (400 MHz, CD₃OD) δ: 8.15 (s, 1H), 8.01 (s, 1H), |
| | 7.70-7.77 (m, 2H), 7.50-7.57 (m, 2H), 7.41-7.48 (m, 1H), |
| | 2.62 (s, 3H), 1.51 (s, 9H) |
| | Mass Spectrum (LCMS, ESI pos.) Calculated For |
| | C₂₂H₂₀F₃N₃O: 400.2 (M + H), Measured: 400.1. |
| 177 | 2-(2-tert-Butyl-4-methyl-oxazol-5-yl)-7-trifluoromethyl-5-(2- |
| | trifluoromethyl-phenyl)-1H-benzimidazole hydrochloride |
| | ¹H-NMR (400 MHz, CD₃OD) δ: 7.83-7.90 (m, 2H), 7.70- |
| | 7.77 (m, 1H), 7.62-7.68 (m, 2H), 7.47-7.53 (m, 1H), 2.62 |
| | (s, 3H), 1.51 (s, 9H) |
| | Mass Spectrum (LCMS, ESI pos.) Calculated For |
| | C₂₃H₁₉F₆N₃O: 468.1 (M + H), Measured: 468.2. |
| 182 | 2-(2-tert-Butyl-4-methyl-oxazol-5-yl)-7-chloro-5-(2-fluoro- |
| | phenyl)-1H-benzimidazole hydrochloride |
| | ¹H-NMR (400 MHz, CD₃OD) δ: 7.89 (d, J = 1.3 Hz, 1H), 7.85- |
| | 7.88 (m, 1H), 7.68-7.74 (m, 2H), 7.49-7.56 (m, 2H), 7.41- |
| | 7.47 (m, 1H), 2.62 (s, 3H), 1.52 (s, 9H) |
| | Mass Spectrum (LCMS, ESI pos.) Calculated For |
| | C₂₁H₁₉ClFN₃O: 384.1 (M + H), Measured: 384.2. |
| 181 | 2-(2-tert-Butyl-4-methyl-oxazol-5-yl)-7-chloro-5-(2-chloro- |
| | phenyl)-1H-benzimidazole hydrochloride |
| | ¹H-NMR (400 MHz, CD₃OD) δ: 7.70 (d, J = 1.5 Hz, 1H), 7.59 |
| | (d, J = 1.5 Hz, 1H), 7.53-7.59 (m, 1H), 7.42-7.50 (m, 3H), |
| | 2.62 (s, 3H), 1.51 (s, 9H) |
| | Mass Spectrum (LCMS, ESI pos.) Calculated For |
| | C₂₁H₁₉Cl₂N₃O: 400.1 (M + H), Measured: 400.2. |
| 183 | 2-(2-tert-Butyl-4-methyl-oxazol-5-yl)-7-chloro-5-(2- |
| | trifluoromethoxy-phenyl)-1H-benzimidazole hydrochloride |
| | ¹H-NMR (400 MHz, CD₃OD) δ: 7.77 (d, J = 1.3 Hz, 1H), 7.66 |
| | (d, J = 1.3 Hz, 1H), 7.46-7.63 (m, 4H), 2.62 (s, 3H), 1.51 (s, |
| | 9H) |
| | Mass Spectrum (LCMS, ESI pos.) Calculated For |
| | C₂₂H₁₉ClF₃N₃O₂: 450.1 (M + H), Measured: 450.1. |
| 180 | 2-(2-tert-Butyl-4-methyl-oxazol-5-yl)-7-chloro-5-phenyl-1H- |
| | benzimidazole hydrochloride |
| | ¹H-NMR (400 MHz, CD₃OD) δ: 7.86 (t, J = 1.4 Hz, 1H), 7.78 |
| | (t, J = 1.4 Hz, 1H), 7.60 (td, J = 7.8, 1.8 Hz, 1H), 7.42-7.52 |
| | (m, 1H), 7.34 (td, J = 7.5, 1.1 Hz, 1H), 7.28 (ddd, J = 11.1, |
| | 8.3, 1.1 Hz, 1H), 2.62 (s, 3H), 1.52 (s, 9H) |
| | Mass Spectrum (LCMS, ESI pos.) Calculated For |
| | C₂₁H₂₀ClN₃O: 366.1 (M + H), Measured: 366.2. |
| 194 | 2-(4-tert-Butyl-5-methyl-oxazol-2-yl)-7-trifluoromethyl-5-(2- |
| | trifluoromethyl-phenyl)-1H-benzimidazole hydrochloride |
| | ¹H-NMR (400 MHz, CD₃OD) δ: 7.81 (d, J = 7.3 Hz, 1H), 7.79 |
| | (s, 1H), 7.68 (t, J = 8.1 Hz, 1H), 7.59 (t, J = 7.5 Hz, 1H), 7.53 |
| | (s, 1H), 7.45 (d, J = 7.8 Hz, 1H), 2.55 (s, 3H), 1.38 (s, 9H) |
| | Mass Spectrum (LCMS, ESI pos.) Calculated For |
| | C₂₃H₁₉F₆N₃O: 468.1 (M + H), Measured: 468.2. |
| 195 | 2-(4-tert-Butyl-5-methyl-oxazol-2-yl)-5-(2-trifluoromethoxy- |
| | phenyl)-7-trifluoromethyl-1H-benzimidazole hydrochloride |
| | ¹H-NMR (400 MHz, CD₃OD) δ: 7.93 (s, 1H), 7.69 (s, 1H), |
| | 7.55-7.59 (m, 1H), 7.46-7.51 (m, 2H), 7.40-7.46 (m, 1H), |
| | 2.55 (s, 3H), 1.38 (s, 9H) |
| | Mass Spectrum (LCMS, ESI pos.) Calculated For |
| | C₂₃H₁₉F₆N₃O₂: 484.4 (M + H), Measured: 484.2. |
| 196 | 2-(4-tert-Butyl-5-methyl-oxazol-2-yl)-5-(2-chloro-phenyl)-7- |
| | trifluoromethyl-1H-benzimidazole hydrochloride |
| | ¹H-NMR (400 MHz, CD₃OD) δ: 7.88 (s, 1H), 7.65 (s, 1H), |
| | 7.51-7.55 (m, 1H), 7.41-7.47 (m, 1H), 7.36-7.41 (m, 2H), |
| | 2.55 (s, 3H), 1.38 (s, 9H) |
| | Mass Spectrum (LCMS, ESI pos.) Calculated For |
| | C₂₂H₁₉ClF₃N₃O: 434.1 (M + H), Measured: 434.2. |

### Example 29

### 2-(4-tert-Butyl-5-methyl-oxazol-2-yl)-5-(2-fluoro-phenyl)-7-trifluoromethyl-1H-benzimidazole hydrochloride (Compound #193)

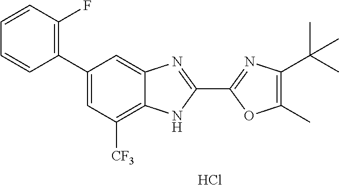

### STEP A. 4-Bromo-2,2-dimethyl-pentan-3-one

A mixture of 4,4-dimethyl-3-pentanone (1.0 g, 8.8 mmol) and NBS (1.6 g, 9.2 mmol) in CCl₄ (10 mL) was treated with NH₄OAc (0.067 g, 0.88 mmol) and the heterogeneous mixture was stirred at 80° C. under an argon atmosphere. After 1 h, the mixture was cooled to room temperature and treated with H₂O (10 mL). The layers were separated and the organic layer was washed with H₂O and brine, dried over Na₂SO₄, and concentrated in vacuo to yield a colorless oil. ¹H-NMR (400 MHz, CDCl₃) δ: 4.73 (q, J=6.8 Hz, 1H), 1.69 (d, J=6.8 Hz, 3H), 1.24 (s, 9H).

### STEP B. Oxalic acid ethyl ester 1,3,3-trimethyl-2-oxo-butyl ester

A mixture of 4-bromo-2,2-dimethyl-pentan-3-one (0.050 g, 0.26 mmol, as prepared in the previous step) and ethyl potassium oxalate (0.048 g, 0.31 mmol) in CH₃CN (1 mL) was stirred at 80° C. for 1 h. The solution was cooled to room temperature and partitioned between EtOAc-H₂O (25-25 mL). The layers were separated, and the organic layer was concentrated in vacuo to yield a residue. ¹H-NMR (400 MHz, CDCl₃) δ: 5.53 (q, J=6.8 Hz, 1H), 4.33 (q, J=7.3 Hz, 2H), 1.48 (d, J=6.8 Hz, 3H), 1.34 (t, J=7.1 Hz, 3H), 1.21 (s, 9H).

### STEP C. 4-tert-Butyl-5-methyl-oxazole-2-carboxylic acid ethyl ester

A mixture of oxalic acid ethyl ester 1,3,3-trimethyl-2-oxo-butyl ester (0.56 g, 0.24 mmol, as prepared in the previous step) and ammonium trifluoroacetate (0.32 g, 2.4 mmol) was stirred at 150° C. After 20 min the solution was cooled to room temperature. The solid residue was partitioned between EtOAc-H₂O (25-25 mL), and the organic layer was separated and concentrated in vacuo to yield a residue. ¹H-NMR (400 MHz, CDCl₃) δ: 4.43 (q, J=7.1 Hz, 2H), 2.47 (s, 3H), 1.39 (t, J=7.1 Hz, 3H), 1.33 (s, 9H). Mass Spectrum (LCMS, ESI pos.) Calculated For C₁₁H₁₇NO₃: 212.1 (M+H), Measured: 212.1.

### STEP D. 4-tert-Butyl-5-methyl-oxazole-2-carboxylic acid

A solution of 4-tert-butyl-5-methyl-oxazole-2-carboxylic acid ethyl ester (0.54 g, 2.5 mmol, as prepared in the previous step) in MeOH (5 mL) was treated with 1 N aqueous NaOH (2.8 mL, 2.8 mmol). The solution was stirred at room temperature for 18 h and neutralized to pH 7 using 1 N HCl. The solution was concentrated and then azeotroped with toluene in vacuo to remove residual water. The resulting residue was used directly in the next step without further purification.

### STEP E. 4-tert-Butyl-5-methyl-oxazole-2-carbonyl chloride

A solution of 4-tert-butyl-5-methyl-oxazole-2-carboxylic acid (0.45 g, 2.5 mmol, as prepared in the previous step) in DCM (20 mL) was treated with oxalyl chloride (0.85 mL, 10 mmol) and DMF (0.010 mL) at room temperature under Ar. After 1 h, the solution was concentrated to yield a residue, which was used directly in the next step without further purification.

### STEP F. 4-tert-Butyl-5-methyl-oxazole-2-carboxylic acid (4-bromo-2-nitro-6-trifluoromethyl-phenyl)-amide

A solution of 4-bromo-2-nitro-6-trifluoromethyl-phenylamine (0.070 g, 0.25 mmol) in THF (5 mL) was treated with NaH (0.030 g, 0.74 mmol, 60% in mineral oil) and stirred for 15 min. under an argon atmosphere. 4-tert-Butyl-5-methyl-oxazole-2-carbonyl chloride (assumed 0.5 mmol, as prepared in the previous step) was added as a solution in THF (2 mL). The solution was stirred at room temperature under an argon atmosphere for 4 h, and the resulting mixture solution was applied to nine 2000 micron SiO₂ prep TLC plates, which were developed using EtOAc-hexanes (1:9) to yield a residue. ¹H-NMR (400 MHz, CDCl₃) δ: 9.12 (s, 1H), 8.28-8.30 (d, J=2.0 Hz, 1H), 8.06-8.08 (d, J=2.0 Hz, 1H), 2.48 (s, 3H), 1.34 (s, 9H). Mass Spectrum (LCMS, ESI pos.) Calculated For C₁₆H₁₅BrF₃N₃O₄: 450.2, 452.2 (M+H), Measured: 450.2, 452.2.

### STEP G. 5-Bromo-2-(4-tert-butyl-5-methyl-oxazol-2-yl)-7-trifluoromethyl-1H-benzimidazole

A solution of 4-tert-butyl-5-methyl-oxazole-2-carboxylic acid (4-bromo-2-nitro-6-trifluoromethyl-phenyl)-amide (0.040 g, 0.090 mmol, as prepared in the previous step) in AcOH (2 mL) was treated with iron powder (0.040 g, 0.71 mmol). The resulting heterogeneous solution was stirred at 110° C. After 1 h the resulting mixture solution was cooled to room temperature and diluted with EtOAc. The organic layer was washed with saturated aqueous NaHCO₃ (2×10 mL), water (10 mL) and brine (10 mL), dried over Na₂SO₄, and concentrated in vacuo to yield a residue. Mass Spectrum (LCMS, ESI pos.) Calculated For C₁₆H₁₅BrF₃N₃O: 402.0, 404.0 (M+H), Measured: 402.2, 404.2.

### STEP H. 2-(4-tert-Butyl-5-methyl-oxazol-2-yl)-5-(2-fluoro-phenyl)-7-trifluoromethyl-1H-benzimidazole

A solution of 5-bromo-2-(4-tert-butyl-5-methyl-oxazol-2-yl)-7-trifluoromethyl-1H-benzimidazole (0.026 g, 0.065 mmol, as prepared in the previous step), 2-fluorophenylboronic acid (0.018 g, 0.13 mmol), PdCl₂(dppf) (0.011 g, 0.013 mmol) in DME (1 mL) and 2 M aqueous Na₂CO₃ (0.26 mL, 0.52 mmol) was stirred at 90° C. for 18 h. The resulting mixture was cooled room temperature. The two-phase system was diluted with EtOAc-H₂O (25-25 mL) and shaken gently. The upper layer was applied to six 2000-micron SiO₂ prep TLC plates, which were developed using EtOAc-hexanes (2:8) to yield a residue. Mass Spectrum (LCMS, ESI pos.) Calculated For C₂₂H₁₉F₄N₃O: 418.1 (M+H), Measured: 418.2.

### STEP I. 2-(4-tert-Butyl-5-methyl-oxazol-2-yl)-5-(2-fluoro-phenyl)-7-trifluoromethyl-1H-benzimidazole hydrochloride

A solution of 2-(4-tert-butyl-5-methyl-oxazol-2-yl)-5-(2-fluoro-phenyl)-7-trifluoromethyl-1H-benzimidazole (0.0057 g, 0.014 mmol, as prepared in the previous step) in diethyl ether (2 mL) was treated with 2 M HCl in diethyl ether (7.5 μL, 0.015 mmol). The resulting solution was stirred at room temperature for a few minutes and was concentrated in vacuo to yield the title compound.

¹H-NMR (400 MHz, CD₃OD) δ: 9.00 (s, 1H), 8.70 (s, 1H), 8.50-8.61 (m, 1H), 8.30-8.40 (m, 1H), 8.10-8.20 (m, 2H), 3.50 (s, 3H), 2.30 (s, 9H). Mass Spectrum (LCMS, ESI pos.) Calculated For C₂₂H₁₉F₄N₃O: 418.1 (M+H), Measured: 418.2.

Following the procedure described in Example 29, and selecting and substituting reagents, starting materials, and conditions as would be known to those skilled in the art, the following compounds of formula (I) of the present invention were prepared:

| ID | |
|---|---|
| No. | Name and Measured Physical Property |
| 194 | 2-(4-tert-Butyl-5-methyl-oxazol-2-yl)-7-trifluoromethyl-5-(2- |
| | trifluoromethyl-phenyl)-1H-benzimidazole hydrochloride |
| | ¹H-NMR (400 MHz, CD₃OD) δ: 7.81 (d, J = 7.3 Hz, 1H), 7.79 |
| | (s, 1H), 7.68 (t, J = 8.1 Hz, 1H), 7.59 (t, J = 7.5 Hz, 1H), 7.53 |
| | (s, 1H), 7.45 (d, J = 7.8 Hz, 1H), 2.55 (s, 3H), 1.38 (s, 9H) |
| | Mass Spectrum (LCMS, ESI pos.) Calculated For |
| | C₂₃H₁₉F₆N₃O: 468.1 (M + H), Measured: 468.2. |
| 195 | 2-(4-tert-Butyl-5-methyl-oxazol-2-yl)-5-(2-trifluoromethoxy- |
| | phenyl)-7-trifluoromethyl-1H-benzimidazole hydrochloride |
| | ¹H-NMR (400 MHz, CD₃OD) δ: 7.93 (s, 1H), 7.69 (s, 1H), |
| | 7.55-7.59 (m, 1H), 7.46-7.51 (m, 2H), 7.40-7.46 (m, 1H), |
| | 2.55 (s, 3H), 1.38 (s, 9H) |
| | Mass Spectrum (LCMS, ESI pos.) Calculated For |
| | C₂₃H₁₉F₆N₃O₂: 484.4 (M + H), Measured: 484.2. |
| 196 | 2-(4-tert-Butyl-5-methyl-oxazol-2-yl)-5-(2-chloro-phenyl)-7- |
| | trifluoromethyl-1H-benzimidazole hydrochloride |
| | ¹H-NMR (400 MHz, CD₃OD) δ: 7.88 (s, 1H), 7.65 (s, 1H), |
| | 7.51-7.55 (m, 1H), 7.41-7.47 (m, 1H), 7.36-7.41 (m, 2H), |
| | 2.55 (s, 3H), 1.38 (s, 9H) |
| | Mass Spectrum (LCMS, ESI pos.) Calculated For |
| | C₂₂H₁₉ClF₃N₃O: 434.1 (M + H), Measured: 434.2. |

### Biological Example 1

### In Vitro Canine TRPM8 Functional Assay

The functional activity of representative compounds of the formula (I) of the present invention was quantified by measuring changes in intracellular calcium concentration using a Ca²⁺-sensitive fluorescent dye. The changes in fluorescent signal were monitored by a fluorescence plate reader, either a FLIPR™ (Molecular Devices) or FDSS (Hamamatsu). Increases in intracellular Ca²⁺ concentration were readily detected upon activation with icilin. HEK293 cells stably expressing canine TRPM8 were routinely grown as monolayers in Dulbecco's minimum essential medium supplemented with 10% FBS, 2 mM L-glutamine, 100 units/mL penicillin, 100 ug/mL streptomycin and 400 μg/mL G418. Cells were maintained in 5% CO₂ at 37° C. At 24 hr prior to assay, cells were seeded in black wall, clear-base poly-D-lysine coated 384-well plates (BD Biosciences, NJ, USA) at a density of 5,000 cells per well in culture medium and grown overnight in 5% CO₂ at 37° C. On assay day, growth media was removed, and cells were loaded with Calcium 3 Dye (Molecular Devices) for 35 min at 37° C., under 5% CO₂ and then incubated for 25 min at room temperature and atmosphere. Subsequently, cells were tested for agonist-induced increases in intracellular Ca²⁺ levels using FLIPR™ or FDSS. Cells were treated with compounds of the formula (I) at varying concentrations and intracellular Ca²⁺ was measured for 5 min prior to the addition of icilin to each well to achieve a final concentration that produces an approximately 80% maximal response. EC₅₀ or IC₅₀ values for compounds of the present invention were determined from eight-point concentration-response studies and represent the concentration of compound required to elicit or inhibit 50% of the maximal response, respectively.

Maximal fluorescence intensity (FI) achieved upon addition of icilin was exported from the FLIPR™ or FDSS software and further analyzed using GraphPad Prism 3.02 (Graph Pad Software Inc., CA, U.S.A.). Basal FI was subtracted prior to normalizing data to percent of maximal response. Curves were generated using the average of quadruplicate wells for each data point using nonlinear regression of either sigmoidal dose response or sigmoidal dose response (variable slope). Finally, IC₅₀ values were calculated with the best-fit curve determined by GraphPad Prism

Representative compounds of the present invention were tested according to the procedure(s) as described in Biological Example 1, above, with results as listed in Table 2, below.

NOTE: Where a compound of the present invention was tested multiple times and/or prepared in multiple batches and/or forms, for example, as a free base and/or as different corresponding salt forms, the biological activity listed in Table 2 is listed as the individually measured values.

TABLE 2
in vitro TRP M8 Activity

| ID No. | IC₅₀ (nM) | % Inh (%) @0.2 μM |
|---|---|---|
| 1 | 111.0 | |
| 2 | 16.0 | |
| 3 | 22.0, 24.0, 29.0, 24.0 | |
| 5 | 22.5 | |
| 6 | 64.4 | |
| 7 | | 10 |
| 8 | 11.1, 8.0 | |
| 9 | 17.4, 12.0 | |
| 10 | 12.0, 15.0, 10.0 | |
| 12 | 13.0 | |
| 13 | 30.0, 13.0 | |
| 14 | 44.0 | |
| 15 | 70.0 | |
| 16 | 58.0 | |
| 17 | 52.0 | |
| 18 | 27.0 | |
| 19 | 18.0 | |
| 20 | 19.0 | |
| 21 | 29.0 | |
| 22 | 32.0 | |
| 23 | 30.0 | |
| 24 | | 63 |
| 25 | | 63 |
| 26 | 39.0 | |
| 27 | 46.0 | |
| 30 | 36.0 | |
| 31 | 84.0 | |
| 32 | 21.0 | |
| 33 | 145.0 | |
| 34 | 72.9 | |
| 37 | 55.9 | |
| 38 | 55.8 | |
| 39 | 20.0 | |
| 40 | 81.5 | |
| 41 | 132.7 | |
| 42 | 151.1 | |
| 43 | 94.6 | |
| 44 | 15.8 | |
| 45 | 51.2 | |
| 46 | 21.0 | |
| 47 | 49.6 | |
| 48 | 18.4 | |
| 49 | 42.3 | |
| 50 | 14.0, 8.2, 2.2 | |
| 51 | 27.0 | |
| 52 | 17.6 | |
| 53 | 41.7 | |
| 54 | 3.1 | |
| 55 | 5.5 | |
| 56 | 10.7 | |
| 57 | 14.6, 20.7 | |
| 58 | 14.0, 17.3 | |
| 59 | 8.3 | |
| 60 | 11.9 | |
| 61 | 7.8, 13.0, 10.0 | |
| 63 | 5.5 | |
| 64 | 8.7 | |
| 65 | 15.5 | |
| 66 | 24.0, 35.7 | |
| 67 | 15.3, 16.7 | |
| 68 | 14.8, 16.3 | |
| 69 | 20.5 | |
| 70 | 133.6 | |
| 76 | 6.0 | |
| 78 | 1.0 | |
| 79 | 5.0, 14.4 | |
| 81 | 2.0, 7.0 | |
| 82 | 11.6, 1.8 | |
| 85 | 3.7, 4.6, 3.8 | |
| 86 | 3.1, 7.7 | |
| 87 | 5.7, 8.6 | |
| 88 | 3.6, 9.7 | |
| 89 | 3.6, 5.2, 2.4 | |
| 90 | 7.4, 5.0, 9.3 | |
| 91 | | 7 |
| 92 | 24.5 | |
| 93 | 10.9 | |
| 94 | 4.5 | |
| 95 | 5.7 | |
| 96 | 6.6 | |
| 97 | 6.9 | |
| 100 | 33.7 | 91 |
| 101 | | 33 |
| 102 | 54.4 | |
| 103 | 4.5 | |
| 104 | 3.9 | |
| 105 | 16.4 | |
| 106 | 21.8 | |
| 107 | 11.9 | |
| 108 | 10.2 | |
| 109 | 13.9 | |
| 110 | 14.3 | |
| 111 | 10.1 | |
| 112 | 5.6, 3.5, 5.9 | 99, 99 |
| 113 | 2.4, 3.5, 4.4 | 99, 100 |
| 114 | 4.7, 10.3, 10.2 | 99, 100 |
| 115 | 2.2, 5.6, 4.9 | 100, 100 |
| 116 | 2.6, 5.8, 5.5 | 99, 100 |
| 117 | 7.0, 11.3, 8.4 | 100, 100 |
| 118 | 9.6 | |
| 119 | 2.0 | |
| 120 | 2.1 | |
| 121 | 3.1 | |
| 122 | 5.5 | |
| 123 | 7.9 | |
| 124 | 7.3, 4.4 | 99 |
| 125 | 3.2, 1.7 | 100 |
| 126 | 3.9, 3.5 | 99 |
| 127 | 11.5, 6.3 | 99 |
| 128 | 2.7, 5.1 | 99 |
| 129 | 6.2, 4.1 | 99 |
| 130 | 10.7, 5.3 | 100 |
| 131 | 42.0 | 100 |
| 132 | | 1 |
| 133 | 12.6, 10.2 | 99 |
| 134 | 5.6, 10.9 | 99 |
| 135 | 4.0, 6.9 | 99 |
| 136 | 8.2, 9.0 | 100 |
| 137 | 3.0, 4.7 | 99 |
| 138 | 7.7, 6.8 | 99 |
| 139 | 5.6, 12.0 | 99 |
| 140 | 4.5, 7.4 | 100 |
| 141 | 5.4, 8.7 | 99 |
| 142 | 3.0, 3.3 | 99 |
| 143 | 2.5, 3.7 | 99 |
| 144 | 3.9, 4.0 | 99 |
| 145 | 3.7, 8.1 | 100 |
| 146 | 12.2, 15.0 | 100 |
| 147 | 5.9, 4.8, 5.1 | 100, 98 |
| 148 | 5.0, 4.1, 2.9 | 100, 99 |
| 149 | 3.8, 5.1, 3.2 | 100, 99 |
| 150 | 5.7, 7.2, 8.3 | 100, 100 |
| 151 | 4.2, 6.0, 3.8 | 100, 100 |
| 152 | 5.3, 6.5, 5.0 | 100, 99 |
| 153 | | 25 |
| 154 | | 57 |
| 155 | | 59 |
| 156 | | 58 |
| 157 | | 41 |
| 158 | | 72 |
| 159 | | 19 |
| 161 | | 36 |
| 163 | | 62 |
| 165 | | 27 |
| 167 | 9.8 | 100 |
| 168 | 2.3, 7.0, 4.3, 4.7, 3.2 | 99, 101, 100, 100, 99 |
| 169 | 8.0 | 100 |
| 170 | 12.4 | 100 |
| 171 | 8.2 | 100 |
| 172 | 8.7 | 100 |
| 173 | 10.7 | 100 |
| 174 | 33.4 | 83 |
| 175 | | 48 |
| 176 | | 60 |
| 177 | | 11 |
| 178 | | 17 |
| 179 | | 18 |
| 180 | | 6 |
| 181 | | 15 |
| 182 | | 26 |
| 183 | | 4 |
| 184 | 6.4 | 101 |
| 185 | 13.8 | 99 |
| 186 | 20.0 | 99 |
| 187 | 14.7 | 100 |
| 188 | 35.0 | 99 |
| 189 | 10.6 | 99 |
| 190 | 30 | 101 |
| 191 | | 51 |
| 192 | 7.5 | 86 |
| 193 | | 18 |
| 194 | | 63 |
| 195 | | 46 |
| 196 | | 39 |

### Biological Example 2

### Inhibition of Icilin-Induced “Wet-Dog” Shakes in Rats

Icilin was initially developed as a “super-cooling” compound by Delmar Chemicals Ltd. Subsequently it was shown to be one of the most potent known agonists of TRPM8 (MCKEMY, D. D., et al “Identification of a cold receptor reveals a general role for TRP channels in thermosensation”, Nature, pp 52-58, Vol. 416 (6876)), having an EC₅₀ value of 0.2 μM in stimulating calcium ion influx into TRPM8 transfected cells (BEHRENDT, H-J., et al., “Characterization of the mouse cold menthol receptor TRPM8 and vanilloid receptor type-1 VR1 using a fluorometric imaging plate reader (FLIPR) assay”, Brit J Pharmacol, 2004, pp 737-745, Vol. 141(4)). Initial in vivo testing of icilin showed it to cause “wet-dog” shakes in rats. Similar shaking or jumping behavior was also evident in mice, rabbits, cats, dogs and monkeys. In humans, icilin produced a sensation of coolness on contact with mucous membranes, cold prickling when 0.1 mg was dropped on the tongue and coldness in the mouth, pharynx and chest lasting 30-60 min when 5-10 mg was ingested orally (WEI, E. T., et al., “AG-3-5: a chemical producing sensations of cold”, J Pharm Pharmacol., 1983, pp 110-112, Vol. 35). The inhibition or reversal of icilin-induced shaking behaviors in rodents provides evidence for the engagement and functional blockade of the TRPM8 channel and thereby for the utility of TRPM8 antagonists in treating or preventing a disease or condition in a mammal in which the disease or condition is affected by the modulation of TRPM8 receptors.

Male Sprague Dawley rats (220-450 g, Charles River Labs, n=6-9/treatment) were used to evaluate the ability of test compounds to block icilin-induced “wet-dog” shakes (WDS). The test compound was administered in 10% hydroxypropyl-β-cyclodextrin (HP-13-CD), p.o., 60 min before icilin. Icilin was then administered in 10% solutol/H₂O, at 3.0 mg/kg, i.p., and spontaneous “wet-dog” shakes were counted 10 min following the icilin injection over a 10-min period. Results for representative compounds of the present invention are presented in Table 3 below as a percent inhibition of shakes, which was calculated as follows:

% Inhibition=[1−(test compound WDS count/vehicle WDS count)]×100.

### Biological Example 3

### Chronic Constriction Injury (CCI)-Induced Model of Neuropathic Pain

### Acetone-Induced Hypersensitivity

Male Sprague-Dawley rats (225-450 g; n=5-8/treatment) were used to evaluate the ability of test compounds to reverse CCl-induced cold hypersensitivity. Four loose ligatures of 4-0 chromic gut were surgically placed around the left sciatic nerve under inhalation anesthesia as described by Bennett et al. (BENNETT, G. J., et al., “A peripheral mononeuropathy in rat that produces disorder of pain sensation like those seen in man”, Pain, 1988, pp 87-107, Vol. 33(1)). Fourteen to 35 days following CCl surgery, subjects were placed in elevated observation chambers containing wire mesh floors, and five applications of acetone (0.05 mL/application separated by approximately 5 min) were spritzed onto the plantar surface of the paw using a multidose syringe. An abrupt withdrawal or lifting of the paw was considered a positive response. The number of positive responses was recorded for each rat over the five trials. Following baseline withdrawal determinations, test compounds were administered in 10% hydroxypropyl-β-cyclodextrin (HP-β-CD), p.o. The number of withdrawals was re-determined at 2 hr after compound administration. Representative compounds of the present invention were administered at 10 mg/kg in 10% HP-β-CD and tested according to this procedure. Results are presented below as a percent inhibition of shakes, which was calculated for each subject and then averaged by treatment as follows:

% Inhibition=[1−(test compound withdrawals/pre-test withdrawals)]×100.

Representative compounds of the present invention were tested according to the procedures as described in Biological Example 2 and Biological Example 3 above, with results as listed in Table 3, below.

TABLE 3
Icilin and CCI Inhibition - Compounds of Formula (I)

| | Icilin % Inhibition | Icilin Dose | CCI % Inhibition |
|---|---|---|---|
| ID No. | @1.5 hrs | (mg/kg) | @ 2 h |
| 3 | 48.3 | 30 | |
| 9 | 66.4 | 10 | |
| 10 | 97.3 | 30 | 57.1 |
| 13 | 98.3 | 30 | 48.6 |
| 44 | 30.0 | 10 | |
| 50 | 47.4 | 10 | |
| 58 | 11.2 | 5.6 | |
| 61 | 38.8 | 10 | |
| 76 | 98.6 | 10 | |
| 78 | 99.3 | 10 | |
| 79 | 20.1 | 5.6 | |
| 85 | 96.4 | 10 | 34.3 |
| 86 | 98.7 | 10 | |
| 89 | 91.5 | 10 | |
| 90 | 52.7 | 5.6 | 22.5 |
| 112 | 21.2 | 3 | |
| 113 | 2.8 | 3 | |
| 115 | 45.0 | 3 | |
| 116 | 47.7 | 3 | |
| 117 | 7.7 | 3 | |
| 120 | 44.8 | 3 | |
| 125 | 24.6 | 3 | |
| 130 | 22.7 | 3 | |
| 133 | 4.6 | 3 | |
| 136 | 61.6 | 3 | |
| 168 | 86.0 | 3 | 74.3 |
| 169 | 34.0 | 3 | |
| 170 | 4.3 | 3 | |

### Formulation Example 1

### Oral Solid Dosage Formulation

### Prophetic Example

As a specific embodiment of an oral composition, 100 mg of Compound #168 is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed:

1. A compound of formula (I) 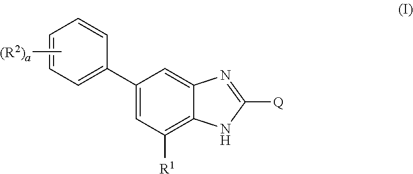
wherein
R¹ is selected from the group consisting of hydrogen, chloro, methyl and trifluoromethyl;
a is an integer from 0 to 2;
each R² is independently selected from the group consisting of fluoro, chloro, C₁₋₄alkyl, C₁₋₄alkoxy, fluorinated C₁₋₄alkyl, fluorinated C₁₋₄alkoxy and cyano;
Q is a substituted ring structure selected from the group consisting of (a) through (l):
(a) 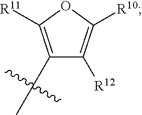
(b) 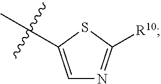
(c) 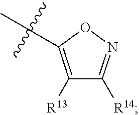
(d) 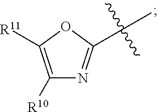
(e) 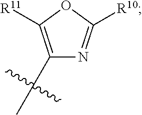
(f) 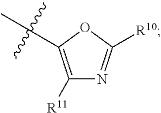
(g) 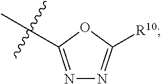
(h) 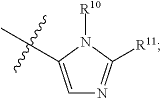
(i) 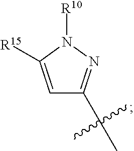
(j) 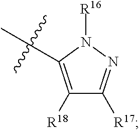
(k) 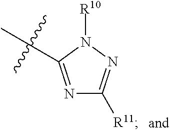
(l) a fused multi-ring structure selected from the group consisting of 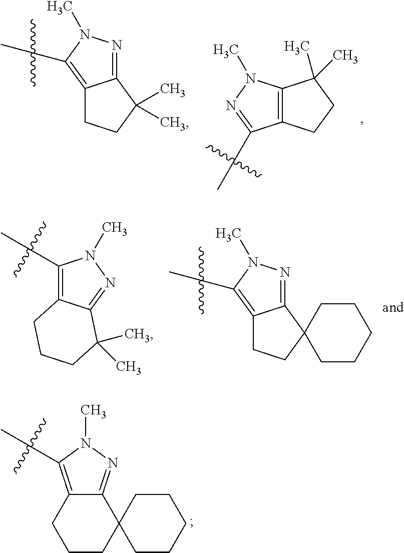
wherein
R¹⁰ and R¹¹ are each independently selected from the group consisting C₁₋₄alkyl;
R¹² is selected from the group consisting of hydrogen and cyano;
R¹³ is selected from the group consisting of hydrogen and C₁₋₄alkyl;
R¹⁴ is selected from the group consisting of chloro, bromo, C₁₋₆alkyl and C₃₋₆cycloalkyl;
R¹⁵ is selected from the group consisting of C₃₋₆cycloalkyl;
R¹⁶ is selected from the group consisting of C₁₋₄alkyl, hydroxy substituted C₁₋₄alkyl and benzyl;
R¹⁷ is selected from the group consisting of C₁₋₄alkyl, trifluoromethyl, C₃₋₆cycloalkyl and 1-methyl-cyclopropyl;
R¹⁸ is selected from the group consisting of hydrogen, chloro, C₁₋₄alkyl, trifluoromethyl, cyano;
or a solvate, hydrate, tautomer or pharmaceutically acceptable salt thereof.

2. A compound as in claim 1, wherein
R¹ is selected from the group consisting of hydrogen, chloro, methyl and trifluoromethyl;
a is an integer from 0 to 2;
each R² is independently selected from the group consisting of fluoro, chloro, C₁₋₄alkyl, C₁₋₄alkoxy, fluorinated C₁₋₄alkyl, fluorinated C₁₋₄alkoxy and cyano;
Q is a substituted ring structure selected from the group consisting of (a) through (l): 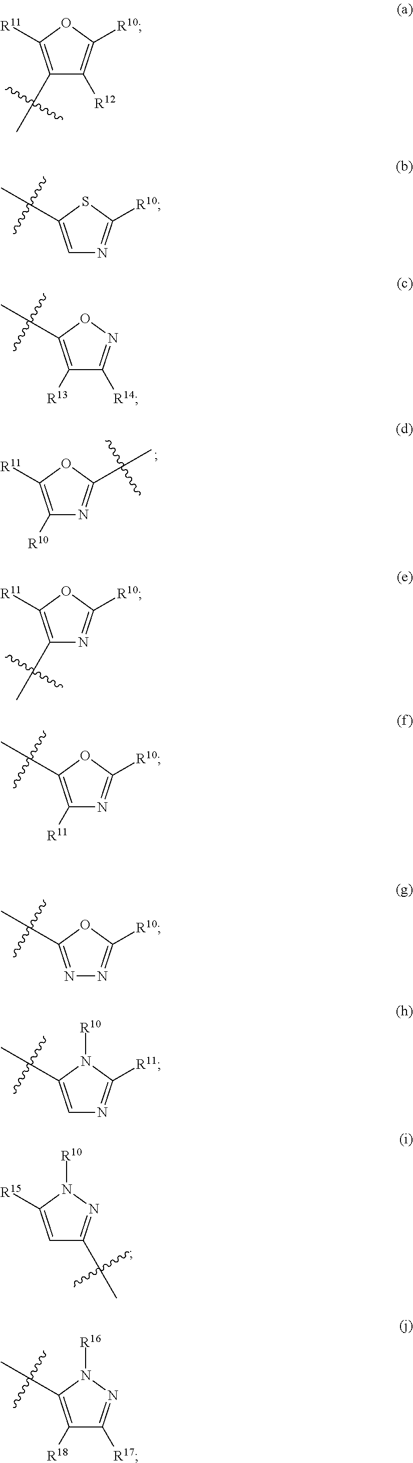 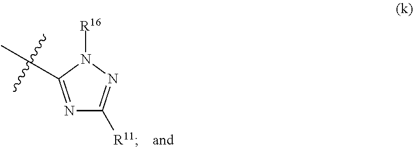
(l) a fused multi-ring structure selected from the group consisting of 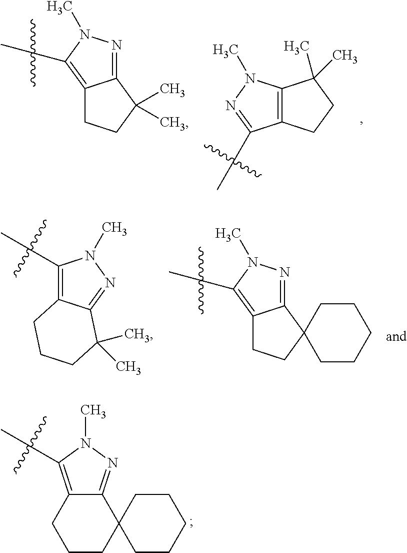
wherein
R¹⁰ and R¹¹ are each independently selected from the group consisting C₁₋₄alkyl;
R¹² is selected from the group consisting of hydrogen and cyano;
R¹³ is selected from the group consisting of hydrogen and C₁₋₄alkyl;
R¹⁴ is selected from the group consisting of chloro, bromo, C₁₋₆alkyl and C₃₋₆cycloalkyl;
R¹⁵ is selected from the group consisting of C₃₋₆cycloalkyl;
R¹⁶ is selected from the group consisting of C₁₋₄alkyl, —(C₁₋₄alkyl)-OH and benzyl;
R¹⁷ is selected from the group consisting of C₁₋₄alkyl, trifluoromethyl, C₃₋₄cycloalkyl and 1-methyl-cyclopropyl;
R¹⁸ is selected from the group consisting of hydrogen, chloro, C₁₋₂alkyl, trifluoromethyl and cyano;
or a solvate, hydrate, tautomer or pharmaceutically acceptable salt thereof.

3. A compound as in claim 2, wherein
R¹ is selected from the group consisting of hydrogen, chloro, methyl and trifluoromethyl;
a is an integer from 0 to 2;
each R² is independently selected from the group consisting of fluoro, chloro, C₁₋₂alkyl, C₁₋₂alkoxy, fluorinated C₁₋₂alkyl, fluorinated C₁₋₂alkoxy and cyano;
Q is a substituted ring structure selected from the group consisting of (a) through (l): 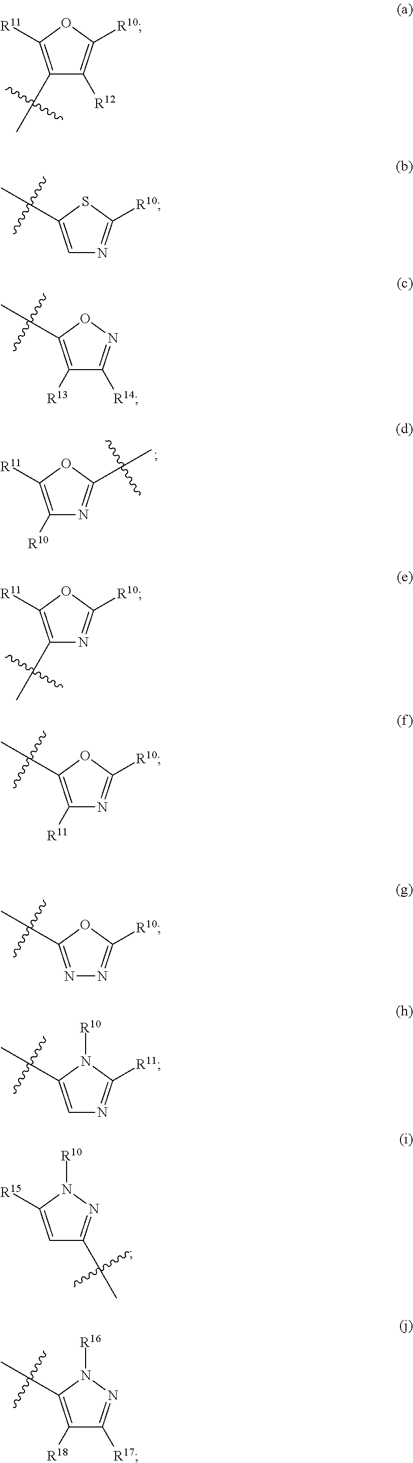 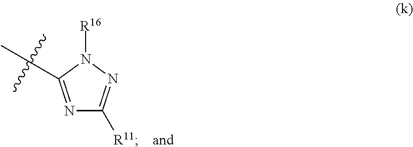
(l) a fused multi-ring structure selected from the group consisting of 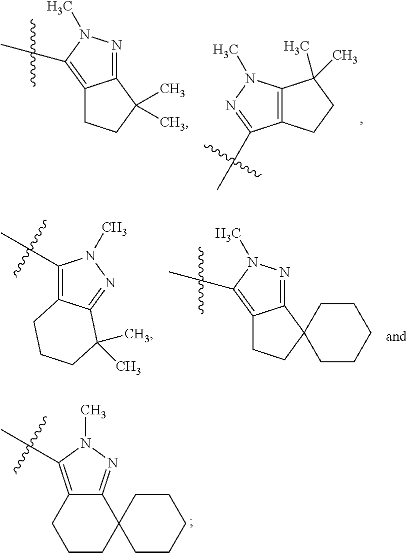
wherein
R¹⁰ and R¹¹ are each independently selected from the group consisting of methyl and t-butyl;
R¹² is selected from the group consisting of hydrogen and cyano;
R¹³ is selected from the group consisting of hydrogen and t-butyl;
R¹⁴ is selected from the group consisting of chloro, bromo, C₃₋₆alkyl and C₃₋₆cycloalkyl;
R¹⁵ is cyclobutyl;
R¹⁶ is selected from the group consisting of C₁₋₄alkyl, —(C₂₋₃alkyl)-OH and benzyl;
R¹⁷ is selected from the group consisting of t-butyl, trifluoromethyl, cyclobutyl and 1-methyl-cyclopropyl;
R¹⁸ is selected from the group consisting of hydrogen, chloro, methyl, trifluoromethyl and cyano;
or a solvate, hydrate, tautomer or pharmaceutically acceptable salt thereof.

4. A compound as in claim 3, wherein
R¹ is selected from the group consisting of hydrogen, chloro, methyl and trifluoromethyl;
a is an integer from 0 to 2;
each R² is independently selected from the group consisting of fluoro, chloro, methyl, ethyl, methoxy, ethoxy, trifluoromethyl, trifluoromethoxy, difluoromethoxy and cyano;
Q is a substituted ring structure selected from the group consisting of
(a) 2-methyl-5-t-butyl-fur-3-yl, 2-methyl-4-cyano-5-t-butyl-fur-3-yl,
(b) 2-t-butyl-thiazol-5-yl,
(c) 3-chloro-isoxazol-5-yl, 3-bromo-isoxazol-5-yl, 3-n-propyl-isoxazol-5-yl, 3-isopropyl-isoxazol-5-yl; 3-isobutyl-isoxazol-5-yl, 3-t-butyl-isoxazol-5-yl, 3-(2,2-dimethyl-propyl)-isoxazol-5-yl, 3-(pentan-3-yl)-isoxazol-5-yl, 3-cyclopropyl-isoxazol-5-yl, 3-cyclopentyl-isoxazol-5-yl, 3-cyclohexyl-isoxazol-5-yl, 3-t-butyl-4-methyl-isoxazol-5-yl,
(d) 4-t-butyl-5-methyl-oxazol-2-yl,
(e) 2-t-butyl-5-methyl-oxazol-4-yl,
(f) 2-t-butyl-4-methyl-oxazol-5-yl,
(g) 5-t-butyl-(1,3,4-oxadiazol-2-yl),
(h) 1-methyl-2-t-butyl-imidazol-5-yl,
(i) 1-methyl-5-cyclobutyl-pyrazol-3-yl,
(j) 1-methyl-3-t-butyl-pyrazol-5-yl, 1-methyl-3-trifluoromethyl-pyrazol-5-yl, 1-methyl-3-t-butyl-4-trifluoromethyl-pyrazol-5-yl, 1-ethyl-3-t-butyl-pyrazol-5-yl, 1-isopropyl-3-t-butyl-pyrazol-5-yl, 1-methyl-3-cyclobutyl-pyrazol-5-yl, 1-m ethyl-3-(1-m ethyl-cyclo pro pyl)-pyrazol-5-yl, 1-benzyl-3-t-butyl-pyrazol-5-yl, 1-methyl-3-t-butyl-4-chloro-pyrazol-5-yl, 1,4-dimethyl-3-t-butyl-pyrazol-5-yl, 1-methyl-3-t-butyl-4-cyano-pyrazol-5-yl, 1-(3-hydroxy-n-propyl)-3-t-butyl-pyrazol-5-yl, 1-(2-hydroxy-ethyl)-3-t-butyl-pyrazol-5-yl,
(k) 1-methyl-3-t-butyl-(1,2,4-triazol-5-yl),
(l) 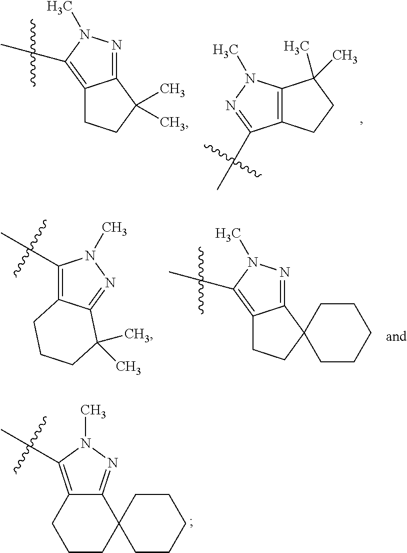
or a solvate, hydrate, tautomer or pharmaceutically acceptable salt thereof.

5. A compound as in claim 4, wherein
R¹ is selected from the group consisting of hydrogen, chloro, methyl and trifluoromethyl;
a is an integer from 1 to 2;
each R² is independently selected from the group consisting of fluoro, chloro, methyl, ethyl, methoxy, ethoxy, trifluoromethyl, trifluoromethoxy, difluoromethoxy and cyano;
Q is a substituted ring structure selected from the group consisting of 2-methyl-5-t-butyl-fur-3-yl, 2-methyl-4-cyano-5-t-butyl-fur-3-yl, 2-t-butyl-thiazol-5-yl, 3-n-propyl-isoxazol-5-yl, 3-isopropyl-isoxazol-5-yl; 3-isobutyl-isoxazol-5-yl, 3-t-butyl-isoxazol-5-yl, 3-(2,2-dimethyl-propyl)-isoxazol-5-yl, 3-(pentan-3-yl)-isoxazol-5-yl, 3-cyclopentyl-isoxazol-5-yl, 3-t-butyl-4-methyl-isoxazol-5-yl, 5-t-butyl-(1,3,4-oxadiazol-2-yl), 1-methyl-3-t-butyl-pyrazol-5-yl, 1-methyl-3-t-butyl-4-trifluoromethyl-pyrazol-5-yl, 1-isopropyl-3-t-butyl-pyrazol-5-yl, 1-methyl-3-cyclobutyl-pyrazol-5-yl, 1-methyl-3-(1-methyl-cyclopropyl)-pyrazol-5-yl, 1-ethyl-3-tert-butyl-pyrazol-5-yl, 1-benzyl-3-t-butyl-pyrazol-5-yl, 1-methyl-3-t-butyl-4-chloro-pyrazol-5-yl, 1,4-dimethyl-3-t-butyl-pyrazol-5-yl, 1-methyl-3-t-butyl-4-cyano-pyrazol-5-yl, 1-methyl-3-t-butyl-(1,2,4-triazol-5-yl), 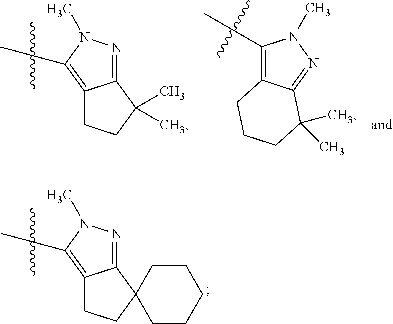
or a solvate, hydrate, tautomer or pharmaceutically acceptable salt thereof.

6. A compound as in claim 5, wherein
R¹ is selected from the group consisting of hydrogen, chloro, methyl and trifluoromethyl;
a is an integer from 1 to 2;
each R² is independently selected from the group consisting of fluoro, chloro, methyl, ethyl, methoxy, ethoxy, trifluoromethyl, trifluoromethoxy, difluoromethoxy and cyano;
Q is a substituted ring structure selected from the group consisting of 2-methyl-4-cyano-5-t-butyl-fur-3-yl, 3-t-butyl-isoxazol-5-yl, 3-t-butyl-4-methyl-isoxazol-5-yl, 5-t-butyl-(1,3,4-oxadiazol-2-yl), 1-methyl-3-t-butyl-pyrazol-5-yl, 1-methyl-3-t-butyl-4-trifluoromethyl-pyrazol-5-yl, 1-methyl-3-t-butyl-4-chloro-pyrazol-5-yl, 1,4-dimethyl-3-t-butyl-pyrazol-5-yl, 1-methyl-3-t-butyl-4-cyano-pyrazol-5-yl, 1-methyl-3-t-butyl-(1,2,4-triazol-5-yl), and 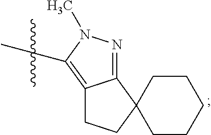
or a solvate, hydrate, tautomer or pharmaceutically acceptable salt thereof.

7. A compound as in claim 6, wherein
R¹ is selected from the group consisting of hydrogen, chloro, methyl and trifluoromethyl;
a is an integer from 1 to 2;
each R² is independently selected from the group consisting of fluoro, chloro, methoxy, trifluoromethyl and trifluoromethoxy;
Q is a substituted ring structure selected from the group consisting of 3-t-butyl-isoxazol-5-yl, 5-t-butyl-(1,3,4-oxadiazol-2-yl), -methyl-3-t-butyl-pyrazol-5-yl, 1-methyl-3-t-butyl-4-chloro-pyrazol-5-yl, 1,4-dimethyl-3-t-butyl-pyrazol-5-yl and 1-methyl-3-t-butyl-4-cyano-pyrazol-5-yl;
or a solvate, hydrate, tautomer or pharmaceutically acceptable salt thereof.

8. A compound as in claim 4, wherein
R¹ is selected from the group consisting of hydrogen and chloro;
a is an integer from 1 to 2;
each R² is independently selected from the group consisting of fluoro, chloro and trifluoromethyl;
Q is a substituted ring structure selected from the group consisting of 1-methyl-3-t-butyl-4-chloro-pyrazol-5-yl, 1,4-dimethyl-3-t-butyl-pyrazol-5-yl and 1-methyl-3-t-butyl-4-cyano-pyrazol-5-yl;
or a solvate, hydrate, tautomer or pharmaceutically acceptable salt thereof.

9. A compound as in claim 4 wherein
R¹ is selected from the group consisting of hydrogen and chloro;
a is an integer from 1 to 2;
each R² is independently selected from the group consisting of fluoro, chloro, trifluoromethyl and trifluoromethoxy;
Q is a substituted ring structure selected from the group consisting of 3-t-butyl-isoxazol-5-yl, 1-methyl-3-t-butyl-pyrazol-5-yl, 1-methyl-3-t-butyl-4-chloro-pyrazol-5-yl and 1-methyl-3-t-butyl-4-cyano-pyrazol-5-yl;
or a solvate, hydrate, tautomer or pharmaceutically acceptable salt thereof.

10. A pharmaceutical composition comprising a pharmaceutically acceptable carrier and a compound of claim 1.

11. A process for making a pharmaceutical composition comprising mixing a compound of claim 1 and a pharmaceutically acceptable carrier.

12. A method for treating inflammatory pain or neuropathic pain comprising administering to a mammal in need of such treatment a therapeutically effective amount of a compound as in claim 1.

13. A method as in claim 12, wherein the inflammatory pain is due to inflammatory bowel disease, visceral pain, migraine, post operative pain, osteoarthritis, rheumatoid arthritis, back pain, lower back pain, joint pain, abdominal pain, chest pain, labor, musculoskeletal diseases, skin diseases, toothache, pyresis, burn, sunburn, snake bite, venomous snake bite, spider bite, insect sting, neurogenic bladder, interstitial cystitis, urinary tract infection, rhinitis, contact dermatitis/hypersensitivity, itch, eczema, pharyngitis, mucositis, enteritis, irritable bowel syndrome, cholecystitis, pancreatitis, postmastectomy pain syndrome, menstrual pain, endometriosis, sinus headache, tension headache, or arachnoiditis.

14. A method as in claim 12, wherein the inflammatory pain is inflammatory hyperalgesia.

15. A method as in claim 14, wherein the inflammatory hyperalgesia is inflammatory somatic hyperalgesia or inflammatory visceral hyperalgesia.

16. A method as in claim 14, wherein the inflammatory hyperalgesia is due to inflammation, osteoarthritis, rheumatoid arthritis, back pain, joint pain, abdominal pain, musculoskeletal diseases, skin diseases, post operative pain, headaches, fibromyalgia, toothache, burn, sunburn, insect sting, neurogenic bladder, urinary incontinence, interstitial cystitis, urinary tract infection, cough, asthma, chronic obstructive pulmonary disease, rhinitis, contact dermatitis/hypersensitivity, itch, eczema, pharyngitis, enteritis, irritable bowel syndrome, Crohn's Disease, or ulcerative colitis.

17. A method as in claim 12, wherein the inflammatory pain is visceral pain.

18. A method as in claim 12, wherein said neuropathic pain is due to cancer, a neurological disorder, spine or peripheral nerve surgery, a brain tumor, traumatic brain injury (TBI), spinal cord trauma, a chronic pain syndrome, fibromyalgia, chronic fatigue syndrome, a neuralgia, lupus, sarcoidosis, peripheral neuropathy, bilateral peripheral neuropathy, diabetic neuropathy, central pain, neuropathies associated with spinal cord injury, stroke, ALS, Parkinson's disease, multiple sclerosis, sciatic neuritis, mandibular joint neuralgia, peripheral neuritis, polyneuritis, stump pain, phantom limb pain, a bony fracture, oral neuropathic pain, Charcot's pain, complex regional pain syndrome I and II (CRPS I/II), radiculopathy, Guillain-barre syndrome, meralgia paresthetica, burning-mouth syndrome, optic neuritis, postfebrile neuritis, migrating neuritis, segmental neuritis, Gombault's neuritis, neuronitis, cervicobrachial neuralgia, cranial neuralgia, geniculate neuralgia, glossopharyngial neuralgia, migrainous neuralgia, idiopathic neuralgia, intercostals neuralgia, mammary neuralgia, Morton's neuralgia, nasociliary neuralgia, occipital neuralgia, red neuralgia, Sluder's neuralgia, splenopalatine neuralgia, supraorbital neuralgia, vulvodynia or vidian neuralgia.

19. A method as in claim 18, wherein the neuralgia is trigeminal neuralgia, glossopharyngeal neuralgia, postherpetic neuralgia, or causalgia.

20. A method as in claim 12, wherein the neuropathic pain is neuropathic cold allodynia.

21. A method as in claim 20, wherein the neuropathic cold allodynia is pain arising from spine and peripheral nerve surgery or trauma, traumatic brain injury (TBI), trigeminal neuralgia, postherpetic neuralgia, causalgia, peripheral neuropathy, diabetic neuropathy, central pain, stroke, peripheral neuritis, polyneuritis, complex regional pain syndrome I and II (CRPS I/II), or radiculopathy.

22. A method for treating cardiovascular disease aggravated by cold, including peripheral vascular disease, vascular hypertension, pulmonary hypertension, Raynaud's disease, and coronary artery disease, comprising administering to a mammal in need of such treatment a therapeutically effective amount of a compound as in claim 1.

23. A compound as in claim 1 for use as a medicament for treating (a) inflammatory pain, (b) neuropathic pain, (c) cardiovascular disease aggravated by cold or (d) pulmonary disease aggravated by cold, in a subject in need thereof.
